(19) United States
(12) Patent Application Publication
(10) Pub. No.: US 2021/0113705 A1
(43) Pub. Date: Apr. 22, 2021

(54) IMPROVED METHODS OF MANUFACTURING PEPTIDE-BASED VACCINES

(71) Applicants: Avidea Technologies, Inc., Baltimore, MD (US); The United States of America, as represented by the Secretary, Department of Health and Human Servic, Bethesda, MD (US)

(72) Inventors: Geoffrey Martin Lynn, Baltimore, MD (US); Andrew Scott Ishizuka, Washington, DC (US); Vincent Coble, Mt. Airy, MD (US); Yaling Zhu, Gaithersburg, MD (US)

(73) Assignees: Avidea Technologies, Inc., Baltimore, MD (US); The United States of America, as represented by the Secretary, Department of Health and Human Servic, Bethesda, MD (US)

(21) Appl. No.: 17/057,658

(22) PCT Filed: May 22, 2019
(86) PCT No.: PCT/US19/33612

§ 371 Date: Nov. 21, 2020

Related U.S. Application Data
(60) Provisional application No. 62/674,752, filed on May 22, 2018.

(51) Int. Cl. A61K 47/64; A61K 47/59; A61K 9/19; A61K 39/00; A61K 47/20; A61K 47/10; A61K 47/60; G01N 21/33

(52) U.S. Cl. CPC A61K 47/6455; A61K 47/59; A61K 9/19; A61K 39/0011; A61K 47/20; A61K 2039/70; A61K 47/60; G01N 21/33; A61K 2039/6093; A61K 2039/627; A61K 47/10

(57) ABSTRACT

A process for producing a peptide antigen conjugate suitable for administration to a mammal is disclosed. The peptide antigen conjugate comprises a peptide antigen linked to a hydrophobic block. The process comprises reacting a hydrophobic block fragment with a peptide antigen fragment comprising the peptide antigen in a pharmaceutically acceptable organic solvent in a hydrophobic block fragment to peptide antigen fragment molar ratio of 1:1 or greater under conditions to directly or indirectly link the peptide antigen to the hydrophobic block and obtaining a product solution comprising the peptide antigen conjugate, unreacted hydrophobic block fragment and pharmaceutically acceptable organic solvent.

### PRIORITY

The present application claims priority from U.S. Provisional Patent Application No. 62/674,752 filed on May 22, 2018, which is hereby incorporated by reference in its entirety.

This invention was created in the performance of a Cooperative Research and Development Agreement with the National Institutes of Health, an Agency of the Department of Health and Human Services. The Government of the United States has certain rights in this invention.

### TECHNICAL FIELD

The present disclosure relates generally to the field of personalized medicine or precision medicine and more specifically to the manufacture of vaccines for use in personalized medicine.

### BACKGROUND

Responses to active pharmaceutical ingredients found in medications can vary from one individual to another and this variability is driving an increasing interest in personalized medicine (also known as precision medicine). Personalized medicine involves the use of information about an individual's genes, proteins, and environment to direct the medical care the individual receives. The continued development of new diagnostic and informatics methods that provide a greater understanding of the molecular basis of disease has meant that patient specific information is increasingly more available.

In cancer therapy, specific information about an individual's tumor can be used to help diagnose, plan treatment, find out how well treatment is working, or make a prognosis. Similarly, specific information about an individual's genes, proteins, and environment can be used to tailor a preventative approach to cancer treatment by developing vaccines based on that information. For the immunological treatment of certain cancers, one preferred immune response is a CD8 T cell response and/or a CD4 T cell response that recognizes a tumor-associated antigen. However, a major challenge to generating effective T cell immunity against cancers is that most current vaccine approaches are hampered by limited antigenic breadth for generating T cell responses against tumor-associated antigens, particularly neoantigens, which are tumor cell specific proteins that are often unique to individual patients. For example, with regard to antigenic breadth, the current gold-standard peptide-based vaccine approaches (i.e. mixing 25 amino acid synthetic long peptides with the adjuvant polyIC:LC) induce CD8 T cell responses against less than 10% of predicted neoantigens (see: S. Kreiter et al., Nature 520, 692-696, 2015). Thus, improved peptide-based vaccine approaches are needed.

Personalized approaches to treat auto-immunity or allergies is possible through the identification of the specific self-antigens or foreign antigens, respectively, that are the cause of immune-mediated pathology. The antigens identified as the cause of the pathology may be administered in the form of a peptide-based vaccine that is capable of inducing tolerance or suppresion against the self-antigens or foreign antigens. For instance, the immune response against a foreign antigen may be of a particular type of immune response that results in pathology, such as allergies. A vaccine against such a foreign antigen may be provided as a peptide-based vaccine to shift the immune response to a type that does not result in pathology.

Immunogenic compositions comprising peptide antigens may be used as vaccines to induce an immune response in a subject, including for the treatment or prevention of cancers or infectious diseases, or even for inducing tolerance and/or immune suppression for the treatment or prevention of auto-immunity or allergies. The compositions of peptide-based vaccines for inducing immune responses for the treatment or prevention of cancers and infectious diseases may contain peptide-based antigens and specific types of adjuvants that promote the induction of antigen-specific cytotoxic T cell responses and/or antibodies that mediate pathogen clearance or killing of virally infected or cancerous cells. In contrast, compositions of peptide-based vaccines for inducing tolerogenic or suppressive responses may contain an antigen and a vehicle (e.g., delivery system such as a particle carrier) and/or immuno-modulators, such as mTOR inhibitors, but will lack specific adjuvants that induce cytotoxic T cells, and may instead induce T cell tolerance or activation of regulatory cells, such as regulatory CD4 T cells, that down-regulate or modulate the qualitative characteristics of the response.

A major challenge to the development of a personalized vaccine approach for cancer treatment is that there is presently no consensus concerning how best to construct a peptide-based vaccine to reliably elicit T cell immunity. Similarly, there is no consensus on how best to construct a peptide-based vaccine for inducing immune tolerance or for shifting an immune response from one that results in symptoms associated with allergies to an innocuous type of response.

Another major challenge facing current peptide-based vaccine delivery platforms is that they do not account for the broad range of possible physical and chemical characteristics of peptide antigens. For example, individualized cancer vaccine approaches may require that a unique set of peptide antigens is generated that is specific to each patient's tumor. Similarly, for individualized approaches for treating auto-immune conditions, multiple different antigens may be identified as the cause of pathology and the specific set of antigens causing pathology may vary between patients. Thus, tolerance-inducing vaccines used for the treatment of auto-immunity may need to contain a set of peptide antigens that are unique to each patient. In each case, the set of peptide antigens that are unique to each patient will have a broad range of possible physical and chemical characteristics that can (i) lead to manufacturing challenges when produced by solid phase peptide synthesis (SPPS) as well as (ii) lead to variability in formulation characteristics arising from unwanted interactions between peptide antigens and/or other components of the vaccine formulation (e.g., delivery system, immuno-modulators, etc.) or even due to aggregation of hydrophobic peptide antigens.

Still a further challenge associated with the manufacture of peptide-based vaccines is ensuring that the peptide antigen can be produced as a particle or formulated within a particle carrier that is of an optimal size (i.e. within the appropriate range of particle sizes) to permit efficient uptake by antigen presenting cells to induce an immune response in vivo (M. F. Bachmann et al., Nat Rev Immunol, 10, 787-796, 2010). As particle size (range) is an important parameter that impacts the ability of immunogenic compositions comprising peptide-based vaccines to induce an immune response in vivo, reliable nanoparticle assembly and control over the particle size (range) is important in any manufacturing process of peptide-based vaccines. However, current methods of manufacturing peptide-based vaccines as particles may be unsatisfactory for any one or more of the following reasons: they involve time-consuming preparation; they give rise to unacceptable levels of variability in particle size (range); and/or they involve the use of organic solvents that are not suitable for administration to patients and may therefore require additional testing to ensure that such potentially harmful solvents have been removed prior to administration to a patient.

The present inventors have previously developed novel peptide-based vaccine compositions that overcome the limitations of current peptide-based vaccine approaches. The novel peptide-based vaccine compositions account for the variability in the physical and chemical properties of peptide antigens and are therefore generalizable for any peptide antigen thereby ensuring formulation consistency and reliable activity for a broad range of possible peptide antigens useful for inducing an immune response in a subject. The novel peptide-based vaccine compositions are particularly useful in the field of personalized cancer treatment, wherein the characteristics of peptide antigens used in a personalized cancer vaccine may vary from patient to patient. However, the applicability of the peptide-based vaccine compositions for accommodating any peptide antigen means that the compositions can also be used in other personalized immunological-based treatments, such as for inducing tolerance or for modulating the immune response to allergens for the treatment of auto-immunity and allergies, respectively.

Having developed novel peptide-based vaccine compositions, there is now a need for methods for manufacturing the peptide-based vaccines that are reliable, generally applicable and/or can accommodate a wide range of variability in the physical and/or chemical properties of peptide antigens.

### SUMMARY

In a first aspect, the present disclosure provides a process for producing a peptide antigen conjugate suitable for administration to a mammal, the peptide antigen conjugate comprising a peptide antigen linked to a hydrophobic block, the process comprising: reacting a hydrophobic block fragment with a peptide antigen fragment comprising the peptide antigen in a pharmaceutically acceptable organic solvent in a hydrophobic block fragment to peptide antigen fragment molar ratio of 1:1 or greater under conditions to directly or indirectly link the peptide antigen to the hydrophobic block; and obtaining a product solution comprising the peptide antigen conjugate, unreacted hydrophobic block fragment and pharmaceutically acceptable organic solvent.

In certain embodiments of the first aspect, the product solution that is formed comprises unreacted hydrophobic block fragment and the unreacted hydrophobic block fragment is not removed from the product solution.

In certain embodiments of the first aspect, the process further comprises sterile filtering the product solution to obtain a sterile product solution comprising peptide antigen conjugate, any unreacted hydrophobic block fragment and pharmaceutically acceptable organic solvent. In certain of these embodiments, the process further comprises adding an excess volume of aqueous buffer to the sterile product solution followed by mixing to generate a sterile aqueous solution of peptide antigen conjugate particles comprising the peptide antigen conjugate, any unreacted hydrophobic block fragment, pharmaceutically acceptable organic solvent and aqueous buffer. The aqueous solution of peptide antigen conjugate particles may comprise unreacted hydrophobic block fragment and the unreacted hydrophobic block fragment that is not removed from the aqueous solution of peptide antigen conjugate particles. In certain of these embodiments, the process does not involve removal of the pharmaceutically acceptable organic solvent.

In certain embodiments of the first aspect, the process further comprises lyophilizing the sterile product solution to obtain a lyophilized sterile product. In certain of these embodiments, the process further comprises adding an excess volume of aqueous buffer to the lyophilized sterile product followed by mixing to generate a sterile aqueous solution of peptide antigen conjugate particles comprising the peptide antigen conjugate, any unreacted hydrophobic block fragment and aqueous buffer.

In certain embodiments of the first aspect, the process further comprises purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent. In certain of these embodiments, the process further comprises sterile filtering the purified peptide antigen conjugate solution to obtain a sterile purified peptide antigen conjugate solution comprising the peptide antigen conjugate and pharmaceutically acceptable organic solvent. In certain of these embodiments, the process further comprises adding an excess volume of aqueous buffer to the sterile purified peptide antigen conjugate solution followed by mixing to generate a sterile aqueous solution of peptide antigen conjugate particles comprising the peptide antigen conjugate, pharmaceutically acceptable organic solvent and aqueous buffer.

In certain embodiments of the first aspect, the process further comprises lyophilizing the sterile purified peptide antigen conjugate solution to obtain a lyophilized sterile purified peptide antigen conjugate. In certain of these embodiments, the process further comprises adding an excess volume of aqueous buffer to the lyophilized sterile purified peptide antigen conjugate followed by mixing to generate a sterile aqueous solution of peptide antigen conjugate particles comprising the peptide antigen conjugate and aqueous buffer.

In certain embodiments of the first aspect, the process further comprises analysing the propensity of the product solution, sterile product solution, lyophilized sterile product, lyophilized purified peptide antigen conjugate, purified peptide antigen conjugate solution, sterile purified peptide antigen conjugate solution and/or lyophilized sterile purified peptide antigen conjugate to form aggregated material upon addition of an aqueous buffer, the analysis comprising: (i) aliquoting a specific volume of the product solution, sterile product solution, purified peptide antigen conjugate solution and/or sterile purified peptide antigen conjugate solution from a first container to a second container, and/or adding a specific mass of the lyophilized sterile product, lyophilized purified peptide antigen conjugate and/or lyophilized sterile purified peptide antigen conjugate from a first container to a second container; (ii) adding a volume of the aqueous buffer to the second container to obtain an aqueous solution of peptide antigen conjugate particles comprising the peptide antigen conjugate and any unreacted hydrophobic block fragment, wherein the concentration of the peptide antigen conjugate is not lower than 0.01 mg/mL; (iii) assessing turbidity of the aqueous solution of peptide antigen conjugate particles by measuring absorbance at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

In certain embodiments of the first aspect, the pharmaceutically acceptable organic solvent is selected from one or more of the group consisting of dimethyl sulfoxide (DMSO), methanol and ethanol. In certain of these embodiments, the pharmaceutically acceptable organic solvent is DMSO.

In certain embodiments of the first aspect, the peptide antigen fragment has a formula selected from [C]-[B1]-A-[B2]-X1, [B1]-A-[B2]-X1([C]), X1-[B1]-A-[B2]-[C] or X1([C])-[B1]-A-[B2] where C is a charged moiety, B1 is an N-terminal extension, A is a peptide antigen, B2 is a C-terminal extension, [ ] denotes that the group is optional, and X1 is a linker precursor comprising a first reactive functional group; and the hydrophobic block fragment has a formula selected from X2-H, X2([C])-H or X2-H([C]) where H is a hydrophobic block, C is a charged moiety, [ ] denotes that the group is optional, and X2 is a linker precursor comprising a second reactive functional group that is reactive with the first reactive functional group, and X1 and X2 undergo a reaction to form a covalent bond that results in a Linker L.

In certain embodiments, the peptide antigen conjugate has the formula [C]-[B1]-A-[B2]-L-H. In certain of these embodiments, the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

In certain embodiments, the peptide antigen conjugate has the formula H-L-[B1]-A-[B2]-[C]. In certain of these embodiments, the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

In certain embodiments of the first aspect, the hydrophobic block comprises a poly(amino acid)-based polymer. In certain of these embodiments, the poly(amino acid)-based polymer comprises aromatic rings or heterocyclic aromatic rings. In certain of these embodiments, the poly(amino acid)-based polymer comprises aryl amines.

The process according to any one of the preceding claims, wherein the hydrophobic block fragment is reacted with the peptide antigen fragment in a hydrophobic block fragment to peptide antigen fragment molar ratio of from about 1:1 to about 3:1.

In certain embodiments of the first aspect, the hydrophobic block fragment is reacted with the peptide antigen fragment in a hydrophobic block fragment to peptide antigen fragment molar ratio of from 1:1 to about 12:10.

In certain embodiments of the first aspect, the process further comprises forming a peptide antigen conjugate mixture or lyophilized peptide antigen conjugate mixture comprising two or more peptide antigen conjugates, the process comprising: combining a specific volume of a first product solution comprising a first peptide antigen conjugate, a first purified peptide antigen conjugate solution comprising a first peptide antigen conjugate, a first sterile product solution comprising a first peptide antigen conjugate and/or a first sterile purified peptide antigen conjugate solution comprising a first peptide antigen conjugate with at least a second product solution comprising a second peptide antigen conjugate, a second purified peptide antigen conjugate solution comprising a second peptide antigen conjugate, a second sterile product solution comprising a second peptide antigen conjugate and/or a second sterile purified peptide antigen conjugate solution comprising a second peptide antigen conjugate to obtain a peptide antigen conjugate mixture comprising at least the first peptide antigen conjugate and the second peptide antigen conjugate, any unreacted hydrophobic block fragment and the pharmaceutically acceptable organic solvent; and/or combining a specific mass of a first lyophilized product comprising a first peptide antigen conjugate, a first lyophilized purified peptide antigen conjugate comprising a first peptide antigen conjugate, a first lyophilized sterile product comprising a first peptide antigen conjugate and/or a first lyophilized sterile purified peptide antigen conjugate comprising a first peptide antigen conjugate with at least a specific mass of a second lyophilized product comprising a second peptide antigen conjugate, a second lyophilized purified peptide antigen conjugate comprising a second peptide antigen conjugate, a second lyophilized sterile product comprising a second peptide antigen conjugate and/or a second lyophilized sterile purified peptide antigen conjugate comprising a second peptide antigen conjugate to obtain a lyophilized peptide antigen conjugate mixture comprising at least the first peptide antigen conjugate and the second peptide antigen conjugate and any unreacted hydrophobic block fragment.

In certain embodiments, the peptide antigen conjugate mixture comprises unreacted hydrophobic block fragment and the unreacted hydrophobic block fragment is not removed from the peptide antigen conjugate mixture.

In certain embodiments the step of combining a specific volume of the first product solution, the first purified peptide antigen conjugate solution, the first sterile product solution and/or the first sterile purified peptide antigen conjugate solution with at least the second product solution, the second purified peptide antigen conjugate solution, the second sterile product solution and/or the second sterile purified peptide antigen conjugate solution comprises selecting and transferring a specific volume of solution to transfer from one container to a second container, the process comprising the steps of. (i) determining the molar concentration of the peptide antigen conjugate in at least the first product solution, the first purified peptide antigen conjugate solution, the first sterile product solution, the first sterile purified peptide antigen conjugate solution, the second product solution, the second purified peptide antigen conjugate solution, the second sterile production solution and/or the second sterile purified peptide antigen conjugate solution; (ii) aliquoting a specific volume of at least the first product solution, the first purified peptide antigen conjugate solution, the first sterile product solution and/or the first sterile purified peptide antigen conjugate solution and the second product solution, the second purified peptide antigen conjugate solution, the second sterile product solution and/or the second sterile purified peptide antigen conjugate solution from the first container to a second container to obtain a specific molar content of each of the first peptide antigen conjugate and the second peptide antigen conjugate.

In certain embodiments, the process of determining the molar concentration of peptide antigen conjugate in at least the first product solution, the first purified peptide antigen conjugate solution, the first sterile product solution, the first sterile purified peptide antigen conjugate solution, the second product solution, the second purified peptide antigen conjugate solution, the second sterile product solution and/or the second sterile purified peptide antigen conjugate solution comprises measuring UV-Vis absorption of the peptide antigen conjugate at a wavelength between about 300 to about 350 nm.

In certain embodiments, the process further comprises adding an excess volume of aqueous buffer to the peptide antigen conjugate mixture followed by mixing to generate an aqueous solution of peptide antigen conjugate particles comprising at least the first peptide antigen conjugate and the second peptide antigen conjugate, any unreacted hydrophobic block fragment, any pharmaceutically acceptable organic solvent and aqueous buffer.

In certain embodiments, the process further comprises lyophilization of the peptide antigen conjugate mixture to obtain a lyophilized peptide antigen conjugate mixture. In certain of these embodiments, the process further comprises adding an excess volume of aqueous buffer to the lyophilized peptide antigen conjugate mixture followed by mixing to generate an aqueous solution of peptide antigen conjugate particles comprising at least the first peptide antigen conjugate and the second peptide antigen conjugate, any unreacted hydrophobic block fragment and aqueous buffer.

In certain embodiments, the process further comprises sterile filtering the peptide antigen conjugate mixture to obtain a sterile peptide antigen conjugate mixture. In certain of these embodiments, the process further comprises adding an excess volume of aqueous buffer to the sterile peptide antigen conjugate mixture product followed by mixing to generate a sterile aqueous solution of peptide antigen conjugate particles comprising at least the first peptide antigen conjugate and the second peptide antigen conjugate, any unreacted hydrophobic block fragment, pharmaceutically acceptable organic solvent and aqueous buffer.

In certain embodiments, the process further comprises lyophilization of the sterile peptide antigen conjugate mixture to obtain a lyophilized sterile peptide antigen conjugate mixture. In certain of these embodiments, the process further comprises adding an excess volume of aqueous buffer to the sterile peptide antigen conjugate mixture followed by mixing to generate a sterile aqueous solution of peptide antigen conjugate particles comprising at least the first peptide antigen conjugate and the second peptide antigen conjugate, any unreacted hydrophobic block fragment and aqueous buffer.

In a second aspect, the present disclosure provides a solid phase peptide synthesis process for producing a peptide antigen conjugate suitable for administration to a mammal, the peptide antigen conjugate comprising a peptide antigen linked to a hydrophobic block, the process comprising: providing a solid phase resin bound hydrophobic block fragment; forming a resin bound peptide antigen conjugate by either sequentially coupling individual amino acids and/or polyamino acid fragments to form a peptide antigen fragment coupled to the resin bound hydrophobic block, or coupling a peptide antigen fragment to the resin bound hydrophobic block; cleaving the peptide antigen conjugate from the resin to obtain a peptide antigen conjugate; and purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent.

In certain embodiments of the second aspect, the process further comprises adding an excess volume of aqueous buffer to the lyophilized purified peptide antigen conjugate followed by mixing to generate an aqueous solution of peptide antigen conjugate particles comprising the peptide antigen conjugate and aqueous buffer, or adding an excess volume of aqueous buffer to the purified peptide antigen conjugate solution followed by mixing to generate an aqueous solution of peptide antigen conjugate particles comprising the peptide antigen conjugate, pharmaceutically acceptable organic solvent and aqueous buffer.

In certain embodiments of the second aspect, the process further comprises sterile filtering the purified peptide antigen conjugate solution to obtain a sterile purified peptide antigen conjugate solution comprising peptide antigen conjugate and pharmaceutically acceptable organic solvent. In certain embodiments, the process further comprises adding an excess volume of aqueous buffer to the sterile purified peptide antigen conjugate solution followed by mixing to generate a sterile aqueous solution of peptide antigen conjugate particles comprising the peptide antigen conjugate, pharmaceutically acceptable organic solvent and aqueous buffer.

In certain embodiments of the second aspect, the process further comprises lyophilizing the sterile purified peptide antigen conjugate solution to obtain a lyophilized sterile purified peptide antigen conjugate. In certain embodiments, the process further comprises adding an excess volume of aqueous buffer to the lyophilized sterile purified peptide antigen conjugate followed by mixing to generate a sterile aqueous solution of peptide antigen conjugate particles comprising the peptide antigen conjugate and aqueous buffer.

In certain embodiments of the second aspect, the process further comprises analysing the propensity of the lyophilized purified peptide antigen conjugate, purified peptide antigen conjugate solution, sterile purified peptide antigen conjugate solution and/or lyophilized sterile purified peptide antigen conjugate to form aggregated material upon addition of an aqueous buffer, the analysis comprising the steps of. (i) aliquoting a specific volume of the purified peptide antigen conjugate solution and/or sterile purified peptide antigen conjugate solution from a first container to a second container, and/or adding a specific mass of the lyophilized purified peptide antigen conjugate and/or lyophilized sterile purified peptide antigen conjugate from a first container to a second container; (ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL; (iii) assessing turbidity of the aqueous solution of peptide antigen conjugate particles by measuring absorbance of the aqueous mixture at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

In certain embodiments of the second aspect, the pharmaceutically acceptable organic solvent is selected from one or more of the group consisting of dimethyl sulfoxide (DMSO), methanol and ethanol. In certain embodiments, the pharmaceutically acceptable organic solvent is DMSO.

In certain embodiments of the second aspect, the peptide antigen fragment has a formula selected from [C]-[B1]-A-[B2] or [B1]-A-[B2]-[C], where C is a charged moiety, B1 is an N-terminal extension, A is a peptide antigen, B2 is a C-terminal extension, and [ ] denotes that the group is optional.

In certain embodiments of the second aspect, the peptide antigen conjugate has the formula [C]-[B1]-A-[B2]-H where H is a hydrophobic block. In certain embodiments the peptide antigen conjugate has a formula selected from the group consisting of A-H, C-A-H, B1-A-H, A-B2-H, C-B1-A-H, C-A-B2-H, and C-B1-A-B2-H.

In certain embodiments of the second aspect, the peptide antigen conjugate has the formula H-[B1]-A-[B2]-[C]. In certain embodiments the peptide antigen conjugate has a formula selected from the group consisting of H-A, H-A-C, H-B1-A, H-A-B2, H-B1-A-C, H-A-B2-C, and H-B1-A-B2-C.

In certain embodiments of the second aspect, the hydrophobic block comprises a poly(amino acid)-based polymer. In certain embodiments, the poly(amino acid)-based polymer comprises aromatic rings or heterocyclic aromatic rings. In certain embodiments, the poly(amino acid)-based polymer comprises aryl amines.

In certain embodiments of the second aspect, the process further comprises forming a peptide antigen conjugate mixture comprising two or more peptide antigen conjugates, the process comprising: combining a specific volume of a first purified peptide antigen conjugate solution comprising a first peptide antigen conjugate and/or a first sterile purified peptide antigen conjugate solution comprising a first peptide antigen conjugate with at least a second purified peptide antigen conjugate solution comprising a second peptide antigen conjugate and/or a second sterile purified peptide antigen conjugate solution comprising a second peptide antigen conjugate to obtain a peptide antigen conjugate mixture comprising at least the first peptide antigen conjugate and the second peptide antigen conjugate and the pharmaceutically acceptable organic solvent; and/or combining a specific mass of a first lyophilized purified peptide antigen conjugate comprising a first peptide antigen conjugate and/or a first lyophilized sterile purified peptide antigen conjugate comprising a first peptide antigen conjugate with at least a specific mass of a second lyophilized purified peptide antigen conjugate comprising a second peptide antigen conjugate and/or a second lyophilized sterile purified peptide antigen conjugate comprising a second peptide antigen conjugate to obtain a peptide antigen conjugate mixture comprising at least the first peptide antigen conjugate and the second peptide antigen conjugate.

In certain embodiments, the step of combining a specific volume of the first purified peptide antigen conjugate solution and/or the first sterile purified peptide antigen conjugate solution with at least the second purified peptide antigen conjugate solution and/or the second sterile purified peptide antigen conjugate solution comprises selecting and transferring a specific volume of solution to transfer from one container to a second container, the process comprising the steps of: (i) determining the molar concentration of the peptide antigen conjugate in at least the first purified peptide antigen conjugate solution, the first sterile purified peptide antigen conjugate solution, the second purified peptide antigen conjugate solution and/or the second sterile purified peptide antigen conjugate solution; (ii) aliquoting a specific volume of at least the first purified peptide antigen conjugate solution, the first sterile purified peptide antigen conjugate solution and the second purified peptide antigen conjugate solution and/or the second sterile purified peptide antigen conjugate solution from the first container to a second container to obtain a specific molar content of each of the first peptide antigen conjugate and the second peptide antigen conjugate.

In certain embodiments, the process of determining the molar concentration of peptide antigen conjugate in at least the first purified peptide antigen conjugate solution, the first sterile purified peptide antigen conjugate solution, the second purified peptide antigen conjugate solution and/or the second sterile purified peptide antigen conjugate solution comprises measuring UV-Vis absorption of the peptide antigen conjugate at a wavelength between about 300 to about 350 nm.

In certain embodiments of the second aspect, the process further comprises adding an excess volume of aqueous buffer to the peptide antigen conjugate mixture followed by mixing to generate an aqueous solution of peptide antigen conjugate particles comprising at least the first peptide antigen conjugate and the second peptide antigen conjugate, any pharmaceutically acceptable organic solvent and aqueous buffer.

In certain embodiments of the second aspect, the process further comprises lyophilization of the peptide antigen conjugate mixture to obtain a lyophilized peptide antigen conjugate mixture product. In certain embodiments, the process further comprises adding an excess volume of aqueous buffer to the lyophilized peptide antigen conjugate mixture followed by mixing to generate an aqueous solution of peptide antigen conjugate particles comprising at least the first peptide antigen conjugate and the second peptide antigen conjugate and aqueous buffer.

In certain embodiments of the second aspect, the process further comprises sterile filtering the peptide antigen conjugate mixture to obtain a sterile peptide antigen conjugate mixture. In certain embodiments, the process further comprises adding an excess volume of aqueous buffer to the sterile peptide antigen conjugate mixture followed by mixing to generate a sterile aqueous solution of peptide antigen conjugate particles comprising at least the first peptide antigen conjugate and the second peptide antigen conjugate, pharmaceutically acceptable organic solvent and aqueous buffer.

In certain embodiments of the second aspect, the process further comprises lyophilization of the sterile peptide antigen conjugate mixture to obtain a lyophilized sterile peptide antigen conjugate mixture. In certain embodiments, the process further comprises adding an excess volume of aqueous buffer to the lyophilized sterile peptide antigen conjugate mixture followed by mixing to generate a sterile aqueous solution of peptide antigen conjugate particles comprising at least the first peptide antigen conjugate and the second peptide antigen conjugate and aqueous buffer.

In a third aspect, the present disclosure provides a process for producing a sterile aqueous solution of peptide antigen conjugate particles, the process comprising: preparing a peptide antigen conjugate solution comprising a peptide antigen conjugate and a pharmaceutically acceptable organic solvent, said peptide antigen conjugate comprising a peptide antigen linked to a hydrophobic block; sterile-filtering the peptide antigen conjugate solution to produce a sterile peptide antigen conjugate solution; and adding an aqueous buffer to the sterile peptide antigen conjugate solution to produce the sterile aqueous solution of peptide antigen conjugate particles.

In certain embodiments of the third aspect, the process further comprises: a′) preparing a second peptide antigen conjugate solution comprising a second peptide antigen conjugate and a pharmaceutically acceptable organic solvent, said second peptide antigen conjugate comprising a second peptide antigen linked to a hydrophobic block; a″) combining a specific volume of each of the peptide antigen conjugate solution and the second peptide antigen conjugate solution to obtain a peptide antigen conjugate mixture comprising two or more different peptide antigen conjugates which is then subjected to steps b) and c).

In certain embodiments of the third aspect, the process further comprises: a′) preparing a second peptide antigen conjugate solution comprising a second peptide antigen conjugate and a pharmaceutically acceptable organic solvent, said second peptide antigen conjugate comprising a second peptide antigen linked to a hydrophobic block; b′) sterile-filtering the second peptide antigen conjugate solution to produce a second sterile peptide antigen conjugate solution; and b″) combining a specific volume of each of the sterile peptide antigen conjugate solution and the second sterile peptide antigen conjugate solution to obtain a combined sterile peptide antigen conjugate solution which is then subjected to step c).

In certain embodiments of the third aspect, the process further comprises analysing the propensity of the peptide antigen conjugate solution, the second peptide antigen conjugate solution, the sterile peptide antigen conjugate solution, the second sterile peptide antigen conjugate solution, the peptide antigen conjugate mixture and/or the sterile peptide antigen conjugate mixture to form aggregated material upon addition of an aqueous buffer, the analysis comprising the steps of: (i) aliquoting a specific volume of the peptide antigen conjugate solution, the second peptide antigen conjugate solution, the sterile peptide antigen conjugate solution, the second sterile peptide antigen conjugate solution, the peptide antigen conjugate mixture and/or the sterile peptide antigen conjugate mixture from a first container to a second container; (ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL; (iii) assessing turbidity of the aqueous solution of peptide antigen conjugate particles by measuring absorbance of the aqueous mixture at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

In certain embodiments of the third aspect, the pharmaceutically acceptable organic solvent is selected from one or more of the group consisting of dimethyl sulfoxide (DMSO), methanol and ethanol. In certain embodiments, the pharmaceutically acceptable organic solvent is DMSO.

In certain embodiments of the third aspect, the peptide antigen conjugate has the formula [C]-[B1]-A-[B2]-H where C is a charged moiety, B1 is an N-terminal extension, A is a peptide antigen, B2 is a C-terminal extension, H is a hydrophobic block, and [ ] denotes that the group is optional. In certain embodiments, the peptide antigen conjugate has a formula selected from the group consisting of A-H, C-A-H, B1-A-H, A-B2-H, C-B1-A-H, C-A-B2-H, and C-B1-A-B2-H.

In certain embodiments of the third aspect, the peptide antigen conjugate has the formula H-[B1]-A-[B2]-[C] where H is a hydrophobic block, B1 is an N-terminal extension, A is a peptide antigen, B2 is a C-terminal extension, C is a charged moiety, and [ ] denotes that the group is optional. In certain embodiments the peptide antigen conjugate has a formula selected from the group consisting of H-A, H-A-C, H-B1-A, H-A-B2, H-B1-A-C, H-A-B2-C, and H-B1-A-B2-C.

In certain embodiments of the third aspect, the peptide antigen conjugate has the formula [C]-[B1]-A-[B2]-L-H, where C is a charged moiety, B1 is an N-terminal extension, A is a peptide antigen, B2 is a C-terminal extension, H is a hydrophobic block, L is a Linker, and [ ] denotes that the group is optional. In certain embodiments, the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

In certain embodiments of the third aspect, the peptide antigen conjugate has the formula H-L-[B1]-A-[B2]-[C], where C is a charged moiety, B1 is an N-terminal extension, A is a peptide antigen, B2 is a C-terminal extension, H is a hydrophobic block, L is a Linker, and [ ] denotes that the group is optional. In certain embodiments, the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

In certain embodiments of the third aspect, the hydrophobic block comprises a poly(amino acid)-based polymer. In certain embodiments, the poly(amino acid)-based polymer comprises aromatic rings or heterocyclic aromatic rings. In certain embodiments, the poly(amino acid)-based polymer comprises aryl amines.

In a fourth aspect, the present disclosure provides a process for analysing the propensity of a peptide antigen conjugate composition comprising a peptide antigen linked to a hydrophobic block to form aggregated material upon addition of an aqueous buffer, the analysis comprising the steps of. (i) aliquoting a specific volume of a peptide antigen conjugate solution from a first container to a second container, and/or adding a specific mass of a peptide antigen conjugate from a first container to a second container; (ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL; (iii) assessing turbidity of the aqueous solution of peptide antigen conjugate particles by measuring absorbance of the aqueous mixture at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

In certain embodiments of the fourth aspect, the peptide antigen conjugate has a formula selected from [C]-[B1]-A-[B2]-H or [B1]-A-[B2]-H([C]) where C is a charged moiety, B1 is an N-terminal extension, A is a peptide antigen, B2 is a C-terminal extension, H is a hydrophobic block, and [ ] denotes that the group is optional. In certain embodiments, the peptide antigen conjugate has a formula selected from the group consisting of A-H, C-A-H, B1-A-H, A-B2-H, C-B1-A-H, C-A-B2-H, and C-B1-A-B2-H.

In certain embodiments of the fourth aspect, the peptide antigen conjugate has the formula H-[B1]-A-[B2]-[C] or H([C)]-[B1]-A-[B2] where B1 is an N-terminal extension, A is a peptide antigen, B2 is a C-terminal extension, C is a charged moiety, H is a hydrophobic block, and [ ] denotes that the group is optional. In certain embodiments, the peptide antigen conjugate has a formula selected from the group consisting of H-A, H-A-C, H-B1-A, H-A-B2, H-B1-A-C, H-A-B2-C, and H-B1-A-B2-C.

In certain embodiments of the fourth aspect, the peptide antigen conjugate has a formula selected from [C]-[B1]-A-[B2]-L-H, [B1]-A-[B2]-L([C])-H or [B1]-A-[B2]-L-H([C]), where C is a charged moiety, B1 is an N-terminal extension, A is a peptide antigen, B2 is a C-terminal extension, H is a hydrophobic block, L is a Linker, and [ ] denotes that the group is optional. In certain embodiments, the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

In certain embodiments of the fourth aspect, the peptide antigen conjugate has a formula selected from H-L-[B1]-A-[B2]-[C], H([C])-L-[B1]-A-[B2] or H-L([C])-[B1]-A-[B2] where C is a charged moiety, B1 is an N-terminal extension, A is a peptide antigen, B2 is a C-terminal extension, H is a hydrophobic block, L is a Linker, and [ ] denotes that the group is optional. In certain embodiments, the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

In certain embodiments of the fourth aspect, the hydrophobic block comprises a poly(amino acid)-based polymer. In certain embodiments, the poly(amino acid)-based polymer comprises aromatic rings or heterocyclic aromatic rings. In certain embodiments, the poly(amino acid)-based polymer comprises aryl amines.

In a fifth aspect, the present disclosure provides a process for producing a peptide antigen conjugate mixture comprising a first peptide antigen linked to a hydrophobic block and at least a second peptide antigen linked to a hydrophobic block, the process comprising: preparing a first peptide antigen conjugate solution comprising a first peptide antigen conjugate and a pharmaceutically acceptable organic solvent; preparing at least a second peptide antigen conjugate solution comprising a second peptide antigen conjugate and a pharmaceutically acceptable organic solvent; combining a specific volume of the peptide antigen conjugate solutions to obtain a peptide antigen conjugate mixture comprising the first peptide antigen conjugate and the at least second peptide antigen conjugate and a pharmaceutically acceptable organic solvent.

In certain embodiments of the fifth aspect, the step of combining a specific volume of the peptide antigen conjugate solutions comprises selecting and transferring a specific volume of each peptide antigen conjugate solution to transfer from one container to a second container, the process comprising the steps of: (i) determining the molar concentration of the peptide antigen conjugate in each of the peptide antigen conjugate solutions; (ii) aliquoting a specific volume of each peptide antigen conjugate solution from the first container to a second container to obtain a specific molar content of each of the peptide antigen conjugates.

In certain embodiments of the fifth aspect, the process of determining the molar concentration of peptide antigen conjugate in each of the peptide antigen conjugate solutions comprises measuring UV-Vis absorption of the peptide antigen conjugate at a wavelength between about 300 to about 350 nm.

In certain embodiments of the fifth aspect, the process further comprises adding an excess volume of aqueous buffer to the peptide antigen conjugate mixture followed by mixing to generate an aqueous solution of peptide antigen conjugate particles comprising the first peptide antigen conjugate and at least the second peptide antigen conjugate, pharmaceutically acceptable organic solvent and aqueous buffer.

In certain embodiments of the fifth aspect, the process further comprises lyophilization of the peptide antigen conjugate mixture to obtain a lyophilized peptide antigen conjugate mixture. In certain embodiments, the process further comprises adding an excess volume of aqueous buffer to the lyophilized peptide antigen conjugate mixture followed by mixing to generate an aqueous solution of peptide antigen conjugate particles comprising the first peptide antigen conjugate and at least the second peptide antigen conjugate, pharmaceutically acceptable organic solvent and aqueous buffer.

In certain embodiments of the fifth aspect, the process further comprises sterile filtering the peptide antigen conjugate mixture to obtain a sterile peptide antigen conjugate mixture. In certain embodiments, the process further comprises adding an excess volume of aqueous buffer to the sterile peptide antigen conjugate mixture followed by mixing to generate a sterile aqueous solution of peptide antigen conjugate particles comprising the first peptide antigen conjugate and at least the second peptide antigen conjugate, pharmaceutically acceptable organic solvent and aqueous buffer.

In certain embodiments of the fifth aspect, the process further comprises lyophilization of the sterile peptide antigen conjugate mixture to obtain a lyophilized sterile peptide antigen conjugate mixture. In certain embodiments, the process further comprises adding an excess volume of aqueous buffer to the lyophilized sterile peptide antigen conjugate mixture followed by mixing to generate a sterile aqueous solution of peptide antigen conjugate particles comprising the first peptide antigen conjugate and at least the second peptide antigen conjugate, pharmaceutically acceptable organic solvent and aqueous buffer.

In certain embodiments of the fifth aspect, the process further comprises analysing the propensity of the peptide antigen conjugate mixture, lyophilized peptide antigen conjugate mixture, sterile peptide antigen conjugate mixture and/or lyophilized sterile peptide antigen conjugate mixture to form aggregated material upon addition of an aqueous buffer, the analysis comprising the steps of. (i) aliquoting a specific volume of the peptide antigen conjugate mixture and/or sterile peptide antigen conjugate mixture from a first container to a second container, and/or adding a specific mass of the lyophilized peptide antigen conjugate mixture and/or lyophilized sterile peptide antigen conjugate mixture from a first container to a second container; (ii) adding a volume of the aqueous buffer to the second container to obtain an aqueous solution of peptide antigen conjugate particles comprising the first peptide antigen conjugate and at least the second peptide antigen conjugate, wherein the concentration of the peptide antigen conjugates is not lower than 0.01 mg/mL; (iii) assessing turbidity of the aqueous solution of peptide antigen conjugate particles by measuring absorbance of the aqueous mixture at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

In certain embodiments of the fifth aspect, the process for selecting the composition and volume of the first peptide antigen conjugate and the at least second peptide antigen conjugate to include in the peptide antigen conjugate mixture comprises any one or both of the steps of: (i) determining the molar concentration of the peptide antigen conjugates in the peptide antigen conjugate solutions; (ii) determining the propensity of each of the peptide antigen conjugate solutions to form aggregated material upon addition of an excess of aqueous buffer to dilute the peptide antigen conjugates to a concentration no lower than 0.01 mg/mL.

In certain embodiments of the fifth aspect, the molar concentration of peptide antigen conjugates derived from the peptide antigen conjugate mixture and/or the sterile peptide antigen conjugate mixture that each individually have the propensity to form aggregated material upon addition of the aqueous buffer comprise 60% or less of the total molar content of peptide antigen conjugates in the peptide antigen conjugate mixture.

In certain embodiments of the fifth aspect, the process of determining the molar concentration of peptide antigen conjugates in the peptide antigen conjugate mixture and/or the sterile peptide antigen conjugate mixture comprises measuring UV-Vis absorption of the peptide antigen conjugates at a wavelength between about 300 to about 350 nm.

In a sixth aspect, the present disclosure provides a peptide antigen conjugate produced by the process of any one of the first and second aspects.

In a seventh aspect, the present disclosure provides an immunogenic composition comprising the peptide antigen conjugate of the sixth aspect.

In an eighth aspect, the present disclosure provides a sterile aqueous solution of peptide antigen conjugate particles produced by the process of the third aspect.

In a ninth aspect, the present disclosure provides a peptide antigen conjugate mixture produced by the process of the fifth aspect.

### BRIEF DESCRIPTION OF FIGURES

Embodiments of the present disclosure will be discussed with reference to the accompanying figures wherein:

FIG. 1 is a schematic outline of a manufacturing process of one or more embodiments of the present disclosure;

FIG. 2 shows one possible synthetic route for producing a peptide-based hydrophobic block fragment (X2-H) entirely by solid-phase peptide synthesis;

FIG. 3 shows four possible synthetic routes for producing a peptide-based hydrophobic block fragment (X2-H) using a combination of solid-phase peptide synthesis and solution phase synthesis;

FIG. 4 shows an example of the reaction scheme 1.4 for producing a hydrophobic block fragment according to embodiments of the present disclosure;

FIG. 5 shows an example of the reaction scheme 1.5 for producing a hydrophobic block fragment according to embodiments of the present disclosure;

FIG. 6 shows HPLC traces of an azide-alkyne reaction in DMSO of a peptide antigen fragment (e.g., C1-B1-A-B2-X1) and a hydrophobic block fragment (X2-H) which resulted in full conversion of the peptide antigen fragment to the peptide antigen conjugate (C-B-A-B2-L-H);

FIG. 7A shows HPLC traces over time of a reaction of a peptide antigen fragment (e.g., C1-B1-A-B2-X1) and a hydrophobic block fragment (X2-H) which resulted in full conversion of the peptide antigen fragment to the peptide antigen conjugate (C-B1-A-B2-L-H), which is driven by the use of excess hydrophobic block fragment;

FIG. 7B shows the reaction kinetics for the formation of a peptide antigen conjugate that results from the reaction of a peptide antigen fragment (e.g., C1-B1-A-B2-X1) and a hydrophobic block fragment (X2-H);

FIG. 8 shows a plot of the kinetics for a reaction between a peptide antigen fragment (e.g., C1-B1-A-B2-X1) and a hydrophobic block fragment (X2-H). The reaction is not influenced by the peptide antigen (A) length or the hydrophobic block (H) composition;

FIG. 9 shows HPLC traces over time for the formation of peptide antigen conjugate that results from the reaction between a peptide antigen fragment (e.g., C1-B1-A-B2-X1) and a hydrophobic block fragment (X2-H), which is accelerated by increasing the reaction temperature and reagent concentration. No by-products or decomposition is observed at increased temperature and reagent concentration;

FIG. 10A is a graph of the reaction kinetics shown in the HPLC traces shown in FIG. 10;

FIG. 10B is a graph that summarizes how reaction temperature and reagent concentrations impact the calculated half-life (T_{1/2}) for the kinetics of conjugation reactions between a peptide antigen fragment (e.g., C1-B1-A-B2-X1) and a hydrophobic block fragment (X2-H);

FIG. 11A shows HPLC traces of a peptide antigen fragment (e.g., C1-B1-A-B2-X1) and a hydrophobic block fragment (X2-H), and the product solution containing excess hydrophobic block fragment (X2-H), which can be removed by chromatographic separation, i.e. HPLC, to generate a purified peptide antigen conjugate solution;

FIG. 11B is a graph that shows the impact of excess hydrophobic block fragment (X2-H) on the size of vaccine particles formed by the product solution as compared with the purified peptide antigen conjugate solution following addition of aqueous buffer. The data shows that removal of the excess hydrophobic block fragment by chromatographic separation has no discernible impact on vaccine particle size;

FIG. 11C is a graph that shows the impact of excess hydrophobic block fragment (X2-H) on CD8 T cell responses following two vaccinations of mice with an aqueous solution of vaccine particles formed from either the addition of aqueous buffer to the product solution comprising peptide antigen conjugate and any unreacted hydrophobic block fragment or formed from the addition of aqueous buffer to the purified peptide antigen conjugate solution;

FIG. 12A shows the HPLC chromatograms of different product solutions obtained by varying the molar ratio of the hydrophobic block fragment (X2-H) used to react with the peptide antigen fragment (e.g., C1-B1-A-B2-X1);

FIG. 12B is a graph that shows how excess (i.e. unreacted) hydrophobic block fragment (X2-H) influences the turbidity of aqueous solutions, or size of vaccine particles in the aqueous solution, formed by the addition of aqueous buffer to product solutions with varying amounts of unreacted hydrophobic block fragment;

FIG. 13 is a series of graphs and table that shows how variations in the charge and hydropathy of the individual peptide antigens (A) comprising peptide antigen conjugate mixtures impacts the turbidity of aqueous solutions of peptide antigen conjugate particles, and size of the vaccine particles, following addition of aqueous buffer to different compositions of peptide antigen conjugate mixtures;

FIG. 14 shows how peptide antigens (A) with extremes of charge, hydropathy, and hydrodynamic behavior (i.e., propensity to form aggregates as indicated by turbidity measurements) impact the size of vaccine particles and turbidity of aqueous solutions of peptide antigen conjugate particles formed following addition of aqueous buffer to different compositions of peptide antigen conjugate mixtures;

FIG. 15A shows HPLC chromatograms of a product solution, comprising a peptide antigen conjugate, before and after sterile filtration using a filter comprising a PTFE membrane;

FIG. 15B is a graph that shows how the sterile filtration technique and filter membrane composition impacts recovery of peptide antigen conjugates following sterile filtration of the product solution to yield a sterile product solution;

FIG. 16 shows the HPLC chromatograms of a peptide antigen conjugate mixture before and after sterile filtration using a filter comprising a PTFE membrane;

FIG. 17 shows a schematic flow diagram for the compounding of peptide antigen conjugates to produce peptide antigen conjugate mixtures; the peptide antigen conjugate mixtures are sterile filtered to produce sterile peptide antigen conjugate mixtures; excess aqueous buffer is then added to the sterile peptide antigen conjugate mixtures to produce an aqueous solution of peptide antigen conjugate particles comprised of nanoparticle micelles;

FIG. 18A is a graph that shows the particle size change over time of peptide antigen conjugate particles, which are nanoparticle micelles in this example, formed by adding excess aqueous buffer to a product solution comprising a single peptide antigen conjugate, unreacted hydrophobic block fragment and DMSO;

FIG. 18B is a graph that shows the particle size change over time of peptide antigen conjugate particles, which are nanoparticles micelles in this example, formed by adding excess aqueous buffer to a peptide antigen conjugate mixture comprising 7 different peptide antigen conjugates, any unreacted hydrophobic block fragment and DMSO;

FIG. 19 shows a graph that demonstrates how pharmaceutically acceptable organic solvent, which is DMSO in this example, impacts (A) particle size, (B&C) immunogenicity and (D&E) systemic toxicity of aqueous solutions of peptide antigen conjugate particles formed by adding excess aqueous buffer to different peptide antigen conjugate mixtures comprising 5 different peptide antigen conjugates, any unreacted hydrophobic block fragment and DMSO. Four sets of peptide antigen conjugate mixtures were either suspended in PBS from DMSO to yield an aqueous solution of peptide antigen conjugate particles in 12.5% v/v DMSO (a-d 12.5% DMSO); or the four sets of peptide antigen conjugate mixtures were lyophilized to yield a solid that was resuspended in PBS to yield an aqueous solution of peptide antigen conjugate particles (or “nanoparticle micelles) without DMSO (a-d; no DMSO);

FIG. 20 shows particle size measurements of aqueous solutions of peptide antigen conjugate particles based on negatively charged peptide antigen conjugates of formula C-[B1]-A-B2-L-H, i.e., Ac-Glu-Glu-Glu-Glu-Glu-Val-Cit-Thr-Ala-Pro-Asp-Asn-Leu-Gly-Tyr-Met-Ser-Pro-Val-Cit-Lys(N3-DBCO)-Glu(2B)-Trp-Glu(2B)-Trp-Glu(2B)-NH2 and Ac-Asp-Asp-Asp-Asp-Asp-Asp-Asp-Asp-Asp-Asp-Asp-Asp-Asp-Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Ser- Pro-Val-Cit-Lys(N3-DBCO)-Glu(2B)-Trp-Glu(2B)-Trp-Glu(2B)-NH2 referred to as Adpgk peptide antigen conjugate and Trp1 peptide antigen conjugate, respectively, with varying molar equivalents of Tris (relative to moles of acid present on the peptide antigen conjugate);

FIG. 21 shows the CD8 T cell response kinetic of C57BL/6 mice (n=5 per group) vaccinated by the SC route with different doses (8 to 100 nmol) of the MC38 mouse tumor neoantigen Adpgk delivered as different compositions of peptide antigen conjugates of formula C-B1-A-B2-L-H, i.e., either (A) a net negatively charged sequence as the Tris ammonium salt (abbreviated (−) w/ Tris), (B) a net negatively charged sequence without Tris (abbreviated (−) w/o Tris) or (C) a positively charged sequence (abbreviated (+)). Mice were vaccinated on day 0, 14, and 28. CD8 T cell responses were measured on day 12, 21, and 35 from whole blood using an intracellular cytokine staining assay for IFN-g production;

FIG. 22 shows the CD8 T cell response kinetic of C57BL/6 mice (n=3 per group) vaccinated by the SC route with different doses (8 and 32 nmol) of the MC38 mouse tumor neoantigen Cpne1 delivered as a peptide antigen conjugate of formula C-B1-A-B2-L-H, either comprising a charged moiety with positive charge, i.e. Lys-Lys-Lys-Lys-Lys-Lys-Lys-Lys-Lys-Val-Arg-Asp-Phe-Thr-Gly-Ser-Asn-Gly-Asp-Pro-Ser-Ser-Pro-Try-Ser-Leu-His-Tyr-Leu-Ser-Pro-Thr-Gly-Val-Asn-Glu-Tyr-Ser-Pro-Val-Cit-Lys(N3-DBCO)-Glu(2B)-Trp-Glu(2B)-Trp-Glu(2B)-NH2 (abbreviated (+) CAH) or a charged moiety with negative charge and a B2 comprised of amino acids bearing aryl amines, i.e., Ac-Glu-Glu-Glu-Glu-Glu-Val-Cit-Asp-Phe-Thr-Gly-Ser-Asn-Gly-Asp-Pro-Ser-Ser-Pro-Try-Ser-Leu-His-Tyr-Leu-Ser-Pro-Thr-Gly-Val-Asn-Glu-Tyr-Ser-Pro-Val-Cit-Phe(NH2)-Phe(NH2)-Phe(NH2)-Phe(NH2)-Lys(N3-DBCO)-Glu(2B)-Trp-Glu(2B)-Trp-Glu(2B)-NH2 (abbreviated (−) CAH), wherein Phe(NH2) is para-amino-phenylalanine. Mice were vaccinated on day 0, 14, and 28. CD8 T cell responses were measured on day 7, 21, 35, and 42 from whole blood using an intracellular cytokine staining assay for IFN-g production; and

FIG. 23 shows CD8 T cell responses at day 21 for C57BL/6 mice (n=4 per group) vaccinated by the SC route with the MC38 mouse tumor neoantigen Adpgk delivered as mosaic particles comprising a peptide antigen conjugate of formula A-B2-L-H and a charged amphiphilic carrier molecule of formula C-L-H (abbreviated AH+CH) or as mosaic particles comprising a peptide antigen conjugate of formula A-B2-L-H and a charged amphiphilic carrier molecule of formula C-B1-A′-B2-L-H, wherein A′ is a conserved antigen (abbreviated AH+CA′H). Mice were vaccinated on day 0 and 14. CD8 T cell responses were measured on day 21 from whole blood using an intracellular cytokine staining assay for IFN-g production.

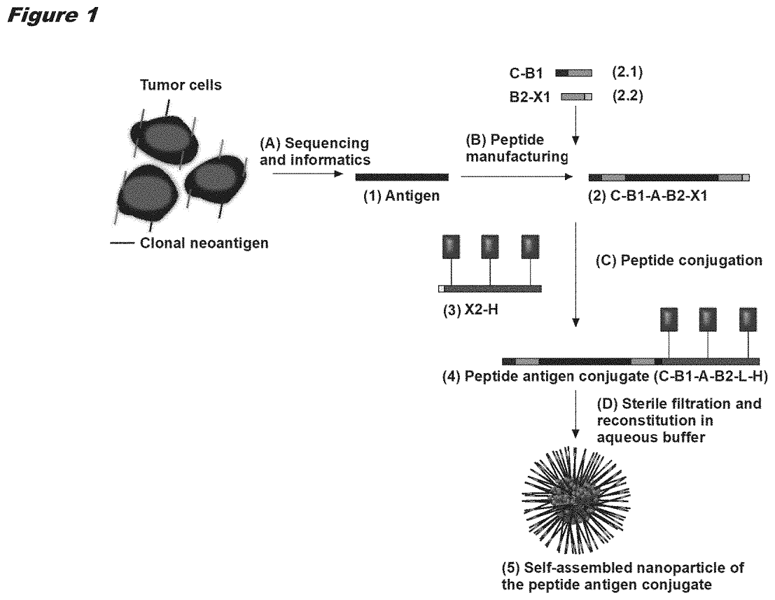

### DESCRIPTION OF EMBODIMENTS

Described herein is a process for producing a peptide antigen conjugate suitable for administration to a mammal. The peptide antigen conjugate comprises a peptide antigen linked to a hydrophobic block. The process comprises reacting a hydrophobic block fragment with a peptide antigen fragment comprising the peptide antigen in a pharmaceutically acceptable organic solvent in a hydrophobic block fragment to peptide antigen fragment molar ratio of 1:1 or greater under conditions to directly or indirectly link the peptide antigen to the hydrophobic block and obtaining a product solution comprising the peptide antigen conjugate, unreacted hydrophobic block fragment and pharmaceutically acceptable organic solvent.

Also described herein is a solid phase peptide synthesis process for producing a peptide antigen conjugate suitable for administration to a mammal. The peptide antigen conjugate comprises a peptide antigen linked to a hydrophobic block.

The process comprises providing a solid phase resin bound hydrophobic block fragment; forming a resin bound peptide antigen conjugate by either sequentially coupling individual amino acids and/or poly(amino acid) fragments to form a peptide antigen fragment coupled to the resin bound hydrophobic block, or coupling a peptide antigen fragment to the resin bound hydrophobic block; cleaving the peptide antigen conjugate from the resin to obtain a peptide antigen conjugate; and purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent. Alternatively, the process comprises providing a solid phase resin bound peptide antigen fragment; forming a resin bound peptide antigen conjugate by coupling the hydrophobic block fragment to the resin bound peptide antigen fragment to form a resin bound peptide antigen conjugate; cleaving the peptide antigen conjugate from the resin to obtain a peptide antigen conjugate; and purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent.

Also described herein is a process for producing a sterile aqueous solution of peptide antigen conjugate particles. The process comprises preparing a peptide antigen conjugate solution comprising a peptide antigen conjugate and a pharmaceutically acceptable organic solvent, said peptide antigen conjugate comprising a peptide antigen linked to a hydrophobic block; sterile-filtering the peptide antigen conjugate solution to produce a sterile peptide antigen conjugate solution; and adding an aqueous buffer to the sterile peptide antigen conjugate solution to produce the sterile aqueous solution of peptide antigen particles.

Also described herein is a process for analysing the propensity of a peptide antigen conjugate composition comprising a peptide antigen linked to a hydrophobic block to form aggregated material upon addition of an aqueous buffer. The analysis comprises the steps of: (i) aliquoting a specific volume of a peptide antigen conjugate solution from a first container to a second container, and/or adding a specific mass of a peptide antigen conjugate from a first container to a second container; (ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL; (iii) assessing turbidity of the aqueous solution of peptide antigen conjugate particles by measuring absorbance of the aqueous mixture at a wavelength greater than 350 nm; and (iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

Also described herein is a process for producing a peptide antigen conjugate mixture comprising a first peptide antigen linked to a hydrophobic block and at least a second peptide antigen linked to a hydrophobic block. The process comprises preparing a first peptide antigen conjugate solution comprising a first peptide antigen conjugate and a pharmaceutically acceptable organic solvent; preparing at least a second peptide antigen conjugate solution comprising a second peptide antigen conjugate and a pharmaceutically acceptable organic solvent; combining a specific volume of the peptide antigen conjugate solutions to obtain a peptide antigen conjugate mixture comprising the first peptide antigen conjugate and the at least second peptide antigen conjugate and a pharmaceutically acceptable organic solvent.

Unless specifically stated otherwise, the following abbreviations are used throughout this specification:
A: peptide antigen
B1: N-terminal extension
B2: C-terminal extension
C: charged moiety
H: hydrophobic block
L: Linker that covalently links the peptide antigen fragment ([C]-[B1]-A-[B2]) and the hydrophobic block fragment (H). Note that this specific form of linker is denoted throughout this specification by a capital “L”. All other linkers used to join other components of the peptide antigen conjugates are denoted by a lower case “1”
X1: linker precursor comprising a first reactive functional group
X2: linker precursor comprising a second reactive functional group

Details of terms used herein are given below for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” means plus or minus 5% from a set amount. For example, “about 10” refers to 9.5 to 10.5. A ratio of “about 5:1” refers to a ratio from 4.75:1 to 5.25:1.

Adjuvant: An “adjuvant” is any material added to vaccines to enhance or modify the immunogenicity of an antigen. The person of ordinary skill in the art is familiar with adjuvants (see: Perrie et al., Int J Pharm 364:272-280, 2008 and Brito et al., Journal of controlled release, 190C:563-579, 2014). Adjuvants can be delivery systems, such as particles based on inorganic salts (e.g., aluminum hydroxide or phosphate salts referred to as alum), water-in-oil or oil-in-water emulsions or polymer particles (e.g., PLGA) in which antigen is simply admixed with or adsorbed, incorporated within or linked indirectly or directly through covalent interactions. Alternatively, adjuvants can be chemically defined molecules that bind to defined receptors and induce downstream signaling pathways, including pattern recognition receptor (PRR) agonists, such as synthetic or naturally occurring agonists of Toll-like receptors (TLRs), stimulator of interferon genes (STING), nucleotide-binding oligomerization domain-like receptors (NLRs), retinoic acid-inducible gene-I-like receptors (RLRs) or C-type lectin receptors (CLRs), as wells as biological molecules (a “biological adjuvant”), such as IL-2, RANTES, GM-CSF, TNF-α, IFN-γ, G-CSF, LFA-3, CD72, B7-1, B7-2, OX-40L, 4-1BBL. Small molecule analogs of nucleotide bases, such as hydroxyadenine and imidazoquinolines, that bind to Toll-like receptors-7 (TLR-7) and TLR-7/8a, respectively, as well as agonists of TLR-2/6, TLR-4, STING and NOD are used as exemplary PRR agonists in the present disclosure. In general, any PRR agonist or biological adjuvant listed herein can be joined to the peptide antigen conjugate of the present disclosure through any suitable means.

Administration: In the context of the present disclosure, “administration” means to provide or give to a subject an agent, for example, an immunogenic composition comprising a peptide antigen conjugate as described herein, by any effective route. Exemplary routes of administration include, but are not limited to, oral, injection (such as subcutaneous, intramuscular, intradermal, intraperitoneal, and intravenous), transdermal (for example, topical), intranasal, vaginal, and inhalation routes.

“Administration of” and “administering a” compound should be understood to mean providing a compound, a prodrug of a compound, or a pharmaceutical composition as described herein. The compound or composition can be administered by another person to the subject or it can be self-administered by the subject.

Antigen: An antigen is any molecule that contains an epitope that binds to a T cell or B cell receptor and can stimulate an immune response, in particular, a B cell response and/or a T cell response in a subject. The epitopes may be comprised of peptides, glycopeptides, lipids or any suitable molecules that contain an epitope that can interact with components of specific B cell or T cell proteins. Such interactions may generate a response by the immune cell. “Epitope” refers to the region of an antigen, such as a peptide antigen, to which B and/or T cell proteins, i.e., B-cell receptors and T-cell receptors, interact.

Aromatic: Aromatic compounds are unsaturated cyclic rings with an odd number of pairs of pi orbital electrons that are delocalized between the carbon or nitrogen atoms forming the ring. Aromatic amino acids include those with a side chain comprising an aromatic group, such as phenylalanine, tyrosine, or tryptophan. Benzene, a 6-carbon ring containing three double bounds is a prototypical aromatic compound. Phenylalanine (Phe) and Tryptophan (Trp) are prototypical aromatic amino acids. Aryl may refer to an aromatic substituent and aryl-amine may refer to an aromatic group comprising an amine. An exemplary aromatic amine is aniline. Aromatic heterocycles refer to aromatic rings comprising cyclic ring structures comprising carbon and another atom, such as nitrogen, oxygen or sulfur. Nucleotide bases, such as adenine and cytosine, are exemplary aromatic heterocycles.

Biocompatible: Materials are considered biocompatible if they exert minimal destructive or host response effects while in contact with body fluids, cells, or tissues, A biocompatible group may contain chemical moieties, including from the following classes: aliphatic, alicyclic, heteroaliphatic, heteroalicyclic, aryl, or heteroaryl. However, depending on the molecular composition, such moieties are not always biocompatible.

The term “biocompatibility” is alternatively taken to mean either minimal interactions with recognition proteins and/or other components of biological systems (e.g., naturally occurring antibodies, cell proteins including glycoproteins, or cells); or substances and functional groups specifically intended to cause interactions with components of biological systems (e.g., drugs and prodrugs), such that the result of the interactions are not substantially negative or destructive.

CD4: Cluster of differentiation 4, a surface glycoprotein that interacts with MHC Class II molecules present on the surface of other cells. A subset of T cells express CD4 and these cells are commonly referred to as helper T cells.

CD8: Cluster of differentiation 8, a surface glycoprotein that interacts with MHC Class I molecules present on the surface of other cells. A subset of T cells express CD8 and these cells are commonly referred to as cytotoxic T cells or killer T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units.

Effective amount: In the context of the present disclosure, “effective amount”, and related terms, means an amount needed to induce a desired response. For example, the amount of an agent, either alone or with one or more additional agents, needed to induce an immune response, for example, of a peptide antigen conjugate.

Graft polymer: May be described as a polymer that results from the linkage of a polymer of one composition to the side chains of a second polymer of a different composition. A first polymer linked through co-monomers to a second polymer is a graft co-polymer. A first polymer linked through an end group to a second polymer may be described as a block polymer (e.g., A-B type di-block) or an end-grafted polymer.

Hydropathy index/GRAVY value: Is a number representing the hydrophobic or hydrophilic characteristics of an amino acid. There are a variety of scales that can be used to describe the relative hydrophobic and hydrophilic characteristics of amino acids comprising peptides. In the present disclosure, the Hydropathy scale of Kyte and Doolittle (Kyte J, Doolittle R F, J. Mol. Biol 157: 105-32, 1983) is used to calculate the grand average of hydropathy (GRAVY) value, sometimes referred to as the GRAVY score, of the sequence of amino acids comprising the peptide antigen fragment and peptide antigen conjugates, including the peptide antigen (A), optional peptide-based N- and C-terminal optional extensions (B iand B2) and the optional charged moiety (C). The GRAVY value of a peptide is the sum of the Hydropathy values of all amino acids comprising the peptide divided by the length (i.e. number of amino acids) of the peptide. The GRAVY value is a relative value. The larger the GRAVY value, the more hydrophobic a peptide sequence is considered, whereas the lower the GRAVY value, the more hydrophilic a peptide sequence is considered.

Hydrophilic: In the context of the present disclosure, “hydrophilic”, and related terms, refers to the tendency of a material to disperse freely in aqueous media. A material is considered hydrophilic if it has a preference for interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble up to 0.1 mg/mL or more.

Hydrophobic: In the context of the present disclosure, “hydrophobic”, and related terms, refers to the tendency of a material to avoid contact with water. A material is considered hydrophobic if it has a preference for interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble down to 0.1 mg/mL or less.

Hydrophobic ligand: Is a molecule that binds to biological receptors and has hydrophobic characteristics. In some embodiments, hydrophobic ligands are arrayed along the backbone of a polymer thereby imparting hydrophobic properties to the polymer to which it is linked. In some embodiments, the hydrophobic ligand is a pattern recognition receptor agonist that has limited water solubility and may therefore be described as hydrophobic. In additional embodiments, the hydrophobic ligand is a TLR-7 or TLR-7/8 agonist, such as an imidazoquinoline.

Immune response: An immune response is a change in the activity of a cell of the immune system, such as a B cell, T cell, or monocyte, as a result of a stimulus, either directly or indirectly, such as through a cellular or cytokine intermediary. In one embodiment, the response is specific for a particular antigen (an “antigen-specific response”). In one embodiment, an immune response is a T cell response, such as a CD4 T cell response or a CD8 T cell response. In one embodiment, an immune response results in the production of additional T cell progeny. In one embodiment, an immune response results in the movement of T cells. In another embodiment, the response is a B cell response, and results in the production of specific antibodies or the production of additional B cell progeny. In other embodiments, the response is an antigen-presenting cell response. “Enhancing an immune response” refers to co-administration of an adjuvant and an immunogenic agent, such as a peptide antigen, as part of a peptide antigen conjugate, wherein the adjuvant increases the desired immune response to the immunogenic agent compared to administration of the immunogenic agent to the subject in the absence of the adjuvant. In some embodiments, an antigen is used to stimulate an immune response leading to the activation of cytotoxic T cells that kills virally infected cells or cancerous cells. In some embodiments, an antigen is used to induce tolerance or immune suppression. A tolerogenic response may result from the unresponsiveness of a T cell or B cell to an antigen. A suppressive immune response may result from the activation of regulatory cells, such as regulatory T cells that downregulate the immune response, i.e. dampen the immune response. Antigens administered to a patient in the absence of an adjuvant are generally tolerogenic or suppressive and antigens administered with an adjuvant are generally stimulatory and lead to the recruitment, expansion and activation of immune cells.

Immunogenic composition: A formulation of materials comprising an antigen and optionally an adjuvant that induces a measurable immune response against the antigen.

Ligand: Is a general term to describe any molecule that binds to a biological receptor. A pattern recognition receptor agonist is a specific type of Ligand that binds to a pattern recognition receptor (PRR) and may also be referred to as an adjuvant or Ligand with adjuvant properties. For instance, a PRR agonist is a Ligand that binds to a PRR, such as a TLR. A Ligand that binds to a PRR (or PRRa) may also be referred to as an adjuvant, molecular adjuvant, adjuvant molecule or Ligand with adjuvant properties. A Ligand that has limited solubility in water may be referred to as a hydrophobic Ligand, while a ligand that is water-soluble may be referred to as a hydrophilic Ligand. A hydrophobic ligand or hydrophilic ligand that has adjuvant properties may be referred to as a hydrophobic adjuvant or hydrophilic adjuvant, respectively.

Linked or coupled: The term “linked” or “coupled” means joined together, either directly or indirectly. A first moiety may be covalently or non-covalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker. The molecules may be linked indirectly by interposition of a component that binds non-covalently to both molecules independently.

As used herein, “linked” and variations thereof, refer to maintaining molecules in chemical or physical association, including after immunization, at least until they contact a cell, particularly an immune cell.

In some embodiments, linked components are associated so that the components are not freely dispersible from one another, at least until contacting a cell, such as an immune cell. For example, two components may be covalently linked to one another so that the two components are incapable of separately dispersing or diffusing. In preferred embodiments, peptide antigen conjugates are comprised of peptide antigens (A) that are covalently linked to a hydrophobic block (H) either directly or indirectly via an extension (B1 or B2). Peptide antigen conjugates comprising a hydrophobic block (H) assemble into particles in aqueous conditions, wherein two or more peptide antigen conjugates associate to form a stable wherein the individual peptide antigen conjugates and components comprising the peptide antigen conjugates are incapable of dispersing or diffusing prior to encountering a cell, such as an immune cell.

Linking is specifically distinguished from a simple mixture of antigen and adjuvant such as may be found, for example, in a conventional vaccine, for example a vaccine that contains a water-soluble peptide antigen mixed with an adjuvant. In a simple mixture, the components can be free to independently disperse within the vaccinated tissue and beyond.

Net charge: The sum of electrostatic charges carried by a molecule or, if specified, a section of a molecule.

Particle: A nano- or micro-sized supramolecular structure comprised of an assembly of molecules. Peptide antigen conjugates of the present disclosure comprise peptide antigens (A) linked to hydrophobic blocks (H) that assemble into micelles or other supramolecular structures or exist as pre-formed particles at the time of attachment. Particles comprising peptide antigen conjugates can be taken up into cells (e.g., immune cells, such as antigen-presenting cells). In some embodiments, the peptide antigen conjugate forms a particle in aqueous solution. In some embodiments, particle formation by the peptide antigen conjugate is dependent on pH or temperature. In some embodiments, the nanoparticles comprised of peptide antigen conjugates have an average diameter between 5 nanometers (nm) to 500 nm. In some embodiments, the nanoparticles comprised of peptide antigen conjugates may be larger than 100 nm. In some embodiments, the nanoparticles comprised of peptide antigen conjugates are included in larger particle structures that are too large for uptake by immune cells (e.g., particles larger than about 5000 nm) and slowly release the smaller nanoparticles comprising the peptide antigen conjugate

In some embodiments, the peptide antigen conjugates comprising a hydrophobic block (H) form nanoparticles. The nanoparticles form by association of peptide antigen conjugates through hydrophobic interactions and may therefore be considered a supramolecular assembly. In some embodiments, the nanoparticle is a micelle. In preferred embodiments, the nanoparticle micelles are between about 5 to 50 nm in diameter. In some embodiments, the peptide antigen conjugate forms micelles and the micelle formation is temperature-, pH- or both temperature- and pH-dependent. In some embodiments, the disclosed nanoparticles comprise peptide antigen conjugates that are comprised of peptide antigens (A) linked to a hydrophobic block (H) comprised of polymers linked to a Ligand with adjuvant properties, e.g. a PRR agonist; linking the peptide antigen together with the PRR agonist in the nanoparticles prevents the PRR agonist from dispersing freely following administration to a subject thereby preventing systemic toxicity.

The particle may be formed by an assembly of individual molecules comprising the peptide antigen conjugates, or in the case of a peptide antigen conjugate comprised of a peptide antigen (A) linked to a pre-formed particle, the particle may be cross-linked through covalent or non-covalent interactions.

Peptide or polypeptide: Two or more natural or non-natural amino acid residues that are joined together through an amide bond. The amino acid residues may contain post-translational modification(s) (e.g., glycosylation and/or phosphorylation). Such modifications may mimic post-translational modifications that occur naturally in vivo or may be non-natural. Any one or more of the components of the peptide antigen conjugate may be comprised of peptides.

There is no conceptual upper limit on the length of a peptide. The length of the peptide is typically selected depending on the application. In several embodiments, the hydrophobic block (H) is comprised of a peptide that can be between 3 to 1,000 amino acids in length, typically no more than 300 amino acids in length. In some embodiments, the N- and/or C-terminal extension (B1 and/or B2) is a peptide between about 1 to 8 amino acids in length. In some embodiments, the charged moiety (C) is a peptide comprised of positively, negatively or both positively and negatively charged amino acids and is typically no more than 16 amino acids in length.

In preferred embodiments, the peptide antigen (A) is a peptide between 5 to about 50 amino acids, typically about 7 to 35 amino acids, such as 7, 8, 9, 10, 11, 12, 13, 14 15, 16, 17, 18, 19, 20, 21, 22, 23, 24, 25, 26, 27, 28, 29, 30, 31, 32, 33, 34, 35 amino acids. In other embodiments, the peptide antigen (A) is about 50 amino acids or more in length. Thus, in some embodiments, the peptide antigen (A) may be considered a protein.

Note that the peptide antigen (A) may be a minimal epitope (sometimes referred to as min or ME) or long peptide (sometimes referred to as an LP or SLP) that comprises a minimal epitope. Therefore, it is understood that when a minimal epitope or long peptide is said to be delivered as a peptide antigen conjugate, then the minimal epitope or the long peptide is the peptide antigen (A), unless stated otherwise.

In some embodiments, the optional charged moiety (C), antigen (A), optional extensions (B1 and B2), and linker precursor X1 are amino acids and may be prepared by solid phase peptide synthesis as a contiguous peptide sequence that is sometimes referred to as a “peptide antigen fragment.” Note that calculation of the net charge or GRAVY of the peptide antigen fragment does not include the linker precursor X1.

Peptide sequences referring to the peptide antigen (A) are designated as “PA”, peptide sequences referring to the N-terminal extension (B1) are designated as “PN”, and peptide sequences referring to the C-terminal extension (B2) are designated as “PC”. Sequences of amino acids comprising peptide antigens (A) are represented by the formula, PA1 . . . PAn, where PA represents any amino acid residue comprising a peptide antigen (A) and n is an integer value. For example, an 8-amino acid peptide antigen (A) may be represented as PA1-PA2-PA3-PA4-PA5-PA6-PA7-PA8. Sequences of amino acids comprising N-terminal extensions (B1) are represented by the formula, PN . . . PNn, where PN represents any amino acid residue comprising an N-terminal extension and n is an integer value. Sequences of amino acids comprising C-terminal extensions (B2) are represented by the formula, PC1 . . . PCn, where PC represents any amino acid residue comprising a C-terminal extension and n is an integer value.

Peptide Modifications: Peptides may be altered or otherwise synthesized with one or more of several modifications as set forth below. In addition, analogs (non-peptide organic molecules), derivatives (chemically functionalized peptide molecules obtained starting from a peptide) and variants (homologs) of these peptides can be utilized in the methods described herein. The peptides described herein are comprised of a sequence of amino acids, analogs, derivatives, and variants, which may be either L- and/or D-versions. Such peptides may contain peptides, analogs, derivatives, and variants that are naturally occurring and otherwise.

Peptides can be modified through a variety of chemical techniques to produce derivatives having essentially the same activity as the unmodified peptides, and optionally having other desirable properties. For example, carboxylic acid groups of the peptide, whether at the carboxyl terminus or at a side chain, can be provided in the form of a salt of a pharmaceutically-acceptable cation or esterified to form a CC₁-C₁₆ ester, wherein CC refers to a carbon chain (and thus, CC1 refers to a single carbon and CC16 refers to 16 carbons), or converted to an amide. Amino groups of the peptide, whether at the amino terminus or at a side chain, can be in the form of a pharmaceutically-acceptable acid addition salt, such as the HCl, HBr, acetic, trifluoroacetic, formic, benzoic, toluene sulfonic, maleic, tartaric and other organic salts, or can be modified or converted to an amide.

An amino acid can be modified such that it contains through a covalent linkage a PRR agonist, such as TLR agonist, e.g., an imidazoquinoline-based TLR-7 or TLR-7/8 agonist.

Peptides may be modified to contain substituent groups that contain a positive or negative charge or both. The positive and/or negative charge may be affected by the pH at which the peptide is present.

Hydroxyl groups of the peptide side chains may be converted to CC₁-C₁₆ alkoxy or to a CC₁-CC₁₆ ester using well-recognized techniques, or the hydroxyl groups may be converted (e.g., sulfated or phosphorylated) to introduce negative charge. Phenyl and phenolic rings of the peptide side chains may be substituted with one or more halogen atoms, such as fluorine, chlorine, bromine or iodine, or with CC₁-CC₁₆ alkyl, CC₁-C₁₆ alkoxy, carboxylic acids and esters thereof, or amides of such carboxylic acids. Methylene groups of the peptide side chains can be extended to homologous CC₂-CC₄ alkylenes. Thiols can be used to form disulfide bonds or thioethers, for example through reaction with a maleimide. Thiols may be protected with any one of a number of well-recognized protecting groups, such as acetamide groups. Those skilled in the art will also recognize methods for introducing cyclic structures into the peptides of this invention to select and provide conformational constraints to the structure that result in enhanced stability. Reference may be made to Greene et al., “Greene's Protective Groups in Organic Synthesis” Fourth Edition, John Wiley & Sons, Inc. 2006 for details of additional modifications that can be made to functional groups.

Peptidomimetic and organomimetic embodiments of the peptide antigen (A) are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic peptide having measurable ability to induce tolerance or immune suppression, or enhanced ability to generate a stimulatory immune response, such as cytotoxic T cell or antibody response.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, such as one or more therapeutic cancer vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polar: A description of the properties of matter. Polar is a relative term, and may describe a molecule or a portion of a molecule that has partial charge that arises from differences in electronegativity between atoms bonded together in a molecule, such as the bond between nitrogen and hydrogen. Polar molecules have a preference for interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may have a preference for interacting with a polar solvent such as water. In specific, non-limiting cases, introduction of additional polar groups may increase the solubility of a portion of a molecule.

Polymer: A molecule containing repeating structural units (monomers). As described in greater detail throughout the disclosure, polymers may be used for any number of components of the peptide antigen conjugate and may be natural or synthetic. In preferred embodiments, a hydrophobic or amphiphilic polymer is used as the hydrophobic block (H) and drives particle assembly of the peptide antigen conjugates. In some embodiments, the peptide antigen (A) is a polymer comprising amino acids. In some embodiments, the extensions (B1 and B2) comprise polymers, such as, for example, PEG, poly(amino acids) or combinations thereof. The polymers included in the disclosed embodiments can form polymer nanoparticles that can be administrated to a subject without causing adverse side effects. The polymers included in the disclosed embodiments can form polymer nanoparticles that can be administered to a subject to cause an immune response or to treat and/or ameliorate a disease. The polymers included in the disclosed embodiments may include a side chain with a functional group that can be utilized, for example, to facilitate linkage to an adjuvant or a molecule used to induce immune suppression or tolerance, such as macrolides, e.g., rapamycin. In several embodiments, the polymer can contain two or more polymer blocks linked through a linker to create a block co-polymer, such as an amphiphilic di-block co-polymer. In several embodiments, a polymer block may be predominantly hydrophobic in character. In several embodiments, the polymer consists of peptides, their analogs, derivatives, and variants. Various compositions of polymers useful for the practice of the invention are discussed in greater detail elsewhere.

Polymerization: A chemical reaction, usually carried out with a catalyst, heat or light, in which monomers combine to form a chainlike, or cross-linked, macromolecule (a polymer). The chains further can be combined by additional chemical synthesis using the appropriate substituent groups and chemical reactions. The monomers may contain reactive substances. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomers resulting in the splitting out of a water molecule. In other forms of polymerization, a monomer is added one at a time to a growing chain through the staged introduction of activated monomers, such as during solid phase peptide synthesis.

Purified: Having a composition that is relatively free of impurities or substances that adulterate or contaminate a substance. The term purified is a relative term and does not require absolute purity. Thus, for example, a purified peptide antigen conjugate is one in which the peptide antigen conjugate is more enriched than the peptide antigen conjugate is in the product solution resulting from a reaction between the hydrophobic block fragment and peptide antigen fragment. In one embodiment, a peptide antigen conjugate is purified such that the peptide antigen conjugate represents at least 50% of the purified material. In some embodiments, the peptide antigen conjugate is at least 60%, 70%, 80%, 90%, 95%, 98%, or 99% pure. Purity may be determined by a variety of different methods. Typically purity is determined by HPLC, elemental analysis or amino acid analysis or a combination thereof.

Soluble: Capable of becoming molecularly or ionically dispersed in a solvent to form a homogeneous solution. When referring to a peptide, a soluble peptide is understood to be a single molecule in solution that does not assemble into multimers or other supramolecular structures through hydrophobic or other non-covalent interactions. A soluble molecule is understood to be freely dispersed as single molecules in solution. In several embodiments, a peptide antigen can be a soluble peptide antigen that dissolves up to at least 0.1 mg/ml in phosphate buffered saline, pH 7.4 at room temperature. In other embodiments, a peptide antigen conjugate may be soluble in dimethylsulfoxide and/or other organic solvent(s) at room temperature, but may not be soluble in aqueous solvent(s), such as phosphate buffered saline, at pH 7.4 at room temperature. Hydrophobic molecules (e.g., hydrophobic blocks (H)) described herein are insoluble down to about 0.1 mg/mL. Solubility can be determined by visual inspection, by turbidity measurements or by dynamic light scattering.

Subject: Refers to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Supramolecular: Refers to two or more molecules that associate through non-covalent interactions. In some embodiments, the molecules associate due to hydrophobic interactions. In some embodiments, the molecules associate due to electrostatic interactions. The association confers a new property to the supramolecular complex that was not shared by either of the constituent molecules, such as increased size, which affects the materials interactions with the immune system and different immune responses. For example, peptide antigen conjugates may aggregate to form supramolecular complexes.

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Telechelic: Is used to describe a polymer that has one or two reactive ends that may be the same or different. The word is derived from telos and chele, the Greek words for end and claw, respectively. A semi-telechelic polymer describes a polymer with only a single end group, such as a reactive functional group that may undergo additional reactions, such as polymerization. A hetero-telechelic polymer describes a polymer with two end groups, such as reactive functional groups, that have different reactive properties.

Herein, hydrophobic blocks (H) may be comprised of polymers with reactive groups at one or both ends. In some embodiments, an adjuvant is placed at one end of the polymer and the other end of the polymer may be reacted with a linker that is linked to a peptide antigen (A) directly or indirectly through an extension (B1 or B2) or a Linker (L). In this example, the polymer is semi-telechelic with respect to the adjuvant, meaning the adjuvant is attached to only one end of the polymer chain comprising the hydrophobic block (H).

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

In one example, a desired response is to induce an immune response that leads to a reduction in the size, volume, rate of growth, or number (such as metastases) of a tumor in a subject. For example, the agent or agents can induce an immune response that decreases the size, volume, or number of tumors by a desired amount, for example by at least 5%, at least 10%, at least 15%, at least 20%, at least 25%, at least 30%, at least 50%, at least 75%, at least 90%, or at least 95% as compared to a response in the absence of the agent.

Tumor or cancer or neoplastic: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply, and may have induced the regulatory T cells (Tregs) and myeloid derived suppressor cells (MDSC).

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art.

Any functional group disclosed herein and/or defined above can be substituted or unsubstituted, unless otherwise indicated herein.

Unless otherwise explained, all technical and scientific terms used herein have the same meaning as commonly understood by one of ordinary skill in the art to which this disclosure belongs.

The singular terms “a,” “an,” and “the” include plural referents unless context clearly indicates otherwise. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

### Processes for Producing a Peptide Antigen Conjugate

The processes described herein are particularly suitable for the solution phase manufacture of peptide antigen conjugates of formula [C]-[B1]-A-[B2]-L-H, such as peptide antigen conjugates having the formula A-L-H, C-A-L-H, B1-A-L-H, A-B2-L-H, C-B1-A-L-H, C-A-B2-L-H, and/or C-B1-A-B2-L-H. Alternatively, the processes described herein are particularly suitable for the manufacture of peptide antigen conjugates of formula H-L-[B1]-A-[B2]-[C] such as peptide antigen conjugates having the formula H-L-A, H-L-A-C, H-L-B1-A, H-L-A-B2, H-L-B1-A-C, H-L-A-B2-C, and/or H-L-B1-A-B2-C. Still further, the processes described herein are particularly suitable for the manufacture of peptide antigen conjugates of the formula [B1]-A-[B2]-L-H-C and [B1]-A-[B2]-L(C)-H, such as peptide antigen conjugates having the formula A-L-H-C, B1-A-L-H-C, A-B2-L-H-C, B1-A-B2-L-H(C), A-L(C)-H, B1-A-L(C)-H, A-B2-L(C)-H and/or B1-A-B2-L(C)-H, wherein the parentheses indicate that the Linker (L) is linked to both the charged moiety (C) and the hydrophobic block (H).

In some embodiments, the peptide antigen (A) may be linked either directly or through an extension (B1 or B2) or charged moiety (C) to the hydrophobic block (H) to obtain a peptide antigen conjugate of formula [C]-[B1]-A-[B2]-[L]-H, [B1]-A-[B2]-[L](C)-H, [B1]-A-[B2]-[C]-[L]-H or [B1]-A-[B2]-[L]-H-(C), wherein the Linker (L) is optional. In these embodiments, the peptide antigen conjugates may be obtained by direct attachment of the peptide antigen (A) to the hydrophobic block (H) either directly or via an extension (B1 or B2) or the charged moiety (C) by solid-phase or solution-phase synthesis.

In some preferred embodiments, the peptide antigen fragment is reacted with the hydrophobic block fragment in a molar ratio of 1 or greater. The molar ratio of peptide antigen fragment to hydrophobic block fragment may be from about 1 to about 3, such as from about 1 to about 1.2. The molar ratio of peptide antigen fragment to hydrophobic block fragment may be 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0.

In preferred embodiments, the peptide antigen fragment is reacted with the hydrophobic block fragment in a pharmaceutically acceptable organic solvent to obtain a product solution. The pharmaceutically acceptable organic solvent can be any organic liquid that is known by the skilled person to be pharmaceutically acceptable and in which the hydrophobic block fragment and the peptide antigen fragment are sufficiently soluble to allow them to react with one another. Suitable pharmaceutically acceptable organic solvents include, but are not limited to, dimethyl sulfoxide (DMSO), methanol and ethanol. In specific embodiments, the pharmaceutically acceptable organic solvent is dimethyl sulfoxide (DMSO).

Unexpectedly our results show that hydrophobic block fragments and peptide antigen conjugates comprising hydrophobic blocks that comprise aromatic rings and heterocyclic aromatic rings, particularly those substituted with amines, i.e. aryl amines, exhibit improved solubility in pharmaceutically acceptable organic solvents as compared with hydrophobic blocks based on aliphatic groups, such as fatty acids, or cholesterol.

The reaction of the peptide antigen fragment and the hydrophobic block fragment may be carried out at a temperature of from about 20° C. to about 150° C., such as from about 20° C. to about 55° C., including but not limited to 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. It will be appreciated by those skilled in the art that the reaction temperature used may depend, at least in part, on the pharmaceutically acceptable organic solvent used and the reactivity of the first and second functional groups on linker precursor X1 and X2.

Under the reaction conditions the first and second functional groups on linker precursor X1 and X2 undergo a reaction to form Linker (L), as described in more detail later.

The product solution that is formed comprises a peptide antigen conjugate, e.g., a peptide antigen conjugate of formula [C]-[B1]-A-[B2]-L-H, unreacted hydrophobic block fragment of formula X2-H and pharmaceutically acceptable organic solvent. The peptide antigen conjugate may be purified from the product solution containing unreacted hydrophobic block fragment to obtain a purified peptide antigen conjugate, which may be provided as a lyophilized solid or may be suspended in a pharmaceutically acceptable organic solvent and stored as a purified peptide antigen conjugate solution. Unexpectedly, our results show that excess hydrophobic block fragment remaining in the product solution has no meaningful impact on particle size, stability, or in vivo activity of nanoparticle micelles formed by the peptide antigen conjugates, suggesting that it is not necessary to remove excess hydrophobic block fragment from the crude product solution and, therefore, that the reaction to form Linker (L) can safely be driven towards completion by using a large molar excess of the hydrophobic block fragment of formula X2-H if required.

The processes described herein are also particularly suitable for the solid phase manufacture of peptide antigen conjugates of formula [C]-[B1]-A-[B2]-H or H-[B1]-A-[B2]-[C]. For example, the processes described herein are particularly suitable for the solid phase manufacture of peptide antigen conjugates of formula [C]-[B1]-A-[B2]-H, such as peptide antigen conjugates having the formula A-H, C-A-H, B1-A-H, A-B2-H, C-B1-A-H, C-A-B2-H, and C-B1-A-B2-H. The processes described herein are also particularly suitable for the solid phase manufacture of peptide antigen conjugates of formula H-[B1]-A-[B2]-[C], such as peptide antigen conjugates having the formula H-A, H-A-C, H-B1-A, H-A-B2, H-B1-A-C, H-A-B2-C, and H-B1-A-B2-C.

The product solutions or purified peptide antigen conjugate solutions produced by the solution or solid phase processes may be stored for further use.

In some embodiments, solutions comprising peptide antigen conjugates, such as the product solutions or purified peptide antigen conjugate solutions, may be analyzed by UV-Vis spectroscopy or chromatographically to determine the absorbance or area-under-the-curve (absorbance over time in the chromatogram) associated with the peptide antigen conjugate and any unreacted hydrophobic block fragment. Unexpectedly, for peptide antigen conjugates comprising a hydrophobic block that comprises one or more aromatic groups, our results show that the relationship between absorbance and/or area-under-the-curve at a particular wavelength, e.g., wavelengths greater than about 300 nm, and the molar concentration of peptide antigen conjugate is approximately equivalent for each peptide antigen conjugate irrespective of the peptide antigen (A) sequence. These unexpected results show that chromatographic analysis, e.g., HPLC, in-line with a UV-Vis detector (e.g., multi-diode array or multi-wavelength detector), is a reliable method for assessing the molar concentration of the peptide antigen conjugate and any unreacted hydrophobic block fragment in the product solution or purified peptide antigen conjugate solution and that the same extinction coefficient may be applied for determining peptide antigen conjugate concentration irrespective of variations in peptide antigen (A) sequence. Thus, in preferred embodiments, the process for determining the molar amount of peptide antigen conjugate in a solution comprising one or more different peptide antigen conjugates is by UV-Vis spectroscopy or chromatography based on a pre-determined molar extinction coefficient that is independent of the peptide antigen composition at a wavelength greater than about 300 nm, between about 300-650 nm, typically about 300 to 350 nm, such as 300, 301, 302, 303, 304, 305, 306, 307, 308, 309, 310, 311, 312, 313, 314, 315, 316, 317, 318.319, 320, 321, 322, 323, 324, 325, 330, 340 or 350.

In preferred embodiments, addition of an aqueous medium, e.g., aqueous buffer such as PBS, to the product solution or purified peptide antigen conjugate solution results in the peptide antigen conjugate spontaneously assembling into stable nanoparticle micelles. Nanoparticle micelles may be of a particle size range that do not appreciably scatter visible light, whereas aggregated material scatters visible light that may be assessed by solution turbidity (i.e. absorbance) measurements. Therefore, in some embodiments, the process further comprises the analysis of the propensity of the purified peptide antigen conjugate solution or product solution comprising peptide antigen conjugate, any unreacted hydrophobic block fragment and pharmaceutically acceptable organic solvent to form aggregated material upon addition to an aqueous buffer, e.g., PBS at pH 7.4, by measuring solution turbidity.

The process of measuring solution turbidity comprises the steps of (i) aliquoting a specific volume of the product solution or purified peptide antigen conjugate solution from a first container to a second container; (ii) adding a volume of the aqueous buffer, e.g., PBS, to the second container to obtain an aqueous mixture of peptide antigen conjugate that is diluted to a concentration not lower than 0.01 mg/mL; (iii) assessing turbidity of the aqueous mixture by measuring absorbance of the aqueous mixture at a wavelength greater than 350 nm; and (iv) confirming the presence or absence of aggregated material in the aqueous mixture based on a comparison of the absorbance of the aqueous mixture as compared with the aqueous buffer alone. The wavelength used to assess turbidity should be selected from wavelengths of light that are not absorbed by the peptide antigen conjugate, any unreacted hydrophobic block or pharmaceutically acceptable organic solvent. In preferred embodiments, the wavelength of light selected to assess turbidity is typically selected from 350 to 650 nm. In preferred embodiments, the wavelength is selected from 350 to 450, such as 350, 360 370, 380, 390, 400, 410, 420, 430, 440 or 450 nm. Unexpectedly, turbidity measurements were found to be a reliable assay for assessing nanoparticle micellization, thus improving the efficiency for characterizing and releasing peptide antigen conjugates used for personalized therapies.

An unexpected finding disclosed herein is that while a product solution or a purified peptide antigen conjugate solution comprising a first peptide antigen conjugate may have the propensity to form aggregated material upon addition of an aqueous buffer, e.g., PBS, a peptide antigen conjugate mixture comprising the first peptide antigen conjugate and two or more additional peptide antigen conjugates may be less prone to forming aggregated material upon addition of an aqueous buffer. Based on these findings, processes described herein were developed that are particularly useful for selecting two or more peptide antigen conjugates to include in a peptide antigen mixture to prevent formation of aggregated material that is unsuitable for clinical use.

In preferred embodiments, the process of selecting two or more different compositions of peptide antigen conjugates to include in a peptide antigen conjugate mixture to be used for a personalized therapy comprises the step of determining the propensity of individual product solutions or individual purified peptide antigen conjugate solutions comprising individual peptide antigen conjugates to form aggregated material upon addition to an aqueous buffer for the full set of peptide antigen conjugates that are specific to each patient. The full set of peptide antigen conjugates that are specific to each patient may be between 1 and 100, typically between about 5 to 20, such as 5, 6, 7, 8, 9, 10, 11, 12, 13, 14, 15, 16, 17, 18, 19, or 20 unique peptide antigen conjugates. In preferred embodiments, the propensity of each peptide antigen conjugate to form aggregated material following addition of an aqueous buffer is assessed. The individual patient-specific peptide antigen conjugates are then aliquoted and mixed together depending on the propensity of the individual peptide antigen conjugates to form aggregated material, such that the molar amount of each of any peptide antigen conjugate that forms aggregated material comprises less than 60% of the total molar amount of peptide antigen conjugates in the peptide antigen conjugate mixture.

In preferred embodiments, a peptide antigen conjugate mixture comprises two or more unique peptide antigen conjugates, such as between 2 to 20, and no more than between 60% of the total molar amount of peptide antigen conjugates in the peptide antigen conjugate mixture are prone to aggregation, such as from about 0, 5, 10, 15, 20, 25, 30, 40, 50 and 60% of the total molar amount of peptide antigen conjugates are prone to aggregation.

The peptide antigen conjugate mixture may be stored for further use. The further use may comprise the steps of analysing the peptide antigen conjugate mixture by UV-Vis spectroscopy or chromatography (in-line with a UV-Vis detector) to determine the molar concentration of each of the two or more peptide antigen conjugates and any unreacted hydrophobic block fragment in the mixture; and/or evaluation of the propensity of the peptide antigen conjugate mixture to form aggregated material upon addition of an aqueous buffer, e.g., PBS.

The product solution, purified peptide antigen conjugate solution or peptide antigen conjugate mixture may be stored for further use. The further use may comprise sterile filtering the product solution, purified peptide antigen conjugate solution or peptide antigen conjugate mixture to provide a sterile product solution, sterile purified peptide antigen conjugate solution or sterile peptide antigen conjugate mixture comprising peptide antigen conjugate(s), any unreacted hydrophobic block fragment and pharmaceutically acceptable organic solvent. While the peptide antigen conjugates are administered to subjects as aqueous mixtures in preferred embodiments, an unexpected finding disclosed herein is that sterile filtering the product solution, purified peptide antigen conjugate or peptide antigen conjugate mixture prior to addition of aqueous buffer (while the peptide antigen conjugate is in a pharmaceutically acceptable organic solvent) results in improved material recovery as compared with sterile filtering aqueous mixtures of peptide antigen conjugate(s). Thus, in preferred embodiments, the process of preparing a personalized therapy comprises the step of sterile filtering the product solution, purified peptide antigen conjugate or peptide antigen conjugate mixture prior to the addition of aqueous buffer.

The sterile product solution, sterile purified peptide antigen conjugate solution or sterile peptide antigen conjugate mixture may be lyophilized and stored for further use.

The sterile peptide antigen conjugate solution, sterile purified peptide antigen conjugate solution or sterile peptide antigen conjugate mixture may be mixed with aqueous buffer, e.g., PBS to obtain a sterile aqueous solution of peptide antigen conjugate particles. Thus, in certain embodiments the process further comprises adding an excess volume of aqueous buffer to the sterile peptide antigen conjugate solution, followed by mixing, to generate an aqueous mixture comprising stable nanoparticle micelles comprising the peptide antigen conjugate(s), any unreacted hydrophobic block fragment and pharmaceutically acceptable organic solvent.

Unexpectedly, our results show that stable nanoparticle micelles are generated by simply adding aqueous buffer (e.g., PBS buffer) to peptide antigen conjugates in pharmaceutically acceptable organic solvents (e.g., DMSO) and that it is not necessary to remove the organic solvent. In these embodiments, the sterile peptide antigen conjugate solution, sterile purified peptide antigen conjugate solution or sterile peptide antigen conjugate mixture, may comprise up to about 50% (v/v) DMSO, such as up to 12.5% (v/v) DMSO, including but not limited to 0.1 (v/v), 0.25% (v/v), 0.5% (v.v), 1% (v/v), 1.5% (v/v), 2% (v/v), 2.5% (v/v), 3% (v/v), 3.5% (v/v), 4% (v/v), 4.5% (v/v), 5% (v/v), 5.5% (v/v), 6% (v/v), 6.5% (v/v), 7% (v/v), 7.5% (v/v), 8% (v/v), 8.5% (v/v), 9% (v/v), 9.5% (v/v), 10% (v/v), 10.5% (v/v), 11% (v/v), 11.5% (v/v), 12% (v/v) or 12.5% (v/v). Thus, this approach offers a simple and reliable method for generating peptide antigen conjugates particles from amphiphilic compounds.

### Peptide Antigens (A)

Peptide antigens for use in embodiments of the present disclosure may be selected from pathogens, cancerous cells, auto-antigens or allergens. In some embodiments, the peptide-based antigen can include a region of a polypeptide or protein from a pathogen (such as a virus, bacteria, or fungi) or a tissue of interest (such as a cancerous cell). In other embodiments, the antigen can be a whole protein or glycoprotein derived from a pathogen, or a peptide or glycopeptide fragment of the protein or glycoprotein. In other embodiments, the antigen can be a protein, or peptide fragments of a protein, that is expressed primarily by tumor tissue (but not healthy tissue) and is a tumor-associated antigen. In other embodiments, the antigen is a protein or peptide that is associated with auto-immunity. In still other embodiments, the antigen is a foreign protein or glycoprotein that is associated with allergies. The peptide antigen (A) may be any antigen that is useful for inducing an immune response in a subject. The immune response may be either pro-inflammatory or tolerogenic depending on the nature of the antigen and the desired immune response. In some embodiments, the antigen (A) is a tumor-associated antigen, such as a self-antigen or neoantigen. In other embodiments, the antigen (A) is an infectious disease antigen, such as an antigen derived from a virus, bacteria, fungi or protozoan microbial pathogen. In still other embodiments, the antigen (A) is a peptide derived from an allergen or a self-antigen mediating auto-immunity.

The peptide antigen (A) is comprised of a sequence of amino acids or a peptide mimetic that can induce an immune response, such as a T cell or B cell response in a subject. In some embodiments, the peptide antigen (A) comprises an amino acid or amino acids with a post-translational modification, non-natural amino acids or peptide-mimetics. The peptide antigen may be any sequence of natural, non-natural or post-translationally modified amino acids, peptide-mimetics, or any combination thereof, that have an antigen or predicted antigen, i.e. an antigen with a T cell or B cell epitope.

Immunogenic compositions may comprise one or more different peptide antigen conjugates each having a different peptide antigen (A) composition. In some embodiments, the immunogenic compositions comprise particles with up to 50 different peptide antigen conjugates each having a unique peptide antigen (A) composition. In some embodiments, the immunogenic compositions comprise mosaic particles that comprise 20 different peptide antigen conjugates. In other embodiments, the immunogenic compositions comprise mosaic particles that comprise 5 different peptide antigen conjugates. In some embodiments, the immunogenic compositions comprise 20 different particle compositions each assembled from a unique peptide antigen conjugate (i.e. each particle contains a single peptide antigen conjugate composition). In other embodiments, the immunogenic compositions comprise 5 different particle compositions each assembled from a unique peptide antigen conjugate (i.e. each particle contains a single peptide antigen conjugate composition). In still other embodiments, the immunogenic compositions comprise a single particle composition comprised of a single peptide antigen conjugate composition.

The length of the peptide antigen (A) depends on the specific application and is typically between about 5 to about 50 amino acids. In preferred embodiments, the peptide antigen (A) is between about 7 to 35 amino acids, e.g., 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. In other embodiments, the peptide antigen is a fragment of a polypeptide. In still other cases, the peptide antigen is a full-length polypeptide, such as a protein antigen that may be recombinantly expressed. Peptide antigens (A) based on tumor-associated antigens, infectious disease antigens, allergens or auto-antigens may be delivered as the full-length sequence, though preferably no more than 50 amino acids in length. In preferred embodiments, the peptide antigen (A) is 7 to 35 amino acids, typically about 25. Thus, for a tumor-associated antigen, infectious disease antigen, allergen or auto-antigen greater than 25 amino acids in length, e.g., a 100 amino acid antigen, the antigen may be divided into 7 to 35 amino acid, e.g., 25 amino acid, peptide antigens (A) wherein each peptide antigen (A) contains a unique composition of amino acids; or, the peptide antigens (A) can be overlapping peptide pools wherein an antigen is divided into a set number of 7 to 35 amino acid, e.g., 25 amino acid, peptide antigens (A) that have overlapping sequences. For example, an overlapping peptide pool comprising a 100 amino acid antigen may be divided into eight 25 amino acid peptide antigens (A) that are each offset by 12 amino acids (i.e., each subsequent 25 amino acid peptide comprising a 100 amino acid peptide sequence starts at the₁₃th amino acid position from the prior peptide). Those skilled in the art understand that many permutations exist for generating a peptide pool from an antigen

In some embodiments, the peptide antigen (A) is a minimal CD8 or CD4 T cell epitope that comprises the portions of a tumor-associated antigen, infectious disease antigen, allergen or auto-antigen that are predicted in silico (or measured empirically) to bind MHC-I or MHC-II molecules. For tumor-associated antigens, the peptide antigen (A) that is a minimal CD8 or CD4 T cell epitope that is predicted in silico (or measured empirically) to bind MHC-I or MHC-II molecules should also be a sequence of amino acids that is unique to the tumor cell. Algorithms for predicting MHC-I or MHC-II binding are widely available (see Lundegaard et al., Nucleic Acids Res., 36:W509-W512, 2008 and http://www.cbs.dtu.dk/services/NetMHC/). In some embodiments of a personalized therapy for a particular subject (e.g., patient), the peptide antigen (A) comprising a peptide antigen conjugate may comprise a minimal CD8 T cell epitope from a tumor-associated antigen, infectious disease antigen, allergen or auto-antigen that is typically a 7-13 amino acid peptide that is predicted to have <1,000 nM binding affinity for a particular MHC-I allele that is expressed by that subject. In some embodiments of a personalized therapy for a particular subject (e.g., patient), the peptide antigen (A) may comprise a minimal CD4 T cell epitope from a tumor-associated antigen, infectious disease antigen, allergen or auto-antigen that is a 10-16 amino acid peptide that is predicted to have <1,000 nM binding affinity for a particular MHC-II allele that is expressed by that subject. In a preferred embodiment, when a minimal CD8 or CD4 T cell epitope cannot be identified for a tumor-associated antigen, infectious disease antigen, allergen or auto-antigen, or when the tumor-associated antigen, infectious disease antigen, allergen or auto-antigen contains multiple CD8 and CD4 T cell epitopes, the peptide antigen (A) may be between 16-35 amino acids or may be up to 50 amino acids, e.g., up to 35 amino acids, up to 25 amino acids, or up to 20 amino acids, or up to 16 amino acids such that it may contain all possible CD8 or CD4 T cell epitopes.

In some embodiments of the present disclosure, the peptide antigen (A) is derived from tumor-associated antigens. Tumor-associated antigens can either be self-antigens that are present on healthy cells but are preferentially expressed by tumor cells, or neoantigens, which are aberrant proteins that are specific to tumor cells and are unique to individual patients. Suitable self-antigens include antigens that are preferentially expressed by tumor cells, such as CLPP, Cyclin-A1, MAGE-A1, MAGE-C1, MAGE-C2, SSX2, XAgE1b/GAGED2a, Melan-A/MART-1, TRP-1, Tyrosinase, CD45, glypican-3, IGF2B3, Kallikrein 4, KIF20A, Lengsin, Meloe, MUC5AC, surviving, prostatic acid phosphatase, NY-ESO-1 and MAGE-A3. Neoantigens arise from the inherent genetic instability of cancers, which can lead to mutations in DNA, RNA splice variants and changes in post-translational modification, all potentially leading to de novo protein products that are referred to collectively as neoantigens or sometimes predicted neoantigens. DNA mutations include changes to the DNA including nonsynonymous missense mutations, nonsense mutations, insertions, deletions, chromosomal inversions and chromosomal translocations, all potentially resulting in novel gene products and therefore neoantigens. RNA splice site changes can result in novel protein products and missense mutations can introduce amino acids permissive to post-translational modifications (e.g. phosphorylation) that may be antigenic. The instability of tumor cells can furthermore result in epigenetic changes and the activation of certain transcription factors that may result in selective expression of certain antigens by tumor cells that are not expressed by healthy, non-cancerous cells.

Peptide antigen conjugates used in personalized cancer vaccines should include peptide antigens (A) that comprise the portions of tumor-associated antigens that are unique to tumor cells. Peptides antigens (A) comprising neoantigens arising from a missense mutation should encompass the amino acid change encoded by 1 or more nucleotide polymorphisms. Peptides antigens (A) comprising neoantigens that arise from frameshift mutations, splice site variants, insertions, inversions and deletions should encompass the novel peptide sequences and junctions of novel peptide sequences. Peptides antigens (A) comprising neoantigens with novel post-translational modifications should encompass the amino acids bearing the post-translational modification(s), such as a phosphate or glycan. In preferred embodiments, the peptide antigen (A) comprises the 0-25 amino acids on either side flanking the amino acid change or novel junction that arises due to a mutation. In one embodiment, the peptide antigen (A) is a neoantigen sequence that comprises the 12 amino acids on either side flanking the amino acid change that arises from a single nucleotide polymorphism, for example, a 25 amino acid peptide, wherein the 13^{t}h amino acid is the amino acid residue resulting from the single nucleotide polymorphism. In some embodiments, the peptide antigen (A) is a neoantigen sequence that comprises the 12 amino acids on either side flanking an amino acid with a novel post-translational modification, for example, a 25 amino acid peptide, wherein the₁₃th amino acid is the amino acid residue resulting from the novel post-translational modification site. In other embodiments, the peptide antigen (A) is a neoantigen sequence that comprises 0-12 amino acids on either side flanking a novel junction created by an insertion, deletion or inversion. In some cases, the peptide antigen (A) comprising neoantigens resulting from novel sequences can encompass the entire novel sequence, including 0-25 amino acids on either side of novel junctions that may also arise.

Tumor-associated antigens suitable as peptide antigens (A) for immunogenic compositions of the present disclosure can be identified through various techniques that are familiar to one skilled in the art. Tumor-associated antigens can be identified by assessing protein expression of tumor cells as compared with healthy cells, i.e., non-cancerous cells from a subject. Suitable methods for assessing protein expression include but are not limited to immunohistochemistry, immunofluorescence, western blot, chromatography (i.e., size-exclusion chromatography), ELISA, flow cytometry and mass spectrometry. Proteins preferentially expressed by tumor cells but not healthy cells or by a limited number of healthy cells (e.g., CD20) are suitable tumor-associated antigens. DNA and RNA sequencing of patient tumor biopsies followed by bio-informatics to identify mutations in protein-coding DNA that are expressed as RNA and produce peptides predicted to bind to MHC-I alleles on patient antigen presenting cells (APCs), may also be used to identify tumor-associated antigens that are suitable as peptide antigens (A) for immunogenic compositions of the present disclosure.

In some embodiments, tumor-associated antigens suitable as peptide antigens (A) for immunogenic compositions are identified using mass spectrometry. Suitable peptide antigens (A) are peptides identified by mass spectrometry following elution from the MHC molecules from patient tumor biopsies but not from healthy tissues from the same subject (i.e., the peptide antigens are only present on tumor cells but not healthy cells from the same subject). Mass spectrometry may be used alone or in combination with other techniques to identify tumor-associated antigens. Those skilled in the art recognize that there are many methods for identifying tumor-associated antigens, such as neoantigens (see Yadav et al., Nature, 515:572-576, 2014) that are suitable as peptide antigens (A) for the practice of the present disclosure.

In certain embodiments, the tumor-associated antigens used as peptide antigens (A) are clonal or nearly clonal within the population of neoplastic cells, which may be considered heterogeneous in other respects.

Tumor-associated antigens selected for use as peptide antigens (A) in personalized cancer vaccination schemes may be selected based on mass spectrometry confirmation of peptide-MHC binding and/or in silico predicted MHC binding affinity and RNA expression levels within tumors. These data provide information on whether or not a tumor-associated antigen is expressed and presented by tumor cells and would therefore be a suitable target for T cells. Such criteria may be used to select the peptide antigens (A) used in a personalized cancer vaccine. Personalized cancer vaccines may contain 1-100 unique peptide antigens (A), such as neoantigens, of between 8-50 amino acids in length. In preferred embodiments, 20 peptide antigens (A) of between 8-16 amino acids are used, such as 8, 9, 10, 11, 12, 13, 14, 15 or 16 amino acids. In other embodiments, 20 peptide antigens (A) of between 16-35 amino acids, are used, each peptide antigen (A) comprising 16, 17, 18, 19, 20, 21, 22, 23, 24, 25, 26, 27, 28, 29, 30, 31, 32, 33, 34, or 35 amino acids, typically no more than 50. In a non-limiting example, 20 peptide antigens (A) of 8 amino acids in peptide sequence length are used as a personalized cancer vaccine. In another non-limiting example, immunogenic compositions comprised of particles formed from 20 different peptide antigen conjugates comprised of peptide antigens (A) of 25 amino acids in length are used as a personalized cancer vaccine. In another non-limiting example, 20 peptide antigens (A) of 8-16 amino acids in peptide sequence length and a peptide antigen (A) comprising a universal CD4 T helper are used as a personalized cancer vaccine.

For patients with highly mutated tumors that have more than 50 tumor-associated neoantigens, a down-selection process may be used to select peptide antigens (A) for use in personalized cancer vaccines comprised of peptide antigen conjugates. In some embodiments, a down-selection process is used to select peptide antigens (A) comprising epitopes predicted to have the highest MHC binding affinity and RNA expression levels within tumor cells. Additional criteria may be applied for the selection of tumor-associated self-antigens or neoantigens. For example, predicted immunogenicity or predicted capacity of the peptide antigen (A) to lead to T cells that react with other self-antigens, which may lead to auto-immunity, are additional criteria considered. For instance, peptide antigens (A) that comprise tumor-associated antigens and have high predicted immunogenicity but also low potential to lead to auto-immunity are criteria used to select potential peptide antigens (A) for use in personalized cancer vaccines. In some embodiments, neoantigens that would be expected to result in T cell or antibody responses that react with self-antigens found on healthy cells are not selected for use as peptide antigens (A). For patients with less than, for example, 20-50 predicted neoantigens, a down selection process may not be critical and so all 20-50 predicted neoantigens might be used as peptides antigens (A) in a personalized cancer vaccine.

Cancer vaccines may include peptide antigens (A) that comprise tumor-associated antigens that are patient-specific and/or tumor-associated antigens that are shared between patients. For example, the tumor-associated antigen can be a conserved self-antigen, such as NY-ESO-1 (testicular cancer) or gp100 (melanoma), or the antigen may be a cryptic epitope, such as Na17 (melanoma) that is not typically expressed by healthy cells but is conserved between patients. Immunogenic compositions of the present disclosure may include peptide antigens (A) that arise from so-called hot-spot mutations that are frequent mutations in certain genes or gene regions that occur more frequently than would be predicted by chance. Non-limiting examples of hot spot mutations include the V600E mutation in BRAF protein, which is common to melanoma, papillary thyroid and colorectal carcinomas, or KRAS G12 mutations, which are among the most common mutations, such as KRAS G12C. A number of suitable self-antigens as well as neoantigens that arise from hotspot mutations are known and are incorporated herein by reference: see Chang et al., Nature Biotechnology, 34:155-163, 2016; Vigneron, N., et al, Cancer Immunology, 13:15-20, 2013.

In some embodiments, the peptide antigen (A) can be from a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the peptide antigen (A) can be from a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the peptide antigen (A) is a tumor-associated antigen from a breast cancer, such as a ductal carcinoma or a lobular carcinoma. In some embodiments, the peptide antigen (A) is a tumor-associated antigen from a prostate cancer. In some embodiments, the peptide antigen (A) is a tumor-associated antigen from a skin cancer, such as a basal cell carcinoma, a squamous cell carcinoma, a Kaposi's sarcoma, or a melanoma. In some embodiments, the peptide antigen (A) is a tumor-associated antigen from a lung cancer, such as an adenocarcinoma, a bronchiolaveolar carcinoma, a large cell carcinoma, or a small cell carcinoma. In some embodiments, the peptide antigen (A) is a tumor-associated antigen from a brain cancer, such as a glioblastoma or a meningioma. In some embodiments, the peptide antigen (A) is a tumor-associated antigen from a colon cancer. In some embodiments, the peptide antigen (A) is a tumor-associated antigen from a liver cancer, such as a hepatocellular carcinoma. In some embodiments, the peptide antigen (A) is a tumor-associated antigen from a pancreatic cancer. In some embodiments, peptide antigen (A) is a tumor-associated antigen from a kidney cancer, such as a renal cell carcinoma. In some embodiments, the peptide antigen (A) is a tumor-associated antigen from a testicular cancer.

In some embodiments, the peptide antigen (A) is a tumor-associated antigen derived from premalignant conditions, such as variants of carcinoma in situ, or vulvar intraepithelial neoplasia, cervical intraepithelial neoplasia, or vaginal intraepithelial neoplasia.

In some embodiments, the peptide antigen (A) is an antigen from an infectious agent, such as a virus, a bacterium, or a fungus. In additional embodiments, the peptide antigen (A) is a peptide or glycopeptide derived from an infectious agent; for example, the HIV Envelope fusion peptide or a V3 or V1/V2 glycopeptide from HIV.

In some embodiments, the peptide antigen (A) represent an auto-antigen. The auto-antigen may be identified and selected on the basis of screening a subject's own T cells for auto-reactivity against self-antigens presented in the context a patient's own MHC-I molecules. Alternatively, the peptide antigens may be selected using in silico methods to predict potential auto-antigens that (i) have a predicted high affinity for binding a subjects' own MHC-I molecules and (ii) are expressed and/or known to be associated with pathology accounting for a subject's auto-immune syndrome. In other embodiments, the peptide antigen represents a CD4 epitope derived from an allergen and is selected on the basis of the peptide antigen having a high binding affinity for a patient's own MHC-II molecules.

Those skilled in the art recognize that any protein or post-translationally modified protein (e.g., glycoprotein) that leads to an immune response can be selected for use as a peptide antigen (A) in the immunogenic compositions of the present invention.

In some embodiments, the peptide antigen (A) comprising the peptide antigen conjugate is specific to an individual patient. The peptide antigen conjugate comprising peptide antigens (A) that are specific to individual patients may be used for personalized therapies, such as for use as a personalized cancer vaccine or personalize tolerance inducing vaccine for treating autoimmunity or allergies.

The selection of peptide antigens (A) for inclusion in a personalized cancer vaccine is a multi-step process, wherein some steps may be dispensable.

The first step involves the identification of tumor-associated antigens that are specific to the tumor, or, relative to normal tissue, are over-expressed by the tumor. Accordingly, tumor tissue and normal tissue are obtained. Tumor tissue and normal tissue may be fixed in formalin and paraffin embedded, or may be freshly isolated tissue. Normal tissue may be blood containing leukocytes. The tumor tissue and normal tissue is processed to isolate DNA. The DNA is further processed and sequenced to identify differences between the tumor DNA and normal tissue DNA. These DNA differences may be single- or di- or higher order nucleotide changes that result in a non-synonymous mutation, insertions and deletions that result in frameshift mutations, splice site mutations that result in alternate splice variants, or stop codons that can be read through resulting in single amino acid deletions. Further mutations may be possible through chromosomal translocations or inversions or duplications. There are numerous ways that changes at the DNA level can give rise to aberrant peptide sequences and/or peptides with aberrant post-translational modifications that are tumor-specific and may be referred to as neoantigens or predicted neoantigens.

The second-step involves the determination of whether or not the tumor-associated antigens identified in step 1 are in fact expressed by the tumor. Tumor RNA is isolated, processed, and sequenced to determine if mutations identified by step 1 are expressed as RNA by the tumor cells. Peptide antigens (A) comprising mutations identified from DNA sequencing of tumor and normal tissue may be selected for inclusion in a personalized cancer vaccine on the basis of RNA expression level. Additionally, tumor associated self-antigens that produce higher levels of RNA in tumor as compared with non-cancerous tissues may be selected as peptide antigens (A) for inclusion. Mutations wherein no RNA transcript is identified are generally not selected as a peptide antigen (A) for inclusion in a vaccine. Peptide antigens (A) comprising mutations or tumor-associated self-antigens may be prioritized on the basis of RNA expression level of the mutant peptide (i.e. neoantigen) or tumor-associated self-antigens, for example, more highly expressed mutations or tumor-associated self-antigens may be prioritized. Multiple criteria may be used simultaneously in the selection of peptide antigens (A) for inclusion in a personalized cancer vaccine. For example, RNA expression level and predicted MHC binding affinity of epitopes contained by a mutant peptide (i.e. neoantigen) or tumor-associated self-antigen may be used together to select the optimal set of peptide antigens (A) for inclusion in a personalized cancer vaccine. In such a scenario, a peptide antigen (A) containing a T cell epitope with moderate binding affinity that is very highly expressed may be prioritized over a different peptide antigen (A) containing a T cell epitope that has a higher binding affinity but is expressed by the tumor at a very low level.

Another consideration is how clonal, or conserved, a mutation or tumor-associated self-antigen is across different tumor cells that comprise a tumor. The clonality of a mutation is assessed by comparing the frequency of the mutation to the frequency of the wildtype variant in the tumor isolated DNA. Tumor-associated neoantigens or self-antigens may be selected for use as peptide antigens (A) for inclusion in personalized cancer vaccines comprised of peptide antigen conjugates on the basis of clonality or near clonality of the mutation they comprise. For example, peptide antigens (A) may be prioritized for inclusion if they are predicted to be present in >50% of tumor cells, >75% of tumor cells, >85% of tumor cells, >95% of tumor cells, or >99% of tumor cells.

In some embodiments, peptide antigens (A) for inclusion in a personalized cancer vaccine comprised of peptide antigen conjugates may be further prioritized on the basis of the predicted binding of the epitopes contained within the antigen for a given MHC class I and/or class II molecule as determined by an in silico binding algorithm. In a non-limiting example, the MHC type of each subject is first identified through sequencing. Then, each mutant peptide (i.e. neoantigen) or tumor-associated self-antigen identified by any suitable means (e.g., DNA sequencing, RNA expression or mass spectrometry) is tested for predicted binding to each MHC molecule present in the subject, which, in the case of human patients, for example, may be up to 6 unique Class I MHC alleles. There are several publicly available algorithms that can be used to predict MHC binding, including the netMHC artificial neural network, the stabilized matrix method, and the Immune Epitope Database (IEDB) Analysis Resource Consensus algorithm. Non-public algorithms may also be used. Peptide antigens (A) that contain an epitope with a high predicted binding affinity are more likely to induce an immune response than peptide antigens (A) containing epitopes with a low predicted binding affinity. An unexpected finding disclosed herein is that greater than 50% of peptide antigens (A), including predicted neoantigens, that have an epitope with predicted binding affinity of less than the 0.5 percentile by the IEDB consensus algorithm are able to generate T cells responses (i.e. CD8 T cell responses) when administered using immunogenic compositions comprising peptide antigen conjugates described herein. Based on this unexpected finding, peptide antigens (A), including peptide antigens (A) comprising predicted neoantigens, that contains an epitope with a binding affinity less than 0.5 percentile with the IEDB consensus algorithm may be selected for use in the personalized cancer vaccines comprising peptide antigen conjugates.

Additionally, mass spectrometry confirmation of antigen binding to MHC, or algorithms trained on mass spectrometry binding of antigens to MHC, may be used to select peptide antigens (A) for inclusion in a personalized cancer vaccine. In this scenario, tumor tissue is processed to identify peptides that are bound to MHC molecules. Peptides that are identified on the surface of tumor cells but not normal cells, which may be mutant peptides (i.e. neoantigen), proteasomal splice-variants or tumor-associated self-antigens, may be prioritized for inclusion in a personalized cancer vaccine. An unexpected finding disclosed herein is that a high proportion (i.e. 7 out of 7) of predicted neoantigens, which were selected for use in a personalized cancer vaccine on the basis of mass spectrometry confirmed binding to MHC-I on tumor cells, led to high magnitude CD8 T cell responses when delivered as a peptide antigen (A) in immunogenic compositions comprising a peptide antigen conjugate, suggesting that mass spectrometry, or predictive algorithms based on mass spectrometry, may be reliable filters for selecting neoantigens for use as peptide antigens (A) in personalized cancer vaccines comprised of peptide antigen conjugates.

Peptide antigens (A) may also be selected for inclusion on the basis of a T cell recognition assay. For example, one may use an assay wherein synthetic peptides (or expression systems that produce the peptide in situ) comprising predicted neoantigens or tumor-associated self-antigens are added to an in vitro culture of T cells derived from the blood, tumor tissue, or other tissue from a subject. T cell recognition of a given peptide could be assessed, for example, by an ELISpot assay, or by flow cytometry. Anitgens recognized in an in vitro T cell assay may be prioritized for inclusion as a peptide antigen (A) in a personalized vaccine comprised of peptide antigen conjugates.

Finally, peptide antigens (A) may be selected based on any number of predictive algorithms. Peptide antigens (A) that are predicted to be immunogenic or efficacious based on predictive algorithms trained on large data sets may be used. Additionally, predictive algorithms may be used to select peptide neoantigens that are predicted to lead to T cell responses that are specific for the mutant epitope but not the wild-type epitope, i.e., T cells that are not cross-reactive for self-antigens.

Any combination of the above methods may be used for the identification and selection of peptide antigens (A) comprising neoantigens or tumor-associated self-antigens or the like for use in a personalized cancer vaccine comprised of peptide antigen conjugates.

A single nucleotide polymorphism that results in a non-synonymous amino acid substitution can appear in any position in a peptide epitope that binds to Class I MHC, which are typically 8-13 amino acids in length. Therefore, to cover all possible epitopes which may bind a given Class I MHC, a peptide antigen (A) comprising a neoantigen for use in a personalized cancer vaccine may include 12 amino acids on either side of the non-synonymous mutation, making a peptide of 25 amino acids in length, with the mutant amino acid as the middle (13th) residue. Alternatively, a peptide antigen (A) may include only the minimal epitope (8-13 amino acids in length) that is predicted to bind to Class I MHC. Alternatively, a personalized cancer vaccine could contain both a peptide antigen (A) comprising the 25 amino acid neoantigen and a peptide antigen (A) comprising only the predicted minimal epitope of the neoantigen.

The peptide binding pocket of MHC Class II typically binds peptides of 12-16 amino acids and in some cases as many as 20 or more amino acids. Therefore, a personalized cancer vaccine that is comprised of peptide antigens (A) that contain only the predicted Class I binding minimal epitopes (which are typically 8-13 amino acids in length) is unlikely to induce CD4 T cell responses. However, a cancer vaccine that contains 25 amino acid (or “25-mer”) peptide antigens (A) may but not always induce CD4 T cell responses targeting a given mutation.

A 25 amino acid (or “25 mer”) peptide antigen (A) in a cancer vaccine may induce lower level CD8 T cell responses compared to a peptide antigen (A) comprising the exact 7 to 12 amino acid minimal epitope, possibly due to differences in the efficiency of processing and presentation of different lengths of peptide antigens. Thus, 25 amino acid peptide antigens (A) included in a cancer vaccine may result in lower magnitude CD8 T cell responses compared to those induced by peptide antigens (A) comprising minimal epitopes. Accordingly, in some embodiments, a 25 amino acid peptide antigen (A) included in a cancer vaccine comprised of peptide antigen conjugates results in no detectable CD8 T cell responses, whereas the 7 to 12 amino acid peptide antigen (A) comprising the exact minimal epitope results in detectable CD8 T cell responses. In additional embodiments, a 25 amino acid peptide antigen (A) included in a cancer vaccine comprised of peptide antigen conjugates results in detectable CD4 T cell responses, whereas a 7 to 12 amino acid peptide antigen (A) comprising the exact minimal epitope results in no detectable CD4 T cell responses. Based on these unexpected findings, in some embodiments, two lengths of peptide antigens (A) comprising the same epitope, both the minimal CD8 T cell epitope (referred to as the “Min” or minimial epitope (ME)) and the 25 amino acid peptide (referred to as a synthetic long peptide (SLP) or long peptide), may be included as peptide antigens (A) in personalized cancer vaccines comprised of peptide antigen conjugates. In some embodiments, peptide antigens (A) that consist of the minimal CD4 and CD8 T cell epitopes are included in a personalized cancer vaccine comprised of peptide antigen conjugates. In additional embodiments, peptide antigens (A) consisting of minimal CD8 T cell and peptide antigens (A) comprising a universal CD4 T cell epitope and optionally peptide antigens (A) consisting of minimal CD4 T cell epitopes are included in personalized cancer vaccines comprised of peptide antigen conjugates. In preferred embodiments, peptide antigens (A) included in a personalized vaccine are comprised of peptide antigen conjugates of 7 to 35 amino acids, typically 15 to 35 amino acids, e.g., 25 amino acids. A single nucleotide polymorphism that results in a non-synonymous amino acid substitution can appear in any position in a peptide epitope that binds to Class I MHC, which are typically 8-13 amino acids in length. Therefore, to cover all possible epitopes which may bind a given Class I MHC, a peptide antigen (A) comprising a neoantigen for use in a personalized cancer vaccine may include 12 amino acids on either side of the non-synonymous mutation, making a peptide of 25 amino acids in length, with the mutant amino acid as the middle (13th) residue. Alternatively, a peptide antigen (A) may include only the minimal epitope (8-13 amino acids in length) that is predicted to bind to Class I MHC. Alternatively, personalized cancer vaccine could contain both a peptide antigen (A) comprising the 25 amino acid neoantigen and a peptide antigen (A) comprising only the predicted minimal epitope of a neoantigen.

### Linker (L), Linker Precursor (X1) and Linker Precursor (X2)

The peptide antigen (A) may be linked to the hydrophobic block (H) either directly or indirectly through the Linker (L), extensions (B1 or B2) or the charged moiety (C) through any suitable means, including any suitable linker.

In preferred embodiments, a Linker (L) joins the peptide antigen (A) to the hydrophobic block (H) optionally via an extension (B1 or B2) and/or charged moiety (C).

In other embodiments, the peptide antigen (A) is joined directly or via an extension (B ior B2) to a hydrophobic block (H) during solid-phase peptide synthesis or via solution-phase fragment condensation. In some embodiments, the cleavable peptide extension (B1 or B2) is heterobifunctional, e.g., an N-terminal amine of a B2 extension is linked to the C-terminus of the peptide antigen (A) and the C-terminal carboxyl group of the B2 extension is linked directly to a hydrophobic block (H). While the extension (B1 or B2) in this example may function as a linker, not all linkers are extensions.

A subset of linkers that perform the specific function of site-selectively coupling, i.e. joining or linking together the peptide antigen (A) with a hydrophobic block (H) are referred to as “Linkers (L).” The Linker (L) forms as a result of the reaction between a linker precursor X1 and a linker precursor X2. For instance, a linker precursor X1 that is linked directly, or indirectly via an extension (B1 or B2) or charged moiety (C) to the peptide antigen (A) may react with a linker precursor X2 attached to the hydrophobic block (H) to form a Linker (L) that links the peptide antigen (A) to the hydrophobic block (H). The linker precursor X1 allows for site-selective linkage of the peptide antigen (A) to a hydrophobic block (H). In some embodiments, a peptide antigen (A) linked either directly or through an extension (B1 or B2) to a linker precursor X1 may be produced and isolated as a peptide antigen fragment and then added separately to a hydrophobic block fragment comprising a hydrophobic block (H) linked to a linker precursor X2 wherein the peptide antigen fragment selectively reacts with the hydrophobic block fragment to form a Linker (L) thereby joining the peptide antigen (A) and the hydrophobic block (H).

A Linker (L) or linker precursor X1 may be linked to a peptide antigen (A) at either the N- or C-terminus of the peptide antigen (A) either directly or indirectly through an N-terminal extension (B1) or C-terminal extension (B2), respectively, or via the charged moiety (C). In preferred embodiments, the Linker (L) or linker precursor X1 is linked to the peptide antigen (A) or an extension (Blor B2) through an amide bond. A linker precursor X1 may be linked to a peptide antigen fragment directly or indirectly through an extension (B1 or B2) typically during solid-phase peptide synthesis. Note that a Linker (L) or linker precursor X1 linked directly to the N- or C-terminus of the peptide antigen (A) is not considered an extension.

The Linker (L) may comprise any suitable bond that joins the peptide antigen (A) to the hydrophobic block (H). In preferred embodiments, the Linker (L) comprises a covalent bond. Non-limiting examples of covalent bonds include those comprising disulfides, amides, thioethers, hydrazones and triazoles. Linkers (L) comprising a disulfide group can be formed by reaction of a disulphide with a thiol. Linkers (L) comprising an amide group can be formed by reaction of carboxylates (e.g., carboxylic acids, esters, carboxylic acid halides, activated carboxylic acids, and the like) with an amine or hydrazine. Linkers (L) comprising a thioether group can be formed by reaction of a maleimide with a thiol. Linkers (L) comprising a hydrazone group can be formed by reaction of a ketone with an amine or hydrazine. Linkers (L) comprising a triazole group can be formed by reaction of an azide with an alkyne. In all cases, each one of the reactive groups in a pair of reactive groups can be the first or the second reactive functional group.

Suitable linker precursors X1 are those that react selectively with a linker precursors X2 on the hydrophobic block (H) without linkages occurring at any other site of the peptide antigen (A), optional extensions (B1 and/or B2) or optional charged moiety (C). This selectivity is important for ensuring a linkage can be formed between the peptide antigen (A) and the hydrophobic block (H) without modification to the peptide antigen (A).

In preferred embodiments, the Linker (L) is formed as a result of a bio-orthogonal “click chemistry” reaction between the linker precursors X1 and X2. In some embodiments, the click chemistry reaction is a catalyst free click chemistry reaction, such as a strain-promoted azide-alkyne cycloaddition reaction that does not require the use of copper or any catalyst. Non-limiting examples of linker precursors X1 that permit bio-orthogonal reactions include molecules comprising functional groups selected from azides, alkynes, tetrazines, transcyclooctenes (TCO) and bicyclononyne (BCN). In some embodiments, a linker precursor X1 comprising an azide reacts with a linker precursor X2 to form a triazole Linker. In other embodiments, a linker precursor X1 comprising a tetrazine reacts with a linker precursor X2 comprising a transcycloooctene (TCO) to form a Linker comprising the inverse demand Diels-Alder ligation product. In preferred embodiments, the linker precursor X1 is a non-natural amino acid bearing an azide functional group that reacts with a linker precursor X2 comprising an alkyne that undergoes 1,3-dipolar cycloaddition to form a stable triazole ring. In preferred embodiments, the X2 linker precursor linked to the hydrophobic block (H) comprises an alkyne that undergoes strain-promoted cycloaddition, such as dibenzocyclooctyne (DBCO). In additional embodiments, the X1 linker precursor comprises an alkyne that reacts with a linker precursor X2 comprising an azide that is present on the hydrophobic block (H). In other embodiments, the X1 linker precursor comprises an azide that reacts with a linker precursor X2 comprising a TCO or BCN group that is present on the hydrophobic block (H).

In other embodiments, linker precursors X1 that permit site-selective reactivity depending on the composition of the peptide antigen (A) may comprise functional groups that include thiols, hydrazines, ketones and aldehydes. In some embodiments, a linker precursor X1 comprising a thiol reacts with a linker precursor X2 comprising a pyridyl-disulfide or maleimide to form a disulfide or thioether Linker (L), respectively. In other embodiments, a linker precursor X1 comprising a hydrazine reacts with a linker precursor X2 comprising a ketone or aldehyde to form a hydrazone Linker (L). In some embodiments, the linker precursor X1 is a natural or non-natural amino acid residue with a thiol functional group, such as a cysteine, that reacts with a linker precursor X2 comprising a thiol reactive functional group such as maleimide or pyridyl disulfide.

In some embodiments, the linker precursor X1 is a peptide sequence that is ligated to another peptide sequence comprising the linker precursor X2 provided on the hydrophobic block (H). The ligation may be enzyme-free native chemical ligation or an enzyme-mediated process, such as ligation promoted by Sortase or Spy Ligase. In other embodiments, the linker precursor X1 binds to a complementary molecule comprising the linker precursor X2 on the hydrophobic block (H) through high affinity, non-covalent, interactions, for example, through coiled-coil interactions or electrostatic interactions. In other embodiments, the linker precursor X1 binds to a protein, for example, biotin, which forms high affinity interactions with a protein, for example, streptavidin. In still other embodiments, the linker precursor X1 is an oligonucleotide or peptide nucleic acid that hybridizes with a complementary nucleotide present on linker precursor X2.

In some embodiments, the linker precursor X1 and linker precursor X2 are each covalently attached to both the moieties being coupled. In some embodiments, linker precursor X1 and linker precursor X2 are bifunctional, meaning the linkers include a functional group at two sites, wherein the functional groups are used to couple the linker to the two moieties. The two functional groups may be the same (which would be considered a homobifunctional linker) or different (which would be considered a heterobifunctional linker). For example, in some embodiments, a linker precursor X2 comprising a heterobifunctional linker further comprising an alkyne and an acid is used to link a hydrophobic block (H) bearing an amine and a peptide antigen (A) linked to a linker precursor X1 that bears an azide; the acid and alkyne of the linker precursor X2 are reacted to form amide and triazole bonds with the amine and azide respectively, thus linking the two heterologous molecules. In some embodiments, the linker precursor X2 comprising a heterobifunctional linker is a dibenzocyclooctyne (DBCO), TCO or BCN molecule linked to an acid. In other embodiments, the linker precursor X2 is an acid linked to a maleimide that joins an amine and thiol or a bis(carboxylic acid) that joins two amines. In still other embodiments, a tri- or multi-functional linker precursor X2 may be used, wherein the linkages are the same or different.

Those skilled in the art recognize that suitable pairs of functional groups, or complementary molecules, selected for linker precursors X1 and X2 may be transposable between X1 and X2. For example, a Linker comprised of a triazole may be formed from linker precursors X1 and X2 comprising an azide and alkyne, respectively, or from linker precursors X1 and X2 comprising an alkyne and azide, respectively. Thus, any suitable functional group pair resulting in a Linker (L) may be placed on either X1 or X2.

Particular linker precursors (X1 and X2) and Linkers (L) presented in this disclosure provide unexpected improvements in manufacturability and improvements in biological activity. Many such linker precursors (X1 and X2) and Linkers (L) may be suitable for the practice of the invention and are described in greater detail throughout.

The linker precursor (X1) may be attached to either the N-terminal amino acid of the peptide antigen fragment, such as the N-terminal amino acid of B1 or directly to the peptide antigen (A) when B1 is not present. Alternatively, the peptide antigen fragment linker precursor (X1) may be attached to the C-terminal amino acid of B2 or the peptide antigen (A) when B2 is not present.

In some embodiments, the linker precursor X1 is an amino acid linked to the C-terminus of the peptide antigen (A) either directly or indirectly through a B2 extension or charged moiety (C) and has the formula:

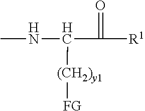

wherein the functional group (FG) of X1 is selected to react specifically with a FG on the linker precursor X2 and is typically selected from amine, azide, hydrazine or thiol; R is typically selected from OH or NH₂ and y1 is any integer, such as 1, 2, 3, 4, 5, 6, 7 or 8; and the alpha amine of the amino acid is typically linked to the C-terminal amino acid of the extension B2 or the C-terminal amino acid of the peptide antigen (A) if there is no B2 extension.

In other embodiments, the linker precursor X1 is linked to the N-terminus of the peptide antigen (A) either directly or indirectly through an extension (B1 or B2) or charged moiety (C) and has the formula:

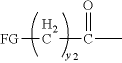

wherein the functional group (FG) is selected to react with the linker precursor X2 and is typically selected from amine, azide, hydrazine or thiol; y2 is any integer, typically 1, 2, 3, 4, 5, 6, 7 or 8; and the carbonyl is typically linked to the alpha amine of the N-terminal amino acid of the extension B1 or the peptide antigen (A) when B1 is not present.

In some embodiments, the linker precursor X1 is an azido amino acid linked to the C-terminus of the peptide antigen (A) either directly or indirectly through a B2 extension or charged moiety (C) and has the formula:

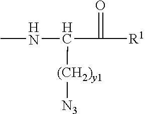

wherein the alpha amine of the amino acid is linked to the C-terminal amino acid of B2, or the peptide antigen (A) if there is no B2 extension; R¹ is selected from OH or NH₂ and y1 is any integer, such as 1, 2, 3, 4, 5, 6, 7, 8. Non-limiting examples of azido containing linker precursors X1 are 5-azido-2-amino pentanoic acid, 4-azido-2-amino butanoic acid and 3-azido-2-amino propanoic acid.

In other embodiments when the azido containing linker precursor X1 is linked to the N-terminal extension (B1), or directly to the N-terminus of the peptide antigen (A) when B1 is not present, the linker precursor X1 has the formula:

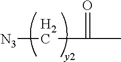

wherein the carbonyl is typically linked to the alpha amine of the N-terminal amino acid of the extension B1 or the peptide antigen (A) when B1 is not present; and y2 is any integer, typically 1, 2, 3, 4, 5, 6, 7 or 8. Non-limiting examples of azido-containing linker precursors include 6-azido-hexanoic acid, 5-azido-pentanoic acid, 4-azido-butanoic acid and 3-azido-propanoic acid.

The linker precursor X2 provided on the hydrophobic block (H) comprises a functional group that is selected to allow for a selective reaction with the linker precursor X1 to form the Linker (L). In some embodiments, functional groups comprising X2 include carbonyls, such as activated esters/carboxylic acids or ketones that react with amines or hydrazines provided on the linker precursor X1 to form Linkers (L) comprising an amide or hydrazone. In other embodiments, functional groups comprising X2 include azides that react with alkynes provided on the linker precursor X1 to form triazoles. In still other embodiments, the functional group comprising X2 is selected from maleimides or disulfides that react with thiols provided on the linker precursor X1 to form Linkers (L) comprising thioethers or disulphides. The linker precursor X2 may be attached to the hydrophobic block (H) through any suitable means. In some embodiments, the hydrophobic block (H) comprises a peptide and X2 is linked to the N-terminus of the peptide-based hydrophobic block (H).

In some embodiments, wherein the linker precursor X1 comprises an azide, the linker precursor X2 comprises an alkyne moiety. Non-limiting examples of alkynes include aliphatic alkynes, cyclooctynes, such as dibenzylcyclooctyne (DBCO or DIBO), difluorooctyne (DIFO), and biarylazacyclooctynone (BARAC). In some specific embodiments, the alkyne containing linker precursor X2 comprises a DBCO molecule. In other embodiments, wherein the linker precursor X1 comprise as an azide, the linker precursor X2 comprises a BCN molecule. In other embodiments, wherein the linker precursor X1 comprise as a tetrrazine, the linker precursor X2 comprises a TCO molecule.

In some embodiments, the C-terminal extension (B2) is linked to the peptide antigen (A) through an amide bond at the C-terminus of the peptide antigen (A) which, in turn, is linked via the Linker (L) to the hydrophobic block (H). An example of such a C-terminal linked peptide antigen conjugate is (A)₇₋₂₅-B2-L-H, where (A)₇₋₃₅ represents a peptide antigen (A) comprising from 7 to 35 amino acids. In a non-limiting example, a peptide antigen (A) with the octapeptide sequence PA8-PA7-PA6-PA5-PA4-PA3-PA2-PA1 is linked at the C-terminus to a tetrapeptide extension (B2), for example, Ser-Leu-Val-Arg that is linked to the azido containing linker precursor (X1) azido-lysine (6-azido 2-amino hexanoic acid, Lys(N3)) which in turn reacts with the dibenzocyclooctyne (DBCO) moiety of the cyclooctyne containing linker precursor (X2) that is linked to the hydrophobic block (H) to produce PA8-PA7-PA6-PA5-PA4-PA3-PA2-PA1-Ser-Leu-Val-Arg-Lys(N3-DBCO-H).

### Other Linkers and Spacer Groups

The peptide antigen conjugate may comprise additional linker moieties in addition to the Linker (L). In this context, a linker is broadly defined as any molecule or group of atoms that links or couples or joins together two or more moieties. The peptide antigen conjugates disclosed herein are complex molecules that comprise multiple different functional components (peptide antigen (A), hydrophobic block (H) Linker (L), optional extensions (B1 and/or B2), optional charged moiety (C), or optional Ligand(s), etc.) that may be linked, or joined together, through any suitable means.

Suitable linker moieties include, but are not limited to, straight or branched-chain carbon linkers, heterocyclic carbon linkers, rigid aromatic linkers, flexible ethylene oxide linkers, peptide linkers, or a combination thereof. In some embodiments, the carbon linker can include a C1-C18 alkane linker, such as a lower alkyl C4; the alkane linkers can serve to increase the space between two or more heterologous molecules, while longer chain alkane linkers can be used to impart hydrophobic characteristics. Alternatively, hydrophilic linkers, such as ethylene oxide linkers, may be used in place of alkane linkers to increase the space between any two or more heterologous molecules and increase water solubility. In other embodiments, the linker can be an aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immuno-proteasome).

In some embodiments, the linker may be comprised of poly(ethylene oxide) (PEG). The length of the linker depends on the purpose of the linker. For example, the length of the linker, such as a PEG linker, can be increased to separate components of an immunogenic composition, for example, to reduce steric hindrance, or in the case of a hydrophilic PEG linker can be used to improve water solubility. The linker, such as PEG, may be a short linker that may be at least 2 monomers in length. The linker, such as PEG, may be between about 2 and about 24 monomers in length, such as 2, 3, 4, 5, 6, 7, 8, 9, 10, 11, 12, 13, 14, 15, 16, 17, 18, 19, 20, 21, 22, 23, 24 monomers in length or more.

In some embodiments, where the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, where the linker comprises a carbon chain, the linker may comprise a chain of between about 12 and about 20 carbons. In some embodiments, where the linker comprises a carbon chain, the linker may comprise a chain of between no more than 18 carbons. In some embodiments, an adjuvant is linked to the hydrophobic block (H) through a suitable linker, such as a lower alkyl.

In some embodiments, the linker is cleavable under intracellular conditions, such that cleavage of the linker results in the release of any component linked to the linker, for example, a peptide antigen (A).

For example, the linker can be cleavable by enzymes localized in intracellular vesicles (for example, within a lysosome or endosome or caveolea) or by enzymes, in the cytosol, such as the proteasome, or immuno-proteasome. The linker can be, for example, a peptide linker that is cleaved by protease enzymes, including, but not limited to proteases (such as a cathepsin) that are localized in intracellular vesicles, such as a lysosomal or endosomal compartment. The peptide linker is typically between 2-10 amino acids, such as 2, 3, 4, 5, 6, 7, 8, 9, 10, or more (such as up to 20) amino acids long, such as 11, 12, 13, 14, 15, 16, 17, 18, 19, 20 or more amino acids long. Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly linker). Other examples of such linkers are described, for example, in U.S. Pat. No. 6,214,345, incorporated herein by reference. In a specific embodiment, the peptide linker cleavable by an intracellular protease is a Val-Cit linker or a Phe-Lys linker (see, for example, U.S. Pat. No. 6,214,345, which describes the synthesis of doxorubicin with the Val-Cit linker).

Particular sequences for the cleavable peptide in the linker can be used to promote processing by immune cells following intracellular uptake. For example, several embodiments of the immunogenic compositions disclosed herein form particles in aqueous conditions, which are internalized by immune cells, such as antigen-presenting cells (e.g., dendritic cells). The cleavable peptide linker can be selected to promote processing (i.e. hydrolysis) of the peptide linker following intracellular uptake by the immune cells. The sequence of the cleavable peptide linker can be selected to promote processing by intracellular proteases, such as cathepsins in intracellular vesicles or the proteasome or immuno-proteasome in the cytosolic space.

In several embodiments, linkers comprised of peptide sequences of the formula Pn . . . P4-P3-P2-P1 are used to promote recognition by cathepsins, wherein P1 is selected from arginine, lysine, citrulline, glutamine, threonine, leucine, norleucine, or methionine; P2 is selected from glycine, leucine, valine or isoleucine; P3 is selected from glycine, serine, alanine, proline or leucine; and P4 is selected from glycine, serine, arginine, lysine, aspartic acid or glutamic acid. In a non-limiting example, a tetrapeptide linker of the formula P4-P3-P2-Plthat is recognized by cathepsins is linked through an amide bond at the C-terminus of P1 to a heterologous molecule and has the sequence Ser-Pro-Leu-Cit. For clarity, the amino acid residues (Pn, wherein n is any integer) are numbered from proximal to distal from the site of cleavage, which is C-terminal to the P1 residue; for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprised of peptide sequences are selected to promote recognition by the proteasome or immuno-proteasome. Peptide sequences of the formula Pn . . . P4-P3-P2-P1 are selected to promote recognition by proteasome or immuno-proteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In a non-limiting example, a cleavable linker of the formula P4-P3-P2-P1 that is recognized by the proteasome is linked through an amide bond at P1 to a heterologous molecule and has the sequence Tyr-Leu-Leu-Leu. Sequences that promote degradation by the proteasome or immuno-proteasome may be used alone or in combination with cathepsin cleavable linkers. In some embodiments, amino acids that promote immuno-proteasome processing are linked to linkers that promote processing by endosomal proteases. A number of suitable sequences to promote cleavage by the immuno-proteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In other embodiments, any two or more components of the peptide antigen conjugates may be joined together through a pH-sensitive linker that is sensitive to hydrolysis under acidic conditions. A number of pH-sensitive linkages are familiar to those skilled in the art and include for example, a hydrazone, semicarbazone, thiosemicarbazone, cis-aconitic amide, orthoester, acetal, ketal, or the like (see, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). In preferred embodiments, the linkage is stable at physiologic pH, e.g., at a pH of about 7.4, but undergoes hydrolysis at lysosomal pH, pH 5-6.5. In some embodiments, a Ligand is linked to a hydrophobic block (H) through a FG that forms a pH-sensitive bond, such as the reaction between a ketone and a hydrazine to form a pH labile hydrazone bond. A pH-sensitive linkage, such as a hydrazone, provides the advantage that the bond is stable at physiologic pH, at about pH 7.4, but is hydrolyzed at lower pH values, such as the pH of intracellular vesicles.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.). In some embodiments, a Ligand is linked to the hydrophobic block (H) that bears a thiol functional group to form a disulfide bond. In some embodiments, two or more hydrophobic blocks are linked together through disulfide bonds.

In yet additional embodiments the linkage between any two components of the peptide antigen conjugate can be formed by an enzymatic reaction, such as expressed protein ligation or by sortase (see: Fierer, et al., Proc. Natl. Acad. Sci., 111:W1176-1181, 2014 and Theile et al., Nat. Protoc., 8:1800-1807, 2013.) chemo-enzymatic reactions (Smith, et al., Bioconjug. Chem., 25:788-795, 2014) or non-covalent high affinity interactions, such as, for example, biotin-avidin and coiled-coil interactions (Pechar, et al., Biotechnol. Adv., 31:90-96, 2013) or any suitable means that are known to those skilled in the art (see: Chalker, et al., Acc. Chem. Res., 44:730-741, 2011, Dumas, et al., Agnew Chem. Int. Ed. Engl., 52:3916-3921, 2013).

### N-Terminal (B1) and C-Terminal (B2) Extensions

Peptide antigen conjugates may comprise N-terminal (B1) and/or C-terminal (B2) extensions wherein B1 or B2 may be linked via Linker (L) to the hydrophobic block (H) that promotes particle formation. Any one of the components may be linked through any suitable means.

The N- and C-terminal extensions B1 and B2 may be comprised of any one or more of the following: amino acids, including non-natural amino acids; hydrophilic ethylene oxide monomers (e.g., PEG); hydrophobic alkane chains; or the like; or combinations thereof. The N- and C-terminal extensions B1 and B2 are linked to the peptide antigen (A) through any suitable means, e.g., through stable amide bonds.

In some embodiments, the extensions (B1 and B2) function to control the rate of degradation of the peptide antigen (A) but may also perform any one or more additional functions. In some embodiments, the N- or C-terminal extension (B1 or B2) may be free (wherein one end of the N- or C-terminal extension is linked to the peptide antigen (A) and the other end is not linked to another molecule) and serve to slow degradation of the peptide antigen (A); for example, a B1 peptide-based extension may be linked to the N-terminus of the peptide antigen (A) through an amide bond to slow degradation. In other embodiments, the N- and/or C-terminal extensions (B1 and/or B2) may be linked to a heterologous molecule and may therefore function as a linker as well as modulate peptide antigen (A) degradation. The N- and/or C-terminal extensions providing a linker function may link the peptide antigen either directly or indirectly through a Linker (L), to a hydrophobic block (H), and/or a charged moiety (C).

In some embodiments, the extensions (B1 and/or B2) function to provide distance, i.e. space, between any two heterologous molecules. In other embodiments, the extensions (B1 and/or B2) function to impart hydrophobic or hydrophilic properties to the peptide antigen conjugate. In still other embodiments, the composition of the extensions (B1 and/or B2) may be selected to impart rigidity or flexibility. In other embodiments, the N- and/or C-terminal extensions (B1 and/or B2) may help stabilize the particles formed by the peptide antigen conjugate.

In some embodiments, the extensions (B1 and/or B2) are comprised of charged functional groups, e.g., charged amino acid residues (e.g., Arginine, Lysine), that impart electrostatic charge at physiologic pH. The number of charged residues present in the extension can be used to modulate the net charge of the peptide antigen conjugate.

In some embodiments, C-terminal extensions (B2) added to peptide antigens (A) are selected to facilitate manufacturing of peptide antigen fragments of the formula [C]-[B1]-A-B2-[X1], wherein [ ] denotes the group is optional, by incorporating amino acid sequences into B2 that disrupt β-sheet formation and prevent sequence truncation during solid-phase peptide synthesis. In a non-limiting example, a C-terminal di-peptide linker (B2), Gly-Ser, is incorporated during solid-phase peptide synthesis as a pseudoproline dipeptide (e.g. Gly-Ser(Psi(Me,Me)pro)). In additional embodiments, a proline is included in the cathepsin cleavable C-terminal extension (B2) sequence, e.g., Ser-Pro-Leu-Arg (SEQ ID NO: 1); whereby the proline is included to both facilitate manufacturing and promote processing of the extension by endosomal proteases.

In some embodiments, the peptide antigen (A) is linked at the C-terminus to a B2 extension that is linked either directly or indirectly through a Linker (L) to a hydrophobic block (H). In some embodiments, a B1 extension is linked to the N-terminus of the peptide antigen (A) and a B2 extension is linked at the C-terminus of the peptide antigen (A), wherein either B1 or B2 are linked either directly or via a Linker (L) to a hydrophobic block (H). In other embodiments, a peptide antigen (A) is linked at the N-terminus to a B1 extension that is linked either directly or via a Linker (L) to a hydrophobic block (H). In some embodiments, a charged moiety (C) is linked to an extension, B1 or B2, that is linked to the N- or C-terminus of the peptide antigen (A), respectively, wherein the extension that is not linked to the charged moiety (C) is linked either directly or via a Linker (L) to the hydrophobic block (H). In additional embodiments, charged moieties (C) are linked to both B1 and B2 extensions that are linked to both the N- and C-termini of the peptide antigen (A), respectively. In additional embodiments, charged moieties (C) are linked to the B1 extension linked to the N-terminus of the peptide antigen (A) but not to the B2 extension attached to the C-terminus of the peptide antigen (A), which may be linked either directly or through a Linker (L) to a hydrophobic block (H). A linker precursor X1 or Linker (L) may be linked to either of the extensions (B1 or B2) through any suitable means, such as an amide bond. In preferred embodiments, the extensions (Bland B2) are peptide sequences that are selected for recognition and hydrolysis by enzymes, such as proteases. The extensions (B1 and B2) are preferably cleavable peptides, including amino acids recognized by endosomal proteases and/or the immuno-proteasome.

As described in greater detail here, the composition of the extensions (B1 or B2) comprised of degradable peptides is dependent on whether the extension is linked to the N-terminus (B1) or C-terminus (B2) of the peptide antigen (A).

By way of non-limiting example, the C-terminal extension (B2) may be a peptide sequence that promotes cleavage by cathepsins, the immuno-proteasome or both cathepsins and the immuno-proteasome. Amino acids linked proximally to the C-terminus of the peptide antigen (A) that are preferentially recognized for processing by the immuno-proteasome include small, non-charged residues, such as glycine, serine and alanine. Amino acids linked proximally to the C-terminus of the peptide antigen (A) that are preferentially recognized for processing by cathepsins include arginine, lysine, citrulline, glutamine, threonine, leucine, norleucine, or methionine.

In some embodiments, the extension (B2) is a degradable peptide linked to the C-terminal residue of the peptide antigen (A) and is comprised of amino acid sequences that are recognized and hydrolyzed by certain proteases. In some embodiments, the C-terminal extension (B2) is a peptide sequence between about 1 to 8 amino acids in length, such as 1, 2, 3, 4, 5, 6, 7, or 8 amino acids, typically no more than 10 amino acids. In preferred embodiments, the C-terminal extension (B2) is linked to the peptide antigen (A) via an amide bond formed between the C-terminal carboxyl group of the peptide antigen (A) and the alpha amine of the N-terminal residue of the extension (B2). The amide bond between B2 and the peptide antigen (A) may be cleaved by enzymes.

Note that peptide sequences referring to the peptide antigen are designated as “PA”, peptide sequences referring to the N-terminal extension (B1) are designated as “PN”, and peptide sequences referring to the C-terminal extension (B2) are designated as “PC”. Sequences of amino acids comprising peptide antigens (A) are represented by the formula, PA1 . . . PAn, where PA represents any amino acid residue comprising a peptide antigen (A) and n is an integer value. For example, an 8-amino acid peptide antigen (A) may be represented as PA1-PA2-PA3-PA4-PA5-PA6-PA7-PA8. Sequences of amino acids comprising N-terminal extensions (B1) are represented by the formula, PN . . . PNn, where PN represents any amino acid residue comprising an N-terminal extension and n is an integer value. Sequences of amino acids comprising C-terminal extensions (B2) are represented by the formula, PC1 . . . PCn, where PC represents any amino acid residue comprising a C-terminal extension and n is an integer value.

It is customary to number the amino acid positions in order of proximal to distal from the cleavage site, with amino acid positions C-terminal to the cleavage site indicated by the prime symbol (e.g., Pn′). For example, for a tetrapeptide extension (PC1′-PC2′-PC3′-PC4′) linked to the C-terminus of an octapeptide antigen (PA8-PA7-PA6-PA5-PA4-PA3-PA2-PA1), e.g., PA8-PA7-PA6-PA5-PA4-PA3-PA2-PA1-PC1′-PC2′-PC3′-PC4′, the amide bond between PA1-PC1′ is recognized and hydrolyzed by an enzyme.

In certain embodiments, C-terminal extensions (B2) are amino acid sequences that are selected to promote immuno-proteasome recognition and cleavage and optionally endosomal protease recognition. As peptide antigens (A) typically contain a C-terminal residue, for example, leucine, that promotes hydrolysis by the immuno-proteasome, e.g., at the amide bond proximal to the C-terminal residue of the peptide antigen (A), extensions linked to the C-terminus of the peptide antigen (A) should be selected to promote immuno-proteasome recognition and cleavage at the amide bond proximal to the C-terminus of the peptide antigen (A). The immuno-proteasome favors small, non-charged amino acids at the PC1′ position adjacent to the C-terminal amino acid, PA1, of the peptide antigen (A), e.g., the amide bond between PA1-PC1′. However, endosomal proteases favor bulky hydrophobic amino acids (e.g., leucine, norleucine, methionine or glutamine) and basic amino acids (i.e., arginine and lysine). Therefore, C-terminal extensions may be selected to promote recognition by either or both classes of proteases.

In some embodiments, a peptide antigen (A) with the sequence PA8-PA7-PA6-PA5-PA4-PA3-PA2-PA1 is linked to a C-terminal peptide extension (B2) with the sequence PC1′ . . . PCn′, wherein n is an integer value from 1 to 8, for example, PA8-PA7-PA6-PA4-PA3-PA2-PA1-PC1′ . . . PCn′. The composition of the C-terminal extension (B2) depends on the length of the extension sequence used. In some embodiments, the C-terminal extension, B2, is a single amino acid PC1′ selected from Gly, Ala, Ser, Arg, Lys, Cit, Gln, Thr, Leu, Nle or Met. In additional embodiments, the C-terminal extension, B2, is a dipeptide, PC1′-PC2′, wherein PC1′ is selected from Gly, Ala or Ser; and PC2′ is selected from Gly, Ala, Ser, Pro, Arg, Lys, Cit, Gln, Thr, Leu, Nle, or Met. In additional embodiments, the C-terminal extension, B2, is a tripeptide, PC1′-PC2′-PC3′, wherein PC1′ is selected from Gly, Ala, or Ser; PC2′ is selected from Gly, Ala, Ser, or Pro; and PC3′ is selected from Gly, Ser, Arg, Lys, Cit, Gln, Thr, Leu, Nle or Met. Note that Cit=citrulline.

In additional embodiments, the C-terminal extension, B2, is a tetrapeptide extension, PC1′-PC2′-PC3′-PC4′, wherein PC1′ is selected from glycine, alanine or serine; PC2′ is selected from glycine, alanine, serine, proline or leucine; PC3′ is selected from glycine, alanine, serine, valine, leucine or isoleucine; and PC4′ is selected from arginine, lysine, citrulline, glutamine, threonine, leucine, norleucine or methionine. In additional embodiments, the C-terminal extension, B2, is a pentapeptide, PC1′-PC2′-PC3′-PC4′-PC5′, wherein PC1′ is selected from glycine, alanine or serine; PC2′ is selected from glycine, alanine, serine, proline, arginine, lysine, glutamic acid or aspartic acid; PC3′ is selected from glycine, alanine, serine, proline or leucine; PC4′ is selected from glycine, alanine, valine, leucine or isoleucine; and PC5′ is selected from arginine, lysine, citrulline, glutamine, threonine, leucine, norleucine or methionine. In additional embodiments, the C-terminal extension, B2, is a hexapeptide, PC1′-PC2′-PC3′-PC4′-PC5′-PC6′, wherein PC1′ is selected from glycine, alanine or serine; PC2′ is selected from glycine, alanine, serine or proline; PC3′ is selected from glycine, serine, proline, arginine, lysine, glutamic acid or aspartic acid; PC4′ is selected from proline or leucine; PC5′ is selected from glycine, alanine, valine, leucine or isoleucine; and PC6′ is selected from arginine, lysine, citrulline, glutamine, threonine, leucine, norleucine or methionine.

Non-limiting examples of hexapeptide C-terminal extensions (B2) include Gly-Gly-Lys-Leu-Val-Arg (SEQ ID NO: 2), Gly-Gly-Lys-Pro-Leu-Arg (SEQ ID NO: 3), Gly-Gly-Ser-Leu-Val-Arg (SEQ ID NO: 4), Gly-Gly-Ser-Leu-Val-Cit (SEQ ID NO:26), Gly-Gly-Ser-Pro-Val-Cit (SEQ ID NO:33), Gly-Gly-Ser-Leu-Val-Leu (SEQ ID NO: 5), Gly-Gly-Glu-Leu-Val-Arg (SEQ ID NO: 6), Gly-Gly-Glu-Leu-Val-Leu (SEQ ID NO: 7).

Non-limiting examples of pentapeptide C-terminal extensions (B2) include Gly-Ser-Leu-Val-Arg (SEQ ID NO: 8), Gly-Ser-Leu-Val-Cit (SEQ ID NO:29), Gly-Lys-Pro-Val-Cit (SEQ ID NO:32), Gly-Lys-Pro-Val-Arg (SEQ ID NO: 9), Gly-Ser-Leu-Val-Leu (SEQ ID NO: 10), Gly-Glu-Leu-Val-Leu (SEQ ID NO: 11).

Non-limiting examples of tetrapeptide C-terminal extensions (B2) include Ser-Leu-Val-Cit (SEQ ID NO:36), Ser-Leu-Val-Leu (SEQ ID NO: 12), Ser-Pro-Val-Cit (SEQ ID NO:27), Glu-Leu-Val-Arg (SEQ ID NO: 13), Ser-Pro-Val-Arg (SEQ ID NO: 14), Ser-Leu-Val-Arg (SEQ ID NO: 15), Lys-Pro-Leu-Arg (SEQ ID NO: 16), Glu-Leu-Val-Cit (SEQ ID NO:28), Glu-Leu-Val-Leu (SEQ ID NO: 17), Glu-Pro-Val-Cit (SEQ ID NO:34), Glu-Gly-Val-Cit (SEQ ID NO:35).

Non-limiting examples of tripeptide C-terminal extensions (B2) include Gly-Ser-Gly, Gly-Ser-Arg, Gly-Ser-Leu, Gly-Ser-Cit, Gly-Pro-Gly, Gly-Pro-Arg, Gly-Pro-Leu, Gly-Pro-Cit.

Non-limiting examples of di-peptide C-terminal extensions (B2) include Gly-Ser, Gly-Pro, Val-Cit, Gly-Arg, Gly-Cit.

Non-limiting examples of single amino acid C-terminal extensions (B2) include Gly, Ser, Ala, Arg, Lys, Cit, Val, Leu, Met, Thr, Gln or Nle. In the above examples, Arg can be replaced with Lys; Lys can be replaced with Arg; Glu can be replaced with Asp; and Asp can be replaced with Glu.

The C-terminal linker (B2) linked to the C-terminus of the peptide antigen (A) may be selected for recognition (i.e. hydrolysis) by both the immuno-proteasome and endosomal proteases. In a non-limiting example, a peptide antigen (A) with the sequence PA8-PA7-PA6-PA5-PA4-PA3-PA2-PA1 is linked at the C-terminus to a C-terminal tetrapeptide extension (B2) with the sequence PC1′-PC2′-PC3′-PC4′, wherein PC1′ is selected from glycine, alanine or serine and PC4′ is selected from arginine, lysine, citrulline, glutamine, threonine, leucine, norleucine, or methionine, for example, Ser-P3-P2-Arg. In some embodiments, a peptide antigen (A) with the sequence PA8-PA7-PA6-PA5-PA4-PA3-PA2-PA1 is linked at the C-terminus to a C-terminal hexapeptide extension (B2) with the sequence PC1′-PC2′-PC3′-PC4′-PC5′-PC6′, wherein PC1′ and PC2′ are selected from glycine, alanine, proline or serine and PC6′ is selected from arginine, lysine, citrulline, glutamine, threonine, leucine, norleucine, or methionine, for example, Gly-Gly-PC3′-PC4′-PC5′-Arg. A non-limiting example of a C-terminal extension (B2) that promotes processing by both the immuno-proteasome and cathepsins that is linked to the C-terminus of the peptide antigen (A) is Gly-Gly-Lys-Pro-Leu-Arg (SEQ ID NO: 3). An additional non-limiting example of a C-terminal extension (B2) that is linked at the C-terminus of a peptide antigen (A) that favors processing by the immuno-proteasome and cathepsins is Gly-Gly-Ser-Leu-Val-Cit (SEQ ID NO:26) or Gly-Gly-Ser-Pro-Val-Cit (SEQ ID NO:33).

In some embodiments, the N-terminal extension (B1) is a peptide sequence between about 1 to 8 amino acids in length, such as 1, 2, 3, 4, 5, 6, 7, or 8 amino acids, typically no more than 10 amino acids in length that is linked to the peptide antigen (A) through, e.g., an amide bond formed between a carboxyl group of the extension (B1) and the alpha amine of the N-terminal residue of the peptide antigen (A). The amide bond between B1 and the peptide antigen (A) may be cleaved by enzymes. It is understood that it is customary to number the amino acid positions in order of proximal to distal from the cleavage site, with amino acid positions C-terminal to the cleavage site indicated by the prime symbol (e.g., Pn′). For example, for a tetrapeptide extension (PN4-PN3-PN2-PN1) linked to the N-terminus of a peptide antigen (A) that is an octapeptide (PA1′-PA2′-PA3′-PA4′-PA5′-PA6′-PA7′-PA8′), e.g., PN4-PN3-PN2-PN1-PA1′-PA2′-PA3′-PA4′-PA5′-PA6′-PA7′-PA8′, the amide bond between PN1-PA1′ is recognized and hydrolyzed by an enzyme.

By way of non-limiting example, the N-terminal extension (B1) may be an enzyme degradable peptide that is recognized by endosomal proteases, wherein the PN1 position of a tetrapeptide extension (PN4-PN3-PN2-PN1) linked to a peptide antigen (A) (e.g. PA1′-PA2′-PA3′-PA4′-PA5′-PA6′-PA7′-PA8′) is selected from arginine, lysine, citrulline, glutamine, threonine, leucine, norleucine, or methionine, for example, PN4-PN3-PN2-Arg. In some embodiments, the N-terminal extension (B1) is an enzyme degradable peptide that is recognized by the immuno-proteasome, wherein the PN1 position of a tetrapeptide (PN4-PN3-PN2-PN1) is selected from isoleucine, leucine, norleucine or valine, for example, PN4-PN3-PN2-Leu. In additional embodiments, the N-terminal extension (B1) is an enzyme degradable peptide that is recognized by both endosomal proteases and the immuno-proteasome, wherein the PN5 and PN1 positions of an octapeptide (PN8-PN7-PN6-PN5-PN4-PN3-PN2-PN1) extension are selected from arginine, lysine, citrulline, glutamine, threonine, leucine, norleucine, or methionine for the PN5 position that is recognized by cathepsins, and isoleucine, leucine, norleucine or valine for the PN1 position recognized by the immuno-proteasome; for example, PN8-PN7-PN6-Arg-PN4-PN3-PN2-Leu. A non-limiting example of an N-terminal extension (B1) recognized by cathepsins and the immuno-proteasome is Lys-Pro-Leu-Arg-Tyr-Leu-Leu-Leu. An additional non-limiting example of an N-terminal extension (B1) recognized by cathepsins and the immuno-proteasome is Ser-Leu-Val-Cit-Tyr-Leu-Leu-Leu.

In some embodiments, the N-terminal extension (B1) is an enzyme degradable tetrapeptide that is recognized by endosomal proteases, wherein the PN1 position of a tetrapeptide extension (e.g., PN4-PN3-PN2-PN1) is preferably selected from arginine, lysine, citrulline, glutamine, threonine, leucine, norleucine, or methionine, for example, PN4-PN3-PN2-Arg; PN2 is selected from glycine, valine, leucine or isoleucine; PN3 is selected from glycine, serine, alanine, proline or leucine; and PN4 is selected from glycine, serine, arginine, lysine, aspartic acid or glutamic acid. In some embodiments, the N-terminal extension (B1) is an enzyme degradable tripeptide that is recognized by endosomal proteases, wherein the PN1 position of a tripeptide extension (e.g., PN3-PN2-PN1) is preferably selected from arginine, lysine, citrulline, glutamine, threonine, leucine, norleucine, or methionine; PN2 is selected from glycine, valine, leucine or isoleucine; and PN3 is selected from glycine, serine, alanine, proline or leucine. In some embodiments, the N-terminal extension (B1) is an enzyme degradable di-peptide that is recognized by endosomal proteases, wherein the PN1 position of a dipeptide extension (e.g., PN2-PN1) is preferably selected from arginine, lysine, citrulline, glutamine, threonine, leucine, norleucine, or methionine; and PN2 is selected from glycine, valine, leucine or isoleucine. In still additional embodiments, the N-terminal extension (B1) is an amino acid that is recognized by endosomal proteases, wherein the PN1 position is preferably selected from arginine, lysine, citrulline, glutamine, threonine, leucine, norleucine, or methionine.

In other embodiments, the N-terminal extension (B1) is an enzyme degradable peptide that is recognized by the immuno-proteasome, wherein the P1 position of a tetrapeptide extension (PN4-PN3-PN2-PN1) is preferably selected from isoleucine, leucine, norleucine or valine, for example, PN4-PN3-PN2-Leu.

In additional embodiments, the N-terminal extension (B1) is an enzyme degradable peptide that is recognized by both endosomal proteases and the immuno-proteasome, wherein the PN5 and PN1 positions of an octapeptide extension (PN8-PN7-PN6-PN5-PN4-PN3-PN2-PN1) are selected from arginine, lysine, citrulline, glutamine, threonine, leucine, norleucine, or methionine for the PN5 position recognized by cathepsins, and isoleucine, leucine, norleucine or valine for the PN1 position recognized by the immuno-proteasome; for example, PN8-PN7-PN6-Arg-PN4-PN3-PN2-Leu. A non-limiting example of an N-terminal extension (B1) recognized by cathepsins and the immuno-proteasome is Lys-Pro-Leu-Arg-Tyr-Leu-Leu-Leu (SEQ ID NO: 18).

Non-limiting examples of tetrapeptide N-terminal extensions (B1) that are recognized by the immuno-proteasome include: Ser-Leu-Val-Cit (SEQ ID NO:36), Ser-Leu-Val-Leu (SEQ ID NO: 19), Ser-Pro-Val-Cit (SEQ ID NO:30), Glu-Leu-Val-Arg (SEQ ID NO: 20), Ser-Pro-Val-Arg (SEQ ID NO: 21), Ser-Leu-Val-Arg (SEQ ID NO: 22), Lys-Pro-Leu-Arg (SEQ ID NO: 23), Lys-Pro-Val-Arg (SEQ ID NO: 24), Glu-Leu-Val-Cit (SEQ ID NO:31), Glu-Leu-Val-Leu (SEQ ID NO: 25), Glu-Pro-Val-Cit (SEQ ID NO:37) and Lys-Pro-Val-Cit (SEQ ID NO:38).

Non-limiting examples of tripeptide N-terminal extensions (B1) include: Leu-Val-Cit, Leu-Val-Leu, Pro-Val-Cit, Leu-Val-Arg, Pro-Val-Arg, Pro-Leu-Arg, Gly-Val-Ser.

Non-limiting examples of di-peptide N-terminal extensions (B1) include: Val-Cit, Val-Leu, Val-Arg, Leu-Arg.

Non-limiting examples of single amino acid N-terminal extensions (B1) include Cit, Arg, Leu or Lys. In the above examples, Arg can be replaced with Lys; Lys can be replaced with Arg; Glu can be replaced with Asp; and Asp can be replaced with Glu. Note that Cit=citrulline.

### Hydrophobic Block (H)

The peptide antigen (A) may be linked either directly or via an extension (B1 or B2), a charged moiety (C), and/or Linker (L) to the hydrophobic block (H). In some embodiments, the linker precursor (X2) is linked to the hydrophobic block (H) and reacts with the linker precursor (X1) on the peptide antigen fragment to form the Linker (L) that is linked to the peptide antigen (A) directly or indirectly via an extension (B1 or B2).

In the present disclosure, the term “hydrophobic block” (H) is used as a general term to describe a molecule with limited water solubility, or amphiphilic characteristics, that can be linked to peptide antigens (A) resulting in a peptide antigen conjugate that forms particles in aqueous conditions. The hydrophobic block (H) in this context promotes particle assembly due to its poor solubility, or tendency to assemble into particles, in aqueous conditions over certain temperatures and pH ranges.

The purpose of the hydrophobic block (H) is to render the peptide antigen conjugate into a particulate format as a means to modulate pharmacokinetics and promote uptake by antigen-presenting cells. The particles formed by peptide antigen conjugates should be a size between about 10 nm to 10,000 nm in diameter. In preferred embodiments, the particles are nanoparticles that are a size that can be taken up into the endosomal system of cells (such as immune cells). The nanoparticles can be in an average size range of about 10 nm to about 500 nm in diameter. Thus, in some embodiments, the nanoparticles can average about 10 nm, about 20 nm, about 30 nm, about 40 nm, about 50 nm, about 100 nm, 200 nm, 300 nm, 400 nm or 500 nm in diameter. In other embodiments, the nanoparticles can average from about 10-50 nm, or about 10-100 nm, or about 10-200 nm or about 10-500 nm in diameter. In preferred embodiments, the particle size ranges from about 20-200 nm in diameter. The particles in the composition can vary in size, but will generally fall within the size ranges set forth herein. For example, greater than 50%, greater than 55%, greater than 60%, greater than 65%, greater than 70%, greater than 75%, greater than 80%, greater than 85%, greater than 90%, greater than 91%, greater than 92%, greater than 93%, greater than 94%, greater than 95%, greater than 96%, greater than 97%, greater than 98% or greater than 99% of the particles in the composition will fall within the size ranges set forth herein. In some embodiments, the peptide antigen (A) may be linked to an extension (B1 or B2) that is linked either directly or via a Linker (L) to a hydrophobic block (H) that assembles into particles that are too large for uptake by immune cells (e.g., particles larger than about 5,000 nm) and that form a depot at the injection site.

In some embodiments, the hydrophobic block (H) may be in the form of a pre-formed particle, i.e., the particle is formed prior to linking the peptide antigen (A). The particle may be comprised of hydrophobic materials, such as certain polymers or lipids, cross-linked hydrophilic polymers, such as hydrogels, or cross-linked hydrophobic polymers, such as cross-linked polystyrene, that retain structure in aqueous conditions. Non-limiting examples of pre-formed particles include, polymer particles, such as poly(lactic-co-glycolic acid) (PLGA), polymersomes or polaxmers; lipid-based micelles, liposomes, or other lipid-based multi-lamellar vesicles; oil in water emulsions, such as mineral oil-in-water and water-in-mineral oil emulsions; inorganic salt particles, such as aluminum phosphate or aluminum hydroxide salt particles (i.e. Alum); metallic nanoparticles, such as iron oxide particles; silica nanoparticles; and polysaccharide based particles. In some embodiments, the pre-formed particle is a liposomal nanoparticle. In other embodiments, the pre-formed particle is an iron particle. In still other embodiments, the pre-formed particle is a polymer particle. It will be appreciated that these particles are already formed prior to linkage to a peptide antigen (A) and the former particles are distinct from the particles formed by assembly of two or more peptide antigen conjugates comprising a hydrophobic block (H).

The efficiency of peptide antigen (A) linkage to pre-formed particles depends on the nature of the peptide antigen (A) and the type of linkage used. For example, peptide antigens (A) may be adsorbed or incorporated inside pre-formed particles and the efficiency of this process may be empirically determined for each peptide antigen (A), as the nature of the peptide antigen (A) can influence adsorption and incorporation. Peptide antigens (A) may be linked to the pre-formed particles through high affinity interactions (e.g., electrostatic) or a covalent bond, wherein pre-formed particle have a set number of reactive sites that will dictate the number of peptide antigens (A) that can be linked to the pre-formed particles. For immunogenic compositions comprising multiple different peptide antigens (A), for example 20 different peptide antigens (A), multiple copies of each type of peptide antigen (A) may be delivered on separate pre-formed particles or multiple copies of each of the 20 types of peptide antigens (A) may be delivered on the same pre-formed particles.

A limitation of pre-formed particles is that the ratio of peptide antigen (A) to particle cannot be easily controlled. Alternatively, in preferred embodiments the peptide antigen (A) is linked either directly or via a Linker (L) to a hydrophobic block (H) prior to particle assembly. The peptide antigen conjugate comprised of a peptide antigen (A) optionally linked through an extension (B1 or B2) and/or Linker (L) to a hydrophobic block (H) is a molecularly defined entity and the ratio of peptide antigens (A) to the hydrophobic block (H) can be precisely controlled. In preferred embodiments, the ratio is 1:1 peptide antigen (A) to hydrophobic block (H). In additional non-limiting examples, the ratio may be from 1:3 to 3:1 peptide antigens (A) to hydrophobic block (H).

In contrast to preformed-particles, the peptide antigen conjugate may be formed by linking the peptide antigen (A) directly or indirectly through an extension (B1 or B2) and/or a Linker (L) to a hydrophobic block (H) producing a chemically defined single molecule.

The hydrophobic block (H) is a molecule with substantially limited water solubility, or is amphiphilic in properties, and capable of assembling into supramolecular structures, e.g., micellar, nano- or micro-particles in aqueous conditions. In preferred embodiments, the hydrophobic block (H) is insoluble, or forms micelles, in aqueous conditions down to about 0.1 mg/mL or about 0.01 mg/mL.

Hydrophobic blocks (H) as described herein are inclusive of amphiphilic molecules that may form supramolecular structures, such as micelles or bilayer-forming lamellar or multi-lamellar structures (e.g., liposomes or polymersomes), as well as compounds that are completely insoluble and form aggregates. The hydrophobic characteristics of the hydrophobic block (H) may be temperature- and/or pH-responsive. In some embodiments, the hydrophobic block (H) is a polymer that is water soluble at low temperatures but is insoluble, or micelle-forming, at temperatures above, for example, 20° C., such as 20, 21, 22, 23, 24, 25, 26, 27, 28, 29, 30, 31, 32, 33, 34, 35, 36, 37, 38, 39 or 40° C. In other embodiments, the hydrophobic block (H) is a polymer that is water soluble at low pH, for example, at a pH below 6.5 but insoluble, for example, at a pH above 6.5.

The hydrophobic block (H) may be chosen from any of higher alkanes, cyclic aromatics, fatty acids, compounds deriving from terpenes/isoprene or polymers with limited water solubility. Exemplary higher alkanes include but are not limited to octane, nonane, decane, undecane, dodecan, tridecane, tetradecane, pentadecane, hexadecane, heptadecane and octadecane. Exemplary cyclic aromatics include but are not limited to benzene and fused benzene ring structures or heterocyclic aromatic molecules. Exemplary saturated and unsaturated fatty acids include but are not limited to myristic acid, palmitic acid, stearic acid or oleic acid. In other embodiments, the hydrophobic block (H) comprises a diacyl lipid, such a s1,2-dioleoyl-sn-glycero-3-phosphoethanolamine or 1,2-distearoyl-sn-glycero-3-phosphoethanolamine or a lipopeptide, e.g., Pam2Cys. In some embodiments, the fatty acid or lipid based hydrophobic block (H) may further comprise a PEG. Exemplary compounds deriving from terpenes/isoprene include sterol derivatives, such as cholesterol, and squalene. In some embodiments, the hydrophobic block (H) comprises cholesterol.

In preferred embodiments, the hydrophobic block (H) is a polymer with limited water solubility or is amphiphilic and capable of assembling into particles, e.g., micelles in aqueous conditions.

The hydrophobic block (H) may comprise a linear or branched polymer. The polymer can be a homo-polymer, a co-polymer or a terpolymer. The polymer can be comprised of one or many different types of monomer units. The polymer can be a statistical copolymer or alternating copolymer. The polymer can be a block copolymer, such as the A-B type, or the polymer can be comprised of a grafted copolymer, whereby two polymers are linked through polymer analogous reaction.

Exemplary polymers include but are not limited to PLGA, hydrophobic poly(amino acids), poly(benzyl glutamate), polystyrene, polaxmers based on ethylene oxide or propylene oxide monomers and temperature-responsive polymers, such as poly(N,N′-diethyl acrylamide), poly(N-n-propylacrlyamide), Poly(N-isopropylacrylamide), poly[di(ethelyene glycol)methacrylate methyl ether] and certain PEGylated poly(amino acids), such as Poly(γ-(2-methoxyethoxy)esteryl-L-glutamate). In some embodiments, the hydrophobic block (H) is a poly(amino acid) comprised of hydrophobic amino acids. In preferred embodiments, the hydrophobic block (H) comprising a poly(amino acid) is comprised of amino acids that comprise aromatic rings. In other embodiments, the hydrophobic block (H) is a poly(amino acid) that is linked to Ligands, such as PRR agonists. In other embodiments, the hydrophobic block (H) is an A-B type di-block co-polymer. In some embodiments, the di-block co-polymer is temperature-responsive and is capable of assembling into particles in response to a temperature shift. In some embodiments, the hydrophobic block (H) comprises an A-B type di-block co-polymer that further comprises Ligands, such as PRR agonist, linked to one block of the di-block co-polymer.

Advantageously, our data suggests that hydrophobic blocks (H) based on polymers comprising aromatic rings, particularly those substituted with amines, i.e. aryl amines, are easier to work with during manufacturing as compared with hydrophobic polymers, lipids or fatty acids based on aliphatics or sterol derivatives. Specifically, hydrophobic blocks (H) based on polymers comprising aromatic rings tend to have improved solubility in most organic solvents, including DMSO, methanol and ethanol and are compatible with standard reverse phase HPLC analytical and purification methods. Therefore, in preferred embodiments the hydrophobic block (H) comprises aromatic rings. In some embodiments, the hydrophobic block (H) is attached to one or more ligands that comprise an aromatic ring structure. In other embodiments, the ligand that is linked to the polymer-based hydrophobic block comprises a heterocyclic aromatic ring. In some embodiments, the ligand that is linked to the polymer-based hydrophobic block comprises an aromatic ring further comprising an aryl amine. In some embodiments, the hydrophobic ligand linked to the polymer is a PRR agonist.

The polymer may include naturally occurring and synthetic monomers and combinations thereof. Natural biopolymers may include peptides comprised of amino acids; a specific example is poly(tryptophan). The natural biopolymer may be chemically modified. For example, biopolymers comprised of glutamic acid or lysine residues may be modified at the gamma carboxyl or epsilon amino groups, respectively. Biopolymers can be polysaccharides, which may include but are not limited to glycogen, cellulose and dextran. Additional examples include polysaccharides that occur in nature, including alginate and chitosan. Polymers may also be comprised of naturally occurring small molecules, such as lactic acid or glycolic acid, or may be a copolymer of the two (i.e., PLGA).

In some embodiments, the hydrophobic block (H) is comprised of an anionic (e.g., poly(acidic)) polymer or cationic (e.g., poly(basic)) polymer or combinations of anionic and cationic polymers. Cationic polymers can bind to negatively charged peptides by electrostatic interaction or may be useful for complexing negatively charged nucleic acids, such as DNA and RNA. In some embodiments, the polymer is a water insoluble zwitterion at pH 7.4 but carries a net positive charge at pH less than about 6 and is water soluble. In some embodiments, the hydrophobic block (H) comprising a first polymer carries a positive or negative charge that is complementary to the negative or positive charge, respectively, on a second polymer and the first and second polymers form an electrostatic complex through charge neutralization that renders the complex insoluble. In some embodiments, the cationic polymer can be a naturally occurring or synthetic poly(amine), such as poly(lysine) or poly(ethylenimine) (PEI). In additional embodiments, the cationic polymer can be a poly(amido amine) (PAA) or poly(beta amino ester) (PBAE) produced from the Michael addition reaction of amines with either bis(acrylamides) or bis(acrylesters). Non-limiting examples of cationic polymers that can be used in the disclosed embodiments include poly(ethylenimine), poly(allylanion hydrochloride; PAH), putrescine, cadaverine, poly(lysine) (PL), poly(arginine), poly(trimethylenimine), poly(tetramethylenimine), poly(propylenimine), aminoglycoside-polyamine, dideoxy-diamino-b-cyclodextrin, spermine, spermidine, cadaverine, poly(2-dimethylamino)ethyl methacrylate, poly(histidine), cationized gelatin, dendrimers, chitosan, and any combination thereof. The cationic polymer may contain a quaternary ammonium group, such as that present on methylated chitosan. Alternatively, the polymer may be an anionic polymer. In some non-limiting examples the polyanionic polymer is poly(glutamic acid). In alternative embodiments the polyanionic polymer is poly(aspartic acid). The polymer can be a polyphoshphoester-based polymer. The polymer may comprise natural anionic polysaccharides, including, e.g., algininc acid, comprised of 1-4)-linked β-D-mannuronate and guluronic acid. The polymer may comprise nucleotides. Other polyanionic polymers may be equally suited.

In some embodiments, the hydrophobic block (H) is a water soluble cationic polymer over certain pH ranges but is uncharged and water insoluble at pH ranges around physiologic pH 7.4. In some embodiments, the hydrophobic block (H) is a polymer that comprises aromatic amines wherein the pKa of the conjugate acid of the aromatic amine is less than 7.5. At pH below the pKa of the aromatic amines, the aromatic amine is protonated and therefore endows the polymer with positive charge. A non-limiting example of a hydrophobic block (H) comprised of a polymer comprising aromatic amines is poly(phenylalanine amine). In some embodiments, the hydrophobic block (H) is a polymer that comprises nitrogen heterocycles wherein the pKa of a nitrogen atom comprising the heterocycle is less than 7.5. At pH below the pKa of a nitrogen atom comprising the heterocycle, the nitrogen is protonated and endows the polymer with positive charge. A non-limiting example of a hydrophobic block (H) comprised of a polymer comprising a heterocycle with protonatable (i.e. basic) nitrogen atoms is poly(histidine). Herein, we report the unexpected finding that hydrophobic blocks comprised of polymers that comprise a protonatable nitrogen (e.g., aromatic amine or “aryl amine”) provide unexpected improvements in manufacturing, particle stability and biological activity.

In some embodiments, the hydrophobic block (H) can be a poly(diethylene glycol methyl ether methacrylate)-(DEGMA) based polymer. In additional embodiments, the hydrophobic block (H) is a polymer that may include monomers of (meth)acrylates, (meth)acrylamides, styryl and vinyl moieties. Specific examples of (meth)acrylates, (meth)acrylamides, as well as styryl- and vinyl-based monomers include N-2-hydroxypropyl(methacrylamide) (HPMA), hydroxyethyl(methacrylate) (HEMA), Styrene and vinylpyrrolidone (PVP), respectively. The polymer can be a thermoresponsive polymer comprised of monomers of N-isopropylacrylamide (NIPAAm); N-isopropylmethacrylamide (NIPMAm); N,N′-diethylacrylamide (DEAAm); N-(L)-(1-hydroxymethyl)propyl methacrylamide (HMPMAm); N,N′-dimethylethylmethacrylate (DMEMA), 2-(2-methoxyethoxy)ethyl methacrylate (DEGMA). In some embodiments, the hydrophobic polymer is a polymer comprising HPMA, or HPMA DEGMA monomers. In some embodiments, the polymer comprising HPMA and DEGMA monomers is an A-B type di-block polymer. An unexpected finding reported herein is that peptide antigens (A) linked to A-B type di-block co-polymers comprising an HPMA hydrophilic block assemble into nanoparticle micelles of uniform size independent of the peptide antigen (A) composition.

The hydrophobic block (H) may also comprise polymers based on cyclic monomers that include cyclic urethanes, cyclic ethers, cyclic amides, cyclic esters, cyclic anhydrides, cyclic sulfides and cyclic amines.

Hydrophobic blocks (H) based on polymers comprising cyclic monomers may be produced by ring opening polymerization and include polyesters, polyethers, polyamines, polycarbonates, polyamides, polyurethanes and polyphosphates; specific examples may include but are not limited to polycaprolactone and poly(ethylenimine) (PEI). Suitable polymers may also be produced through condensation reactions and include polyamides, polyacetals and polyesters.

In some embodiments, the hydrophobic block (H) is a polymer that can include from 3 to 10,000 monomer units. In preferred embodiments, the polymer includes from about 3 to 300 monomer units, such as from 3 to 10, e.g., 3, 4, 5, 6, 7, 8, 9 10 monomer units; or from about 10 to 100 monomer units, e.g., 10, 20, 30, 40, 50, 60, 70, 80, 90, 100; or from about 100 to 200 monomer units; or from about 200 to 300 monomer units, typically no more than 1,000 monomer units. In some embodiments, the polymer may comprise up to 1,000 to 10,000 monomer units. Typically, at least five monomers are needed to form a sufficient size of the hydrophobic block (H) to promote particle formation of the peptide antigen conjugate, though, unexpectedly, hydrophobic blocks (H) comprised of polymers with as few as 3 monomers that include aromatic rings were sufficient to drive particle assembly of peptide antigen conjugates. Increasing the length of the polymer from 3 to 5 and 5 to 10 monomers increases the strength of the forces promoting particle formation, leading to more stable and larger sized particles formed by the peptide antigen conjugates. In some embodiments, the peptide antigen conjugate comprise a hydrophobic block that comprises a polymer of between 5-100 monomers, which results in the formation of approximately 10-300 nm diameter particles in aqueous conditions. In additional embodiments, the polymer comprising the hydrophobic block (H) is comprised of about 300 monomers and results in peptide antigen conjugates that assemble into particles between about 20 to 500 nm, or about 100-500 nm.

In some embodiments, the average molecular weight of the polymer comprising the hydrophobic block (H) may be between about 1,000 to 1,000,000 g/mol. In preferred embodiments, the average molecular weight of the polymer is between about 1,000 and 60,000 g/mol. In some embodiments, the polymer molecular weight is between about 1,000 and 5,000, or between about 5,000 and 10,000, or between about 10,000 and 20,000, or between about 20,000 and 30,000, or between about 25,000, and 60,000. In some embodiments, the hydrophobic block (H) is an A-B type di-block polymer with an average molecular weight of between about 10,000 g/mol to about 60,000 g/mol, such as about 10,000 g/mol, 20,000 g/mol, 30,000 g/mol, 40,000 g/mol, 50,000 g/mol or 60,000 g/mo. In some embodiments, the polymer is an A-B type di-block polymer wherein the ratio of the molecular weights of the A block and B blocks are about 1:5 to about 5:1. In non-limiting examples, the A-B type di-block polymer with an average molecular weight of about 60,000 g/mol is comprised of an A block with an average molecular weight of about 10,000 g/mol and a B block with an average molecular weight of about 50,000 g/mol; an A block with an average molecular weight of about 20,000 g/mol and a B block with an average molecular weight of about 40,000 g/mol; an A block with an average molecular weight of about 30,000 g/mol and a B block with an average molecular weight of about 30,000 g/mol; an A block with an average molecular weight of about 40,000 g/mol and a B block with an average molecular weight of about 20,000 g/mol; an A block with an average molecular weight of about 50,000 g/mol and a B block with an average molecular weight of about 10,000 g/mol.

The polydispersity, Mw/Mn, of the polymer may range from about 1.0 to about 5.0. Polymers may be formed by a variety of polymerization techniques. Peptide and nucleotide-based polymers may be prepared by solid-phase synthesis and will have polydispersity of 1.0 as the polymers are molecularly defined. Polymers formed by chain growth polymerization will have polydispersities>1.0. Polymers may be synthesized by living polymerization techniques or solution free radial polymerization. In preferred embodiments, peptide based biopolymers are synthesized by solid-phase peptide synthesis. Peptide (or “poly(amino acid)”) based polymers comprising amino acids with aromatic rings, such as tryptophan, though hydrophobic in aqueous conditions, provided unexpected improvements in manufacturing by solid-phase synthesis as compared with peptides (or “poly(amino acids)”) without aromatic rings. Thus, in preferred embodiments, hydrophobic blocks (H) based on peptides produced by solid phase synthesis include amino acids comprising aromatic rings. In additional embodiments, acrylamide- and acrylate-based polymers are synthesized by reversible addition-fragmentation chain-transfer (RAFT) polymerization. In additional embodiments, poly(amino acids) and poly(phosphoesters) are synthesized by ring opening polymerization.

In some embodiments, the hydrophobic block (H) may comprise a polymer that further comprises a Ligand or Ligands, such as PRR agonists. In some embodiments, the polymer-based hydrophobic block (H) may include monomers that comprise at least one functional group that can be coupled to a Ligand, or to a linker that can be coupled to a Ligand. In other embodiments, the polymer-based hydrophobic block (H) may comprise Ligands that are linked to the ends and/or side chains of the polymer.

In preferred embodiments of immunogenic compositions for the treatment or prevention of cancer and infectious diseases, the hydrophobic block (H) is a polymer that is linked to Ligands. In some embodiments, the Ligand comprises an aromatic ring structure, such as heterocyclic aromatic ring, additionally, wherein the Ligand may optionally be a hydrophobic Ligand that promotes increased stability of the particles formed by the peptide antigen conjugate in aqueous conditions. In other embodiments, the Ligand that is linked to the polymer-based hydrophobic block (H) may still further comprise a heterocyclic aromatic ring. In some embodiments, the Ligand that is linked to the polymer-based hydrophobic block (H) comprises an aromatic ring further comprising an aryl amine. In some embodiments, the Ligand attached to the hydrophobic block (H) is a hydrophobic ligand comprising a heterocyclic aromatic ring, optionally wherein the hydrophobic Ligand further comprises an aromatic amine (i.e. Ar—NH₂). In embodiments wherein the hydrophobic block (H) comprises a Ligand that comprises an aromatic group, optionally further comprising a heterocycle and/or aryl amine, we report the unexpected finding that such hydrophobic blocks (H) are highly soluble in pharmaceutically acceptable organic solvents, such as DMSO and ethanol, but insoluble in aqueous buffers.

In some embodiments, the Ligand linked to the polymer-based hydrophobic block (H) is a pattern recognition receptor agonist (PRRa), such as an agonist of STING, NOD receptors or TLRs that has adjuvant properties. The Ligand with adjuvant properties linked to the polymer may be, or be derived from, any suitable adjuvant compound, such as a PRR agonist. Suitable Ligands with adjuvant properties includes compounds that include small organic molecules, i.e., molecules having a molecular weight of less than about 3,000 Daltons, although in some embodiments the adjuvant may have a molecular weight of less than about 700 Daltons and in some cases the adjuvant may have a molecular weight from about 200 Daltons to about 700 Daltons.

The hydrophobic block (H) in preferred embodiments of immunogenic compositions used for the treatment or prevention of cancer or infectious diseases is a polymer linked to Ligands with adjuvant properties. The Ligands with adjuvant properties, such as PRR agonists, can be linked to the side chains or end groups of the polymer through any suitable linker. In some embodiments, monomers comprising a polymer-based hydrophobic block (H) comprise a side chain comprising at least one functional group that can be coupled to a Ligand with adjuvant properties, or to a linker that can be coupled to a Ligand with adjuvant properties. In some embodiments, wherein the polymer-based hydrophobic block (H) comprises a Ligand with adjuvant properties, all of the monomers of the polymer are linked to the Ligand with adjuvant properties. In other embodiments, wherein the hydrophobic block (H) comprises a Ligand with adjuvant properties, not all of the monomers in the polymer are linked to the adjuvant.

In some embodiments, wherein the hydrophobic block (H) comprises a polymer linked to a Ligand with adjuvant properties through monomer units distributed along the backbone of the polymer, increasing the density of the Ligand on the polymer leads to an unexpected improvement in immune responses to the peptide antigen (A).

In certain embodiments, the mole ratio of Ligands with adjuvants properties, such as PRR agonists, to monomers of the polymer may be selected from about 1:100 to 1:1 mol/mol (or about 1 mol % to about 100 mol %), such as from 1:2.5 to 1:1 mol/mol.

The density of the Ligand with adjuvant properties, such as PRR agonists, linked to the polymer-based hydrophobic block (H) can be varied as needed for particular applications. The Ligand with adjuvant properties, such as PRR agonists, may be linked to the polymer from 1 to 100 mol %, such as from 1 to 10 mol % or from 50-100 mol %. Mol % refers to the percentage of monomers comprising the polymer that are linked to Ligand with adjuvant properties, such as PRR agonists. For example, 10 mol % Ligand (e.g., PRR agonists) is equal to 10 monomer units linked to the Ligand from a total 100 monomer units. The remaining 90 may be macromolecule-forming monomeric units, which are not linked to the Ligand.

The density of Ligands, such as Ligands with adjuvant properties, linked to a polymer-based hydrophobic block (H) should be selected to ensure that the peptide antigen conjugate (i) is soluble in pharmaceutically acceptable organic solvents, such as DMSO; (ii) can form stable nanoparticles in aqueous conditions at physiologic temperature and pH; and/or (iii) is capable of inducing an immune response, particularly a T cell response, in a subject.

The optimal density of Ligands, such as PRR agonists, linked to the polymer depends on the polymer composition, polymer length, as well as the composition of the Ligand. When the Ligand is a hydrophobic/amphiphilic molecule with low water solubility, such as an imidazoquinoline-based Toll-like receptor −7 and −8 agonist (TLR-7/8a) and the polymer alone is water soluble (i.e. the polymer not linked to the Ligand is water soluble), the Ligand is typically linked to the polymer at a density of about 20-100 mol % when the polymer is comprised of between about 5-30 monomer units; 10-50 mol % when the polymer is comprised of between 30-100 monomer units; or at a density of between 5-20 mol % when the polymer is comprised of between 100-300 monomer units. In general, the mol % of the Ligand with adjuvant properties is higher for shorter polymers and lower for longer polymers.

The optimal density of the Ligand with adjuvant properties, e.g. PRRa, attached to hydrophilic or temperature-responsive polymers that are greater than 10,000 g/mol and based on co-monomers selected from N-2-hydroxypropyl(methacrylamide) (HPMA), hydroxyethyl(methacrylate) (HEMA), Styrene, vinylpyrrolidone (PVP), N-isopropylacrylamide (NIPAAm), N-isopropylmethacrylamide (NIPMAm), N,N′-diethylacrylamide (DEAAm), N-(L)-(1-hydroxymethyl)propyl methacrylamide (HMPMAm), N,N′-dimethylethylmethacrylate (DMEMA), 2-(2-methoxyethoxy)ethyl methacrylate (DEGMA) or substituted poly(phosphoesters) is from 1 to 25%, e.g., the density of the adjuvant attached to the polymer can be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25%.

In some embodiments, the hydrophobic block (H) is an amphiphilic A-B type di-block co-polymer wherein one block is hydrophobic and the other block is hydrophilic. In some embodiments, where the hydrophobic block (H) is an amphiphilic A-B type di-block co-polymer and the Ligand is hydrophobic, such as an imidazoquinoline TLR-7/8 agonist, the Ligand is preferably linked to the hydrophobic block at a density of between about 1 to 50 mol %, typically about 1 to 20 mol %. In some embodiments, where the hydrophobic block (H) is an amphiphilic A-B type di-block co-polymer and the Ligand is hydrophilic, the Ligand is preferably linked to the hydrophilic block at a density of between about 1 to 20 mol %, or at the hydrophilic end of the hydrophilic block and therefore the A-B type di-block co-polymer is semi-telechelic with respect to the Ligand. In a non-limiting example, the hydrophobic block (H) comprises a temperature-responsive A-B type di-block co-polymer comprising an HPMA block and a DEGMA block and an imidazoquinoline TLR-7/8 agonist is linked to the DEGMA block at a density of between about 1 to 5 mol %. In an additional non-limiting example, the hydrophobic block (H) comprises an A-B type di-block polymer comprising an HPMA co-polymer hydrophobic block and an HPMA homopolymer hydrophilic block and an imidazoquinoline TLR-7/8 agonist is linked to the HPMA co-polymer hydrophobic block at a density of between about 20 mol %. In some embodiments, the hydrophobic block is a tri-block co-polymer, e.g., A-B-A or other multi-block co-polymers compositions.

An unexpected finding reported herein is that peptide antigens (A) linked to A-B type di-block co-polymers comprising an HPMA hydrophilic block linked to Ligands with adjuvant properties assemble into nanoparticles micelles of uniform size independent of the peptide antigen (A) composition and this improved reliability of nanoparticle micelle formation was associated with increased magnitude of T cell immunity. In a non-limiting example, a peptide antigen (A) is linked to a di-block co-polymer comprised of an HPMA co-polymer hydrophobic block (i.e., p[(HPMA)-co-(MA-b-Ala-2B)]) and an HPMA homopolymer hydrophilic block (i.e., p(HPMA)) through a triazole linker (Lys(N3)-DBCO) to form a peptide antigen conjugate p{[(HPMA)-co-(MA-b-Ala-2B)]-b-p(HPMA)}-DBCO-(Lys(N3))-A, wherein 2B is a TLR-7/8a also referred to as Compound 1. In additional embodiments, a peptide antigen (A) is linked to a di-block co-polymer comprised of a DEGMA co-polymer hydrophobic block (i.e., p[(DEGMA)-co-(MA-b-Ala-2B)]) and an HPMA homopolymer hydrophilic block (i.e., p(HPMA)) through a triazole linker (Lys(N3)-DBCO) to form a peptide antigen conjugate p{[(DEGMA)-co-(MA-b-Ala-2B)]-b-p(HPMA)}-DBCO-(Lys(N3))-A, wherein 2B is a TLR-7/8a also referred to as Compound 1. In other embodiments, a peptide antigen (A) is linked to a di-block co-polymer comprised of a DEGMA homopolymer linked to a ligand with adjuvant properties (i.e., 2BXy-p[(DEGMA)) and an HPMA homopolymer hydrophilic block (i.e., p(HPMA)) through a triazole linker (Lys(N3)-DBCO) to form a peptide antigen conjugate 2BXy-p{[(DEGMA)-co-(MA-b-Ala-2B)]-b-p(HPMA)}-DBCO-(Lys(N3))-A, wherein the peptide antigen (A) and 2BXy (TLR-7/8a also referred to as Compound 2) are linked at a single site on opposite ends of the polymer, which makes the polymer hetero-telechelic.

In several embodiments, the hydrophobic block (H) is a poly(amino acid)-based polymer that is comprised of co-monomers of glutamic acid or aspartic acid and aromatic and/or hydrophobic amino acids, such as phenylalanine, amino phenylalanineamine (or “phenylalanineamine”), tryptophan, tyrosine, benzyl glutamate, histidine, leucine, isoleucine, norleucine and valine, and one or more Ligands with adjuvant properties, e.g., PRRa, are attached to the polymer through the gamma carboxylic acid of the glutamic acid or the beta carboxylic acid of aspartic acid. In preferred embodiments, the hydrophobic block (H) is a poly(amino acid)-based polymer comprised of co-monomers of glutamic acid and tryptophan, wherein one or more Ligands with adjuvant properties, e.g., PRRa, are linked to the glutamic acid residues through the gamma carboxylic acid. In additional embodiments, the hydrophobic block (H) is a poly(amino acid)-based polymer compromised of co-monomers of lysine and aromatic and/or hydrophobic amino acids, such as phenylalanine, amino phenylalanine, histidine, tryptophan, tyrosine, benzyl glutamate, leucine, isoleucine, norleucine and valine, wherein one or more Ligands with adjuvant properties, e.g., PRRa, are attached to the polymer through the epsilon amine of lysine. In preferred embodiments, the hydrophobic block (H) is a poly(amino acid)-based polymer comprised of co-monomers of lysine and tryptophan, wherein one or more Ligands with adjuvant properties, e.g., PRRa, are linked to lysine through the epsilon amine. In preferred embodiments, wherein the hydrophobic block (H) is a poly(amino acid) co-polymer linked to one or more Ligands with adjuvant properties, e.g., PRRa, the polymer is between 5-30 amino acids in length and the adjuvant is attached at a density from 20 to 100 mol %, such as 30%, 50%, 60%, 80% and 100 mol %. In additional embodiments, the Ligand is attached only to a single end of the poly(amino acid) polymer, i.e., the polymer is semi-telechelic with respect to the Ligand.

Herein, we report the unexpected finding that hydrophobic block (H) comprised of poly(amino acid)-based co-polymers that further comprise aromatic groups, such as aromatic amino acids (e.g., phenylalanine, amino phenylalanine, histidine, tryptophan, tyrosine, benzyl glutamate) and/or aromatic Ligands (e.g., imidazoquinolines) linked to the polymer, result in unexpected improvements in manufacturability, through improved organic solvent solubility, and improved particle stability and biological activity of peptide antigen conjugates, as compared with poly(amino acids) predominantly comprised of aliphatic amino acids or aliphatic Ligands. An additional unexpected finding is that peptide antigen conjugates comprising hydrophobic blocks (H) that comprise 1-methyl tryptophan result in increased magnitude of immune responses, possibly due to the capacity of 1-methyl tryptophan to inhibit indoleamine 2,3-dioxygenase. Thus, in preferred embodiments, hydrophobic blocks (H) comprised of poly(amino acids), or other classes of polymers, include one or more aromatic amino acids and/or Ligands that comprise an aromatic group.

In additional embodiments, the hydrophobic block (H) is a poly(amino acid)-based polymer comprised entirely of glutamic acid, aspartic acid or non-natural amino acid residues bearing a carboxylic acid wherein the Ligand with adjuvant properties is linked to all of the glutamic acid, aspartic acid or non-natural amino acid residues, i.e., the Ligand with adjuvant properties is attached at a density of 100 mol %. In additional embodiments, the hydrophobic block (H) is a poly(amino acid)-based polymer comprised entirely of Lysine or non-natural amino acids bearing a free amine and the Ligand with adjuvant properties is linked to all of the lysine or non-natural amino acid residues, i.e., the adjuvant is attached at a density of 100 mol %. In additional embodiments, PEGylated co-monomers, such as γ-(2-methoxyethoxy)esteryl-L-glutamate) are included to endow the co-polymer with temperature-responsive properties. In other embodiments, temperature-responsive polymers may be grafted to the pendant side chains of the poly(amino acid) to form a graft co-polymer. In additional embodiments, a temperature-responsive polymer may be linked to the end of the poly(amino acid) polymers to form a temperature-responsive di-block polymer. In still additional embodiments, a second polymer that is hydrophobic may be linked to the poly(amino acid) polymer that is linked to Ligands with adjuvant properties either through pendant side groups to form a graft co-polymer, or to the end of the poly(amino acid) to form a di-block co-polymer.

In some embodiments, the hydrophobic block (H) is a poly(amino acid)-based polymer linked to a hydrophobic ligand (“Ligand”), such as a hydrophobic adjuvant, and has the formula:

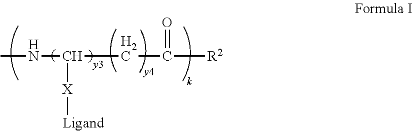

In Formula I, R² is typically selected from one of hydrogen, hydroxyl or amine. In some embodiments, R² is linked to an adjuvant or another polymer through any suitable linker molecule. The integer, y3, is typically 1 to 6, such as 1, 2, 3, 4, 5, or 6. The number of methylene units, y4, is typically 0 to 6, such as 0, 1, 2, 3, 4, 5, or 6. The number of monomer repeats is indicated by k, and is typically between 3 and 300. A Ligand is linked to backbone of the poly(amino acid) through the linker, X. In preferred embodiments of formula I, the Ligand is a hydrophobic ligand (“Ligand”), such as a hydrophobic adjuvant. In some embodiments, the linker X can be linked to a second polymer that is linked to Ligands. In some embodiments, the monomers k may be each linked to the same Ligand or to two or more different ligands.

The N-terminus of the poly(amino acid) of Formula I may be linked through the Linker (L) to the peptide antigen (A) through any suitable means. In some embodiments, the N-terminus of the poly(amino acid) of Formula I is linked directly to the C-terminus of the peptide antigen (A) or to the C-terminus of the B2 extension through an amide bond. In other embodiments, the N-terminus of the poly(amino acid) of Formula I is linked to a linker precursor (X2) that reacts with a linker precursor (X1) that is linked directly or through an extension (B1 or B2) to the peptide antigen (A). In some embodiments, a cyclooctyne containing linker precursor (X2) is attached to the N-terminus of the poly(amino acid) of Formula I and links to azido containing linker precursor (X1) of the peptide antigen fragment.

In some embodiments, the integer, y3, in Formula I is equal to 1 and Formula I reduces to Formula I(a).

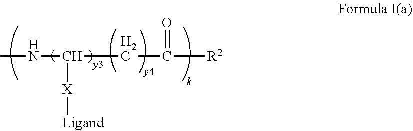

In some embodiments, the integer, y4, is equal to 0 and Formula I(a) further reduces to Formula I (b).

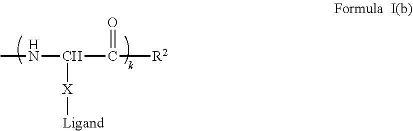

In preferred embodiments, the linker, X, joining the poly(amino acid) backbone to the Ligand is an alkyl chain terminated with a Functional Group (FG). Formula I(b) can thus be elaborated to give Formula I(c).

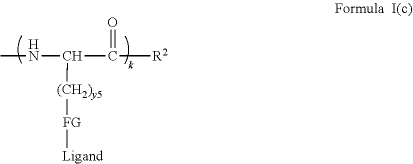

In Formula I(c), the integer, y5, is typically 0 to 6 such as 0, 1, 2, 3, 4, 5, or 6. The Functional Group (FG) included in Formula I(c) is typically selected from carboxylic acid, amine, thiol, aldehyde, ketone, hydrazine, azide, or alkyne. In preferred embodiments, the FG links the Ligand to the poly(amino acid) backbone either directly or through a linker. In some embodiments, the FG can be linked to a second polymer.

For clarity, any references to Formula I disclosed herein refer to any possible embodiments of poly(amino acids) of Formula I, including Formula I, Formula I(a), Formula I(b) or Formula I(c), unless specifically stated otherwise.

Optionally, the hydrophobic blocks (H) may be a poly(amino acid)-based polymer comprised of four different classes of co-monomers, namely hydrophobic monomers ( [symbol] ), spacer monomers (m), charged amino acid monomers ( [symbol] ) for charge compensation, and functional group containing monomers (o) for ligand attachment. The different co-monomers may be included in the hydrophobic block (H) for different reasons. Hydrophobic monomers ( [symbol] ) comprising aromatic containing amino acids are selected to increase the hydrophobic properties of the backbone. Spacer monomers (m) such as glycine, serine and alanine can be selected to increase spacing between monomers of Ligands attached through the monomer (o). Charged co-monomers (n) are selected to balance a charged Ligand such that the overall charge of the hydrophobic block (H) is zero. Functional group containing monomers (o) are used for Ligand attachment.

The hydrophobic blocks (H) may be a poly(amino acid)-based polymer comprising one or more hydrophobic monomers ( [symbol] ), optionally one or more spacer monomers (m), optionally one or more charged amino acid monomers (n), and optionally one or more ligand containing monomers (o). The hydrophobic monomers, spacer monomers, charged amino acid monomers and ligand containing monomers can be assembled in any combination and any order.

In some embodiments, the hydrophobic block (H) is a poly(amino acid)-based polymer that has the formula:

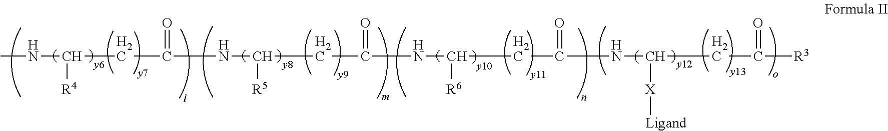

The poly(amino acid)-based polymer of Formula II typically comprises the monomer [symbol] and optional monomers, m, n and o. R³ is typically selected from one of hydrogen, hydroxyl or amine. In some embodiments, R³ is a Ligand, such as a Ligand with adjuvant properties, or another polymer that is linked to the hydrophobic block (H) through any suitable linker molecule. The number of side groups comprising each monomer is indicated by integers represented by y6, y8, y10, and y12 and are typically from 1 to 6 such as 1, 2, 3, 4, 5, or 6. The number of methylene units denoted by y7, y9, y11, and y13, is typically 0 to 6, such as 0, 1, 2, 3, 4, 5, or 6. The N-terminal amine of the poly(amino acid) of Formula II is typically linked to the linker precursor X2 or may be linked to the peptide antigen (A) either directly or via an extension (B1 or B2). In typical embodiments, the poly(amino acid)-based polymer of Formula II comprises monomer(s) [symbol] that are selected from any natural or non-natural amino acid wherein R⁴ is selected from lower alkyl or aromatic groups and endow the polymer backbone with hydrophobic properties. In some embodiments, the R⁴ included in Formula II can be selected from

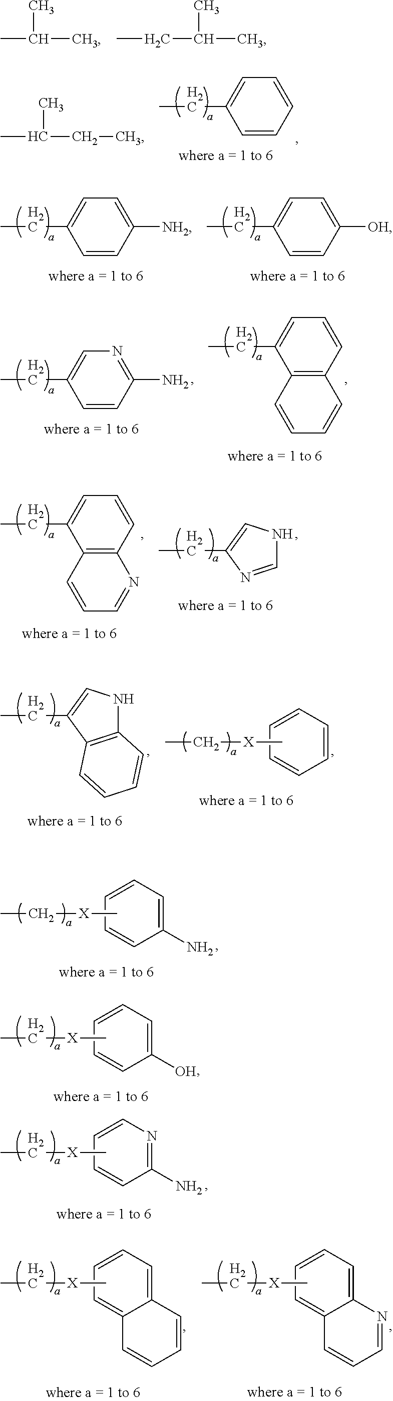

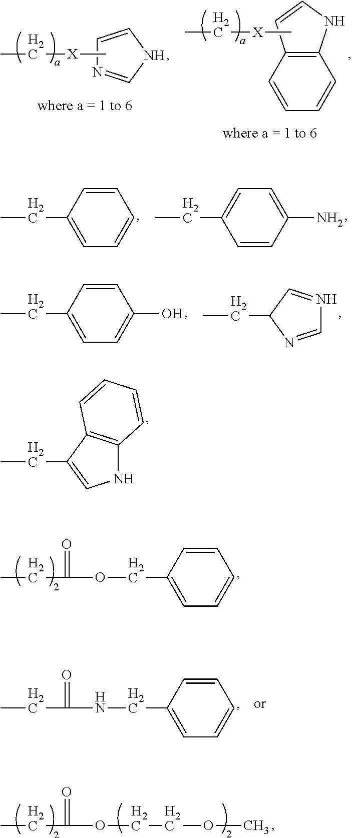

wherein X of R⁴ is any suitable linker.

In some embodiments, the poly(amino acid)-based polymer of Formula II comprises optional co-monomer(s) m that are selected from any natural or non-natural amino acid, such as a PEG amino acid spacer (e.g., m of Formula II is —NH—(CH₂—CH₂—O)_{y14}—(CH₂)_{y15}—(CO)—, wherein y14 is an integer typically between 1 and 24 and y15 is an integer typically between 1 and 3) or an amino acid with a small substituent, wherein, e.g., R⁵ is selected from Hydrogen, lower alkyl or a lower alkyl comprising a hydroxyl and is provided to increase the spacing or flexibility of the polymer backbone. In some embodiments, the R⁵ included in Formula II can be selected from

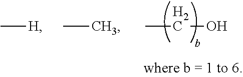

In some embodiments, the poly(amino acid)-based polymer of Formula II comprises optional co-monomer(s) n that are selected from any natural or non-natural amino acid, wherein R⁶ is selected from any group comprising a functional group that carriers charge either permanently or at a specific pH. In some embodiments, the R⁶ included in Formula II can be selected from

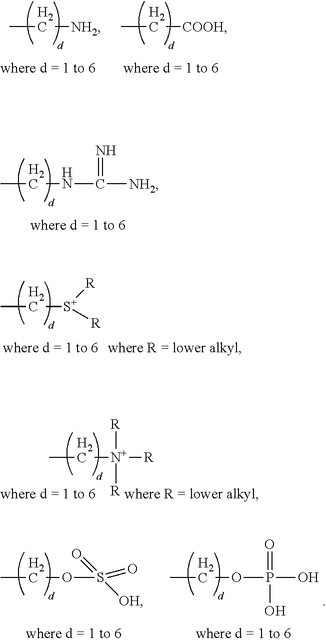

In some embodiments, the poly(amino acid)-based polymer of Formula II comprises optional co-monomer(s) o that are selected from any natural or non-natural amino acid, wherein a Ligand is linked through any suitable linker, X, to the monomer o. The Ligand may be a Ligand with adjuvant properties. The Ligand linked to poly(amino acids) of Formula II may be hydrophobic, hydrophilic, amphiphilic, charged or neutral in properties. The poly(amino acid)-based polymer of Formula II comprising monomer(s) o may further comprise monomer units, [symbol] , m and n, that compensate for the properties of the Ligand attached to monomer o.

In poly(amino acid)-based polymers of Formula II, the number of monomer repeats is indicated by [symbol] , m, n and o, wherein the sum of [symbol] , m, n and o is typically any integer between 3 and 300. Each of the different types of monomers [symbol] , m, n or o, may be the same or different. The monomers denoted by “ [symbol] ” endow the poly(amino acid)-based polymer of Formula II with hydrophobic properties, i.e., render the polymer a water insoluble hydrophobic block (H). The hydrophobic monomers [symbol] may be the same or different and typically comprise an aromatic ring. In preferred embodiments, the hydrophobic monomers [symbol] comprise a heterocyclic and/or amine-substituted aromatic ring. The optional co-monomer(s) denoted by “m” may be used to increase the flexibility or spacing of different monomers comprising the polymer backbone. The optional co-monomer(s) denoted by “n” comprise charged functional groups. The optional co-monomer(s) denoted by “o” are used for the attachment of a Ligand, such as a PRR agonist. In some embodiments, the Ligand linked to the monomer o through any suitable linker is a PRR agonist. In some embodiments, the monomer o is linked to a Ligand that carries a positive or negative charge and is adjacent to a monomer n of the opposite charge. In some embodiments, a charged co-monomer n is placed adjacent to a co-monomer o comprising a functional group of the opposite charge of the functional group comprising the co-monomer n and the opposing charges result in zero net charge, thus the monomer n functions to neutralize charge carried by the Ligand attached to monomer o.

The percentage of monomers, [symbol] , m, n and o comprising the poly(amino acid)-based polymer of Formula II depends on the specific application. In some embodiments, the poly(amino acid)-based polymer of Formula II is comprised entirely of the monomer [symbol] . In other embodiments, the poly(amino acid)-based polymer of Formula II is comprised of co-monomers, [symbol] and o, such as between 5 to 95 mol % monomer [symbol] and about 95 to 5 mol % monomer o. In some embodiments, the poly(amino acid)-based polymer of Formula II comprises co-monomers [symbol] and m, wherein m provides space, i.e. distance, between the hydrophobic monomers [symbol] and may reduce polymer rigidity. In other embodiments, the poly(amino acid)-based polymer of Formula II comprises monomers [symbol] , m and o wherein monomers m provide space between the bulky substituents comprising monomers [symbol] and o. In other embodiments, the poly(amino acid)-based polymer of Formula II comprises monomers [symbol] and o and optionally monomers m and n, wherein monomer n is used to modulate the charge of the polymer backbone. In certain embodiments, the poly(amino acid)-based polymer of Formula II is comprised entirely of monomers m and o. In other embodiments the poly(amino acid)-based polymer of Formula II is comprised entirely of monomers m, n and o, or just n and o. In still other embodiments, the poly(amino acid)-based polymer of Formula II comprises monomers [symbol] , m, n and o.

Wherein the poly(amino acid)-based polymer of Formula II comprises a Ligand with adjuvant properties, the percentage of monomers comprising the polymer represented by the monomer o which is linked to a Ligand with adjuvant properties via any suitable linker is typically 10 to 60%, for example, between 2 to 12 amino acids of a polymer that is 20 amino acids in length are monomer o. In some embodiments of the poly(amino acid) polymers of Formula II, [symbol] is the majority monomer unit. In additional embodiments, the poly(amino acid)-based polymer of Formula II is comprised entirely of the [symbol] monomer, i.e. all monomers are the [symbol] monomer, optionally wherein the adjuvant is attached to the end of the poly(amino acid) either directly or indirectly via a second polymer or through any suitable linker molecule.

In some embodiments of poly(amino acid)-based hydrophobic blocks of Formula (II), the integers y6, y8, y10, and y12 are equal to 1 and Formula II reduces to:

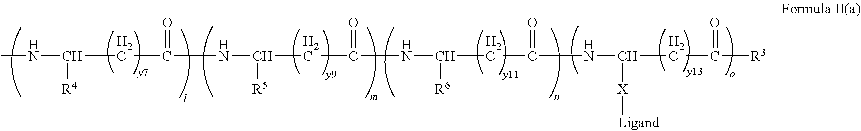

In some embodiments, the integers y7, y9, y11, and y13 are equal to 0 and Formula II(a) further reduces to Formula II(b).

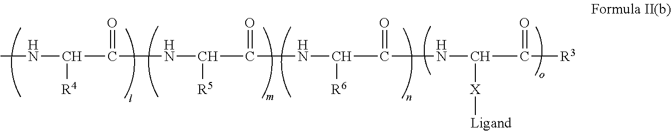

In preferred embodiments, the linker, X, is comprised of an alkyl chain with a Functional Group (FG). Formula II(b) can thus be elaborated to give Formula II(c).

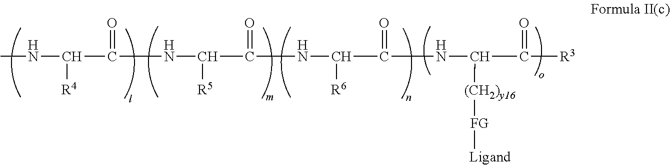

In Formula II(c), the integer, y16, is typically 0 to 6 such as 0, 1, 2, 3, 4, 5, or 6. The Functional Group (FG) included in Formula II(c) is typically selected from carboxylic acid, amine, thiol, aldehyde, ketone, hydrazine, azide, or alkyne. In preferred embodiments, the FG links the Ligand to the poly(amino acid) backbone either directly or through a linker. In some embodiments, the FG can be linked to a second polymer.

For clarity, any references to Formula II disclosed herein refer to any possible embodiment of poly(amino acids) of Formula II, including Formula II, Formula II(a), Formula II(b) and/or Formula II(c), unless specifically stated otherwise.

Ligands with adjuvant properties may be linked to any of the hydrophobic blocks (H) of the present disclosure. In certain embodiments, Ligands with adjuvant properties are linked to polymers of Formula II. Ligands with adjuvant properties may be linked to poly(amino acid)-based polymers of Formula II through pendant functional groups (FG) on the o monomers; at the ends of the polymer; or indirectly through another molecule or polymer that is grafted to the pendant functional groups (FG) or at the ends of the polymer. In preferred embodiments, the functional group (FG) of monomers o link the Ligands with adjuvant properties, or other Ligand molecules, to the poly(amino acid) backbone through a covalent bond. In some embodiments, the FG comprising monomer o of Formula II can be linked to a second polymer. The FG included in Formula II can be selected from carboxylic acid, aldehyde, ketone, amine, hydrazine, thiol, azide or alkyne, or any suitable functional group that can be used to link a Ligand or another polymer to the polymer backbone.

In preferred embodiments, the N-terminus of the poly(amino acid) of Formula II is linked through the Linker (L) to the peptide antigen (A), either directly or through an extension (B1 or B2) through the reaction of the linker precursors X1 and X2. In some embodiments, the N-terminus of the poly(amino acid) of Formula II is linked directly (i.e. no Linker (L) is present) to the C-terminus of the peptide antigen (A) or to the C-terminus of the B2 extension through an amide bond. In other embodiments, the N-terminus of the poly(amino acid) of Formula II is linked to a cyclooctyne group (e.g., DBCO) containing linker precursor (X2) that reacts with azido containing linker precursor (X1) that is linked either directly or through an extension (B1 or B2) to the peptide antigen (A).

The poly(amino acid) of Formula I or Formula II is a hydrophobic block (H) that may be linked either through the N-terminal amine, C-terminal carboxylic acid or optionally through side chains distributed along the backbone of the poly(amino acid) through a Linker (L) either directly, or indirectly through an extension (B1 or B2), to a peptide antigen (A).

In some embodiments, the poly(amino acid) of Formula I or Formula II is a hydrophobic block (H) that is linked at the N-terminus via the Linker (L) to a C-terminal extension (B2) that is linked to a peptide antigen (A) to provide a peptide antigen conjugate of formula A-B2-L-H.

In some other embodiments, the poly(amino acid) of Formula I or Formula II is a hydrophobic block (H) that is linked at the N-terminus via the Linker (L) that is linked to a C-terminal extension (B2) that is linked to the C-terminus of peptide antigen (A) that is linked at the N-terminus to an N-terminal extension (B1) that is linked to a charged moiety (C) to provide a peptide antigen conjugate of formula C-B1-A-B2-L-H.

In some other embodiments, the charged moiety (C) may be linked directly to the hydrophobic block (H) comprised of a poly(amino acid) of Formula I or Formula II, or via the Linker (L) that is linked to the peptide antigen (A) either directly or via an extension. Here, for A-B2-L(C)-H or A-B2-L-H(C), it is intended that the parenthesis notation indicates that C is linked directly to either L or H, wherein L and H are also linked together.

An unexpected finding disclosed herein is that the reaction rate for attachment of the X2 linker precursor to the N-terminal position of peptide-based hydrophobic blocks can be increased by increasing the number of methylene units between the amide and the N-terminal amine of the terminal amino acid. Importantly, these findings are not limited to the reactivity of the hydrophobic block (H) and suggest that amino acid-based linkers, whenever possible, should comprise two or more methylene units. Therefore, in preferred embodiments, the N-terminal amino acid of hydrophobic blocks of Formula I and II comprise two or more, typically between 2 and 7, such as 1, 2, 3, 4, 5, 6, 7 methylene units. For clarity, an amino acid with 2 methylene units is beta-alanine and an amino acid with 5 methylene units is amino-hexanoic acid. In preferred embodiments, the N-terminal amino acid of peptide-based hydrophobic blocks of Formula I and II is amino-hexanoic acid. In other embodiments, the N-terminal amino acid of peptide-based hydrophobic blocks of Formula I and II is beta-alanine.

In some embodiments, a Ligand is attached to the hydrophobic block (H) of the peptide antigen conjugate. In preferred embodiments, the Ligand attached to the hydrophobic block (H) is a hydrophobic ligand comprising an aromatic ring. In some embodiments, the Ligand attached to the hydrophobic block (H) is a hydrophobic ligand comprising a heterocyclic aromatic ring, optionally wherein the hydrophobic ligand further comprises an aromatic amine (i.e. Ar—NH₂). In such embodiments, the hydrophobic aromatic ring, optionally comprising a heterocycle and/or aryl amine, provides the unexpected properties that peptide antigen conjugates comprising such structures are highly soluble in pharmaceutically acceptable organic solvents, such as DMSO and ethanol, but insoluble in aqueous buffers.

In preferred embodiments, the hydrophobic block (H) comprises a poly(amino acid) wherein a hydrophobic ligand is attached to side groups distributed along the backbone of the poly(amino acid). In some embodiments, the hydrophobic ligand comprises an aromatic ring, optionally further comprising a heterocycle or aryl amine.

In some embodiments, the Ligand attached to the hydrophobic block is an immuno-modulator or other Ligand with adjuvant properties, such as a PRR agonist. The adjuvant may either be hydrophobic or hydrophilic in properties. In preferred embodiments, the Ligand comprises an aromatic heterocycle.

In several embodiments, the Ligand with adjuvant properties can be a pattern recognition receptor (PRR) agonist. Non-limiting examples of pattern recognition receptor (PRR) agonists include TLR-1/2/6 agonists (e.g., lipopeptides and glycolipids, such as Pam2cys or Pam3cys lipopeptides); TLR-3 agonists (e.g., dsRNA, such as PolyI:C, and nucleotide base analogs); TLR-4 agonists (e.g., lipopolysaccharide (LPS) derivatives, for example, monophosphoryl lipid A (MPL) and small molecules based on pyrimidoindole); TLR5 agonists (e.g., Flagellin); TLR-7 & -8 agonists (e.g., ssRNA and nucleotide base analogs, including derivatives of imidazoquinolines, hydroxy-adenine, benzonapthyridine and loxoribine); TLR-9 agonists (e.g., unmethylated CpG); Stimulator of Interferon Genes (STING) agonists (e.g., cyclic dinucleotides, such as cyclic diadenylate monophosphate); C-type lectin receptor (CLR) agonists (such as various mono, di, tri and polymeric sugars that can be linear or branched, e.g., mannose, Lewis-X tri-saccharides, etc.); RIG-I-like receptor (RLR) agonists; NOD-like receptor (NLR) agonists (such as peptidogylcans and structural motifs from bacteria, e.g., meso-diaminopimelic acid and muramyl dipeptide); and combinations thereof. In several embodiments, the pattern recognition receptor agonist can be a TLR agonist, such as an imidazoquinoline-based TLR-7/8 agonist. For example, the Ligand with adjuvant properties can be Imiquimod (R837) or Resiquimod (R848), which are approved by the FDA for human use.

In several embodiments, the Ligand with adjuvant properties can be a TLR-7 agonist, a TLR-8 agonist and/or a TLR-7/8 agonist. Numerous such agonists are known, including many different imidazoquinoline compounds.

Imidazoquinolines are of use in the methods disclosed herein. Imidazoquinolines are synthetic immunomodulatory drugs that act by binding Toll-like receptors 7 and 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod and/or is not resiquimod.

In some embodiments, the Ligand with adjuvant properties can be a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the ligand with adjuvant properties is an imidazoquinoline with the formula:

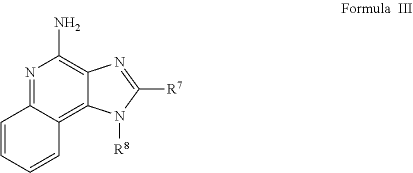

In Formula III, R is selected from one of hydrogen, optionally-substituted lower alkyl, or optionally-substituted lower ether; and R⁸ is selected from one of optionally substituted arylamine, or optionally substituted lower alkylamine. R⁸ may be optionally substituted to a linker that links to a polymer. An unexpected finding was that in some compounds wherein R⁸ was selected from a lower alkylamine, while the compound was less potent than R⁸ selected from an arylamine, the quality of response was improved. Thus, moderate potency Adjuvants of Formula III led to better quality responses. Note: Adjuvant(s) of Formula III are a type of Ligand and may be referred to as Adjuvants of Formula III or Ligands with adjuvant properties.

In some embodiments, the R⁷ included in Formula III can be selected from hydrogen,

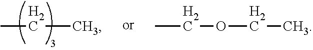

In some embodiments, R⁸ can be selected from,

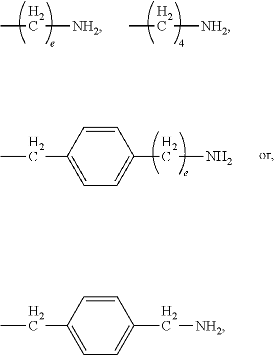

wherein e denotes the number of methylene units and is an integer from 1 to 4.

In some embodiments, R⁸ can be

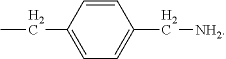

In some embodiments, R⁸ can be

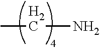

In some embodiments, R⁷ can be

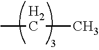

### and R⁸ can be

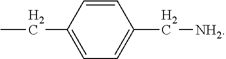

Adjuvants of Formula III, wherein

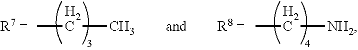

are referred to as Compound 1, while adjuvants of Formula III wherein

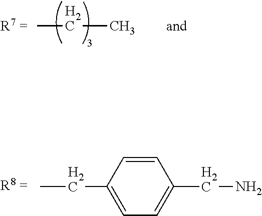

are referred to as Compound 2.

Non-limiting examples of hydrophobic blocks (H) comprised of poly(amino acids) of Formula I linked to adjuvants of Formula III include:

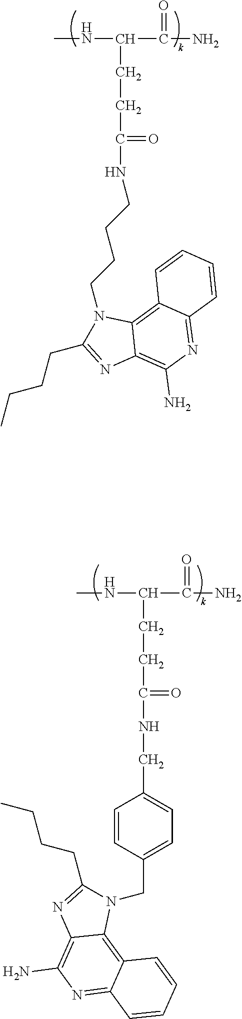

wherein k is between 3-300. For example, when k=5, the peptide is comprised of 5 amino acids linked to Adjuvants of Formula III. In some embodiments, hydrophobic blocks (H) comprised of poly(amino acids) of Formula I linked to adjuvants of Formula III may be linked either directly or indirectly via a Linker (L) and/or extension (either B1 or B2) to a peptide antigen (A) that is optionally linked to a charged moiety (C) through any suitable means to form a peptide antigen conjugate. In some embodiments, the N-terminus of the poly(amino acid) of Formula I linked to adjuvants of Formula III is linked directly to the C-terminus of the peptide antigen (A) or to the C-terminus of the B2 extension through an amide bond. In other embodiments, the N-terminus of the poly(amino acid) of Formula I linked to adjuvants of Formula III is linked to a linker precursor X2 bearing a clickable group, e.g., alkyne or DBCO, or a thiol-reactive linker precursor X2, e.g., maleimide, that reacts with a linker precursor X1 that is linked directly or through an extension (B1 or B2) to the peptide antigen (A). In preferred embodiments, a linker precursor X2 bearing a DBCO molecule is attached to the N-terminus of the poly(amino acid) of Formula I linked to adjuvants of Formula III and is used to react with an azide bearing linker precursor X1 that is linked either directly or through an extension (B1 or B2) to a peptide antigen (A).

A non-limiting example of a hydrophobic block (H) comprised of poly(amino acids) of Formula II linked to adjuvants of Formula III includes:

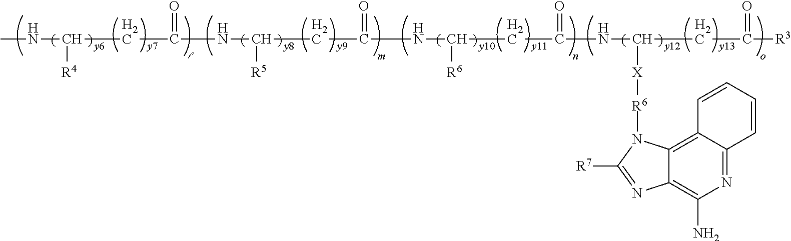

For example:

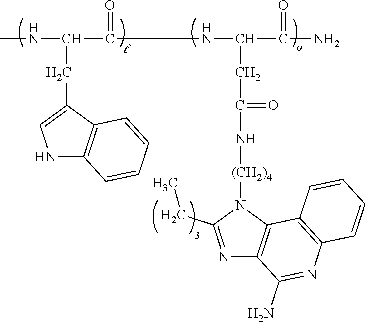

wherein co-monomer [symbol] is typically an integer between 3-300, and optional co-monomer o is typically an integer between 3-300 amino acid residues, wherein the sum of [symbol] and o is typically between about 3-300. Alternatively, o is 0 and the polymer is entirely comprised of [symbol] , i.e., the polymer is a poly(tryptophan) polymer that is not linked to adjuvants. In some embodiments, the N-terminus of the poly(amino acid) of Formula II linked to Adjuvants of Formula III is linked either directly or indirectly through a Linker (L) and/or extension (B1 or B2) to a peptide antigen (A) through any suitable means. In some embodiments, the N-terminus of the poly(amino acid) of Formula II linked to Adjuvants of Formula III is linked directly to the C-terminus of the peptide antigen (A) or to the C-terminus of the B2 extension through an amide bond. In other embodiments, the N-terminus of the poly(amino acid) of Formula II linked to adjuvants of Formula III is linked to a linker precursor X2 bearing a clickable group, e.g., DBCO, or a thiol-reactive linker precursor X2, e.g., maleimide, that reacts with a linker precursor X1 that is linked either directly or through an extension (B1 or B2) to the peptide antigen (A). In preferred embodiments, a DBCO linker precursor X2 is attached to the N-terminus of the poly(amino acid) of Formula II linked to Adjuvants of Formula III and is used to react with a linker precursor X1 bearing an azide functional group.

The length of the polymer comprising the hydrophobic block (H) and the number and potency of Ligands (e.g., Ligand with adjuvant properties, such as PRR agonists) attached are potentially important parameters that impact the activity of peptide antigen conjugates. The hydrophobic block (H) comprised of poly(amino acids) that are comprised of hydrophobic amino acids and/or amino acids linked to Ligands should be sufficiently long to permit particle formation when linked to any peptide antigen (A), including highly hydrophilic peptide antigens (A) that will counter the tendency of the poly(amino acid)-based hydrophobic block (H) to drive particle assembly. Poly(amino acids) of insufficient length, for example, may not provide sufficient hydrophobic surface area to promote particle formation in aqueous conditions when linked to certain peptide antigens (A), particularly hydrophilic peptide antigens (A) with high charge density. Therefore, as disclosed herein, poly(amino acids) of Formula I or Formula II should be greater than 3 amino acids in length, preferably between 5-30 amino acids in length, such as 5, 6, 7, 8, 9, 10, 20 or 30 amino acids in length. While poly(amino acids) greater than 30 amino acids, such as about 30, 40 or 50 amino acids in length bearing multiple hydrophobic amino acids or hydrophobic ligands may be considered difficult to produce by solid-phase peptide synthesis, an unexpected finding reported herein, is that poly(amino acids) comprising amines, aromatic groups and/or aryl amines result in improved manufacturability as compared with sequences without these groups.

In a non-limiting example, a peptide antigen (A) is linked to a hydrophobic block (H) and may additionally comprise optional extensions (B1 and/or B2) and an optional Linker (L) to yield a peptide antigen conjugate of Formula IV, wherein [ ] denotes that the group is optional:

[B1]-A-[B2]-[L]-H or H-[L]-[B1]-A-[B2] Formula IV

The peptide antigen (A) of Formula IV is comprised of an integer number of amino acids, n, wherein n is typically between 7-35, such as 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids, and the hydrophobic block (H) is typically a poly(amino acid) of Formula I or II linked to an Adjuvant of Formula III.

A non-limiting example of a peptide antigen conjugate of Formula IV comprised of a peptide antigen (A) that is optionally linked at the N-terminus to a cathepsin cleavable tetrapeptide extensions (B1=Lys-Pro-Leu-Arg SEQ ID NO: 16) and at the C-terminus to a combined immuno-proteasome and cathepsin cleavable hexapeptide extension (B2=Gly-Gly-Ser-Leu-Val-Arg SEQ ID NO: 4) that is linked to a triazole Linker (L) that is linked to a hydrophobic block (H) comprised of a poly(amino acid) of Formula I that is linked to an Adjuvant of Formula III is shown here as an example:

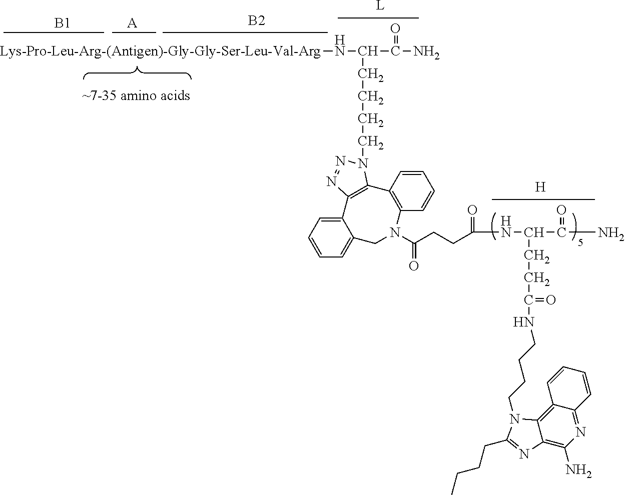

### Charged Moiety (C)

Peptide antigens (A) exhibit a broad range of physical and chemical properties that can influence the size and stability of particles formed by peptide antigen conjugates. Without compensating for the range of physical and chemical properties that are possible for different peptide antigens (A), the peptide antigen conjugate may exhibit a range of hydrodynamic behaviours, including existing as nano-sized supramolecular associates (e.g., micelles), sub-micron or micron-sized particles or aggregates in aqueous conditions. To permit greater control over the hydrodynamic behaviour (i.e. size and stability) of particles formed by peptide antigen conjugates comprised of peptide antigens (A) linked to hydrophobic blocks (H), charged moieties (C) may be linked either directly to the peptide antigen (A), or indirectly either through the extensions (B1 and/or B2); through the linker (L), or through the hydrophobic block (H) to the peptide antigen (A). The purpose of the charged moiety (C) is to provide control over the overall charge and stability of the particles formed by the peptide antigen conjugates in aqueous conditions.

The immunogenic compositions disclosed herein comprising peptide antigens linked to the hydrophobic block (H) that assemble into particles in aqueous conditions may flocculate without sufficient surface charge to stabilize the particles. Thus, charged moieties (C) may be optionally linked to peptide antigen conjugates as a means to stabilize the particles and prevent flocculation.

The charged moieties (C) are selected based on the predicted charge of the peptide antigen at physiologic conditions, a pH of about 7.4, and the required net charge for the peptide antigen conjugate. The charged moieties (C) bear functional groups that impart electrostatic charge. In some embodiments, the charged moieties (C) are peptides comprised of natural or non-natural amino acids that contain either basic or acidic functional groups that impart either positive or negative charge, respectively.

A charged moiety (C) refers to any molecule that has one or more functional groups that are positively or negatively charged in aqueous buffers at a pH of about 7.4. The functional groups comprising the charged moiety (C) may be partial or full integer values of charge. A charged moiety (C) may be a molecule with a single charged functional group or multiple charged functional groups. The net charge of the charged moiety (C) may be positive, negative or neutral. The charge of functional groups comprising the charged moiety (C) may be dependent or independent of the pH of the solution in which the charged moiety (C) is dispersed, such is the case, for example, for tertiary amines and quaternary ammonium compounds that are pH dependent and pH independent, respectively. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule.

The charged moiety (C) may either carry a net negative, net positive or neutral charge and depends on the net charge of the peptide antigen conjugate needed for the specific application of the invention disclosed herein. For example, most cell surfaces are known to carry a net negative charge. Thus, net positively charged particles may interact with all cell surfaces without a high degree of specificity. In contrast, net negatively charged particles will be electrostatically repulsed from most cell surfaces but have been shown to promote selective uptake by certain antigen-presenting cell populations. For example, positively charged particles delivered intravenously into the circulation have been found to accumulate in the liver and lungs as well as within antigen-presenting cells in the spleen, whereas negatively charged particles have been found to preferentially accumulate in antigen-presenting cells in the spleen following intravenous administration. Thus, the net charge of the charged moiety (C) can be adjusted to meet the specific demands of the application.

In some embodiments, the charged moiety (C) has a net negative charge and is comprised of functional groups that carry a negative charge at physiologic pH, at a pH of about 7.4. Suitable charged moieties (C) that carry a net negative charge include molecules bearing functional groups (e.g., functional groups with a pKa less than about 6.5) that occur as the conjugate base of an acid at physiologic pH, at a pH of about 7.4. These include but are not limited to molecules bearing carboxylates, sulfates, phosphates, phosphoramidates, and phosphonates. The charged moiety (C) bearing a carboxylate can be but is not limited to glutamic acid, aspartic acid, pyruvic acid, lactic acid, glycolic acid, glucuronic acid, citrate, isocitrate, alpha-keto-glutarate, succinate, fumarate, malate, and oxaloacetate and derivatives thereof. In preferred embodiments, the negatively charged moiety (C) is comprised of a molecule with between 1-20 negatively charged functional groups, such as 1, 2, 3, 4, 5, 6, 7, 8, 9, 10, 11, 12, 13, 14, 15, 16, 17, 18, 19 or 20 negatively charged functional groups, though, typically no more than 16 negatively charged functional groups. In some embodiments, the charged moiety (C) is a poly(glutamic acid) peptide of between 2-6 amino acids in length. A poly(glutamic acid) sequence comprised of 1, 2, 3, 4, 5 or 6 amino acids would be expected to carry a negative charge of −1, −2, −3, −4, −5 and −6 at pH 7.4, respectively. In additional embodiments, the charged moiety (C) is phosphoserine or sulfoserine.

In certain embodiments, the charged moiety (C) has a net negative charge and is comprised of 1 or more negatively charged amino acids. In preferred embodiments, the charged moiety (C) with a net negative charge is comprised of between 1 to 20 negatively charged amino acids, e.g., 1, 2, 3, 4, 5, 6, 7, 8, 9, 10, 11, 12, 13, 14, 15, 16, 17, 18, 19 or 20. In a non-limiting example, a charged moiety (C) is comprised of 16 aspartic acid monomers, e.g., Asp-Asp-Asp-Asp-Asp-Asp-Asp-Asp-Asp-Asp-Asp-Asp-Asp-Asp-Asp-Asp, is used to prepare a charged moiety (C) with a net negative charge of −16; a charged moiety (C) comprised of 15 aspartic acid monomers, e.g., Asp-Asp-Asp-Asp-Asp-Asp-Asp-Asp-Asp-Asp-Asp-Asp-Asp-Asp-Asp, is used to prepare a charged moiety (C) with a net negative charge of −15; a charged moiety (C) comprised of 14 aspartic acid monomers, e.g., Asp-Asp-Asp-Asp-Asp-Asp-Asp-Asp-Asp-Asp-Asp-Asp-Asp-Asp, is used to prepare a charged moiety (C) with a net negative charge of −14; a charged moiety (C) comprised of 13 aspartic acid monomers, e.g., Asp-Asp-Asp-Asp-Asp-Asp-Asp-Asp-Asp-Asp-Asp-Asp-Asp, is used to prepare a charged moiety (C) with a net negative charge of −13; a charged moiety (C) comprised of 12 aspartic acid monomers, e.g., Asp-Asp-Asp-Asp-Asp-Asp-Asp-Asp-Asp-Asp-Asp-Asp, is used to prepare a charged moiety (C) with a net negative charge of −12; a charged moiety (C) comprised of 11 aspartic acid monomers, e.g., Asp-Asp-Asp-Asp-Asp-Asp-Asp-Asp-Asp-Asp-Asp, is used to prepare a charged moiety (C) with a net negative charge of −11; a charged moiety (C) comprised of 10 aspartic acid monomers, e.g., Asp-Asp-Asp-Asp-Asp-Asp-Asp-Asp-Asp-Asp, is used to prepare a charged moiety (C) with a net negative charge of −10; a charged moiety (C) comprised of 9 aspartic acid monomers, e.g., Asp-Asp-Asp-Asp-Asp-Asp-Asp-Asp-Asp, is used to prepare a charged moiety (C) with a net negative charge of −9; a charged moiety (C) comprised of 8 aspartic acid monomers, e.g., Asp-Asp-Asp-Asp-Asp-Asp-Asp-Asp, is used to prepare a charged moiety (C) with a net negative charge of −8; a charged moiety (C) comprised of 7 aspartic acid monomers, e.g., Asp-Asp-Asp-Asp-Asp-Asp-Asp, is used to prepare a charged moiety (C) with a net negative charge of −7; a charged moiety (C) comprised of 6 aspartic acid monomers, e.g., Asp-Asp-Asp-Asp-Asp-Asp, is used to prepare a charged moiety (C) with a net negative charge of −6; a charged moiety (C) comprised of 5 aspartic acid monomers, e.g., Asp-Asp-Asp-Asp-Asp, is used to prepare a charged moiety (C) with a net negative charge of −5; a charged moiety (C) comprised of 4 aspartic acid monomers, e.g., Asp-Asp-Asp-Asp, is used to prepare a charged moiety (C) with a net negative charge of −4; a charged moiety (C) comprised of 3 aspartic acid monomers, e.g., Asp-Asp-Asp, is used to prepare a charged moiety (C) with a net negative charge of −3; a charged moiety (C) comprised of 2 aspartic acid monomers, e.g., Asp-Asp, is used to prepare a charged moiety (C) with a net negative charge of −2; a charged moiety (C) comprised of 1 aspartic acid monomer, e.g., Asp, is used to prepare a charged moiety (C) with a net negative charge of −1. In the above examples, aspartic acid (Asp) may be replaced with any suitable negatively charged amino acid, including but not limited to glutamic acid, sulfo-serine, or phospho-serine, wherein the negatively charged amino acids may be the same or different.

In some embodiments the charged moiety (C) has a net positive charge and is comprised of positively charged functional groups. Suitable positively charged moieties (C) include those with functional groups that carry positive charge at physiologic pH, at a pH of about 7.4, such as the conjugate acid of weak bases, wherein the pKa of the conjugate acid of the base is greater than about 8.5. Suitable positively charged moieties (C) include but are not limited to molecules bearing primary, secondary and tertiary amines, as well as quaternary ammonium, guanidinium, phosphonium and sulfonium functional groups. Suitable molecules bearing ammonium functional groups include, for example, imidazolium, and tetra-alkyl ammonium compounds. In some embodiments, the charged moiety (C) is comprised of quaternary ammonium compounds that carry a permanent positive charge that is independent of pH.

Non-limiting examples of positively charged functional groups that have charge independent of pH include:

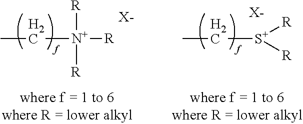

wherein X′ is any suitable counter anion.

In additional embodiments, the charged moiety (C) is comprised of functional groups that occur as the conjugate acid of a base at physiologic pH, such as, for example, primary, secondary and tertiary amines. In preferred embodiments, the positively charged moiety (C) is comprised of between 1-20 positively charged functional groups, such as 1, 2, 3, 4, 5, 6, 7, 8, 9, 10, 11, 12, 13, 14, 15, 16, 17, 18, 19 or 20 positively charged functional groups, though, typically no more than 16 charged functional groups. In some embodiments, the charged moiety (C) is a poly(lysine) peptide of between 1-6 amino acids in length. A poly(lysine) sequence comprised of 1, 2, 3, 4, 5 or 6 amino acids would be expected to carry a positive charge of +1, +2, +3, +4, +5 or +6 respectively, at pH 7.4. In additional embodiments, the charged moiety (C) is a poly(arginine) peptide of between 2-6 amino acids in length.

In certain embodiments, the charged moiety (C) has a net positive charge and is comprised of 1 or more positively charged amino acids. In preferred embodiments, the charged moiety (C) with a net positive charge is comprised of between 1 to 20 positively charged amino acids, e.g., 1, 2, 3, 4, 5, 6, 7, 8, 9, 10, 11, 12, 13, 14, 15, 16, 17, 18, 19 or 20. In a non-limiting example, a charged moiety (C) comprised of 16 lysine monomers, e.g., Lys-Lys-Lys-Lys-Lys-Lys-Lys-Lys-Lys-Lys-Lys-Lys-Lys-Lys-Lys-Lys, is used to prepare a charged moiety (C) with a net positive charge of +16; a charged moiety (C) comprised of 15 lysine monomers, e.g., Lys-Lys-Lys-Lys-Lys-Lys-Lys-Lys-Lys-Lys-Lys-Lys-Lys-Lys-Lys, is used to prepare a charged moiety (C) with a net positive charge of +15; a charged moiety (C) comprised of 14 lysine monomers, e.g., Lys-Lys-Lys-Lys-Lys-Lys-Lys-Lys-Lys-Lys-Lys-Lys-Lys-Lys, is used to prepare a charged moiety (C) with a net positive charge of +14; a charged moiety (C) comprised of 13 lysine monomers, e.g., Lys-Lys-Lys-Lys-Lys-Lys-Lys-Lys-Lys-Lys-Lys-Lys-Lys, is used to prepare a charged moiety (C) with a net positive charge of +13; a charged moiety (C) comprised of 12 lysine monomers, e.g., Lys-Lys-Lys-Lys-Lys-Lys-Lys-Lys-Lys-Lys-Lys-Lys, is used to prepare a charged moiety (C) with a net positive charge of +12; a charged moiety (C) comprised of 11 lysine monomers, e.g., Lys-Lys-Lys-Lys-Lys-Lys-Lys-Lys-Lys-Lys-Lys, is used to prepare a charged moiety (C) with a net positive charge of +11; a charged moiety (C) comprised of 10 lysine monomers, e.g., Lys-Lys-Lys-Lys-Lys-Lys-Lys-Lys-Lys-Lys, is used to prepare a charged moiety (C) with a net positive charge of +10; a charged moiety (C) comprised of 9 lysine monomers, e.g., Lys-Lys-Lys-Lys-Lys-Lys-Lys-Lys-Lys, is used to prepare a charged moiety (C) with a net positive charge of +9; a charged moiety (C) comprised of 8 lysine monomers, e.g., Lys-Lys-Lys-Lys-Lys-Lys-Lys-Lys, is used to prepare a charged moiety (C) with a net positive charge of +8; a charged moiety (C) comprised of 7 lysine monomers, e.g., Lys-Lys-Lys-Lys-Lys-Lys-Lys, is used to prepare a charged moiety (C) with a net positive charge of +7; a charged moiety (C) comprised of 6 lysine monomers, e.g., Lys-Lys-Lys-Lys-Lys-Lys, is used to prepare a charged moiety (C) with a net positive charge of +6; a charged moiety (C) comprised of 5 lysine monomers, e.g., Lys-Lys-Lys-Lys-Lys, is used to prepare a charged moiety (C) with a net positive charge of +5; a charged moiety (C) comprised of 4 lysine monomers, e.g., Lys-Lys-Lys-Lys, is used to prepare a charged moiety (C) with a net positive charge of +4; a charged moiety (C) comprised of 3 lysine monomers, e.g., Lys-Lys-Lys, is used to prepare a charged moiety (C) with a net positive charge of +3; a charged moiety (C) comprised of 2 lysine monomers, e.g., Lys-Lys, is used to prepare a charged moiety (C) with a net positive charge of +2; a charged moiety (C) comprised of 1 lysine, e.g., Lys, is used to prepare a charged moiety (C) with a net positive charge of +1. In the above examples, Lysine (Lys) may be replaced with any suitable positively charged amino acid, including but not limited to trimethyl-lysine or arginine, wherein the positively charged amino acids may be the same or different.

Charged moieties (C) may additionally comprise small non-charged, hydrophilic amino acids, or hydrophilic linkers, e.g., ethylene oxide that function to i) improve water solubility and ii) increase the distance between charged functional groups to prevent incomplete ionization. For instance, ionization of one functional group on a polymer may impact the pKa of neighboring functional groups through local effects. For example, protonation of an amine in close proximity to a second amine may lower the pKa of the conjugate acid of the second amine. To reduce the impact of local effects on the ionization potential of neighboring functional groups, a linker molecule may be used to increase the distance between charged functional groups comprising the charged moiety. The linker molecule may comprise between 1-5 small, non-charged hydrophilic amino acids, e.g., 1, 2, 3, 4, and 5 amino acids. Alternatively, the linker may comprise an ethylene oxide (i.e, PEG) linker between 1-4 monomers units, e.g., 1, 2, 3, or 4 ethylene oxide monomers in length. In preferred embodiments, 1 to 2 small, non-charged hydrophilic amino acids are placed between neighboring charged amino acids comprising the charged moiety (C), wherein the amino acids are linked through amide bonds. In certain embodiments, a serine is placed between each charged amino acid comprising a charged moiety (C) with a net positive charge. In preferred embodiments, the charged moiety (C) is comprised of repeating dipeptides of lysine and serine, i.e. (Lys-Ser), where n is typically any integer between 1-20, e.g., 1, 2, 3, 4, 5, 6, 7, 8, 9, 10, 11, 12, 13, 14, 15, 16, 17, 18, 19 or 20. As other examples, a serine is placed between each charged amino acid of a tripeptide charged moiety (C) with a net +2 charge, e.g., Lys-Ser-Lys; a serine is placed between each charged amino acid of a 5 amino acid charged moiety (C) with a net +3 charge, e.g., Lys-Ser-Lys-Ser-Lys; a serine is placed between each charged amino acid of a 7 amino acid charged moiety (C) with a net +4 charge, e.g., Lys-Ser-Lys-Ser-Lys-Ser-Lys. In the above examples, Lysine (Lys) may be replaced with any suitable positively charged amino acid, including but not limited to trimethyl-lysine or arginine, wherein the positively charged amino acids may be the same or different.

In certain embodiments, a serine is placed between each charged amino acid comprising a charged moiety (C) with a net negative charge. In preferred embodiments, the charged moiety is comprised of repeating dipeptides of aspartic acid and serine, i.e. (Asp-Ser)ₙ, where n is typically any integer between 1-20, e.g., 1, 2, 3, 4, 5, 6, 7, 8, 9, 10, 11, 12, 13, 14, 15, 16, 17, 18, 19 or 20. For example, a serine is placed between each charged amino acid of a tripeptide charged moiety (C) with a net −2 charge, e.g., Asp-Ser-Asp; a serine is placed between each charged amino acid of a 5 amino acid charged moiety (C) with a net −3 charge, e.g., Asp-Ser-Asp-Ser-Asp; a serine is placed between each charged amino acid of a 7 amino acid charged moiety (C) with a net −4 charge, e.g., Asp-Ser-Asp-Ser-Asp-Ser-Asp. In the above examples, aspartic acid (Asp) may be replaced with any suitable negatively charged amino acid, including but not limited to glutamic acid, sulfo-serine, or phospho-serine, wherein the negatively charged amino acids may be the same or different.

In additional embodiments, the charged moiety (C) is comprised of both negatively and positively charged amino acids. Di-peptides comprised of amino acids of opposite charge, e.g., Lys-Asp, are referred to as zwitterion dipeptides because they are predicted to have a net neutral, 0, charge at pH 7.4. One or more zwitterion dipeptides can be included in the charged moiety (C) as a means to i) improve water solubility and ii) provide a prevailing charge (e.g., net negative or net positive) over certain pH ranges. For instance, a zwitterion di-peptide can be used to increase the hydrophilic character of a peptide sequence without increasing or decreasing the charge of a peptide sequence at pH 7.4. However, the zwitterion can be used to impart a net charge at a particular pH. For instance, excluding the contribution of the N-terminal amine and the C-terminal carboxylic acid in this example, the zwitterion di-peptide, Lys-Asp, has a net charge of 0 at pH 7.4, but a net charge of +1 at pH<4 and a net charge of −1 at pH>10. One or more zwitterion di-peptides can be added to the sequence of charged moieties (C); for example, one di-peptide, Lys-Asp; two di-peptides Lys-Asp-Lys-Asp; three di-peptides, Lys-Asp-Lys-Asp-Lys-Asp and so forth. In the above examples, Lysine (Lys) may be replaced with any suitable positively charged amino acid, including but not limited to trimethyl-lysine or arginine, and aspartic acid (Asp) may be replaced with any suitable negatively charged amino acid, including but not limited to glutamic acid, sulfo-serine, or phospho-serine, wherein the positively or negatively charged amino acids may be the same or different.

The composition of the charged moiety (C) is selected to provide the net charge needed of a peptide antigen conjugate for the specific application. In several embodiments disclosed herein, the charged moiety (C) is a positively charged poly(amino acid) comprised of lysines or arginines, or lysines or arginines and non-charged amino acids. In some embodiments the charged moiety comprise sulfonium or quaternary ammonium functional groups that carry pH independent positive charge. In several embodiments disclosed herein, the charged moiety (C) is a negatively charged poly(amino acid) comprised of glutamic acid or aspartic acid, or glutamic acid or aspartic acid and non-charged amino acids. In some embodiments the charged moiety comprises phosphate or sulfate groups, such as sulfoserine or phosphoserine. In additional embodiments, the charged moiety is comprised of lysines or arginines and glutamic acid or aspartic acid, or lysines or arginines and glutamic acid or aspartic acid as well as non-charged amino acids. Both positive and negatively charged functional groups may be included on the same charged moiety (C). The charged moiety (C) may be positive, negative or neutral but the net charge of the peptide antigen conjugate should be non-zero, for example, greater than +3 or less than −3 net charges are preferred and depend on the specific application.

In some embodiments, the peptide antigen conjugate comprises a single charged moiety (C). In other embodiments, the peptide antigen conjugate comprises two charged moieties. For peptide antigen conjugates with two charged moieties (C), the charged moiety proximal to the N-terminus of the peptide antigen (A) is referred to as C1 and the charged moiety proximal to the C-terminus of the peptide antigen is referred to as C2. In some embodiments, C1 and C2 may be the same charged moieties, while in other embodiments, C1 and C2 may be different charged moieties.

The composition of the charged moieties (C1 and C2) and extension sequences (B1 and B2) can be selected to provide a particular number of charged residues that provide the desired net charge and hydropathy. In preferred embodiments, the number of charged functional groups comprising the charged moiety (C) is modulated such that the net charge of the peptide antigen conjugate comprising the charged moiety (C), peptide antigen (A), optional extensions (B1 and/or B2), Linker (L) and hydrophobic block (H) is between about −3 to −10 or between +3 to +10.

The charged moiety (C) may be linked to the peptide antigen (A) either directly, or indirectly through an extension (B1 or B2), Linker (L) and/or hydrophobic block (H).

In preferred embodiments, the charged moiety (C) is linked to an extension (B1) that is linked to the N-terminus of a peptide antigen (A) that is linked at the C-terminus to an extension (B2) that is linked either directly or via a Linker (L) to the hydrophobic block (H) to yield a peptide antigen conjugate of Formula V:

C-[B1]-A-[B2]-[L]-H or H-[L]-[B1]-A-[B2]-C Formula V

In several embodiments, the charged moiety (C) is placed at the N-terminus of a peptide antigen conjugate of Formula V, wherein the charged moiety (C) is linked to an N-terminal extension (B1) comprised of a cathepsin cleavable extension typically between 1 to 4 amino acids in length (B1=PN1, PN2-PN1, PN3-PN2-PN1 or PN4-PN3-PN2-PN1) that is linked to the N-terminus of a peptide antigen (A) that is linked at the C-terminus to a C-terminal extension (B2) comprised of an immuno-proteasome, cathepsin or combined immuno-proteasome and cathepsin cleavable extension typically between 1 to 6 amino acids in length (B2=PC1′, PC1′-PC2′, PC1′-PC2′-PC3′, PC1′-PC2′-PC3′-PC4′, PC1′-PC2′-PC3′-PC4′-PC5′, or PC1′-PC2′-PC3′-PC4′-PC5′-PC6′) that is linked to a Linker (L) that is linked to the hydrophobic block (H). The peptide antigen (A) of a peptide antigen conjugate of Formula V is comprised of an integer number of amino acids, n, wherein n is typically between 7-35 amino acids or up to 50 amino acids, and the hydrophobic block (H) is typically a poly(amino acid) of Formula I or II linked to an adjuvant of Formula III.

A non-limiting example of a peptide antigen conjugate of Formula V comprising a charged moiety (C=Lys-Lys) linked to a cathepsin cleavable tetrapeptide extension (B1=Lys-Pro-Leu-Arg) at the N-terminus of a peptide antigen (A) that is linked at the C-terminus to a cathepsin cleavable hexapeptide extension (B2=Gly-Gly-Ser-Leu-Val-Arg) that is linked to a Linker (L) that is linked to a hydrophobic block (H) comprised of a poly(amino acid) of Formula I that is linked to an adjuvant of Formula III is:

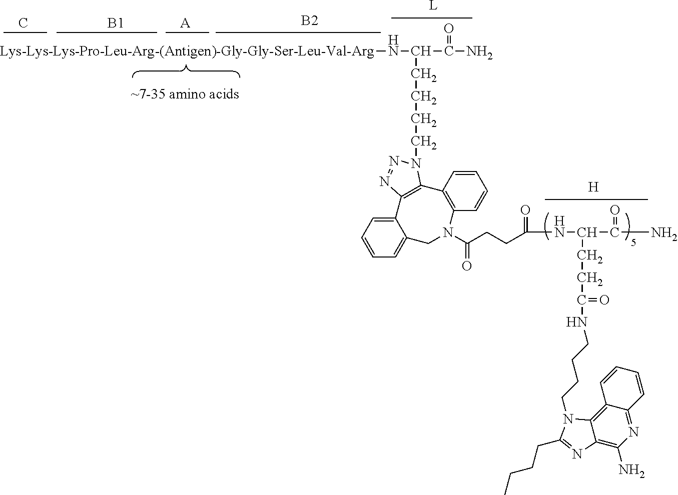

In a non-limiting example of a peptide antigen conjugate of Formula V, C-B1-(A)₇₋₃₅-B2-L-H, a peptide antigen (A) with the sequence Ala-Lys-Phe-Val-Ala-Ala-Trp-Thr-Leu-Lys-Ala-Ala-Ala is linked to an N-terminal extension (B1) with the sequence Ser-Leu-Val-Arg that is linked to a charged moiety (C) comprised of a dipeptide with the sequence Glu-Lys and a C-terminal extension (B2) with the sequence Ser-Leu-Val-Arg that is linked to the linker (L) (Lys(N3-DBCO) that is linked to the hydrophobic block (H), for example: Glu-Lys-Ser-Leu-Val-Arg-Ala-Lys-Phe-Val-Ala-Ala-Trp-Thr-Leu-Lys-Ala-Ala-Ala-Ser-Leu-Val-Arg-Lys(N3-DBCO-H), resulting in a peptide antigen conjugate with a net charge of +5 at pH 7.4. For clarity, the hydrophobic block (H) in this example is assumed to have a negligible contribution to the charge and the C-terminus is amidated.

In additional embodiments, a charged moiety (C; or C2 when there are two charged moieties present) may be linked directly to the hydrophobic block (H) or to the Linker (L) that is linked to the C-terminal extension (B2) that is linked to the C-terminus of a peptide antigen (A) that is optionally linked at the N-terminus to an N-terminal extension (B1) that is optionally linked to an additional optional charged moiety (C1) to yield a peptide antigen conjugate of Formula VI:

[B1]-A-[B2]-L(C)-H, [B1]-A-[B2]-L-H(C), [C1]-[B1]-A-[B2]-L(C2)-H, [C1]-[B1]-A-[B2]-L-H(C2), H-L(C)-[B1]-A-[B2], H(C)-[B1]-A-[B2], H-L(C1)-[B1]-A-[B2]-C2 or H(C1)-[B1]-A-[B2]-C2 Formula VI

In several embodiments, the charged moiety (C) is placed at the C-terminus of a peptide antigen conjugate of Formula VI, wherein the charged moiety (C) is linked to a Linker (L) that is linked to a C-terminal extension (B2) comprised of an immuno-proteasome, cathepsin or combined immuno-proteasome and cathepsin cleavable extension typically between 1 to 6 amino acids in length (B2=PC1′, PC1′-PC2′, PC1′-PC2′-PC3′, PC1′-PC2′-PC3′-PC4′, PC1′-PC2′-PC3′-PC4′-PC5′, or PC1′-PC2′-PC3′-PC4′-PC5′-PC6′) that is linked to the C-terminus of a peptide antigen (A) that is optionally linked at the N-terminus to a cathepsin cleavable extension typically between 1 to 4 amino acids in length (B1=PN1, PN2-PN1, PN3-PN2-PN1 or PN4-PN3-PN2-PN1), wherein the Linker (L) is additionally linked to a hydrophobic block (H), shown here:

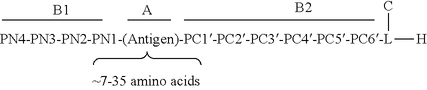

The peptide antigen (A) of the peptide antigen conjugate of Formula VI is comprised of an integer number of amino acids, n, wherein n is typically between 7-35 amino acids, or up to 50 amino acids, and the hydrophobic block (H) is typically a poly(amino acid) of Formula I or II linked to an adjuvant of Formula III.

A non-limiting example of a peptide antigen conjugate of Formula VI comprised of a charged moiety (C=Lys-Lys) linked via an amide bond to the C-terminus of a Linker (L) that is linked to a combined immuno-proteasome and cathepsin cleavable hexapeptide C-terminal extension (B2=Gly-Gly-Ser-Leu-Val-Arg) that is linked to the C-terminus of a peptide antigen (A) that is linked at the N-terminus to a cathepsin cleavable tetrapeptide N-terminal extension (B1=Lys-Pro-Leu-Arg), wherein the Linker (L) is additionally linked to a hydrophobic block (H) that is comprised of a poly(amino acid) of Formula I that is linked to an adjuvant of Formula III is provided:

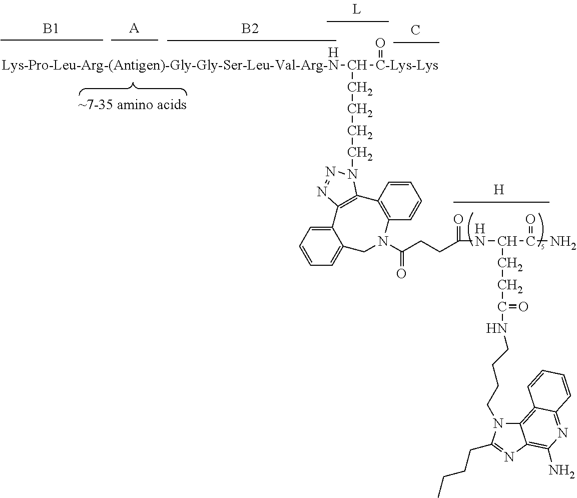

An additional non-limiting example of a peptide antigen conjugate of Formula VI, B1-A-B2-L-H(C), is a charged moiety (C=Lys-Lys-Lys-Lys-Lys) linked via a linker to a hydrophobic block (H) that is comprised of a poly(amino acid) of Formula I that is linked to an adjuvant of Formula III that is linked to a Linker (L) that is linked to a combined immuno-proteasome and cathepsin cleavable hexapeptide C-terminal extension (B2=Gly-Gly-Ser-Leu-Val-Arg) that is linked to the C-terminus of a peptide antigen (A) and wherein the N-terminus of the peptide antigen is linked to a cathepsin cleavable tetrapeptide N-terminal extension (B1=Lys-Pro-Leu-Arg):

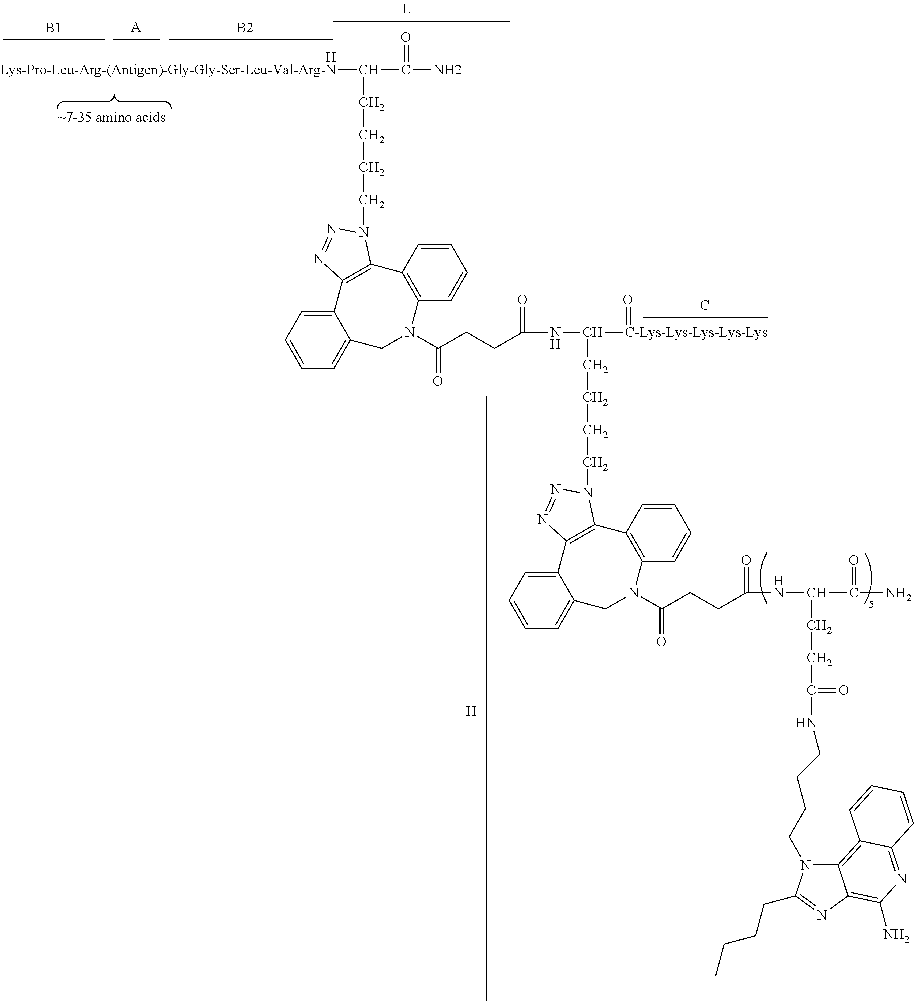

In a non-limiting example of a peptide antigen conjugate of Formula VI, B1-(A)₇₋₃₅-B2-L(-C)-H, a peptide antigen (A) with the sequence Ala-Lys-Phe-Val-Ala-Ala-Trp-Thr-Leu-Lys-Ala-Ala-Ala is linked to an N-terminal extension (B1) with the sequence Ser-Leu-Val-Arg and a C-terminal extension (B2) with the sequence Ser-Leu-Val-Arg that is linked to a linker precursor X1, e.g., Lys(N3), that is linked to both a charged moiety (C) comprised of a dipeptide with the sequence Glu-Lys and a linker precursor X2, comprising a DBCO molecule that is linked to the hydrophobic block (H), for example: Ser-Leu-Val-Arg-Ala-Lys-Phe-Val-Ala-Ala-Trp-Thr-Leu-Lys-Ala-Ala-Ala-Ser-Leu-Val-Arg-Lys(N3-DBCO-H)-Glu-Lys, wherein the Glu-Lys sequence is linked to the C-terminus of the Linker (L) (Lys(N3-DBCO), resulting in a peptide antigen conjugate with a predicted net charge of +4 at pH 7.4. Here, the hydrophobic block (H) is assumed to have a negligible contribution to the charge of the peptide antigen conjugate.

Peptide antigen conjugates of Formula VI, wherein the charged moiety (C) is linked to the hydrophobic block (H), may be advantageous for the rapid production of personalized therapies, such as cancer vaccines. The hydrophobic block (H) that is linked to a charged moiety (C) and linker precursor X2 (e.g, X2 comprising a cyclooctyne) can be prepared in bulk and then readily combined with any peptide antigen (A) bearing a linker precursor X1 (e.g., X1 comprising an azide) to form a peptide antigen conjugate of the Formula VI, [B1]-A-[B2]-L-H(C), wherein [ ] denotes the group is optional.

The function of the charged moiety (C) is to stabilize nanoparticles formed by peptide antigen conjugates in aqueous conditions. While the hydrophobic block (H) induces particle formation of peptide antigen conjugates, the optional charged moiety (C) provides a countervailing force that prevents flocculation and, in some embodiments, drives the peptides antigen conjugates to assemble into nanoparticle micelles with a surface charge provided by the charged moiety (C).

For certain applications, positively charged peptide antigen conjugates are required. In some embodiments, a peptide antigen conjugate of Formula V comprised of a charged moiety (C) linked to an N-terminal peptide extension (B1) is linked to a peptide antigen (A) that is linked to a C-terminal peptide extension (B2) that is linked through linker (L) to the hydrophobic block (H) and the resulting peptide antigen conjugate has a net positive charge, wherein the hydrophobic block (H) is comprised of poly(amino acids) of Formula I or Formula II linked to adjuvants of Formula III. The hydrophobic block (H) linked through the C-terminus of the peptide antigen (A) functions to induce particle formation and the charged moiety (C) linked through the N-terminus of the peptide antigen (A) functions to stabilize the particles through high positive charge density at the surface of those particles. Thus, B2 extensions placed proximal to the hydrophobic block (H) of net positively charged peptide antigen conjugates of Formula V, wherein the charged moiety (C) is linked through the N-terminus of the peptide antigen (A), are preferably comprised of non-charged and hydrophobic amino acids that help to promote particle formation and are typically selected from single amino acids, such as glycine, serine, citrulline, leucine, norlecuine or methionine; dipeptides, such as Gly-Cit, Gly-Ser or Gly-Leu; tripeptides, such as Gly-Ser-Cit, Gly-Pro-Cit, or Gly-Pro-Gly; tetrapeptides, such as Ser-Pro-Val-Cit or Gly-Pro-Gly-Cit; pentapeptides, such as Gly-Ser-Val-Leu-Cit, Gly-Pro-Val-Leu-Cit; or hexapeptides, such as Gly-Gly-Ser-Leu-Val-Cit, or Gly-Gly-Ser-Pro-Val-Cit. In some embodiments, a C-terminal extension (B2) that includes a single charged amino acid is used and is typically selected from single amino acids, such as arginine or lysine; dipeptides, such as Gly-Arg or Gly-Lys; tripeptides, such as Gly-Ser-Arg or Gly-Ser-Lys; tetrapeptides such as Gly-Pro-Gly-Arg, Gly-Ser-Val-Arg or Ser-Leu-Val-Arg (where Arg can be replaced with Lys); pentapeptides, such as Gly-Ser-Leu-Val-Arg (where Arg can be replaced with Lys); and hexapeptides such as Gly-Gly-Ser-Leu-Val-Arg or Gly-Gly-Ser-Pro-Val-Arg (where Arg can be replaced with Lys). B1 extensions placed proximal to the charged moiety (C) of positively charged peptide antigen conjugates of Formula V, wherein the charged moiety (C) is linked through the N-terminus of the peptide antigen (A), preferably contain a charged amino acid, such as an Arg or Lys, and are typically selected from single amino acids selected from Arg or Lys; dipeptides selected from Val-Arg or Leu-Arg (where Arg can be replaced with Lys); tripeptides selected from Pro-Val-Arg or Gly-Val-Arg (where Arg can be replaced with Lys); or tetrapeptides selected from Lys-Leu-Val-Arg, Lys-Pro-Val-Arg, Lys-Pro-Leu-Arg, Ser-Leu-Val-Arg and Ser-Pro-Val-Arg (where Arg can be replaced with Lys). The different extensions, B1 and B2 can be combined with any charged moiety (C), peptide antigen (A) and hydrophobic block (H) to achieve the Grand average of hydropathy value and net charge of the peptide antigen conjugate needed.

For certain applications, negatively charged peptide antigen conjugates are required. In some embodiments, a peptide antigen conjugate of Formula V comprised of a charged moiety (C) with net negative charge is linked to an N-terminal peptide extension (B1) that is linked to a peptide antigen (A) that is linked to a C-terminal peptide extension (B2) that is linked through a linker (L) to the hydrophobic block (H) and the resulting peptide antigen conjugate has a net negative charge, wherein the hydrophobic block (H) is comprised of poly(amino acids) of Formula I or Formula II linked to adjuvants of Formula III. The hydrophobic block (H) linked through the C-terminus of the peptide antigen (A) functions to induce particle formation and the charged moiety (C) linked through the N-terminus of the peptide antigen (A) functions to stabilize the particles through high negative charge density. Thus, B2 extensions placed proximal to the hydrophobic block (H) of net negatively charged peptide antigen conjugates of Formula V, wherein the charged moiety (C) is linked through the B1 extension at the N-terminus of the peptide antigen (A), are preferably comprised of non-charged and hydrophobic amino acids that help to promote particle formation and are typically selected from single amino acids, such as glycine, serine, citrulline, leucine, norlecuine or methionine; dipeptides, such as Gly-Ser, Gly-Cit or Gly-Leu; tripeptides, such as Gly-Ser-Cit, Gly-Pro-Cit, or Gly-Pro-Gly; tetrapeptides, such as Ser-Pro-Val-Cit, Ser-Pro-Val-Cit or Gly-Pro-Gly-Cit; pentapeptides, such as Gly-Ser-Val-Leu-Cit, Gly-Pro-Val-Leu-Cit; or hexapeptides, such as Gly-Gly-Ser-Leu-Val-Cit, or Gly-Gly-Ser-Pro-Val-Cit or Gly-Gly-Ser-Pro-Leu-Cit. In some embodiments, a C-terminal extension (B2) that includes a single charged amino acid is used and is typically selected from single amino acids, such as arginine or lysine; dipeptides, Gly-Arg (where Arg can be replaced with Lys); tripeptides, such as Gly-Ser-Arg (where Arg can be replaced with Lys); tetrapeptides such as Gly-Pro-Gly-Arg, Gly-Ser-Val-Arg, Ser-Leu-Val-Arg, Asp-Leu-Val-Cit or Asp-Leu-Val-Leu (where Asp can be replaced with Glu and Arg can be replaced with Lys); pentapeptides, such as Gly-Ser-Leu-Val-Arg or Gly-Asp-Leu-Val-Leu or Gly-Asp-Leu-Val-Arg; and hexapeptides such as Gly-Gly-Ser-Leu-Val-Arg or Gly-Gly-Ser-Pro-Val-Arg (where Asp can be replaced with Glu and Arg can be replaced with Lys); or Gly-Ser-Glu-Leu-Val-Arg or Gly-Gly-Asp-Pro-Val-Arg (where Asp can be replaced with Glu and Arg can be replaced with Lys). B1 extensions placed proximal to the charged moiety (C) of net negatively charged peptide antigen conjugates of Formula V, wherein the charged moiety (C) is linked through the N-terminus of the peptide antigen via the B1 extension preferably contain a charged amino acid, such as an Asp or Glu, and are typically selected from single single amino acids such as Cit, Leu, Arg or Lys; dipeptides selected from Val-Cit, Leu-Cit, Val-Arg or Leu-Arg (where Arg can be replaced with Lys); tripeptides selected from Pro-Val-Arg, Pro-Val-Cit, Gly-Val-Arg or Gly-Val-Cit (where Arg can be replaced with Lys); or tetrapeptides selected from Ser-Leu-Val-Arg, Ser-Pro-Val-Arg, Ser-Pro-Leu-Cit, Ser-Leu-Val-Cit, Ser-Pro-Val-Cit, Asp-Leu-Val-Arg, Asp-Pro-Val-Arg, Asp-Leu-Val-Cit, Asp-Leu-Val-Leu, Ser-Gly-Val-Cit or Asp-Pro-Val-Cit (where Arg can be replaced with Lys and Asp can be replaced with Glu). The different extensions, B1 and B2 can be combined with any charged moiety (C), peptide antigen (A) and hydrophobic block (H) to achieve the Grand average of hydropathy value and net charge of the peptide antigen conjugate needed.

### Use of Charged Moieties (C) Based on Poly(Anions)

A challenge for producing peptide antigen conjugates with charged moieties (C) comprised of anions based on acids, e.g., peptide-based oligomers or polymers based on aspartic acid, glutamic acid, sulfoserine or phosphoserine, is that the protonated forms of these acids are typically poorly soluble in aqueous solutions and often even have poor solubility in water-miscible organic solvents. The limited solubility of acids can create challenges to manufacturing and handling peptide antigen fragments and/or peptide antigen conjugates with charged moieties (C) comprised of acids. For instance, peptide antigen conjugates of formula C-[B1]-A-[B2]-[L]-H and H-[L]-[B1]-A-[B2]-C, wherein C is a charged moiety comprised of one or more amino acids bearing an amine functional group provide improved manufacturing over peptide antigen conjugates of formula C-[B1]-A-[B2]-[L]-H and H-[L]-[B1]-A-[B2]-C, wherein C is a charged moiety comprised of one or more amino acids bearing an acid functional group. However, there may be instances where multiple acid functional groups are needed to provide net negative charge of the peptide antigen conjugate (when the acids are deprotonated, i.e., in the form of a conjugate base of the acid). For example, high positive charge can cause peptide antigen conjugates to non-specifically interact with negatively charged cell surfaces. Therefore, for certain applications, it may be beneficial to avoid these non-specific interactions by using peptide antigen conjugates with net negative charge provided by one or more conjugate bases of weak acids (e.g., COO— from COOH) present on the charged moiety (C).

Based on a need to improve manufacturability of peptide antigen fragments and peptide antigen conjugates with charged moieties comprising acids, two strategies were developed and led to unexpected improvements in manufacturability. These two strategies, relating to the choice of counter-ion and use of charged amphiphilic carrier molecules, are described below.

### Choice of Counter-Ion

The choice of counter-ions of conjugate bases of acids comprising charged moieties was found to impact peptide antigen fragment and peptide antigen conjugate manufacturability.

While use of alkali metal ions, such as sodium (Na+) and potassium (K+), as the counter-ions of conjugate bases of acids provided salts (e.g., the sodium salt of carboxylate) with good water solubility, such salts were generally found to have insufficient solubility in water-miscible solvents, such as DMSO, DMF, methanol, ethanol and acetone, which are preferred solvent systems for solubilizing the hydrophobic block (H) during the reaction of the hydrophobic block with the peptide antigen fragment.

In contrast, the conjugate acid of organic bases, such as those based on alkyl amines, particularly tri-alkyl amines, were found to improve solubility of peptide antigen fragments and peptide antigen conjugates in both water and water-miscible organic solvents. Therefore, in certain embodiments, peptide antigen fragments, peptide antigen conjugates as well as amphiphilic carrier molecules that comprise acids are prepared as the ammonium salt form of the acid. Suitable amines used to form the ammonium salt include but are not limited to ammonium, primary amines, such as tris(hydroxymethyl)aminomethane, secondary amines based on di-alkyl amines, such as dimethyl amine and diethyl amine, tertiary amines based on tri-alkyl amines, such as trimethylamine, di-isopropryl ethalymine (DIPEA) and triethylamine (TEA), as well as quaternary ammonium compounds. Unexpectedly, tris(hydroxymethyl)aminomethane (or Tris) as the ammonium salt of acids present on peptide antigen fragment, peptide antigen conjugates and amphiphilic carrier molecules improved solubility of such molecules in both water-miscible organic solvents, such as DMSO, DMF, acetone and ethanol, and aqueous solutions; additionally, the ammonium salts of peptide antigen fragments, peptide antigen conjugates and amphiphilic carrier molecules prepared from tris(hydroxymethyl)aminomethane had minimal impact on the pH of the aqueous buffer, such as PBS, pH 7.4, when such salts were suspended in aqueous buffers. For these reasons, the protonated form of tris(hydroxymethyl)aminomethane is a preferred counter-ion to use in the preparation of salts of conjugate bases of acids present on peptide antigen fragments, peptide antigen conjugates and amphiphilic carrier molecules.

### Poly(Anion)-Based Amphiphilic Carrier Molecules

It was also found that the use of specific compositions of amphiphilic carrier molecules, e.g., of formula C-[B]-[L]-H or C-[B1]-[A′]-[B2]-[L]-[H], which include a charged moiety bearing one or more acids (or salts of the conjugate base), can be simply mixed with any patient-specific peptide antigen conjugate to form mosaic particles, thereby circumventing the need to add the charged moiety, comprising acids, on the patient-specific peptide antigen conjugate. By adding the charged moiety to the conserved, non-patient-specific portion, i.e., the amphiphilic carrier molecule, this greatly simplifies manufacturing, particularly the challenges associated with the manufacture of amphiphiles comprised of peptide-based poly(acids).

This approach was identified to be particularly well-suited for the preparation of immunogenic compositions of nanoparticle micelles with net negative charge comprised of peptide antigen conjugates and amphiphilic carrier molecules, wherein the net negative charge is provided by negatively charged functional groups comprised of the conjugate base of acids, such as carboxylate, sulfonate, sulfate, phosphonate and/or phosphate, present on amphiphilic carrier molecules.

Therefore, in certain embodiments of immunogenic compositions of nanoparticle micelles with net negative charge comprised of peptide antigen conjugates and amphiphilic carrier molecules, peptide antigen conjugates of formula A-[B2]-[L]-H or H-[L]-[B1]-A are mixed at an appropriate molar ratio with amphiphilic carrier molecules of formula C-[B]-[L]-H or H-[L]-[B]-C in a water-miscible organic solvent and then suspended in aqueous solution, preferably an aqueous buffer with pH of about 7.4.

A non-limiting example of an immunogenic composition of nanoparticle micelles with net negative charge comprised of peptide antigen conjugates of formula A-B2-L-H and amphiphilic carrier molecules of formula C-B-L-H is shown below for clarity:

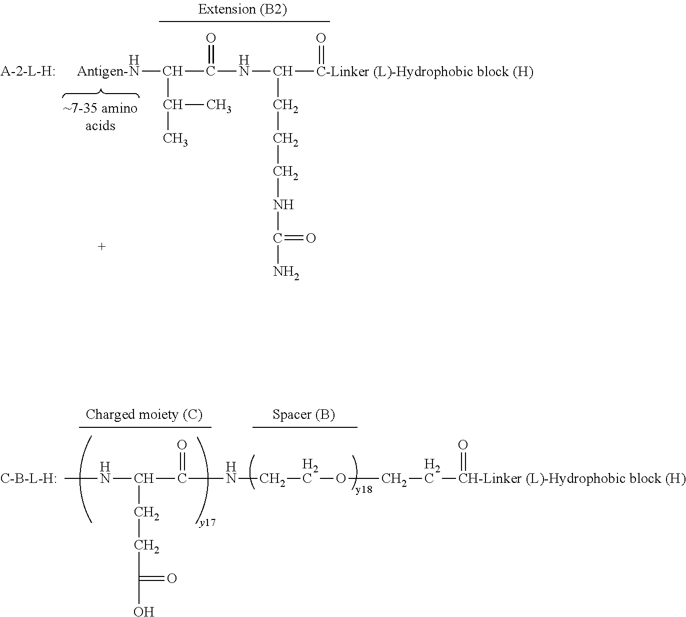

Wherein y17 is an integer number of repeating units of monomers comprising the charged moiety (C), typically selected from between about 1 to 16, such as 1, 2, 3, 4, 5, 6, 7, 8, 9, 10, 11, 12, 13, 14, 15 or 16; y18 is an integer number of repeating units of monomers comprising the spacer, which is typically between about 4 to about 200; and, the amine of the N-terminal amino acid for peptide-based charged moieties is either in the form of the free amine or is capped, e.g., with an acyl group. For greater clarity, in the non-limiting example above, wherein the hydrophobic block is poly(tryptophan), the Linker (L) is Lys(N3-DBCO), and the negatively charged moiety (C) is in the form of a Tris salt, the structure is:

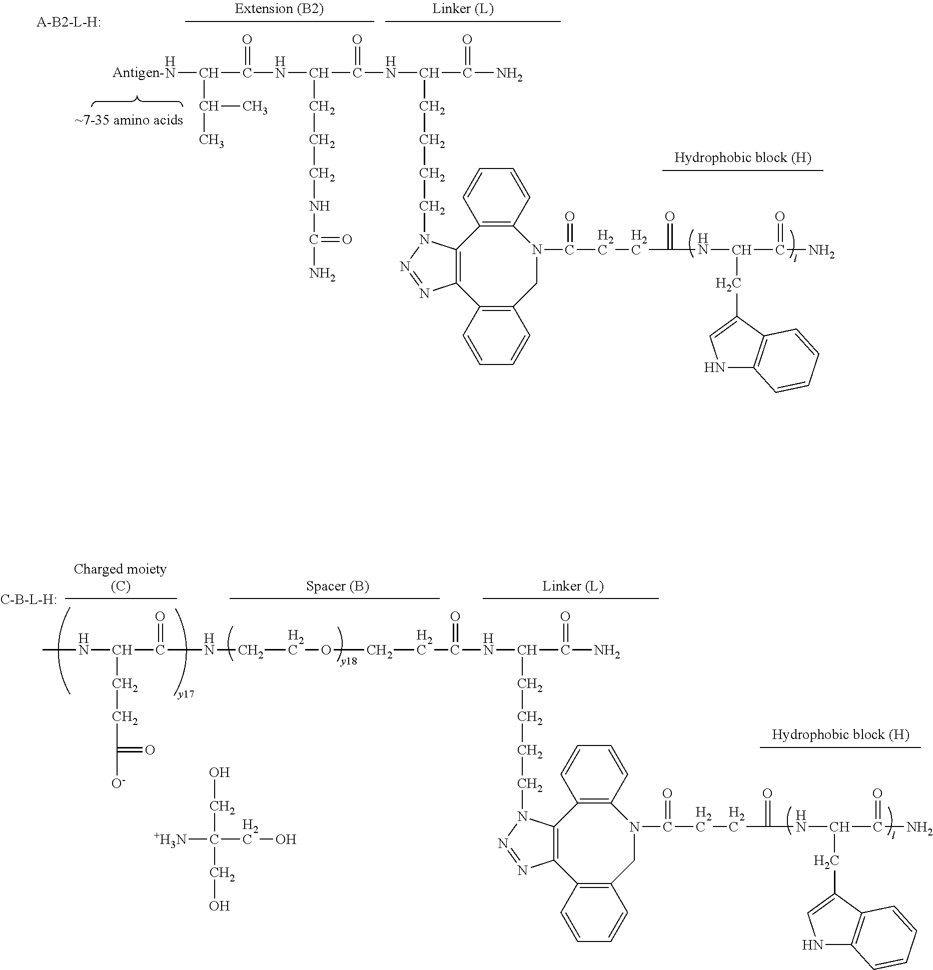

### Considerations for the Preparation of Nanoparticle Micelles Comprised of Peptide Antigen Conjugates and Amphiphilic Carrier Molecules

An unexpected finding reported herein is that peptide antigen conjugates bearing a charged moiety (e.g., C-[B1]-A-[B2]-[L]-H) can form stable micelles in the presence of a high molar excess, up to 4-fold higher excess hydrophobic block fragment (X2-H). A non-binding explanation is that the peptide antigen conjugates bearing the charged moiety form stable micelles and that the hydrophobic block fragment is incorporated within the hydrophobic core of such micelle nanoparticles.

These findings motivated the evaluation of a strategy whereby peptide antigen conjugates without a charged moiety, e.g., [B1]-A-[B2]-[L]-H or H-[L]-[B1]-A-[B2] are combined with an amphiphilic carrier molecule, e.g., of formula S-[B]-[L]-H or S-[B1]-[A′]-[B2]-[L]-H, wherein the solubilizing group, S, may comprise a charged moiety, e.g., C-[B]-[L]-H or C-[B1]-[A′]-[B2]-[L]-H, that is used to stabilize micelles that incorporate the peptide antigen conjugate. Note: the solubilizing group, S, may be any hydrophilic molecule, including any charged molecules, which are referred to herein as charged moieties (C).

A non-limiting example for the preparation of an immunogenic composition of nanoparticle micelles comprised of peptide antigen conjugates and amphiphilic carrier molecules is to combine one or more peptide antigen conjugates of formula [B1]-A-[B2]-[L]-H, e.g., A-B2-L-H, with an amphiphilic carrier molecule of formula C-[B]-[L]-H, e.g., C-B-H at a molar ratio of 4:1 moles of peptide antigen conjugate to moles of amphiphilic carrier molecule in DMSO and then add aqueous solution, e.g., buffer to the DMSO solution.

### Considerations of the Composition of the Amphiphilic Carrier Molecule

In some embodiments, the peptide antigen conjugate does not comprise a charged moiety, such as [B1]-A-[B2]-[L]-H, where [ ] denotes that the group is optional. Non-limiting examples include, A-H, A-L-H, A-B2-H, A-B2-L-H and B1-A-B2-L-H.

Certain peptide antigen conjugates that do not comprise a charged moiety may undergo aggregation in aqueous conditions, e.g., aqueous buffers such as PBS at pH 7.4, unless such conjugates are stabilized or formulated within a carrier. Thus, one means of generating stable nanoparticle micelles with peptide antigen conjugates that do not comprise a charged moiety (C), is to combine such peptide antigen conjugates with either or both peptide antigen conjugate and/or amphiphilic carrier molecules that do form stable nanoparticle micelles.

In some embodiments, a first peptide antigen conjugate that does not comprise a charged moiety (C) (e.g., [B1]-A-[B2]-[L]-H) is mixed with a second peptide antigen conjugate comprising a charged moiety (e.g., C-[B1]-A-[B2]-[L]-H) in a DMSO solution and then resuspended in aqueous conditions to form stable nanoparticles. In other embodiments, a peptide antigen conjugate that does not comprise a charged moiety (C) (i.e. [B1]-A-[B2]-[L]-H) is mixed with a hydrophobic block linked to a charged moiety, such as C-H, in a DMSO solution and then resuspended in aqueous conditions to form stable nanoparticles.

In some embodiments, a peptide antigen conjugate that does not comprise a charged moiety, such as [B1]-A-[B2]-[L]-H, where [ ] denotes that the group is optional, is combined with an amphiphilic carrier, C-[B1]-[A′]-[B2]-[L]-H, wherein [ ] denotes the group is optional and optional A′ is a conserved antigen (i.e. not patient-specific).

In some embodiments, a peptide antigen conjugate that does not comprise a charged moiety, such as [B1]-A-[B2]-[L]-H, where [ ] denotes that the group is optional, is combined with an amphiphilic carrier of formula, e.g., S-[B]-[L]-H, wherein S is a solubilizing group, which may optionally comprise a charged moiety, e.g., C-[B]-[L]-H.

In still other embodiments, a peptide antigen conjugate comprising a charged moiety (C) is combined with an amphiphilic carrier that serves to further improve and ensure stability of the nanoparticle micelles formed by peptide antigen conjugates.

Amphiphilic carrier molecules may be neutral or include functional groups that carry charge at physiologic pH, and may therefore be referred to as charged amphiphilic carrier molecules or sometimes charged carrier molecules.

In some embodiments, the amphiphilic carrier molecule is neutral and has the formula S-[B]-[L]-H or H-[L]-[B]-S. In other embodiments, the amphiphilic carrier molecule of formula S-[B]-[L]-H or H-[L]-[B]-S has a net negative or net positive charge and can therefore be represented by the formula C-[B]-[L]-H or H-[L]-[B]-C, wherein the solubilizing group, S, is a charged moiety (C).

Amphiphilic carrier molecules with net positive or net negative charge typically comprise 2 or more, typically not more than 10, charged functional groups, such as 2, 3, 4, 5, 6, 7, 8, 9 and 10, which may comprise either positive and/or negatively charged functional groups (at physiologic pH 7.4). Positively charged functional groups are typically selected from amine and guanidine bases, and/or quaternary ammonium or sulfonium groups. Negatively charged functional groups are typically selected from carboxylates, sulfonates, sulfates, phosphonates and phosphates.

The number of charged functional groups included in a charged amphiphilic carrier molecule is selected to ensure stable nanoparticle micelle formation and to prevent formation of aggregates. In some embodiments, 4 or more amine or guanidine functional groups are needed to ensure stable nanoparticle micelle formation with amphiphilic carrier molecules of formula S—B—H, wherein H is comprised of 5 or more hydrophobic amino acids, such as 5 or more Tryptophan amino acids. In other embodiments, 4 or more carboxylate functional groups are needed to ensure stable nanoparticle micelle formation with amphiphilic carrier molecules of formula S—B—H, wherein H is comprised of 5 or more hydrophobic amino acids, such as 5 or more Tryptophan amino acids. Unexpectedly, amphiphilic carrier molecules of formula S—B—H, wherein H is comprised of 5 or more hydrophobic amino acids, such as 5 or more Tryptophan amino acids, were found to form stable nanoparticle micelles with as few as two or more functional groups comprised of sulfonates, sulfates, phosphonates and/or phosphates.

Moreover, the number of charged functional groups was also found to be dependent on the composition of the spacer, B, and the architecture of the amphiphilic carrier molecule.

Linear amphiphilic carrier molecules of formula S—B—H, wherein B is comprised of small and/or hydrophilic amino acids and H is comprised of 5 or more hydrophobic amino acids, such as 5 or more Tryptophan amino acids, typically required charged moieties with a greater number of charged functional groups, such as 6 or more, sometimes 10 or more amines, guanidines and/or carboxylates, or 3 or more, sometimes 4 or more, sulfonates, sulfates, phosphonates and/or phosphates. In contrast, linear amphiphilic carrier molecules of formula S—B—H, wherein B is comprised of a hydrophilic polymer, such as PEG or HPMA, and H is comprised of 5 or more hydrophobic amino acids, such as 5 or more Tryptophan amino acids, typically required charged moieties with fewer charged functional groups, such as 4 or more, sometimes 8 or more amines, guanidines and/or carboxylates, or 2 or more, sometimes 3 or more, sulfonates, sulfates, phosphonates and/or phosphates.

Additionally, the association between amphiphilic carrier molecule net charge and nanoparticle micelle formation was also found to be strongly dependent on the architecture of the amphiphilic carrier molecule. Notably, an unexpected finding reported herein is that amphiphilic carrier molecules with brush architecture required fewer charged functional groups than those of linear architecture. Amphiphilic carrier molecules with brush architecture may be prepared by linking hydrophobic blocks to amplifying linkers that provide two or more attachment points for C-B, for instance, (C-B)y19-K-[L]-H, wherein K is an amplifying linker and y19 denotes that there are an integer number, typically between 2 and 8, of charged blocks (C) linked to spacers (B) attached to the amplifying linker, which is attached either directly or through a Linker (L) to a hydrophobic block.

A non-limiting example of an amphiphilic carrier molecule with brush architecture of formula (C-B)y19-K-H, wherein y19 is 4, is provided here for clarity:

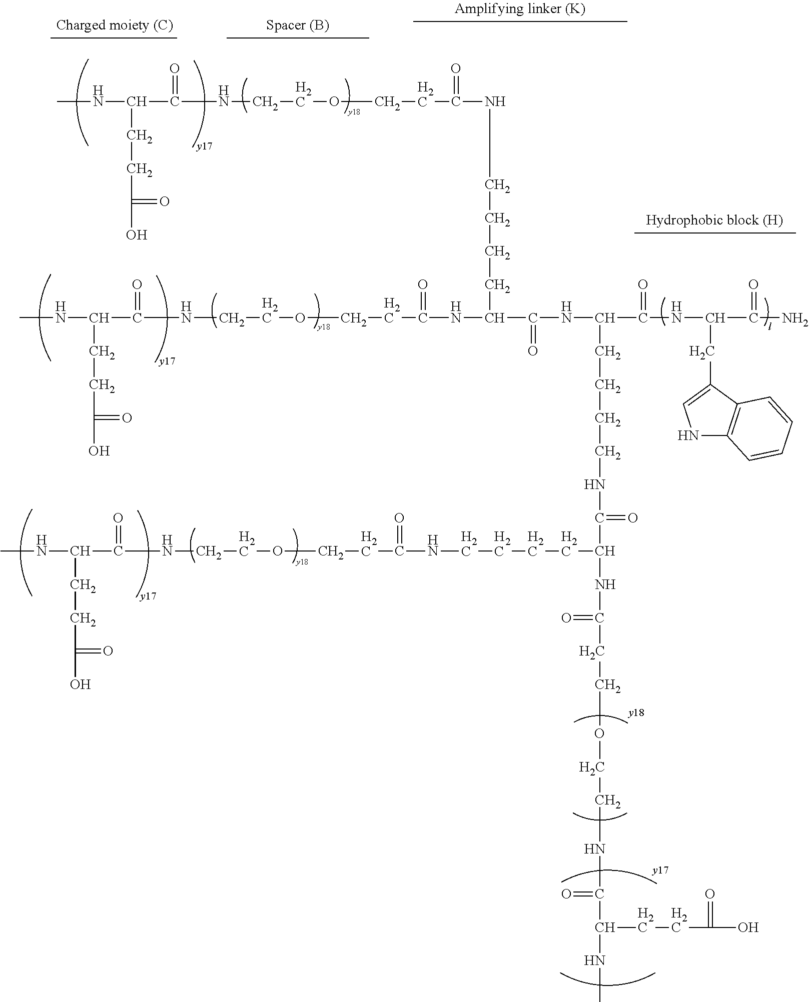

Wherein y17 is an integer number of repeating units of monomers comprising the charged moeity (C), typically selected from between about to 16, such as 1, 2, 3, 4, 5, 6, 7, 8, 9, 10, 11, 12, 13, 14, 15, 16; y18 is an integer number of repeating units of monomers comprising the spacer, which is typically between about 4 to about 200; and, the amine of the N-terminal amino acid for peptide-based charged moieties (C), as shown in this example, is either in the form of the free amine or is capped, e.g., with an acyl group

### Selection of the Molar Ratio of Peptide Antigen Conjugate to Amphiphilic Carrier Molecule

A notable and unexpected finding was that a broad range of different amphiphilic carrier molecule architectures and compositions were capable of leading to stable nanoparticle formation even in the presence of a high molar excess of peptide antigen conjugate.

In general, amphiphilic carrier molecules that form stable nanoparticle micelles when suspended in aqueous buffer alone were found to tolerate about a 4-fold molar excess of peptide antigen conjugate to amphiphilic carrier molecule, i.e. a molar ratio of about 1 to 4 moles of peptide antigen conjugate to moles of amphiphilic carrier molecule, such as about 1.1, 1.2 1.3, 1.4, 1.5, 1.6, 1.7, 1.8, 1.9, 2.0, 2.1, 2.2, 2.3, 2.4, 2.5, 2.6, 2.7. 2.8, 2.9, 3.0, 3.1, 3.2, 3.3, 3.4, 3.5, 3.6, 3.7, 3.8, 3.9 to about 4.0. Though, the ability of amphiphilic carrier molecules to tolerate excess peptide antigen conjugate was found to depend on several factors, including the net charge, architecture and composition of the amphiphilic carrier molecules.

Linear amphiphilic carrier molecules of formula C-[B]-[L]-H, H-[L]-[B]-C, C-[B1]-[A′]-[B2]-[L]-H and H-[L]-[B1]-[A′]-[B2]-C, with a charged moiety comprised of amino acids and having a net charge of greater than +6 or less than −6, in general were found to tolerate up to a 4-fold molar excess of peptide antigen conjugate.

Improved Manufacturability of Peptide Sequences Bearing Amino Acids with Amines

The present inventors unexpectedly observed that the enrichment of peptide antigen conjugates with amino acids comprising amines, e.g., lysine and arginine (with guanidine group), led to improved manufacturability of otherwise difficult to manufacture peptide-based hydrophobic blocks, peptide antigen fragments and peptide antigen conjugates.

Such improvements were not observed when peptide sequences were enriched with amino acids bearing carboxylic acids, e.g., aspartic acids or glutamic, or hydroxyl groups, e.g., serine, tyrosine and threonine, and, indeed, enrichment of peptides with certain hydrophobic amino acids, particularly, aliphatic amino acids, e.g., leucine, isoleucine and valine, even decreased manufacturability. A non-binding explanation for these findings is that amines and guanidine groups (while bearing protecting groups) allow adequate solubility and chain extension during solid-phase peptide synthesis and (after cleavage and deprotection) promote solubility in water and aqueous miscible organic solvents commonly used in peptide purification and handling, including DMSO, DMF, acetonitrile, methanol and ethanol. Moreover, as acids, such as formic acid, acetic acid and trifluoroacetic acid, are commonly added to HPLC solvents, the amine bearing amino acids also help to solubilize peptides during purification and even can improve peak shape to aid purification.

Therefore, in certain embodiments, peptide antigen fragments, peptide antigen conjugates and/or hydrophobic blocks are synthesized with one or more amino acids bearing amine functional groups. In certain embodiments of peptide antigen fragments, the peptide antigen fragment comprises one or more amino acids bearing an amine functional group. An unexpected finding reported herein is that whereas certain peptide antigens were non-manufacturable as the native sequence, i.e., without any amino acid residues flanking the natural peptide antigen sequence, those same peptide antigens synthesized as peptide antigen fragments and peptide antigen conjugates bearing one or more, typically between 1 and 30, such as 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, amino acids bearing amine functional groups could be manufactured.

Thus, in some preferred embodiments the peptide antigen fragment has the formula [C]-[B1]-A-[B2]-X1, [B1]-A-[B2]-X1([C]), X1-[B1]-A-[B2]-[C] or X1([C])-[B1]-A-[B2], where C is a charged moiety, B1 is an N-terminal extension, A is a peptide antigen, B2 is a C-terminal extension, [ ] denotes that the group is optional, and X1 is a linker precursor comprising a first reactive functional group, and [C], [B1] and/or [B2] comprise amino acids bearing an amine functional group. In such embodiments, the composition of the amino acid bearing an amine functional group may be selected based on the proximity of the amino acid to the charged moiety and hydrophobic block.

For amino acids bearing an amine functional group placed at or proximal to the charged moiety, the amino acid (when present in a peptide) should be water soluble at physiologic pH. Suitable amino acids include but are not limited to those comprising a lower alkyl amine or guanidine. Non-limiting examples are shown here for clarity:

Wherein, R¹⁰ is typically selected from

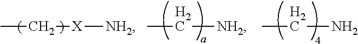

wherein X is any linker and a is any integer, though, typically an integer between 1 and 6. In preferred embodiments, the amino acid bearing a lower alkyl amine is lysine.

In some embodiments, the peptide antigen fragment of formula C-[B1]-A-[B2]-X1 or X1-[B1]-A-[B2]-C or the peptide antigen conjugate of formula C-[B1]-A-[B2]-[L]-H or H-[L]-[B1]-A-[B2]-C includes a charged moiety that comprises hydrophilic amino acids bearing amine functional groups, e.g., amino acids bearing a lower alkyl amine or guanidine. In other embodiments, the peptide antigen fragment of formula [C]-B1-A-[B2]-X1 or X1-[B1]-A-B2-[C] or the peptide antigen conjugate of formula [C]-B1-A-[B2]-[L]-H or H-[L]-[B1]-A-B2-[C] includes an N-terminal or C-terminal extension, respectively, that comprises amino acids bearing amine functional groups, e.g., amino acids bearing a lower alkyl amine or guanidine. In still other embodiments, the peptide antigen fragment of formula C-B1-A-[B2]-X1 or X1-[B1]-A-B2-C or the peptide antigen conjugate of formula C-B1-A-[B2]-[L]-H or H-[L]-[B1]-A-B2-C includes a charged moiety and an N-terminal or C-terminal extension, respectively, that comprises amino acids bearing amine functional groups, e.g., amino acids bearing a lower alkyl amine or guanidine.

Incorporation of amino acids bearing amine functional groups, e.g., amino acids bearing a lower alkyl amine or guanidine, such as lysine or arginine, in peptide antigen fragments and peptide antigen conjugates, unexpectedly improved manufacturability. Non-binding explanations include that the amine functional groups facilitate solubility as well as purification during reverse-phase HPLC purification.

### Placement of Amino Acids Bearing Aryl Amines at or Near the Hydrophobic Block

Amino acids comprising a lower alkyl amine or guanidine carry positive charge at physiologic pH (˜pH 7.4) that helps to improve solubility in aqueous solutions at or near physiologic pH, but such properties, i.e. solubility at physiologic pH, may not be desirable when such amino acids are placed at or near the hydrophobic block (H). Therefore, the current challenge that the inventors of the present disclosure sought to address is the need to improve manufacturability of peptide-based hydrophobic blocks, peptide antigen fragments and peptide antigen conjugates without adversely impacting the capacity of amphiphiles based on such materials to form stable particles in aqueous solutions around physiologic pH.

Recognizing this challenge, the inventors of the present disclosure introduced two novel approaches to leverage the benefits of incorporating amino acids bearing amine functional groups at or near the hydrophobic block without adversely impacting the hydrophobic characteristics of the hydrophobic block (H) or disrupting particle formation by the amphiphilic peptide antigen conjugates described herein. One approach was to introduce alkyl amines into peptide-based hydrophobic blocks during manufacturing but to cap (e.g., acylate) the alkyl amine groups prior to the incorporation of such hydrophobic blocks into peptide antigen conjugates. Another approach was to incorporate amino acids bearing aryl amines, which carry a positive charge at pH below physiologic pH, e.g., pH less than 6.5, but are neutral (non-charged) at physiologic pH, into peptide sequences, e.g., peptide-based hydrophobic blocks, peptide antigen fragments and peptide antigen conjugates.

An unexpected finding disclosed herein is that the incorporation of one or more amino acids, such as between 1 and 30, bearing an aryl amine functional group into peptides, e.g., peptide antigen fragments, peptide-based hydrophobic blocks and/or peptide antigen conjugates, during solid-phase peptide synthesis led to improved manufacturability as compared with peptides lacking amino acids bearing the aryl amine functional group. These findings were unexpected as amino acids comprising aromatic groups are often considered difficult to manufacture owing to their hydrophobic characteristics. However, unexpectedly, as reported herein, addition of amino acids with aromatic amines (aryl amines) to peptide sequences led to improved manufacturability comparable to that observed with the addition of lower alkyl amines, as described above.

While amino acids bearing an aryl amine improved manufacturability, such amino acids are typically highly hydrophobic in aqueous conditions at physiologic pH (˜pH 7.4). Therefore, such amino acids should be placed at or near the hydrophobic block but preferably not placed at or near the charged moiety (if present) of peptide antigen conjugates. In preferred embodiments, amino acids bearing an aryl amine group are placed at or proximal to the hydrophobic block to promote particle assembly. Suitable hydrophobic amino acids include but are not limited to those comprising an aryl amine. Non-limiting examples of amino acids bearing an aryl amine (as well as suitable heterocycles with protonatable nitrogens) are shown here for clarity:

Wherein, R¹¹ is typically selected from

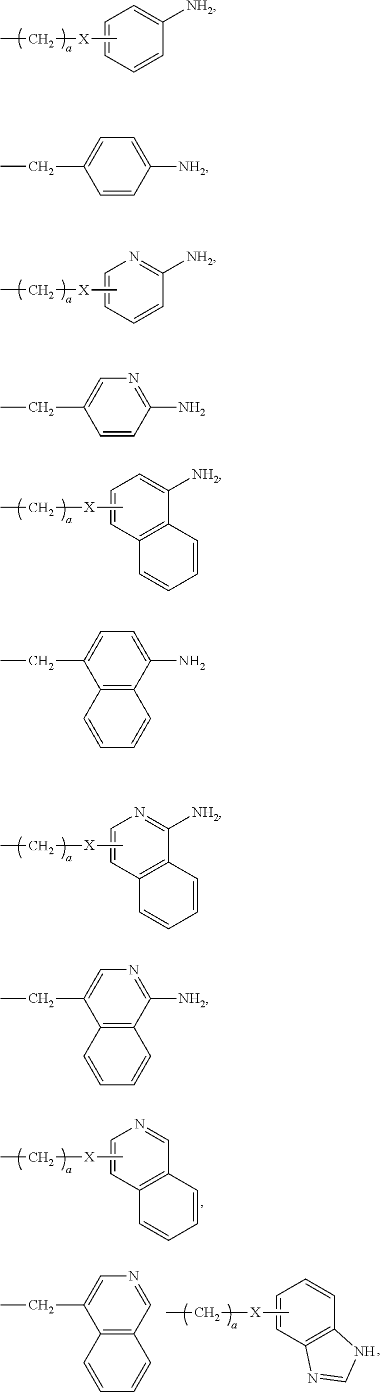

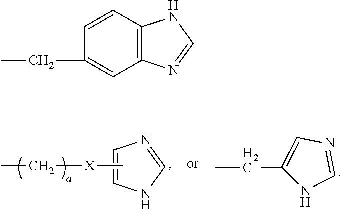

wherein X is any linker and a is any integer, though, typically an integer between 1 and 6. In preferred embodiments, the hydrophobic amino acid bearing an aryl amine placed at or near the hydrophobic block (H) is para-amino-phenylalanine.

In some embodiments, the peptide antigen fragment of formula [C]-[B1]-A-B2-X1 or X1-B1-A-[B2]-[C] or the peptide antigen conjugate of formula [C]-[B1]-A-B2-[L]-H or H-[L]-B1-A-[B2]-[C] includes a C-terminal or N-terminal extension, respectively, that comprises one or more hydrophobic amino acids bearing an aryl amine group.

In some embodiments, the peptide antigen fragment has the formula C-B1-A-B2-X1 and the C-terminal extension (B2) comprises one or more hydrophobic amino acids comprising an aryl amine group.

In other embodiments, the peptide antigen fragment has the formula A-B2-X1 and the C-terminal extension comprises one or more amino acids bearing an aryl amine group.

In some embodiments, the peptide antigen fragment has the formula X1-B1-A-B2-C and the N-terminal extension (B1) comprises one or more amino acids bearing an aryl amine group. In other embodiments, the peptide antigen fragment has the formula X1-B1-A and the N-terminal extension comprises one or more amino acids bearing aryl amine functional groups.

The unexpected findings related to the impact that the incorporation of amino acids bearing aryl amine functional groups have on peptide manufacturability led to the development of novel compositions and methods of manufacturing immunogenic compositions, e.g., vaccines, based on peptide antigen conjugates, wherein a peptide, e.g., a peptide antigen fragment, bearing an N- and/or C-terminal extension, which comprises one or more aryl amines, e.g., [C]-[B1]-A-[B2]-[X1], is produced by solid-phase peptide synthesis and then reacted with a hydrophobic block e.g., [X2]-H to produce a peptide antigen conjugate with the formula [C]-[B1]-A-[B2]-[L]-H. Unexpectedly, peptide antigen fragments of formula [C]-[B1]-A-[B2]-[X1] or X1-[B1]-A-[B2]-C, e.g., A-B2-X1 and X1-B1-A, and peptide antigen conjugates of formula [C]-[B1]-A-[B2]-[L]-H or [H]-[B1]-A-[B2]-[C], e.g., A-B2-L-H and H-L-B1-A, comprising N- or C-terminal extensions with one or more amino acids bearing aryl amine groups led to improved manufacturability, including improved solubility in organic solvents, as compared with peptide antigens alone or peptide antigen fragments, e.g., [C]-[B1]-A-[B2]-[X1], and/or peptide antigen conjugates, e.g., [C]-[B1]-A-[B2]-[L]-H, without aryl amines.

### Hydrophobic Blocks (H) Comprising Amino Acids Bearing Aryl Amines

Based on the unexpected finding that the incorporation of one or more amino acids bearing amine functional groups leads to improved manufacturing of peptides, e.g., peptide antigen fragments and peptide antigen conjugates, a novel strategy was developed by the inventors of the present disclosure wherein one or more, typically between 3 and 30, amino acids comprising aryl amines, were incorporated into the hydrophobic blocks of peptide antigen conjugates of the formula [C]-[B1]-A-[B2]-[L]-H or H-[L]-[B1]-A-[B2]-[C] on resin during solid-phase-peptide synthesis. Whereas synthesis of peptide antigen conjugates of formula [C]-[B1]-A-[B2]-[L]-H or H-[L]-[B1]-A-[B2]-[C] with hydrophobic blocks (H) based on conventionally used lipophilic molecules, such as fatty acids, lipids or peptides comprised of amino acids with aliphatic side chains, or aromatic groups without amines, were found to be difficult to synthesize and/or purify, it was found unexpectedly that incorporation of hydrophobic blocks, which comprise one or more amino acids bearing an aromatic amine, on-resin during solid-phase synthesis led to improved synthesis of the peptide antigen conjugate. Importantly, the peptide antigen conjugates of the formula [C]-[B1]-A-[B2]-[L]-H or H-[L]-[B1]-A-[B2]-[C] with hydrophobic blocks (H) comprising one or more, typically between 3 to 30, amino acids bearing aryl amines were also found to reliably assemble into particles upon suspension in an aqueous buffer owing to the hydrophobic properties of the amino acids bearing the aryl amine groups.

Historically, highly hydrophobic peptide sequences have been challenging to manufacture because of low coupling efficiency during synthesis and/or limited solubility that complicates purification. Importantly, the aforementioned results highlight the unexpected finding that such a challenge can be overcome through the use of amino acids bearing aryl amine groups that improve manufacturing and solubility in water-miscible organic solvents, but are sufficiently hydrophobic in aqueous conditions around physiologic pH, i.e. pH 7.4, to retain the hydrophobic characteristics required to drive particle formation when used as the hydrophobic portion within amphiphilic compounds.

In some embodiments, peptide antigen conjugates of formula [C]-[B1]-A-[B2]-[L]-H or H-[L]-[B1]-A-[B2]-[C] produced on-resin are synthesized with hydrophobic blocks comprising one or more, typically between 3 and 30 hydrophobic amino acids bearing aryl amine groups.

In still other embodiments, the peptide antigen conjugate of formula [C]-[B1]-A-B2-[L]-H or H-[L]-B1-A-[B2]-[C] includes a C-terminal or N-terminal extension, respectively, as well as a hydrophobic block that comprises amino acids bearing an aryl amine functional group.

In other embodiments, hydrophobic blocks bearing an X2 linker precursor and comprising one or more, typically between 3 and 30 hydrophobic amino acids bearing aryl amine groups, are reacted in solution-phase with a peptide antigen fragment of [C]-[B1]-A-[B2]-X1 or X1-[B1]-A-[B2]-[C] to produce peptide antigen conjugates of formula [C]-[B1]-A-[B2]-L-H and H-L-[B1]-A-[B2]-[C], respectively.

Peptide antigens conjugates that lack a charged moiety, e.g., A-B2-[L]-H and H-[L]-B1-A, and include one or more amino acids bearing an aryl amine at the C- and N-terminal extensions, respectively, are highly hydrophobic and, in preferred embodiments of immunogenic compositions, are typically combined with an amphiphilic carrier molecule, e.g., a charged amphiphilic carrier molecule, that functions to solubilize the peptide antigen conjugate in aqueous solutions, e.g., aqueous buffers, and promotes incorporation of the peptide antigen conjugates into micelles.

In the preparation of some embodiments of immunogenic compositions of nanoparticles micelles comprising peptide antigens conjugates that lack a charged moiety, e.g., A-B2-[L]-H and H-[L]-B1-A and include one or more amino acids bearing an aryl amine at the C- and/or N-terminal extensions, such peptide antigen conjugates are suspended in a water-miscible organic solvent (e.g., DMSO, DMF, ethanol or acetone) and mixed with a charged amphiphilic carrier molecule, e.g., a charged amphiphilic carrier molecule of formula C-[B]-[L]-H or C-[B1]-A′-[B2]-[L]-H to form a mixture that is diluted with an aqueous solution to generate an aqueous solution of nanoparticle micelles comprised of peptide antigen conjugates, e.g., A-B2-[L]-H or H-[L]-B1-A, and charged amphiphilic carrier molecules, e.g., C-[B]-[L]-H or C-[B1]-A′-[B2]-[L]-H.

A non-limiting example of an immunogenic composition of nanoparticles micelles comprising charged amphiphilic carrier molecules and peptide antigen conjugates of formula A-B2-L-H wherein one or more amino acids bearing an aryl amine are present in the C-terminal extension (B2), is provided here for clarity:

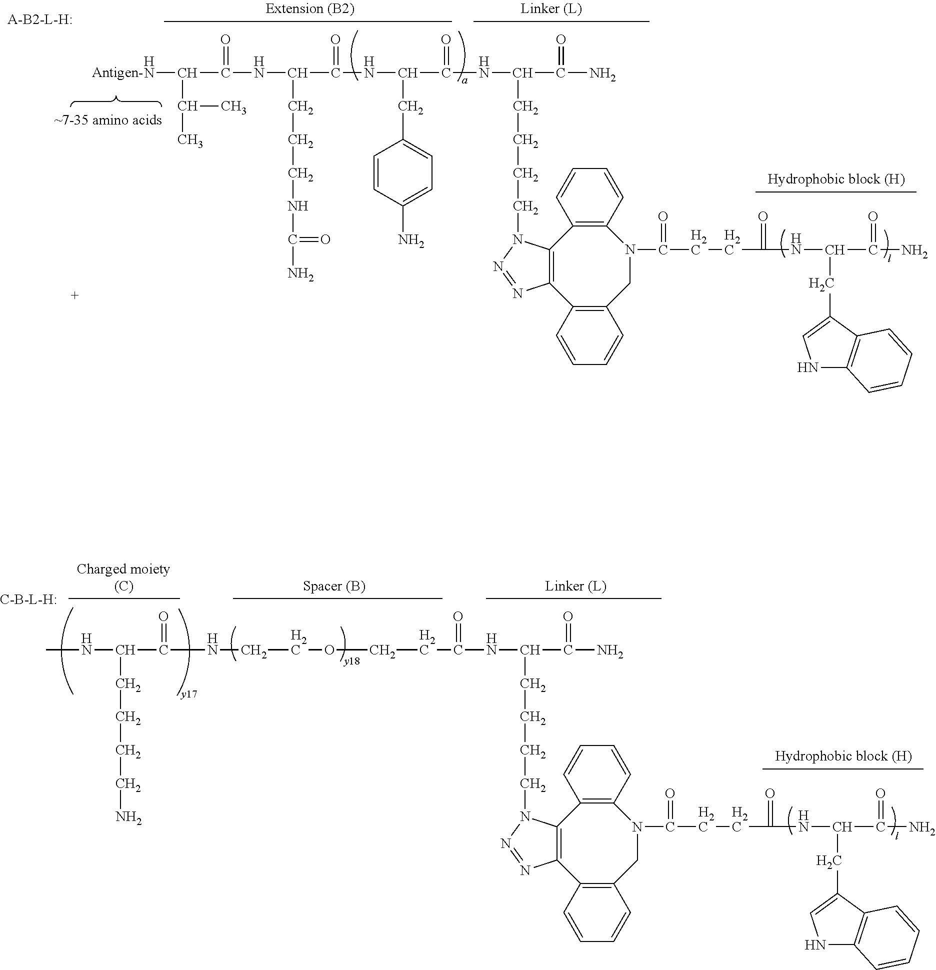

Wherein a is any integer, typically between about 1 to 10; y17 is an integer number of repeating units of monomers comprising the charged moeity (C), typically selected from between about 1 to 16, such as 1, 2, 3, 4, 5, 6, 7, 8, 9, 10, 11, 12, 13, 14, 15, 16; y18 is an integer number of repeating units of monomers comprising the spacer group (B) of the amphiphilic carrier molecule, which is typically between about 4 to about 200; and, the amine of the N-terminal amino acid for peptide-based charged moieties is either in the form of the free amine or is capped, e.g., with an acyl group.

### Selection of the Number of Amino Acids Bearing Aryl Amines

The choice of the number of amino acids bearing aryl amines depends on whether such amino acids are placed on an extension sequence or on the hydrophobic block. The incorporation of amino acids bearing aryl amines onto extensions (B1 or B2) that are proximal to the linker precursor (X1) of peptide antigen fragments, or on extensions (B1 or B2) that are proximal to the linker (L) or hydrophobic block (H) of peptide antigen conjugates should be of a high enough number to improve solubility of the peptide sequence in aqueous miscible organic solvents. Unexpectedly, the inventors of the present disclosure found that between 1 and 10, such as 1, 2, 3, 4, 5, 6, 7, 8, 9 and 10 amino acids bearing aryl amine groups were sufficient to improve manufacturability and solubility of peptide antigen fragments and peptide antigen conjugates. Therefore, in preferred embodiments of peptide antigen fragments and peptide antigen conjugates with N- or C-terminal extensions comprising amino acids bearing aryl amines, the number of amino acids bearing aryl amines is typically selected to be between 1 and 10, such as 1, 2, 3, 4, 5, 6, 7, 8, 9 and 10 amino acids bearing an aryl amine.

The choice for the specific number of amino acids bearing aryl amines depends, in part, on the length of the overall peptide sequence. For synthesis of peptide antigen fragments of formula [C]-[B1]-A-B2-X1 or X1-B1-A-[B2]-[C] of between a total of 10 to 50 amino acids, amino acids bearing aryl amine groups are typically placed at the B2 or B1 positions, respectively, and are typically an integer number of between 1 and 6.

The incorporation of amino acids bearing aryl amines onto peptide-based hydrophobic blocks (H), both when the hydrophobic block is produced alone or on-resin as a peptide antigen conjugate, should be of a high enough number to improve solubility of the peptide sequence in aqueous miscible organic solvents; and/or when used as the dominant and/or majority monomer unit of the hydrophobic block, should be of a high enough number to drive particle assembly when present on peptide antigen conjugates or amphiphilic carrier molecules, e.g., S-[B]-[L]-H, wherein S is a solubilizing group, which may comprise a charged moiety, e.g., C-[B]-[L]-H.

Unexpectedly, the inventors of the present disclosure found that between 1 and 10, such as 1, 2, 3, 4, 5, 6, 7, 8, 9 and 10 amino acids bearing aryl amine groups were sufficient to improve manufacturability and solubility of peptide-based hydrophobic blocks and peptide antigen conjugates, but that between 3 and 30, such as 3, 4, 5, 6, 7, 8, 9, 10, 11, 12, 13, 14, 15, 16, 17, 18, 19, 20, 21, 22, 23, 24, 25, 26, 27, 28, 29 or 30, were preferable when such amino acids were the dominant and/or majority monomer unit in the hydrophobic block. Therefore, in preferred embodiments, the number of amino acids bearing aryl amines incorporated into peptide-based hydrophobic blocks produced on-resin is typically selected to be between 3 and 30. Of note, though, peptide-based hydrophobic blocks (H) comprising more than 30 amino acids, typically more than 50 amino acids, are preferably prepared by convergent assembly of two or more peptides produced by solid-phase peptide synthesis or are prepared by an alternative route, such as by ring opening polymerization.

### Immunogenic Compositions

The peptide antigen conjugates disclosed herein may be used in immunogenic compositions to treat tumors, infectious diseases, auto-immunity or allergies.

The peptide antigen conjugates may be used alone or in combination with other therapies. For the treatment of cancers, immunogenic compositions comprised of peptide antigen conjugates may be used prior to, during or after treatment surgery, radiation therapy or chemotherapy. In preferred embodiments, the immunogenic compositions comprising the peptide antigen conjugates are used in combination with immuno-modulators, such as cytokines (e.g., IL-2), anti-tumor antibodies, checkpoint inhibitors (such as anti-PD1) antibodies, or other small molecules or biologics that reverse immune-suppression, directly kill tumor cells or potentiate the immune response against the tumor. The peptide antigen conjugates disclosed herein may also be used in heterologous prime-boost immunizations, such as a prime or boost with a peptide antigen conjugate and a prime or boost with a heterologous vaccine, such as a viral vector.

The immunogenic compositions disclosed herein can be formulated as pharmaceutical compositions prepared for administration to a subject and which include a therapeutically effective amount of one or more of the immunogens as described herein. The therapeutically effective amount of a disclosed compound will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Immunogenic compositions for administration to a subject can be pharmaceutical compositions and can include at least one further pharmaceutically acceptable additive such as carriers, thickeners, diluents, buffers, preservatives, surface active agents and the like in addition to the molecule of choice. Immunogenic compositions can also include one or more additional active ingredients such as antimicrobial agents, anesthetics, and the like. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

To formulate the immunogenic compositions, the disclosed nanoparticle components or a solution containing the disclosed nanoparticle components can be combined with various pharmaceutically acceptable additives, as well as a base or vehicle for dispersion of the nanoparticles. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The immunogenic compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

The instant disclosure also includes kits, packages and multi-container units containing the herein described immunogenic compositions, active ingredients, and/or means for administering the same for use in the prevention and treatment of diseases and other conditions in mammalian subjects. In one embodiment, these kits include a container or formulation that contains one or more of the immunogenic compositions described herein. In one example, the immunogenic composition is formulated in a pharmaceutical preparation for delivery to a subject. The immunogenic composition is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

### EXAMPLES

### Example 1—Synthesis of Peptide-Based Hydrophobic Blocks of Formula II Linked to Bioactive Ligand Molecules (Ligands) Through Pendant Side Chains

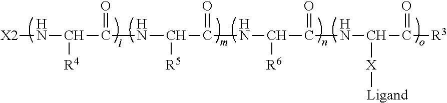

### Hydrophobic Block Fragment Based on a Hydrophobic Block (H) of Formula H(b)

Hydrophobic block fragments comprising bioactive ligand molecules (“Ligands”) and the linker precursor X2, such as the example of the hydrophobic block of Formula II(b) shown above, must be prepared in a synthetic scheme that does not destroy the Ligand or the linker precursor X2. Peptide-based hydrophobic blocks (H) may be synthesized by solid-phase peptide synthesis (SPPS) or ring-opening polymerization of amino acid N-carboxyanhydrides (NCAs). While ring-opening polymerization can be performed in mild conditions with low risk for degradation of the Ligand and linker precursor X2, SPPS provides the advantage over ring-opening polymerization that the peptide length, as well as distribution and composition of monomers, is precisely chemically defined by the programmable step-wise addition of amino acids to a resin.

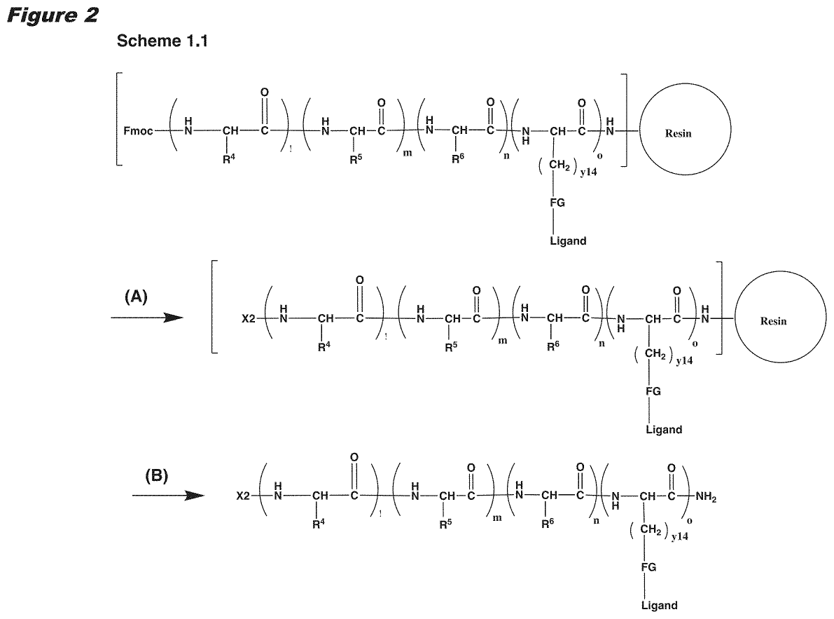

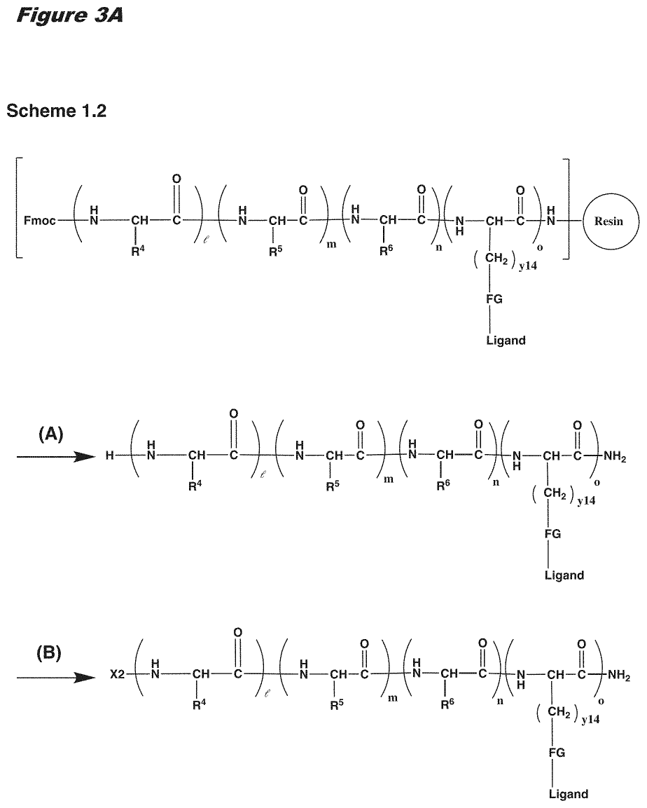

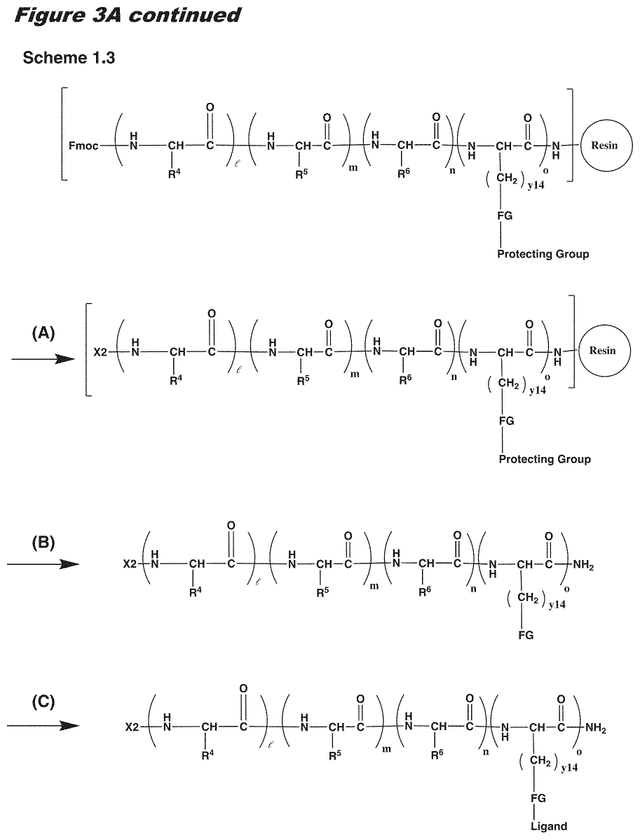

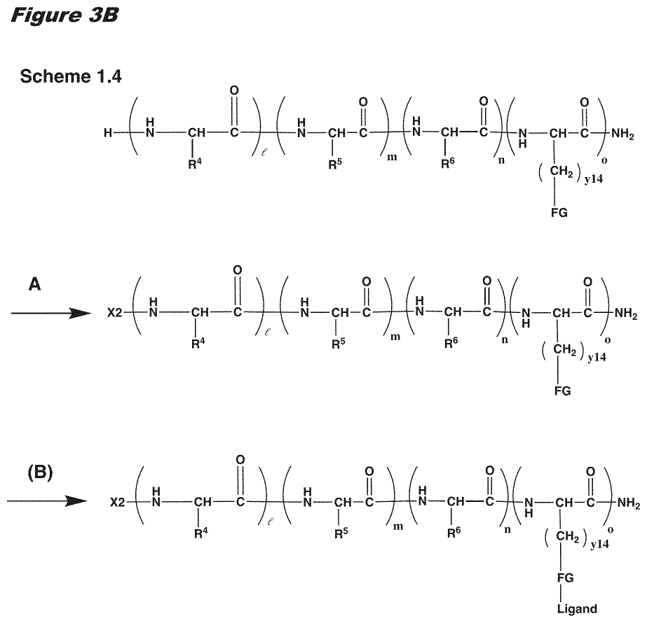

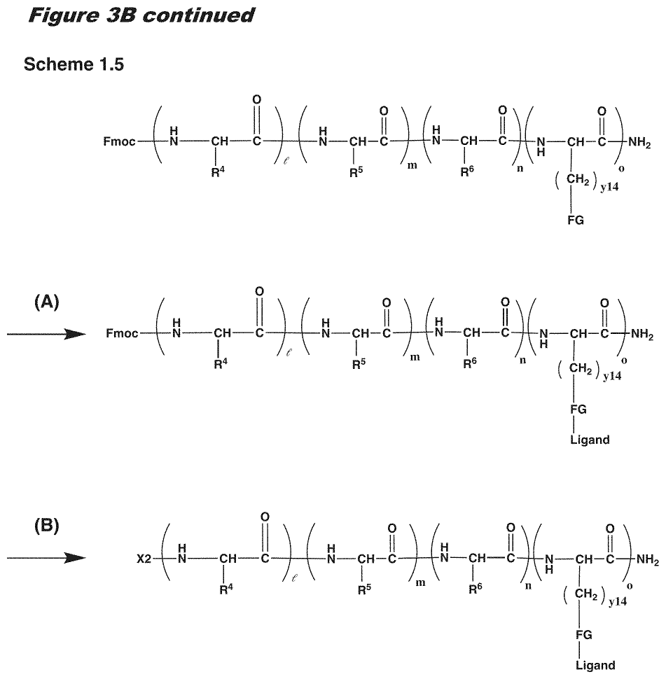

There are several different routes for producing peptide-based hydrophobic block fragments, including those based on hydrophobic blocks (H) of Formula I and Formula II. One route is to perform the entire synthesis by SPPS (Scheme 1.1, FIG. 2). In scheme 1.1, the Ligand molecule is attached to individual amino acids prior to coupling or is coupled to amino acids on-resin, while the linker precursor X2, can be simply linked to the N-terminal position after chain elongation is complete. However, the Ligand molecule and/or linker precursor X2 may not be stable during the acidic or basic conditions needed for deprotection and cleavage of peptides from solid-phase resins. Additionally, the Ligand molecule may be reactive during the coupling steps, making the (un-protected) Ligand unsuitable to include on the peptide chain during chain elongation, or requiring protection of the Ligand to make such a strategy suitable. To avoid unwanted reactions or decomposition, either or both the Ligand molecule and linker precursor X2 can be linked to the peptide following cleavage of the peptide from the resin. Schemes 1.2 and 1.3 (FIG. 3) describe the partial on-resin coupling, where either the linker precursor X2 or the Ligand, respectively, are coupled to the peptide-based hydrophobic block (H) in solution. Finally, if both the linker precursor X2 and the Ligand are unsuitable for on-resin coupling, the coupling of both the linker precursor X2 and the Ligand may occur in solution as shown in Schemes 1.4 and 1.5 (FIG. 3).

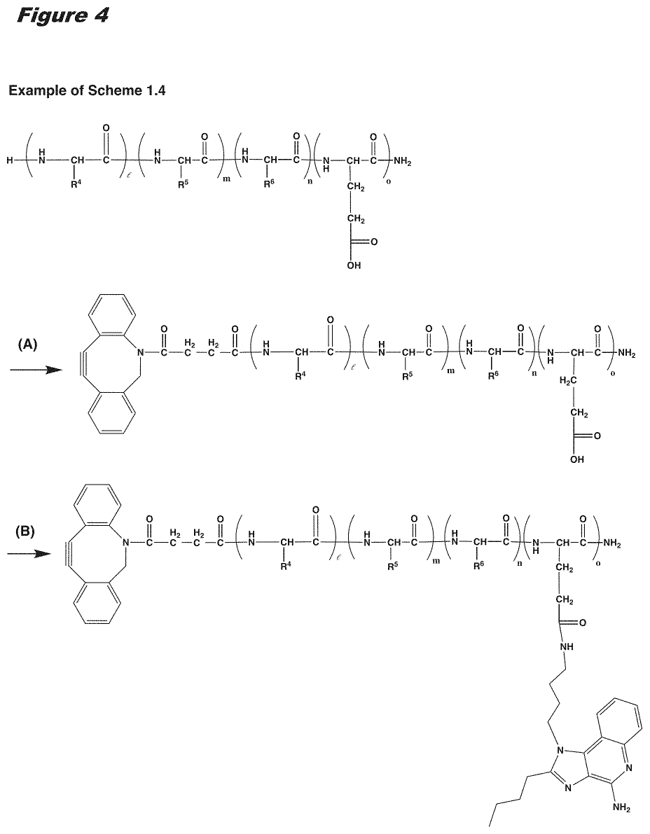

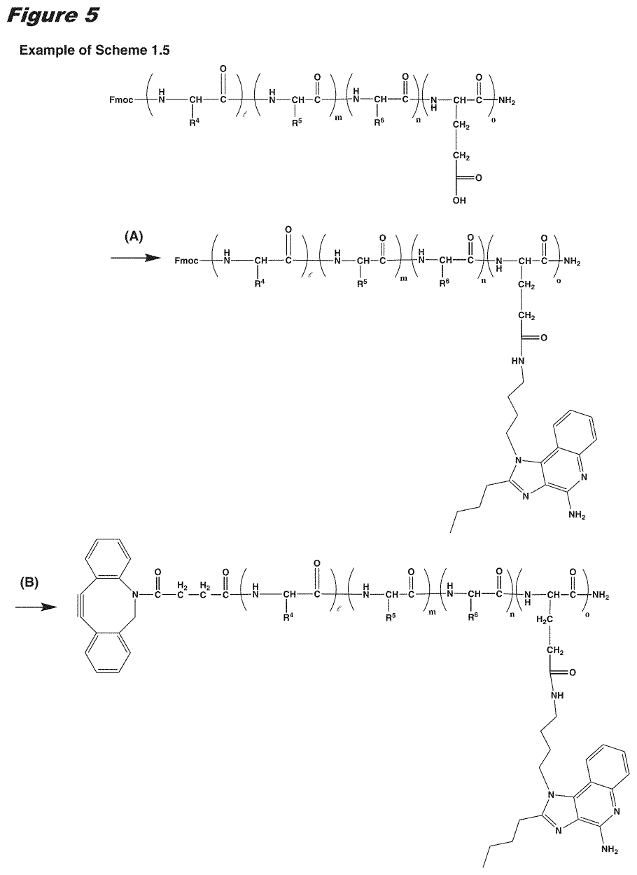

In certain embodiments, the hydrophobic block (H) of Formula II is linked to adjuvants of Formula III (imidazoquinoline TLR-7/8a, referred to as “TLR-7/8a”) and a linker precursor X2 that comprises a clickable group, e.g., DBCO. As the TLR-7/8a contains an aryl amine that is reactive in coupling conditions employed for SPPS and as we found, unexpectedly, that the DBCO molecule is unstable at highly basic (20% piperidine/DMF) and acidic conditions (>30% TFA/DCM), it is preferable to produce such a hydrophobic block fragment using Schemes 1.4 and 1.5, where coupling of the TLR-7/8a and DBCO molecule occurs after the peptide is cleaved from the resin (FIGS. 4 and 5).

### Example 2—Synthesis of Peptide Antigen Conjugates

### Conjugation by Strain-Promoted Azide-Alkyne Cycloaddition

A preferred route for the synthesis of peptide antigen conjugates is through convergent synthesis where the peptide antigen fragment, comprising an antigen (A) and a linker precursor (X1) containing a “clickable group,” and optionally C, B1 and B2, is produced. Separately, in a parallel scheme, a hydrophobic block (H) linked to a linker precursor (X2) that is reactive towards the linker precursor X1 is produced. The peptide antigen fragment and hydrophobic block fragment are then linked together via a reaction between X1 and X2 to form a Linker (L).

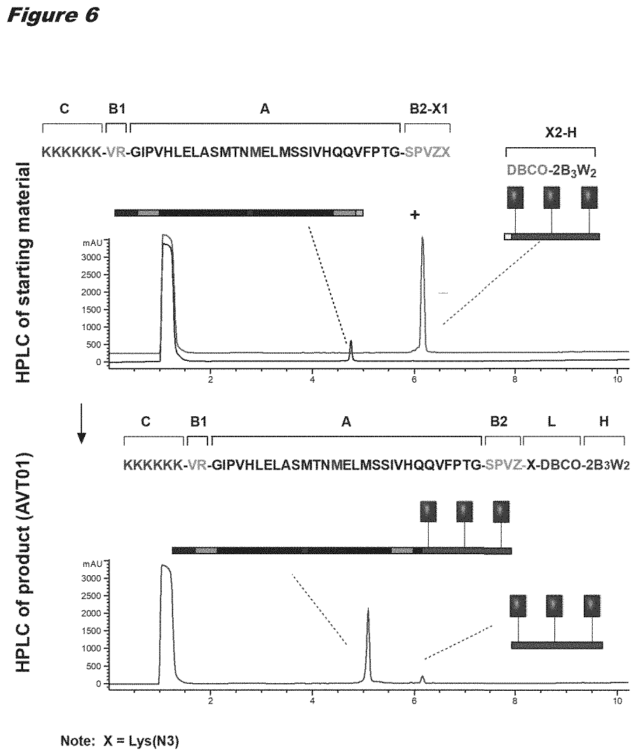

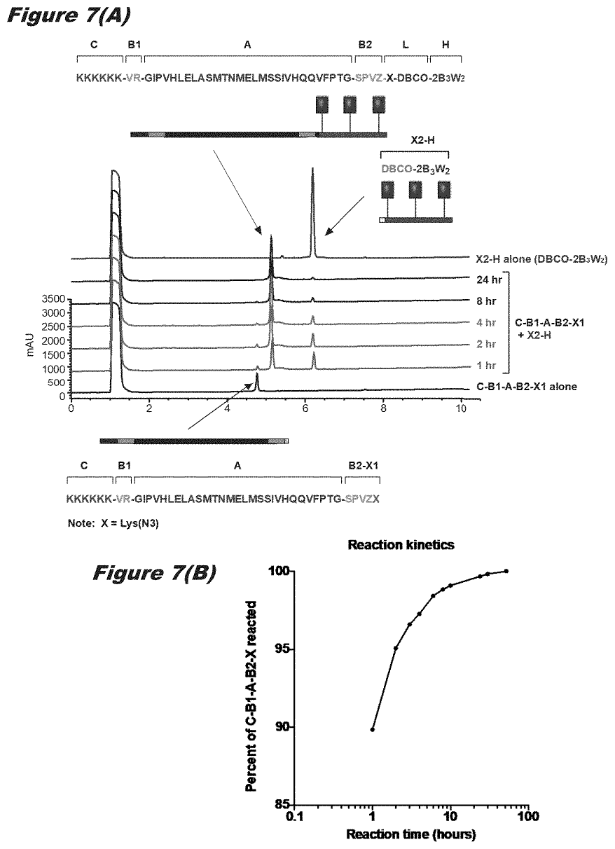

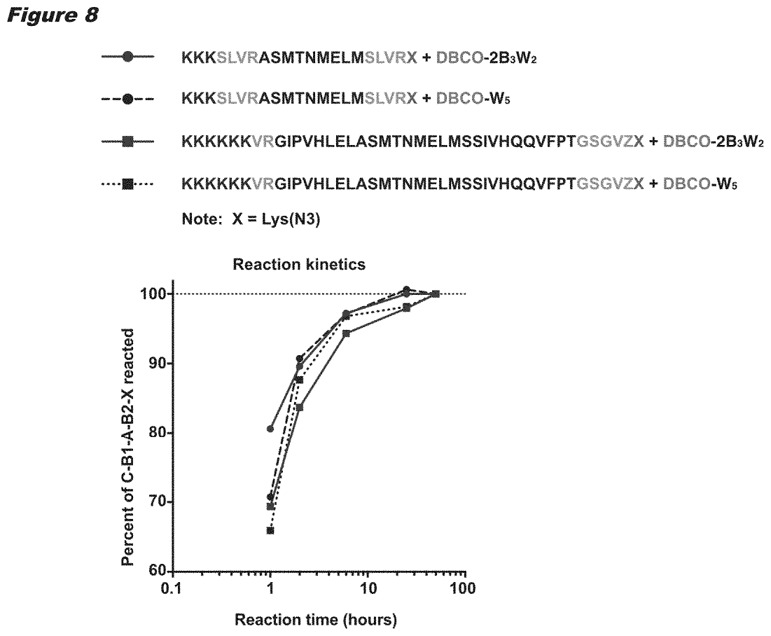

In one example, the peptide antigen fragment linker precursor (X1) is an azido amino acid, e.g., azidolysine at the C-terminal position of the peptide antigen fragment (C-B1-A-B2-X1), and the linker precursor (X2) on the hydrophobic block fragment (X2-H) is DBCO and is placed at the N-terminus of the hydrophobic block (H). As shown in FIG. 6, a peptide antigen fragment (e.g., C1-B1-A-B2-X1) and a hydrophobic block fragment (X2-H) were reacted at a molar ratio of 1 to 1.2 at room temperature in DMSO and these conditions resulted in full conversion of the peptide antigen fragment to the peptide antigen conjugate (C-B1-A-B2-L-H), which is driven by the use of excess hydrophobic block fragment (FIG. 7). Importantly, similar reaction kinetics were observed using different length peptide antigen fragments (C-B1-A-B2-X1) as well as different compositions of hydrophobic blocks (H) (FIG. 8), indicating that the copper-free click chemistry conjugation of peptide antigen fragments to the hydrophobic block (H) to generate the peptide antigen conjugate is a robust and reliable reaction.

### Reaction Kinetics

As personalized cancer vaccines require rapid manufacturing, it is beneficial to increase the rate of the reaction for the formation of the peptide antigen conjugate. Reaction kinetics may be increased by increasing the reaction temperature and/or increasing reagent concentration, as well as through the use of catalysts, chaperone molecules or by identifying optimal solvent and salt concentrations as may be the case for larger macromolecules.

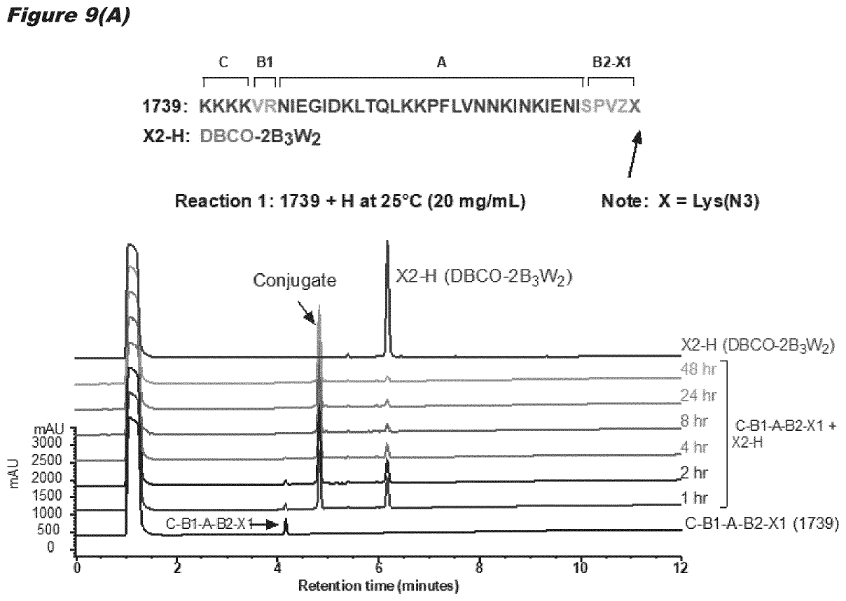

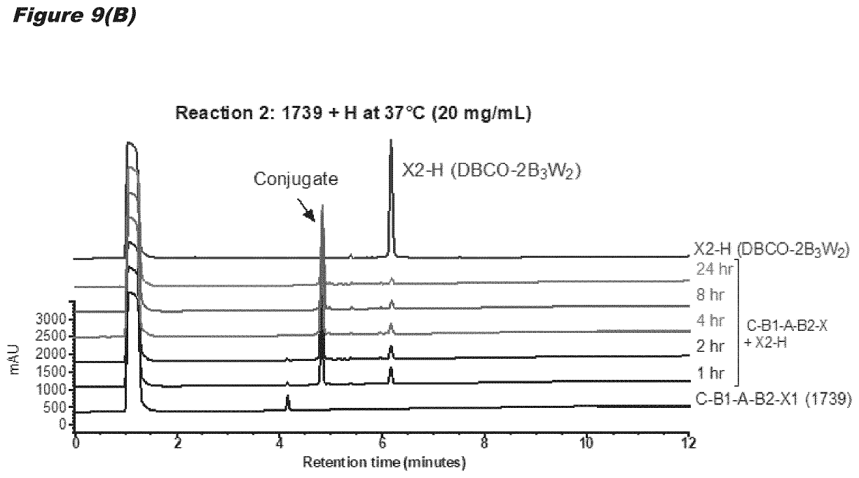

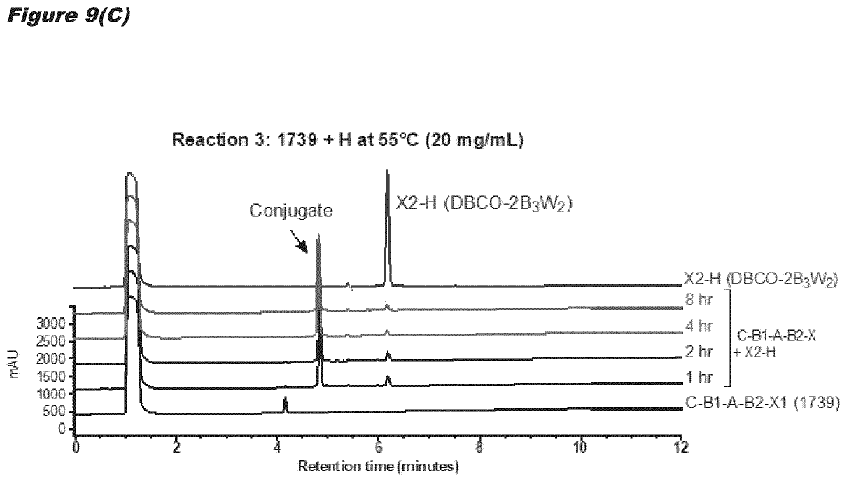

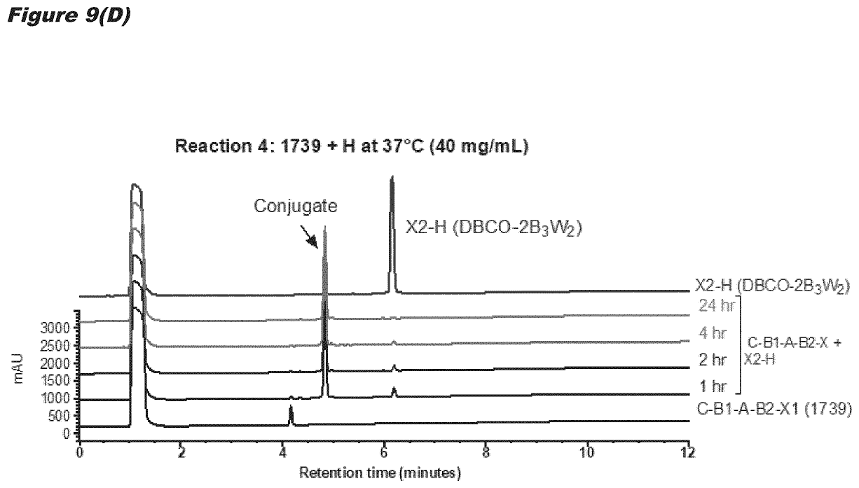

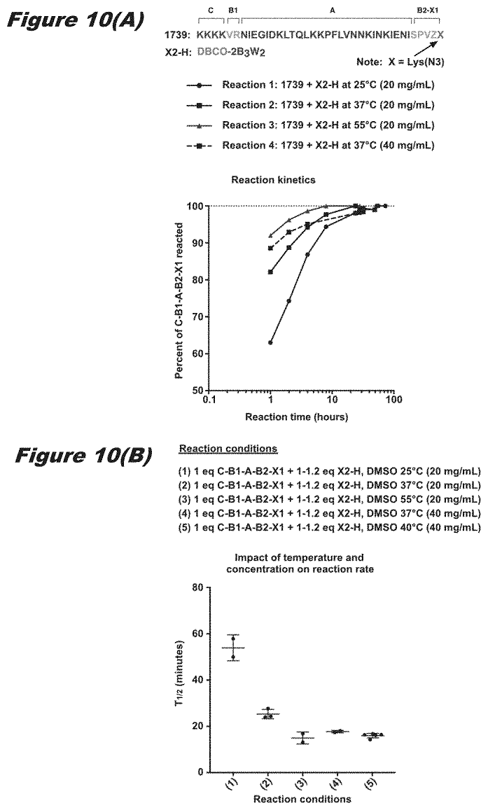

However, it was not known a priori how increasing the temperature of the reaction would impact the stability of the peptide antigen fragment, the hydrophobic block fragment, or the resulting peptide antigen conjugate. Our results show that increasing the reaction temperature up to 55° C. results in about a 3-fold decrease in the reaction half-life without resulting in byproducts or decomposition of the starting materials (FIGS. 9 and 10). A similar improvement in the reaction kinetics was observed by increasing the concentration of the starting materials (FIG. 10). As shown in FIG. 10B, the reaction half-life shortens with increasing reactant concentration and reaction temperature across a diverse range of peptide antigen fragments tested, indicating that these results are broadly applicable to the synthesis of peptide antigen conjugates.

### Impact of Excess Hydrophobic Block (H) on Particle Stability and In Vivo Activity

A molar excess (0-20%) of hydrophobic block fragment was used when conjugating the peptide antigen fragment to the hydrophobic block fragment in order to achieve maximal (near 100%) peptide conversion. Removal of the excess (i.e. unreacted) hydrophobic block fragment can be achieved by chromatographic separation or the use of a scavenger that can selectively remove the unreacted DBCO-modified hydrophobic block fragment. However, such purification steps can reduce overall peptide antigen conjugate yield and significantly increase manufacturing costs and time.

While it would be more efficient to forgo the costly and time consuming purification steps to remove unreacted hydrophobic block fragment, it is not known how excess hydrophobic block fragment impacts hydrodynamic behaviour (i.e., particle size, which is defined as the average hydrodynamic diameter measured by DLS, and stability) or immunogenicity of the product solution comprising the peptide antigen conjugate, any unreacted hydrophobic block fragment (sometimes referred to as unreacted hydrophobic block) and pharmaceutically acceptable organic solvent. Indeed, particle size affects pharmacokinetics and biodistribution of particles in vivo. Thus, it is important to control the size of the particles formed by the peptide antigen conjugates and ensure consistent size distribution across different compositions of peptide antigen conjugates.

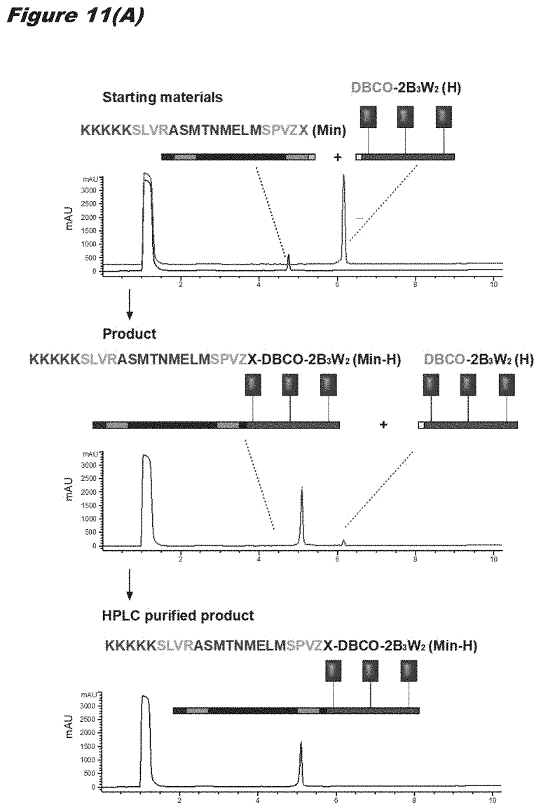

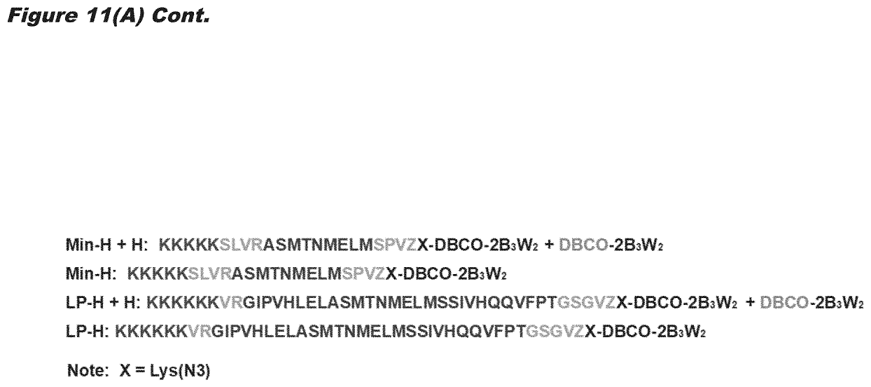

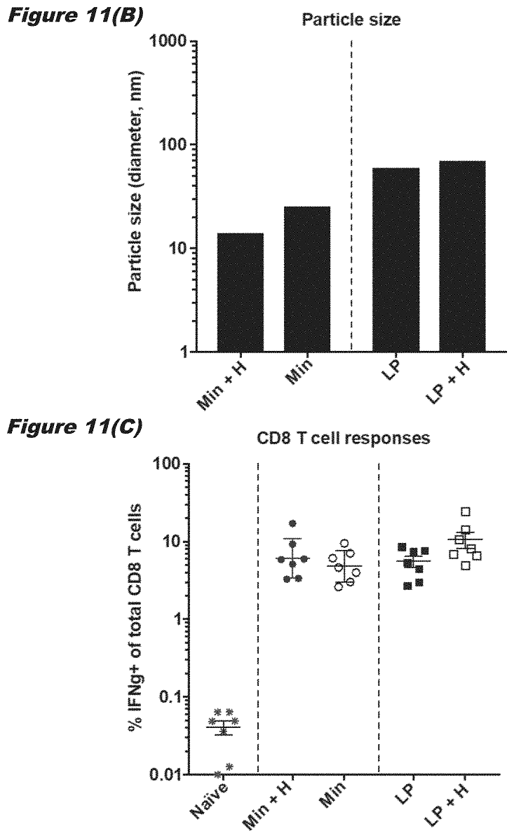

To evaluate how excess (i.e. unreacted) hydrophobic block fragment impacts particle size and in vivo activity, we synthesized two different peptide antigen conjugates, differing in peptide antigen fragment length, and split the reaction mixtures (i.e. product solutions) of each into two portions of equal volume (FIG. 11). One portion was immediately re-suspended in PBS to provide an aqueous mixture of peptide antigen conjugate particles and used for particle size and in vivo testing, and the other portion was purified by HPLC to remove excess (i.e. unreacted) hydrophobic block (H) and then lyophilized to generate a lyophilized purified peptide antigen conjugate that was resuspended in PBS and then evaluated for particle size and in vivo activity. Both the crude conjugation product (product solution) containing up to 20% of free hydrophobic block and the HPLC purified material (lyophilized purified peptide antigen conjugate) resulted in the formation of nano-sized particles (aqueous solution of peptide antigen conjugate particles) of comparable size in aqueous buffer (FIG. 11B), indicating that excess hydrophobic block fragment has minimal impact on the sizes of particles formed by the peptide antigen conjugate particles in aqueous buffer. Importantly, the immune responses induced by both the aqueous mixtures of the crude reaction mixture (product solution) and the HPLC purified peptide antigen conjugate resulted in comparable magnitude of CD8 T cell responses that were statistically significant as compared with naïve animals, suggesting that the excess hydrophobic block fragment has minimal impact on the in vivo activity of the peptide antigen conjugate.

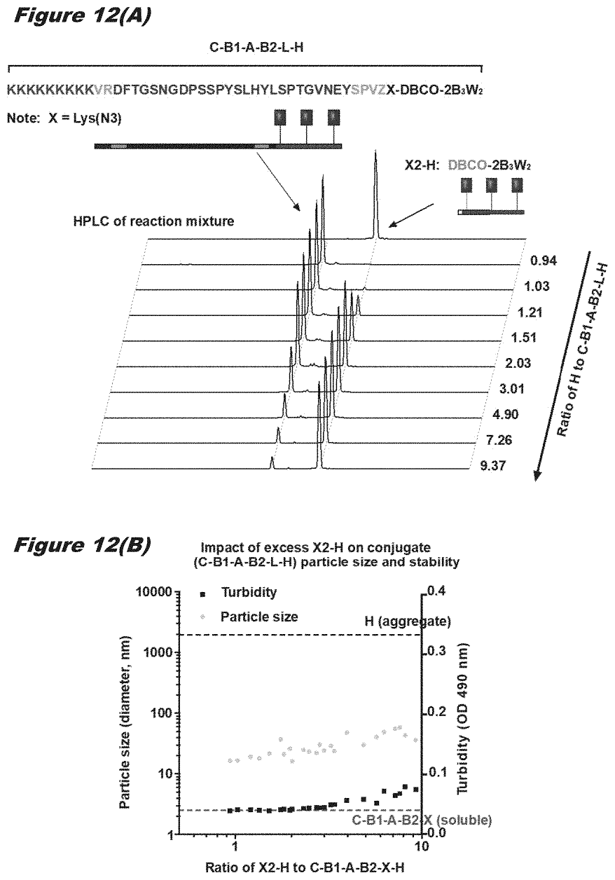

To extend our findings, we evaluated how extremes of excess (unreacted) hydrophobic block fragment impact the size and stability of particles formed by peptide antigen conjugates (FIG. 12). We reacted a peptide antigen fragment (C-B1-A-B2-X1, wherein X1=azidolysine) with different molar ratios of a hydrophobic block fragment (DBCO-2B₃W2, sometimes referred to as “hydrophobic block (H)”) ranging from a 1 to 0.94 molar ratio of peptide antigen fragment to hydrophobic block fragment up to a molar ratio of 1 to 9.37 (FIG. 12A). Unexpectedly, product solutions comprising peptide antigen conjugates of Formula V formed stable nanoparticle micelles with up to about a 3-fold excess of the hydrophobic block fragment (FIG. 12B), with minimal impact on size and stability of nanoparticle micelles formed by product solutions comprising up to a 10-fold excess of unreacted hydrophobic block fragment. These results suggest that the excess hydrophobic block fragment is likely encapsulated within the micellar nanoparticle structure formed by the peptide antigen conjugates in aqueous solutions. The hydrodynamic stability of the micelles formed by the peptide antigen conjugate therefore provides a means to solubilize the uncreated hydrophobic block fragment, as well as possibly other hydrophobic drugs.

These results show clearly the novel finding that excess (i.e. unreacted) hydrophobic block fragment has limited impact on particle size, stability, or in vivo activity of peptide antigen conjugates, suggesting that it is not necessary to remove unreacted hydrophobic block fragment from the product solution. This conclusion was drawn based on two observations: 1) the hydrodynamic size of the particles formed by the peptide antigen conjugates in PBS buffer was the same for both the crude reaction mixture (i.e. product solution) and the HPLC purified peptide antigen conjugate (FIG. 11B) as well as the crude reaction mixtures (product solutions) of peptide antigen conjugates with a broad range of amounts of unreacted hydrophobic block fragment (FIG. 12); and 2) the immunogenicity induced in vivo was the same for both the product solution as well as HPLC purified peptide antigen conjugate (FIG. 11C). The implication of these unexpected findings is that no additional purification following the conjugation step is necessary, meaning the crude reaction mixture (product solution) can be advanced for further use, such as for characterization, sterile filtration, formulation and then administration to a subject.

### Example 3—Characterization of Product Solutions and Purified Peptide Antigen Conjugate Solutions

### Assessing the Peptide Antigen Conjugate and Hydrophobic Block Fragment Concentration

The product solution or purified peptide antigen conjugate solution may be analyzed by UV-Vis spectroscopy or chromatographically to determine the absorbance or area-under-the-curve (absorbance over time in the chromatogram) associated with the peptide antigen conjugate and any unreacted hydrophobic block fragment. For peptide antigen conjugates and hydrophobic block fragments comprising a chromophore, such as adjuvants of Formula III, referred to as an imidazoquinoline TLR-7/8a, the absorbance that is distinct to the chromophore may be used to assess the concentration of the molecules to which the chromophore is attached. Unexpectedly, we report that absorbance measurements between 315 to 330 nm by HPLC and/or UV-Vis spectroscopy can be used to assess the concentration of peptide antigen conjugate and hydrophobic block fragment comprising an imidazoquinoline TLR-7/8a.

The methods described here for assessing peptide antigen conjugate and/or hydrophobic block content in solution are based on the Beer-Lambert law relationship, which relates the absorbance of chromophores at specific wavelengths to concentration, as provided here:

$$C = \frac{A}{l \times ɛ} Where C = concentration ; A = asbsorbance ;$$

$$I = path length ; ɛ = extinction coefficient$$

Use of UV-Vis spectroscopy to determine the content (concentration) of peptide antigen conjugates, as well as hydrophobic block (H), in a solution relies on the presence of groups on the hydrophobic block that have UV-Vis wavelength absorbance that falls outside the range of wavelengths absorbed by amino acids (i.e., 200-300 nm) comprising the patient-specific portion of the peptide antigen conjugate. While hydrophobic blocks based on lipids, fatty acids and cholesterol, as well as commonly used hydrophobic polymers, such as PLGA and poly(caprolactone) do not appreciably absorb light above 300 nm, hydrophobic blocks comprising aromatic groups, such as the hydrophobic blocks described herein comprising imidazoquinoline-based TLR-7/8a, are good chromophores with strong absorbance above 300 nm, which is outside of the range of absorbance of typical peptides.

It was currently unknown whether such a technique could be used to determine peptide antigen conjugate concentration, and whether or not each peptide antigen conjugate would have the same molar absorption coefficient, irrespective of peptide antigen (A) sequence. Therefore, it was first necessary to validate the method by producing a “test set” of peptide antigen conjugates with peptide antigens (A) having a broad range of physicochemical properties—with charge ranging from +6 to −6 and hydropathy from +2 to −2, representative of up to 98% of neoantigens (Table 1)—were produced using the hydrophobic block, referred to as 2B3W2.

TABLE 1
Composition of ″test set″ of peptide antigens

| Neo- | | | | |
|---|---|---|---|---|
| antigen | Amino acid sequence | Length | Charge | Gravy |
| A1 | ETLGEISFLLSLDLHFTDGDYSAGD | 25 | -6 | 0.016 |
| A2 | DDEGDYTCQFTHVENGTNYIVTATR | 25 | -4 | -0.904 |
| A3 | GIPVHLELASMTNMELMSSIVHQQVFPT | 28 | -2 | 0.429 |
| A4 | VVDRNPQFLDPVLAYLMKGLCEKPLAS | 27 | 0 | 0.196 |
| A5 | NIEGIDKLTQLKKPFLVNNKINKIENI | 27 | 2 | -0.474 |
| A6 | MAAALTFRRLLTLPRAARGFGVQVS | 25 | 4 | 0.560 |
| A7 | GRGHLLGRLAAIVGKQVLLGRKVVVVR | 27 | 6 | 0.659 |
| A8 | QGTDVVIAIFIILAMSFVPASFVVF | 25 | -1 | 2.000 |
| A9 | LKSSPERNDWEPLDKKVDTRKYRAE | 25 | 1 | -1.908 |
| A10 | QLRVGNDGIFMLPFFMAFIFNWLGF | 25 | 0 | 0.992 |

Importantly, peptide antigen conjugates with hydrophobic blocks comprising imidazoquinoline TLR-7/8a, such as 2B3W2, have strong absorbance at 325 nm due to the three imidazoquinoline-based TLR-7/8a attached to each AVT01 conjugate, which falls outside the range of wavelengths absorbed by amino acids (i.e., 200-300 nm) and is expected to allow for a molar absorption coefficient of the conjugates (at wavelengths>300 nm) independent of antigen composition.

To confirm that the molar extinction coefficient, (c), due to absorbance of certain hydrophobic blocks (H) at wavelengths>300 nm are independent of the underlying neoantigen composition for any possible peptide antigen conjugates, the “test set” of peptide antigen conjugates were synthesized, HPLC-purified and evaluated for content by EA and AAA in accordance with previously established procedures; (2) each peptide antigen conjugate was then serially diluted in DMSO solution at known concentrations and assessed for absorbance at 325 nm by both UV-Vis spectroscopy (OD 325 nm) and UPLC-MS (AUC at 325 nm) to generate standard curves (slope=ε) relating absorbance to concentration.

Importantly, the mean molar extinction coefficient (E) for each of the peptide antigen conjugates from the test set were determined to be equivalent, which confirmed the suitability of UV-Vis spectroscopy for determining the concentration of peptide antigen conjugates, which comprise hydrophobic blocks that absorb>300 nm, in solutions.

### Assessing the Peptide Antigen Conjugate Stability in Aqueous Buffer

Addition of an aqueous buffer, e.g., PBS, to the product solution or purified peptide antigen conjugate solution results in the peptide antigen conjugate spontaneously assembling into stable nanoparticle micelles providing an aqueous solution of peptide antigen conjugate particles. While the exact size range of particles formed by the peptide antigen conjugates may be assessed by DLS or microscopy, these measurements do not provide a clear means of assessing the peptide antigen conjugate stability (i.e. the propensity of peptide antigen conjugates to form aggregated material) in aqueous buffer. As the nanoparticle micelles formed by peptide antigen conjugates are too small to appreciably scatter visible light, it's possible to assess for the propensity of peptide antigen conjugates to form aggregated material by using turbidity measurements by assessing absorbance of the peptide antigen conjugates at a wavelength that is not absorbed by chromophore groups comprising the peptide antigen conjugate or hydrophobic block fragment. Therefore, any absorbance measured is due to light scattered by aggregated formed by peptide antigen conjugate particles in the aqueous solution. An unexpected finding reported herein is that turbidity measurements performed by assessing absorbance at wavelengths between 350 and 650 nm provided a sensitive and specific approach for determining the propensity of peptide antigen conjugates to form aggregated material.

### Example 4—Process of Selecting Antigens for Multi-Antigen Particles

### Selection of Peptide Antigen Conjugates for Peptide Antigen Conjugate Mixtures

A vaccine based on peptide antigen conjugates, especially a personalized cancer vaccine, will include multiple different peptide antigen conjugates wherein the peptide antigen (A) portions are variable. Therefore, multiple peptide antigen conjugates will likely need to be administered together in the same solution.

The interaction of peptide antigen conjugates of different underlying compositions may affect formation of stable nanoparticle micelles. However, unexpectedly, we have found that single peptide antigen conjugates that are insoluble alone form stable nanoparticles when co-formulated as multi-peptide antigen conjugate particles (sometimes referred to as peptide antigen conjugate mixtures). Thus, aqueous mixtures of peptide antigen conjugates (based on peptide antigen conjugate mixtures) form multi-peptide antigen conjugate particles that overcome the propensity of certain single peptide antigen conjugates to aggregate in aqueous conditions.

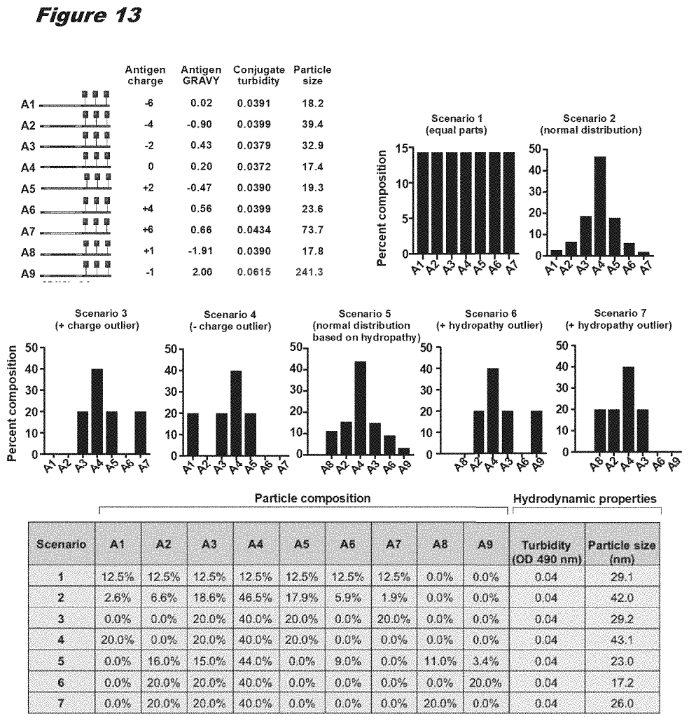

As we have previously disclosed in our co-pending application, International Patent Application No. PCT/US2018/026145, for peptide antigen conjugates of the formula C-B1-A-B2-X1, the charge provided by the C-B1-moiety can be modulated to achieve a net charge of the peptide antigen conjugate required to achieve nanoparticle micellization as an aqueous mixture of peptide antigen conjugates. The composition of the C-B1-moiety is selected on the basis of the underlying properties of the peptide antigen (A) portion to allow for the greatest likelihood that the resulting peptide antigen conjugate, e.g., C-B1-A-B2-L-H, will form a stable nanoparticle micelle. Our analysis showed that most peptide antigens generated from the human genome formed stable nanoparticle micelles as single peptide antigen conjugates (FIG. 13). Still, approximately 10% of single peptide antigen conjugates aggregated in PBS buffer (i.e. turbidity exceeded>0.05 OD at 490 nm, or particle size>200 nm). Despite the potentially undesirable property of aggregation when working with these sequences as individual peptide antigen conjugates, these conjugates can be accommodated to form stable micelles when co-formulated with other peptide antigens conjugates as peptide antigen conjugate mixtures that form multi-peptide antigen conjugate particles as aqueous mixtures. To evaluate how different combinations of the test antigens, A1-A9, with varying charge and hydropathy, delivered together in the same peptide antigen conjugate particle impacts the size and stability of the resulting multi-antigen particles, we evaluated 7 unique compositions of peptide antigen conjugate mixtures representing different possible scenarios (FIG. 13). Notably, irrespective of the peptide antigen conjugate composition, all mixtures assembled into nanoparticle micelles with diameter between about 20-40 nm diameter. This novel finding suggests that delivery of peptide antigen conjugates as peptide antigen conjugate mixtures that form multi-peptide antigen conjugate particles as aqueous mixtures could be a reliable strategy for ensuring peptide antigen conjugates delivering peptide antigens (A) with a broad range of physical properties assemble into stable nanoparticles micelles.

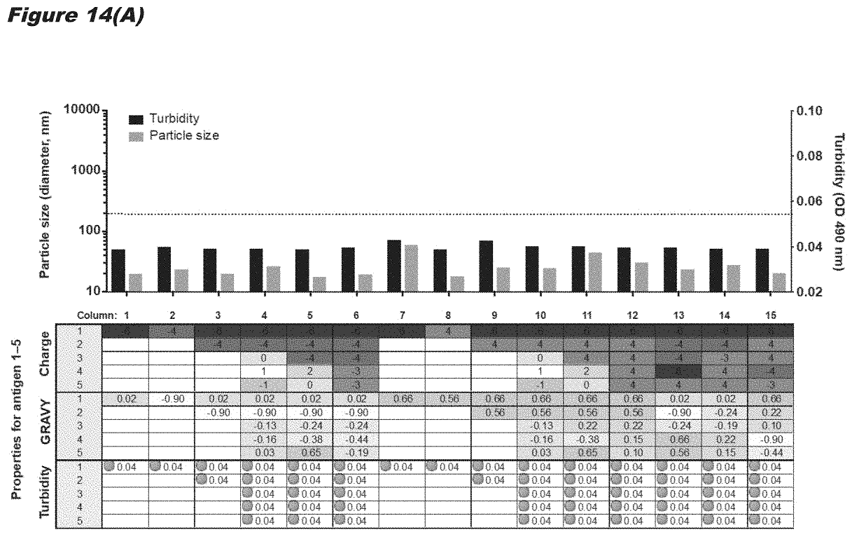

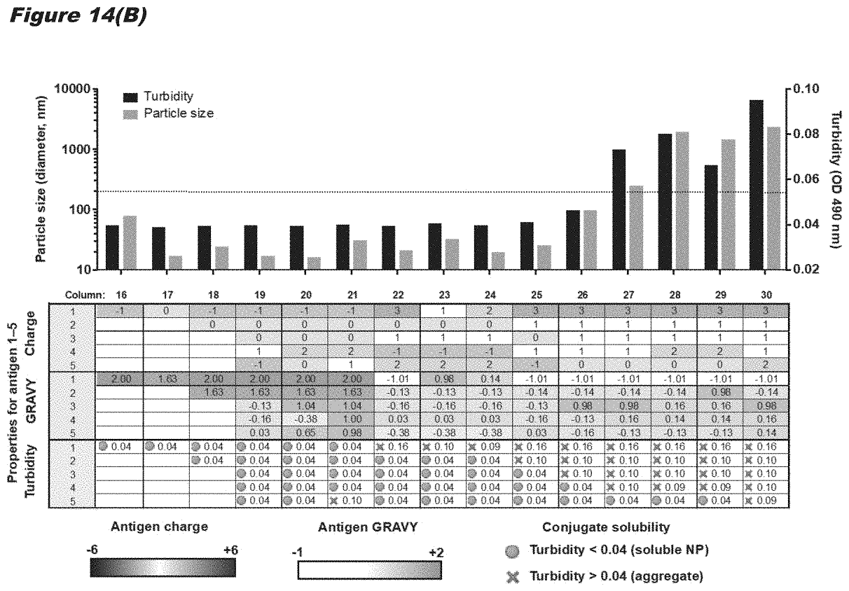

We had previously identified antigen sequences that—despite being produced as peptide antigen conjugates of the formula C-B1-A-B2-L-H—do not form stable nanoparticle micelles and instead aggregate when reconstituted in aqueous buffer as individual peptide antigen conjugates (so-called ‘difficult’ sequences). We had also previously identified peptide antigen conjugate sequences of the formula C-B1-A-B2-L-H that do form stable nanoparticle micelles when reconstituted individually (so-called ‘well-behaved’ sequences). We therefore sought to determine to what extent such ‘difficult’ sequences could be mixed with ‘well-behaved’ sequences in peptide antigen conjugate mixtures (“multi-antigen particle formulation”) to form stable nanoparticle micelles. Therefore, to evaluate the tolerance of multi-peptide antigen conjugate particles for delivering different compositions of peptide antigen conjugates, we evaluated the size and stability of multi-peptide antigen conjugate particles formed following the addition of aqueous buffer to peptide antigen conjugate mixtures with up to 5 unique peptide antigen conjugates (in equimolar amounts) comprising peptide antigens (A) with varying charge and hydropathy (FIG. 14).

We found that stable micelles were formed for peptide antigen conjugate mixtures containing up to 60 mol % of difficult peptide antigen conjugate sequences. When this number went up to 80 mol %, aggregation occurred. Since less than 10% of peptide antigen conjugates are ‘difficult’ (aggregated in PBS when reconstituted individually), the probability that any 5-peptide antigen conjugate mixture (i.e. 5 unique peptide antigen conjugates) will comprise 4 or more peptide antigen conjugates that have a propensity to aggregate (“difficult” peptide antigen conjugate sequences) is about 1 in 2,000. These results then provide the unexpected finding that increasing the number of different peptide antigen conjugates in a peptide antigen conjugate mixture can improve particle stability. Accordingly, our results suggest that greater than 99.95% of peptide antigen conjugate mixtures incorporating 5 or more unique peptide antigen conjugates will form stable 20-40 nm micelles with a turbidity less than 0.05 as aqueous mixtures.

Therefore, to avoid a scenario wherein >60 mol % of ‘difficult’ sequences are included in a peptide antigen conjugate mixture, the peptide antigen conjugates can be assessed first individually for turbidity and particle size in aqueous buffer. In the setting, wherein a subject is to receive 20 unique antigens (split across 4 pools consisting of 5 unique peptide antigen conjugates each), it would be expected that approximately 2 antigens (˜10%) will be ‘difficult’. Such ‘difficult’ sequences can be first identified by turbidity measurements and then intentionally separated into different pools (e.g., 1 difficult sequence per pool) to avoid>60 mol % of any given pool being comprised of ‘difficult’ sequences. Moreover, the exact molar concentration of each of the peptide antigen conjugates can be aliquoted precisely based on the absorbance measurements methods described above to ensure that the resulting peptide antigen conjugate mixture comprises a precisely defined ratio of each of the different peptide antigen conjugates.

Selection of antigens to include in each pool of peptide antigen conjugate mixtures may also be based on predicted MHC binding affinity. When administering multiple antigens into the same site, there may be ‘antigenic competition’, which is the phenomenon wherein the magnitude of the immune response (i.e., a T cell response) to a specific antigen is reduced when administered as a multi-antigen particle, which is an aqueous mixture of the peptide antigen conjugate mixture, compared to when an antigen is administered alone as a single peptide antigen conjugate.

Additionally, machine learning algorithms from this empirical data may be used to refine the process for selecting individual peptide antigen conjugates to include in peptide antigen conjugate mixtures as means to mitigate the potential of choosing incompatible conjugates that will lead to the propensity of the multi-antigen particles to aggregate when the peptide antigen conjugate is diluted with an aqueous buffer to form an aqueous mixture of the peptide antigen conjugates.

In sum, the putative issue of a subset of antigens of the formula C-B1-A-B2-L-H failing to form stable nanoparticle micelles when reconstituted alone in aqueous buffer may be ameliorated by combining multiple different peptide antigen conjugates together according to the selection process described above in organic solvent (e.g., DMSO) prior to reconstituting in aqueous buffer (e.g., PBS).

### Example 5—Sterile Filtration and Reconstitution of Peptide Antigen Conjugates

Filtration and Reconstitution

Prior to administration to a patient, the peptide antigen conjugates (e.g., product solutions, purified peptide antigen conjugate solutions or peptide antigen conjugate mixtures) should undergo sterile filtration as a means to ensure product sterility and removal of any particulate debris that could be introduced during manufacturing. The sterile filtration should occur at a point that is most proximal to administration to a patient but is also at a point where sterile filtration is feasible to implement. Immediately prior to patient administration, the next most proximal step is the formation of the nanoparticle micelles by suspension of the peptide antigen conjugates in aqueous buffer to obtain the aqueous mixture of peptide antigen conjugates. To provide the greatest certainty of sterility of the product, the sterile filtration step should then occur immediately prior to or after suspension of the peptide antigen conjugates (i.e. product solutions, purified peptide antigen conjugate solutions or peptide antigen conjugate mixtures) in aqueous media.

The solvent system, drug concentration, filter membrane composition and filter technique can all impact product recovery following sterile filtration. Therefore, we evaluated peptide antigen conjugate recovery following sterile filtration at various concentrations of peptide antigen conjugates in different solvents (DMSO and PBS buffer) using two types of filter devices (centrifugal filter tubes and traditional syringe filters) with a PTFE membrane.

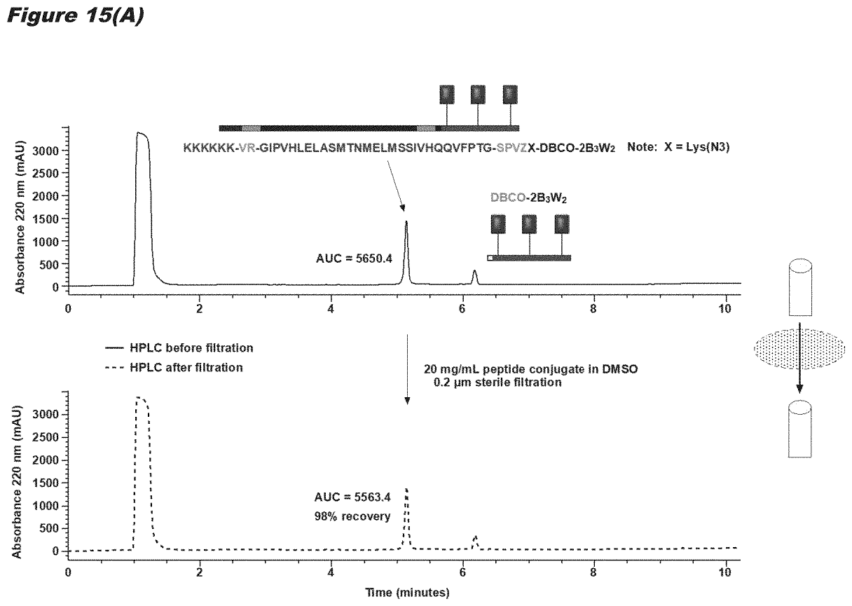

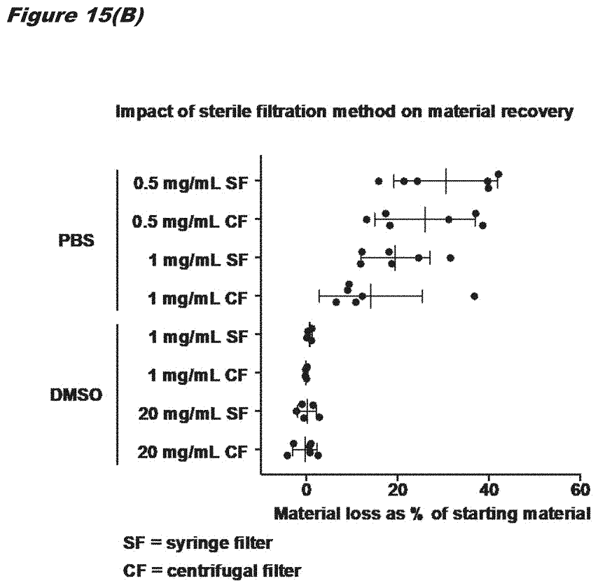

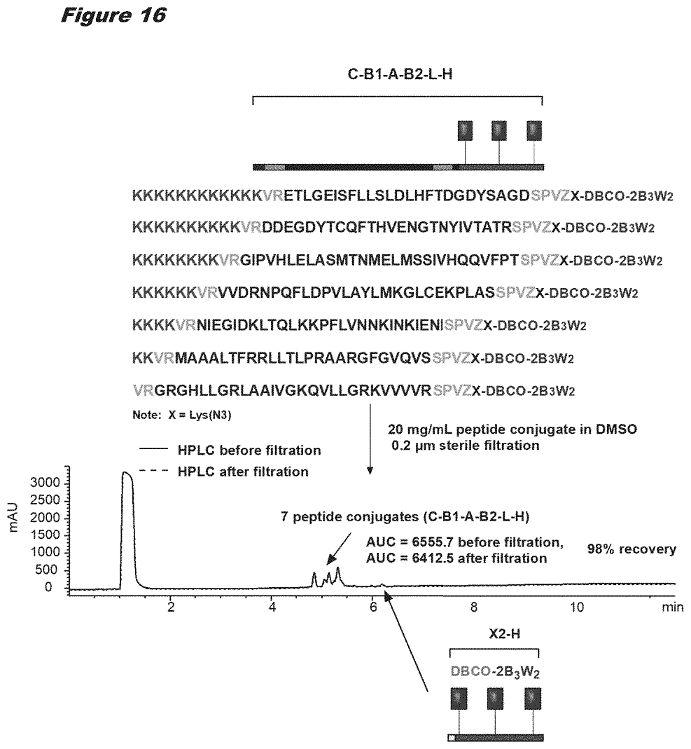

The recovery of peptide antigen conjugate using different filtration protocols was determined using HPLC by calculating peak area of the peptide antigen conjugate prior to and after sterile filtration (FIG. 15). Our results show that the solvent system has a major impact on peptide antigen conjugate recovery. Accordingly, while peptide antigen conjugate in DMSO at concentrations of up to 20 mg/mL showed greater than 95% recovery when sterile filtered by syringe filter or centrifugal filter techniques (FIG. 15B), the same peptide antigen conjugates in aqueous buffer (i.e. PBS) exhibited between about 20-40% material loss (FIG. 15B). The high efficiency of recovery was also observed when multiple peptide antigen conjugates were present in the same DMSO solution (as a peptide antigen conjugate mixture), as no differences in recovery were observed for different compositions of peptide antigen conjugates in the same mixture (FIG. 16). These results highlight the unexpected finding that sterile filtration of the peptide antigen conjugate in DMSO consistently results in high efficiency for material recovery regardless of the conjugate composition concentration and percentage of excess hydrophobic block fragment. Moreover, while both centrifugal filtration and syringe filtration result in recovery of material with the same concentration of peptide antigen conjugate as before filtration, small volumes of solution can be retained on syringe filters, which may substantially reduce recovery when working with low sample volumes. Therefore, centrifugal filtration is preferred when working with low sample volumes.

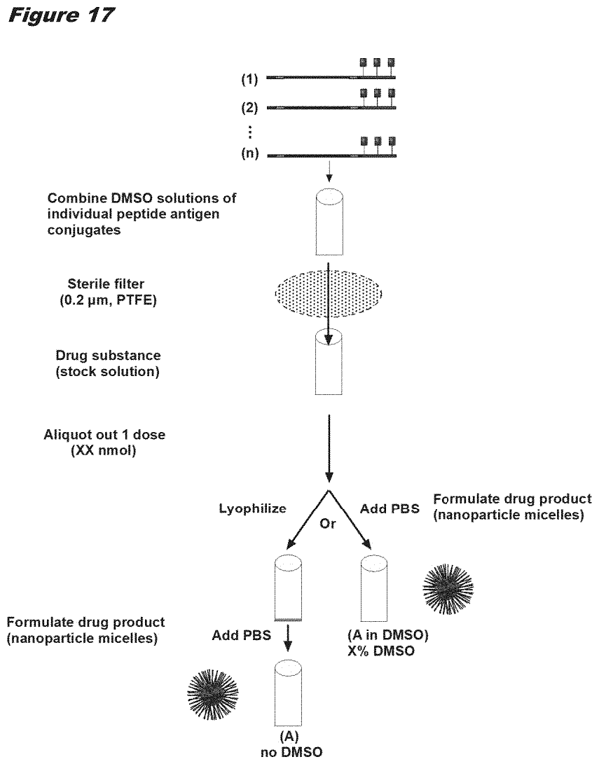

Importantly, our results inform when and how sterile filtration should be performed. Our results indicate that optimal peptide antigen conjugate recovery is achieved when the peptide antigen conjugate is sterile filtered while in an organic solvent. Therefore, sterile filtration should occur following the completion of the conjugation reaction while the peptide antigen conjugate is still present in an organic solvent, e.g., DMSO (FIG. 17). The sterile filtration may be performed on a single peptide antigen conjugate (FIG. 15), or multiple peptide antigen conjugates may be compounded into a mixture (e.g., peptide antigen conjugate mixture) and sterile filtered (FIGS. 16 and 17). The sterile filtered organic solution of peptide antigen conjugate (i.e. sterile product solution, sterile purified peptide antigen conjugate solution or sterile peptide antigen conjugate mixture) may then be stored, lyophilized or mixed with PBS (FIG. 17).

### Filtration of Peptide Antigen Conjugate Mixtures and Particle Stability Over Time

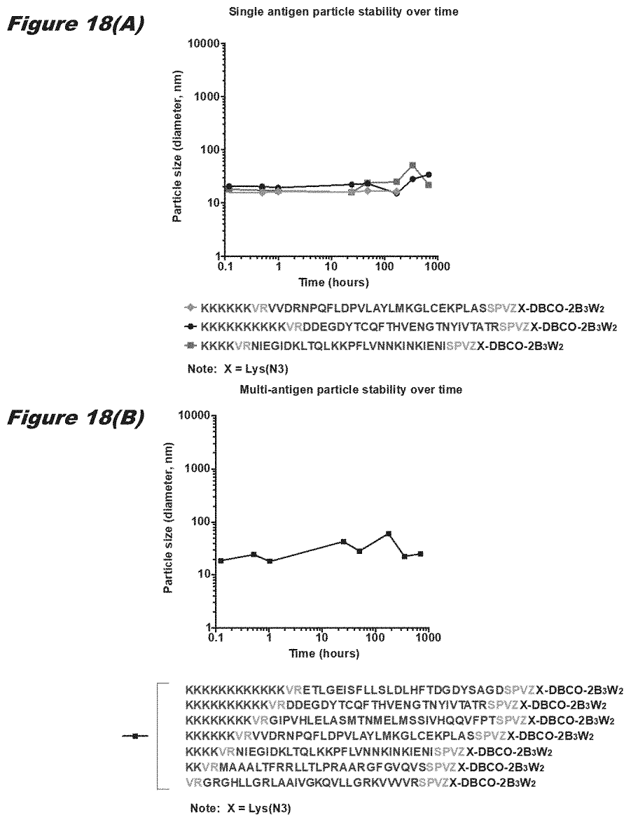

For sterile filtration of peptide antigen conjugate mixtures, we showed a high recovery (98%) after the sterile filtration of the DMSO solution containing equal mass of 7 unique peptide antigen conjugates (FIG. 17). Once reconstituted in PBS buffer, the mixture immediately formed nano-sized micelles (particle size ˜15-40 nm diameter) that were stable against aggregation for at least 1 week at r.t. (FIG. 18). Therefore, mixing different peptide antigen conjugates prior to reconstituting in aqueous buffer had no adverse influence on the sterile filtration process, or the size and stability of nanoparticle micelles.

### Effect of the Use of DMSO on Particle Size, Stability and In Vivo Activity

Following sterile filtration of the peptide antigen conjugates in organic solvent, e.g., DMSO (FIG. 17), the organic solvent can either be removed by evaporation with the subsequent dry powder directly resuspended in aqueous buffer, or the peptide antigen conjugate in a pharmaceutically acceptable organic solvent (e.g., DMSO) can be immediately suspended in aqueous buffer (e.g., PBS) to obtain an aqueous mixture of the peptide antigen conjugates that comprises nanoparticle micelles formed by the peptide antigen conjugates. As the solvent evaporation process can increase costs and manufacturing time, the latter approach of mixing the peptide antigen conjugates suspended in organic solvent with an aqueous buffer would be preferred, but the impact of this process on nanoparticle assembly is unknown. Indeed, the two common methods for forming nanoparticle micelles from amphiphilic molecules in organic solvents are to use dialysis or co-solvent evaporation. Dialysis requires specialized devices and the process typically takes 2 or more days. In contrast, co-solvent evaporation involves the addition of the organic solvent in which a drug is dissolved to an aqueous buffer, after which the organic solvent is evaporated to enable the formation of nanoparticle micelles.

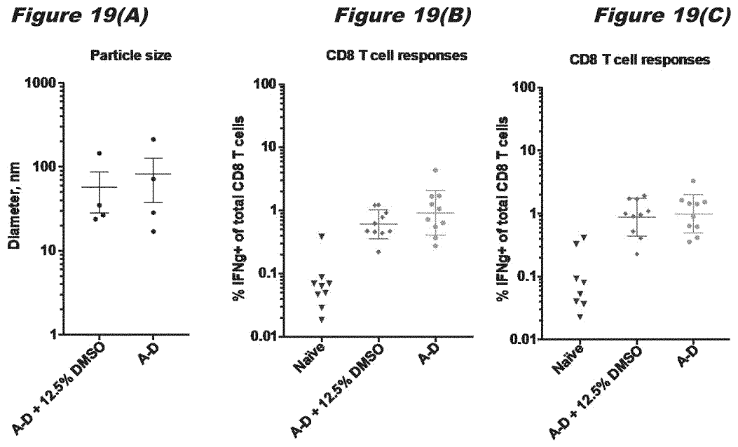

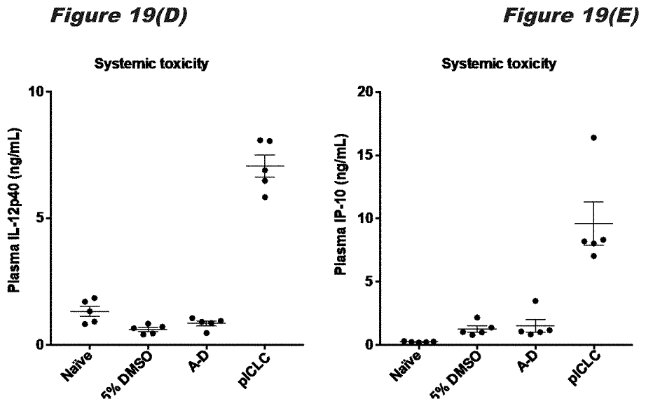

As an alternative to the co-solvent evaporation process, our approach was to simply dilute the sterile peptide antigen conjugate mixture in DMSO (comprising multiple different production solutions) with an aqueous buffer to generate a sterile aqueous solution of peptide antigen conjugate particles that assembled into nanoparticle micelles. An unexpected finding is that the nanoparticle micelles form immediately following the addition of aqueous buffer to the peptide antigen conjugates in either the form of a solid or DMSO solution (FIG. 19A). Importantly, the aqueous mixture of peptide antigen conjugates comprising DMSO up to 12.5% (v/v) of the solution, was found to have no impact on particle size or in vivo activity of the peptide antigen conjugates (FIG. 19A-E). This data suggest that stable nanoparticle micelles are generated by simply reconstituting peptide antigen conjugates from DMSO solution in PBS buffer and that it is not necessary to remove the organic solvent, i.e. DMSO. Thus, this approach offers a simple and reliable method for generating nanoparticle micelles from amphiphilic compounds, such as the peptide antigen conjugates described herein.

An additional consideration is the sequence at which the peptide antigen conjugate in organic solvent is combined with the aqueous buffer. Another unexpected finding was that, in order to ensure reliable assembly of nanoparticle micelles, a large volume of aqueous buffer had to be added to a small volume of the peptide antigen conjugate in organic solvent (such as the product solution, purified peptide antigen conjugate solution or peptide antigen conjugate mixture), rather than the addition of a small volume of the peptide antigen conjugate in organic solvent to a larger volume of aqueous buffer.

Therefore, in a preferred manufacturing scheme, the peptide antigen conjugate or peptide antigen conjugate mixture is in DMSO and a larger volume of aqueous buffer is added to this solution, followed by rapid mixing, to generate stable nanoparticle micelles.

### Example 6—Impact of Counter-Ion on Solubility in DMSO and Aqueous Solutions

To evaluate the impact of the counter-ion on the solubility of peptide antigen fragments and peptide antigen conjugates comprised of charged moieties with negatively charged functional groups, the impact that different counter-ions has on the solubility of a model peptide antigen fragment of formula C-B1-A-B2-X1, Ac-Glu-Glu-Glu-Glu-Glu-Ser-Leu-Val-Cit-Ala-Gln-Leu-Asn-Asp-Val-Val-Leu-Ser-Pro-Val-Cit-Lys(N3)-NH₂, wherein Lys(N3) is azido-lysine, was evaluated.

The protons of the acids comprising the peptide antigen fragment, Ac-Glu-Glu-Glu-Glu-Glu-Ser-Leu-Val-Cit-Ala-Gln-Leu-Asn-Asp-Val-Val-Leu-Ser-Pro-Val-Cit-Lys(N3)-NH₂, were either exchanged with sodium using a cation exchange column or by neutralizing the acid with sodium hydroxide, the protons were neutralized with the addition of ammonia or an organic base, triethylamine (TEA), di-isopropylethylamine (DIPEA) or tris(hydroxymethyl)aminomethane (Tris), and the resulting salt was assessed for solubility in water and DMSO.

As shown below (Table 2), only the ammonium and Tris salts ensured solubility of the peptide antigen fragment in DMSO, a water-miscible solvent and water.

TABLE 2
impact of charged moiety counter-ion on solubility

| Counter Ion | Soluble in water? | Soluble in DMSO? |
|---|---|---|
| None (protonated) | NO | YES |
| Sodium | YES | NO |
| TEA | YES | NO |
| DIPEA | YES | NO |
| Ammonium | YES | YES |
| Tris | YES | YES |

To evaluate the suitability of Tris salts of peptide antigen conjugates for use in immunogenic compositions, the Tris salt of two peptide antigen conjugates of formula C-[B1]-A-B2-L-H, i.e., Ac-Glu-Glu-Glu-Glu-Glu-Val-Cit-Thr-Ala-Pro-Asp-Asn-Leu-Gly-Tyr-Met-Ser-Pro-Val-Cit-Lys(N3-DBCO)-Glu(2B)-Trp-Glu(2B)-Trp-Glu(2B)-NH2 and Ac-Asp-Asp-Asp-Asp-Asp-Asp-Asp-Asp-Asp-Asp-Asp-Asp-Asp-Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Ser- Pro-Val-Cit-Lys(N3-DBCO)-Glu(2B)-Trp-Glu(2B)-Trp-Glu(2B)—NH2 referred to as Adpgk conjugate and Trp1 conjugate, respectively, were evaluated.

To prepare the Tris salts of the peptide antigen conjugates, each of the peptide antigen conjugates was suspended in DMSO and then acids of the peptide antigen conjugates with neutralized with either 1.25 or 2.5 equivalents of Tris and then suspended in an aqueous buffer, PBS pH 7.4, at a concentration of either 80 or 320 μM and assessed for particle size and stability.

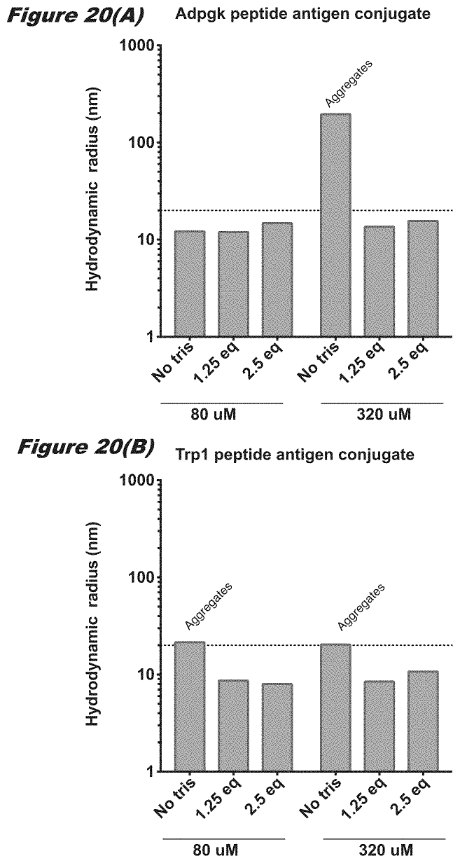

Notably, while the peptide antigen conjugates as the Tris salt (with both 1.25 and 2.5 equivalents of Tris) formed stable nanoparticle micelles, the peptide antigen conjugates in the protonated form aggregated in solution (FIG. 20). These results verify the suitability of Tris salts of peptide antigen conjugates to ensure stable nanoparticle micellization in aqueous buffer.

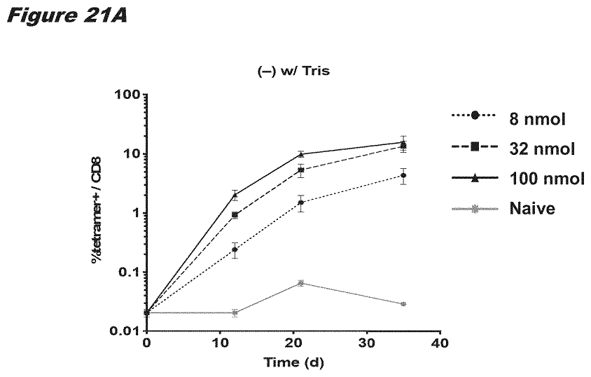

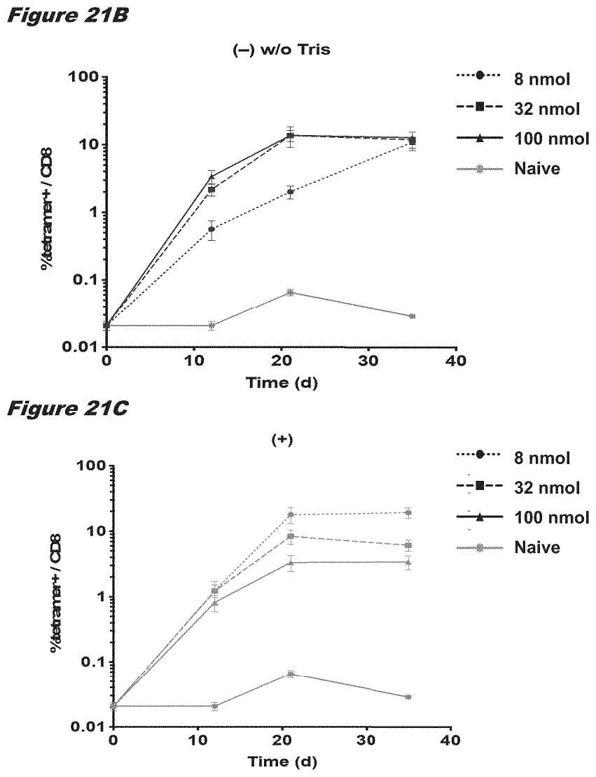

To evaluate the suitability of the Tris salts for use in immunogenic compositions, mice were either vaccinated with peptide antigen conjugates of formula C-B1-A-B2-L-H, either comprising a charged moiety with positive charge, i.e., Lys-Lys-Lys-Lys-Lys-Lys-Lys-Lys-Lys-Val-Arg-Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Ser-Pro-Val-Cit-Lys(N3-DBCO)-Glu(2B)-Trp-Glu(2B)-Trp-Glu(2B)—NH2, or a charged moiety with negative charge and a B2 comprised of amino acids bearing aryl amines, i.e., Ac-Glu-Glu-Glu-Glu-Glu-Glu-Glu-Val-Cit-Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Ser- Pro-Val-Cit-Phe(NH2)-Phe(NH2)-Phe(NH2)-Phe(NH2)-Lys(N3-DBCO)-Glu(2B)-Trp-Glu(2B)-Trp-Glu(2B)—NH2, wherein Phe(NH2) is para-amino-phenylalanine. Importantly, CD8 T cell responses were induced in all animals that received the Tris salts of the negatively charged peptide antigen conjugate (FIG. 21).

### Example 7—Synthesis and Characterization of Hydrophobic Blocks (H) and Amphiphilic Carrier Molecules

A combinatorial library of different amphiphilic carrier molecules was prepared by generating a series of hydrophobic blocks (H) comprising an X1 linker precursor bearing an alkyne and reacting these in a combinatorial manner with different S-B compositions bearing a linker precursor X2 bearing an azide group to form amphiphilic carrier molecules of the formula C-B-L-H. The synthesis of the hydrophobic blocks and resulting charged amphiphilic carrier molecules of formula C-B-L-H are described below.

### Synthesis of Hydrophobic Blocks

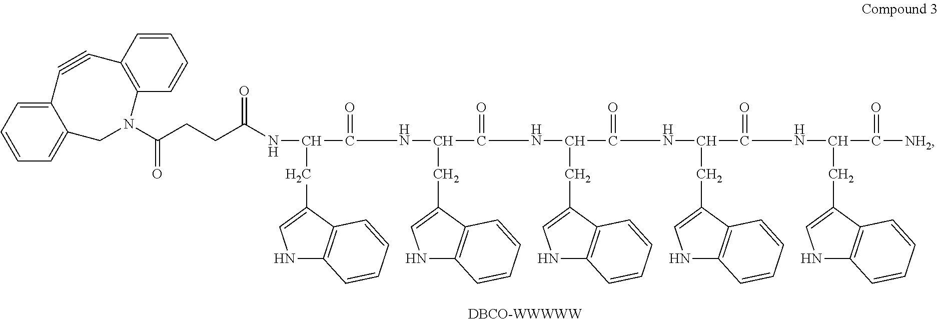

Compound 3, referred to as DBCO-W₅, W₅ or DBCO-(Trp)₅ was synthesized by reacting 137.6 mg (0.15 mmol, 1 eq) of the precursor NH₂-(Trp)—NH₂ that was prepared by solid phase peptide synthesis with 146.1 mg of DBCO-NHS (0.057 mmol, 2.5 eq) and 14.7 mg of triethylamine (0.15 mmol, 1.1 eq) in 3.0 mL of DMSO. Compound 3 was purified on a preparatory HPLC system using a gradient of 52-72% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain 75.1 mg (42% yield) of a spectroscopically pure (>95% AUC at 254 nm) white powder. MS (ESI) calculated for C₇₄H₆₆N₁₂O₇ m/z 1234.52, found 1235.6 (M+H)⁺.

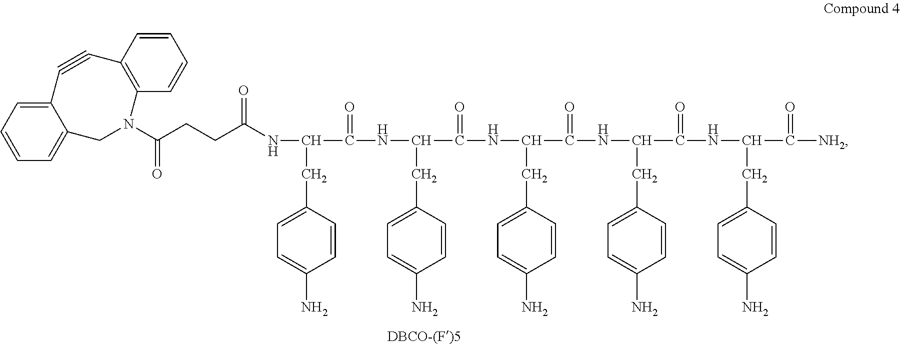

Compound 4, referred to as DBCO-F′₅ or F′₅ was synthesized by reacting 49.8 mg (0.06 mmol, 1 eq) of the precursor NH₂-(F′)—NH₂, which was prepared by solid phase peptide synthesis, with 24.5 mg of DBCO-TT (0.057 mmol, 1.0 eq) and 30.3 mg of NaHCO₃ (0.36 mmol, 6.0 eq) in 1.0 mL of DMF. The reaction was run overnight at room temperature and HPLC indicated that the reaction was complete by 24 hours. Compound 4 was purified on a preparatory HPLC system using a gradient of 10-30% acetonitrile/H₂O (0.05% TFA) over 10 minutes on an Agilent Prep-C18 column, 30×100 mm, 5 m. The product eluted at ˜3.4 minutes and the resulting fractions were collected, frozen and then lyophilized to obtain 25.8 mg (38.4% yield) of a spectroscopically pure (>95% AUC at 254 nm) white powder. MS (ESI) calculated for C₆₄H₆₆N₁₂O₇ m/z 1114.52, found 1116.1 (M+H)⁺.

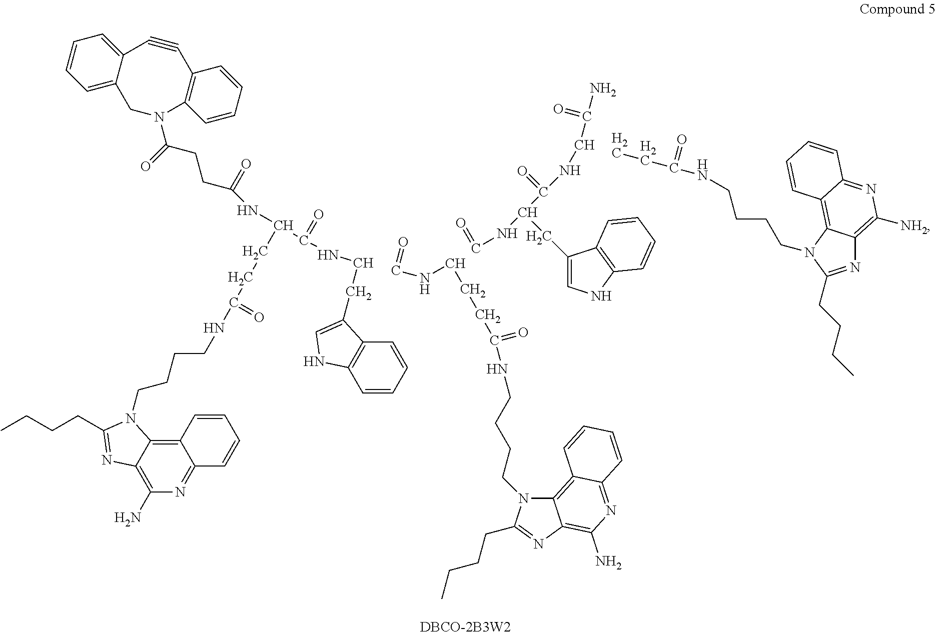

Compound 5, referred to as DBCO-2B₃W₂, 2B₃W₂ or DBCO-(Glu(2B)₃(Trp)₂), was synthesized as described in PCT/US2018/026145 to obtain spectroscopically pure (>95% AUC at 254 nm) white powder. MS (ESI) calculated for C₁₁₀H₁₂₆N₂₄O₁₀ m/z 1943.01, found 973.0 (M/2)⁺.

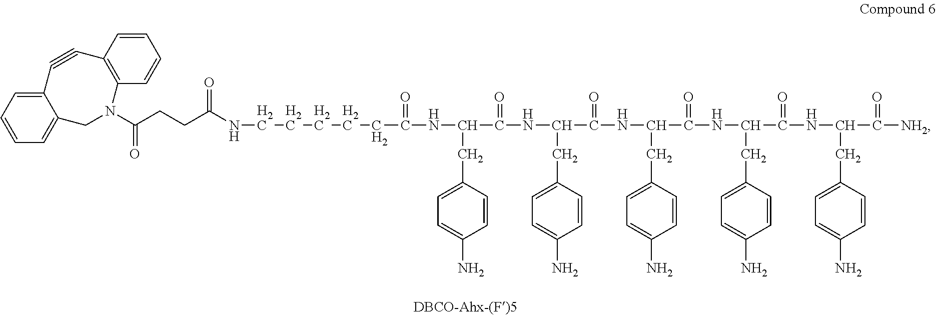 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCCCCNC(=O)CCC(=O)N1Cc2ccccc2C#Cc2ccccc21]

Compound 6, referred to as DBCO-Ahx-F′₅ or Ahx-F′₅ was synthesized by reacting 400 mg (0.4 mmol, 1 eq) of the precursor (6-hydroxyhexanoyl)-(F′)₅—NH₂, which was prepared by solid phase peptide synthesis, with 171.05 mg of DBCO-NHS (0.4 mmol, 1.0 eq) and 258.1 mg of Triethylamine (2.55 mmol, 6.0 eq) in 3.7 mL of DMSO. The DBCO-NHS was added in 4 increments of 0.25 eq. The reaction was run overnight at room temperature and HPLC indicated that the reaction was complete by 24 hours. Compound 6 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.7 minutes and the resulting fractions were collected, frozen and then lyophilized to obtain 217.0 mg (41.5% yield) of a spectroscopically pure (>95% AUC at 254 nm) white/yellow powder. MS (ESI) calculated for C₇₀H₇₆N₁₂O₉ m/z 1228.59, found 1228.7 (M+H)⁺.

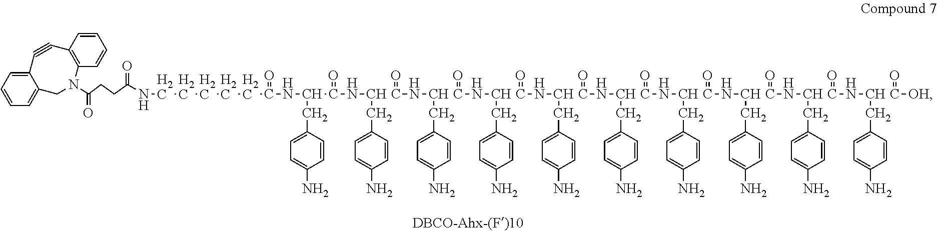 [SMILES: Nc1ccc(CC(NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)C(Cc2ccc(N)cc2)NC(=O)CCCCCNC(=O)CCC(=O)N2Cc3ccccc3C#Cc3ccccc32)C(=O)O)cc1]

Compound 7, referred to as DBCO-Ahx-F′₁₀ or Ahx-F′₁₀ was synthesized by reacting 450 mg (0.26 mmol, 1 eq) of the precursor (6-hydroxyhexanoyl)-(F′)₁₀-NH₂, which was prepared by solid phase peptide synthesis, with 103.4 mg of DBCO-NHS (0.26 mmol, 1.0 eq) and 286.1 mg of Triethylamine (2.83 mmol, 11.0 eq) in 3.3 mL of DMSO. The DBCO-NHS was added in 4 increments of 0.25 eq. The reaction was run overnight at room temperature and HPLC indicated that the reaction was complete by 24 hours. Compound 7 was purified on a preparatory HPLC system using a gradient of 15-45% acetonitrile/H₂O (0.05% TFA) over 12 minutes on an Agilent Prep-C18 column, 50×100 mm, 5 m. The product eluted at ˜5.1 minutes and the resulting fractions were collected, frozen and then lyophilized to obtain 265.4 mg (50.6% yield) of a spectroscopically pure (>95% AUC at 254 nm) red/copper powder. MS (ESI) calculated for C₂₀₅H₂₂₆N₄₂O₂₄ m/z 3659.78, found 1221.3 (M+3H)⁺.

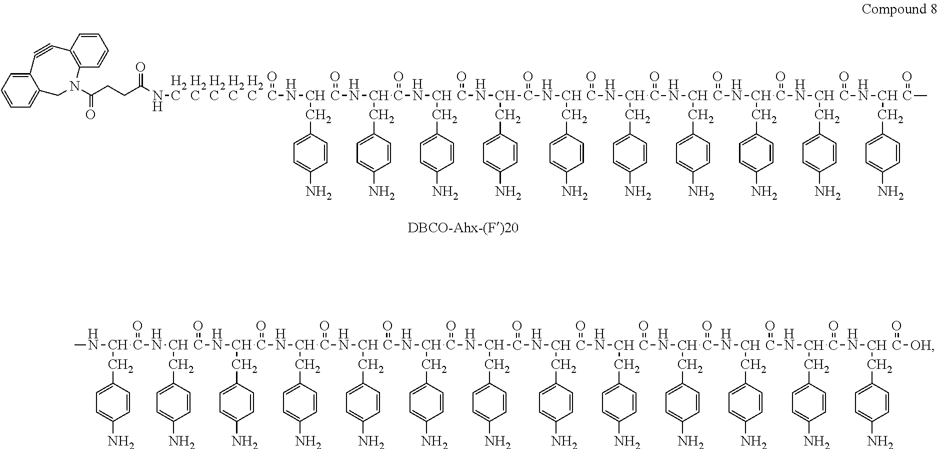

Compound 8, referred to as DBCO-Ahx-F′₂₀ or Ahx-F′₂₀ was synthesized by reacting 480 mg (0.14 mmol, 1 eq) of the precursor (6-hydroxyhexanoyl)-(F′)₂₀—NH₂, which was prepared by solid phase peptide synthesis, with 57.3 mg of DBCO-NHS (0.14 mmol, 1.0 eq) and 302.4 mg of Triethylamine (2.99 mmol, 21.0 eq) in 3.0 mL of DMSO. The DBCO-NHS was added in 4 increments of 0.25 eq. The reaction was run overnight at room temperature and HPLC indicated that the reaction was complete by 24 hours. Compound 8 was purified on a preparatory HPLC system using a gradient of 13-43% acetonitrile/H₂O (0.05% TFA) over 12 minutes on an Agilent Prep-C18 column, 50×100 mm, 5 m. The product eluted at ˜5.5 minutes and the resulting fractions were collected, frozen and then lyophilized to obtain 106.6 mg (20.5% yield) of a spectroscopically pure (94.4% AUC at 254 nm) brown/copper powder. MS (ESI) calculated for C₁₁₅H₁₂₆N₂₂O₁₄ m/z 2039.99, found 1020.5 (M+2H)⁺.

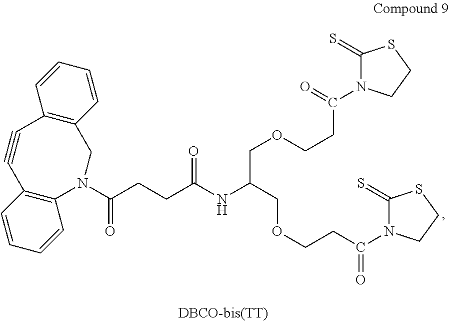 [SMILES: O=C(CCC(=O)N1Cc2ccccc2C#Cc2ccccc21)NC(COCCC(=O)N1CCSC1=S)COCCC(=O)N1CCSC1=S]

Compound 9, referred to as DBCO-2-Amino-1,3-bis(carboxylethoxy)propane(TT)2 or DBCO-bis(TT) was synthesized by reacting 385.6 mg (0.74 mmol, 1 eq) of the precursor DBCO-2-Amino-1,3-bis(carboxylethoxy)propane, with 193.4 mg of 2-Thiazoline-2-thiol (1.62 mmol, 2.2 eq) and 367.5 mg of 1-Ethyl-3-(3-dimethylaminopropyl) carbodiimide (1.92 mmol, 2.6 eq) in and 4-Dimethylaminopyridine in 4.0 mL of DCM. The reaction was run overnight at room temperature and HPLC indicated that the reaction was complete by 24 hours. The product eluted at 6.8 minutes on an Agilent analytical C18 column, 4.6×100 mm, 2.7 μm. Compound 9 was extracted with ethyl acetate and 1M HCl and was dried on the rotovap to obtain 317.1 mg (59.3% yield) of an impure (27.0% AUC at 254 nm) yellow powder. MS (ESI) calculated for C₃₄H₃₆N₄O₆S₄ m/z 724.15, found 725.3 (M+H)⁺

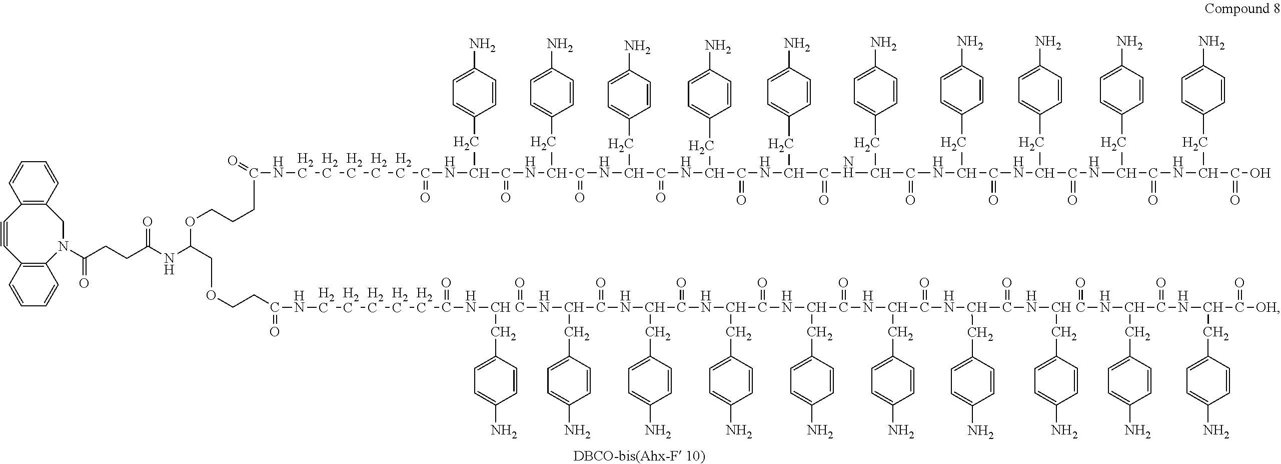

Compound 10, referred to as DBCO-2-Amino-1,3-bis(carboxylethoxy)propane(Ahx-F′10)2 or DBCO-bis(Ahx-F′10) was synthesized by reacting 13.0 mg (0.018 mmol, 1 eq) of the precursor DBCO-2-Amino-1,3-bis(carboxylethoxy)propane(TT)2, Compound 10, with 314.2 mg of (6-hydroxyhexanoyl)-(F′)₁₀—NH₂ (0.18 mmol, 10 eq) that was prepared by solid phase peptide synthesis and 199.5 mg of Triethylamine (1.97 mmol, 11.0 eq) in 1.8 mL of DMSO. The reaction was run overnight at room temperature and HPLC indicated that the reaction was complete by 24 hours. Compound 10 was purified on a preparatory HPLC system using a gradient of 5-25-35% acetonitrile/H₂O (0.05% TFA) over 14 minutes on an Agilent Prep-C18 column, 50×100 mm, 5 m. The product eluted at ˜9.8 minutes and the resulting fractions were collected, frozen and then lyophilized to obtain 19.16 mg (26.8% yield) of a spectroscopically pure (83.4% AUC at 254 nm) orange powder. MS (ESI) calculated for C₂₂₀H₂₅₂N₄₄O₃₀ m/z 3989.95, found 1330.8 (M+3H)⁺.

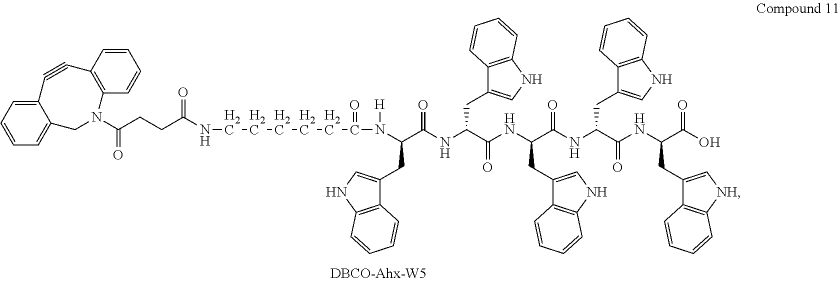 [SMILES: O=C(CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 11, referred to as DBCO-Ahx-W5 was synthesized by reacting 14.2 mg (0.035 mmol, 1 eq) of the precursor DBCO-NHS, with 37.5 mg of (6-hydroxyhexanoyl)-(W)₅-NH₂ (0.035 mmol, 1 eq) that was prepared by solid phase peptide synthesis and 3.93 mg of Triethylamine (0.039 mmol, 1.1 eq) in 0.5 mL of DMSO. The reaction was run overnight at room temperature and HPLC indicated that the reaction was complete by 24 hours. Compound 11 was crashed out in twice 1M HCL and once in H2O to obtain 34.3 (71.9% yield) of a spectroscopically pure (92.6% AUC at 254 nm) pink powder. MS (ESI) calculated for C₈₀H₇₆N₁₂O₉ m/z 1348.59, found 1348.4 (M+H)⁺.

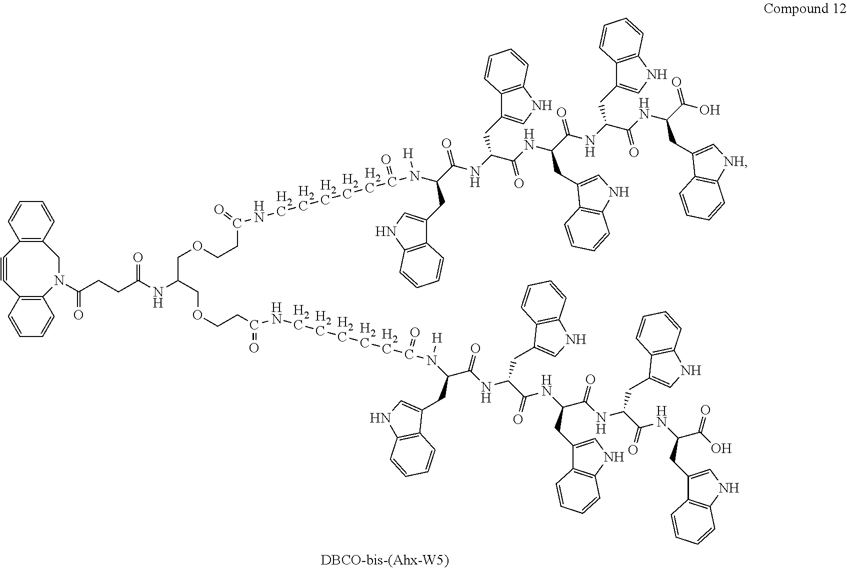 [SMILES: O=C(CCOCC(COCCC(=O)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O)NC(=O)CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 12, referred to as DBCO-2-Amino-1,3-bis(carboxylethoxy)propane(Ahx-W5)2 or DBCO-bis-(Ahx-W5) was synthesized by reacting 13.0 mg (0.018 mmol, 1 eq) of the precursor DBCO-2-Amino-1,3-bis(carboxylethoxy)propane(TT)2, with 41.3 mg of (6-hydroxyhexanoyl)-(W)₅-NH₂ (0.039 mmol, 2.2 eq) that was prepared by solid phase peptide synthesis and 9.1 mg of Triethylamine (0.09 mmol, 2.3 eq) in 0.3 mL of DMSO. The reaction was run overnight at room temperature and HPLC indicated that the reaction was complete by 24 hours. Compound 12 was purified on a preparatory HPLC system using a gradient of 15-60-90% acetonitrile/H₂O (0.05% TFA) over 16 minutes on an Agilent Prep-C18 column, 30×100 mm, 5 m. The product eluted at ˜12.7 minutes and the resulting fractions were collected, frozen and then lyophilized to obtain 12.5 mg (30.8% yield) of a spectroscopically pure (>95% AUC at 254 nm) pink powder. MS (ESI) calculated for C₁₅₀H₁₅₂N₂₄O₂₀ m/z 2609.16, found 1305.0 (M+2H)+.

### Synthesis of Amphiphilic Carrier Molecules

A combinatorial library of different C-B-L-H compositions was prepared by reacting different compositions of hydrophobic blocks bearing a linker precursor X1, bearing an alkyne, with different compositions of C-B bearing a linker precursor X2, bearing an azide. Each of the precursors, X1-H and C-B-X2, were first suspended in DMSO at greater than 20 mg/mL DMSO, depending on the solubility of the specific composition, sometimes up to 100 mg/mL DMSO, and then combined in a reaction vessel at a molar ratio of about 1.05 moles of X1-H for every 1.0 moles of C-B2-X2. The reactions were performed at room temperature and, for compositions wherein X1 comprises a DBCO group, without any additional reagents. Reaction were monitored by LC-MS and determined to be complete after the C-B-X2 fragment was fully converted to the C-B-L-H.

This reaction scheme was used to prepare different compositions of amphiphilic carrier molecules that were chararacterized for the capacity to form stable nanoparticle micelles in aqueous buffer, PBS pH 7.4, at a concentration of 0.5 mg/mL of amphiphilic carrier molecule. The results of these studies are summarized below according to the chemical composition and architecture of the amphiphilic carrier molecule.

### Linear Peptide

A series of linear amphiphilic carriers of formula C-B-L-H, wherein the charged moiety (C) and spacer (B) comprises peptides, i.e., poly(lysine) and poly(serine-co-glycine), respectively, with varying hydrophobic block composition were evaluated for particle size and stability by dynamic light scattering. The results show that nanoparticle micellization is highly dependent on the net charge of these compositions, with C-B-L-H with net charge of +8 and comprising hydrophobic blocks with up to 20 hydrophobic amino acids based on Phe(NH2), i.e., phenylalanine-amine, sometimes abbreviated F′, forming stable nanoparticle micelles, whereas those with +4 net charge were found to aggregate (Table 3).

TABLE 3
Peptide-based linear amphiphilic carrier molecules

| | | | Size |
|---|---|---|---|
| Composition (C-B-L-H), L = | Net | | (diameter, |
| (Lys(N3-DBCO)) | charge | MW | nm) |
| KKK- | 4 | 3660.44 | 1013 |
| SGSGSGSGSGSGSGSGSGSGSGSG- | | | |
| (Lys(N3-DBCO))-Ahx-(F′)5 | | | |
| KKK- | 4 | 4470.64 | 3839 |
| SGSGSGSGSGSGSGSGSGSGSGSG- | | | |
| (Lys(N3-DBCO))-Ahx-(F′)10 | | | |
| KKK- | 4 | 6092.46 | 1521 |
| SGSGSGSGSGSGSGSGSGSGSGSG- | | | |
| (Lys(N3-DBCO))-Ahx-(F′)20 | | | |
| KKK- | 4 | 6419.44 | 2683 |
| SGSGSGSGSGSGSGSGSGSGSGSG- | | | |
| (Lys(N3-DBCO))-Bis(Ahx-F′10)2 | | | |
| KKKKKKK- | 8 | 4173.14 | 477 |
| SGSGSGSGSGSGSGSGSGSGSGSG- | | | |
| (Lys(N3-DBCO))-Ahx-(F′)5 | | | |
| KKKKKKK- | 8 | 4983.34 | 16 |
| SGSGSGSGSGSGSGSGSGSGSGSG- | | | |
| (Lys(N3-DBCO))-Ahx-(F′)10 | | | |
| KKKKKKK- | 8 | 6605.16 | 32 |
| SGSGSGSGSGSGSGSGSGSGSGSG- | | | |
| (Lys(N3-DBCO))-Ahx-(F′)20 | | | |
| KKKKKKK- | 8 | 6932.14 | 56 |
| SGSGSGSGSGSGSGSGSGSGSGSG- | | | |
| (Lys(N3-DBCO))-(Ahx-(F′)10)2 | | | |

Note:
single letter abbreviations for amino acids are used in the above table.

### Linear PEG

A series of linear amphiphilic carriers of formula C-B-L-H, wherein the charged moiety (C) comprises peptides and the spacer (B) comprises a hydrophilic polymer, i.e. PEG, with varying hydrophobic block composition were evaluated for particle size and stability by dynamic light scattering.

Similar to the results observed with amphiphilic carrier molecules with peptide-based spacers, nanoparticle micellization was highly dependent on the net charge, with C-B-L-H with net charge of +8 and comprising hydrophobic blocks with up to 20 hydrophobic amino acids based on F′ forming stable nanoparticle micelles (Table 4). Though, notably, several C-B-L-H compositions with B comprised of a 24 monomer unit ethylene oxide (PEG) formed stable nanoparticle micelles with as little as +4 net charge, which suggests that spacer groups (B) based on hydrophilic polymers do not require as high charge as those amphiphilic carrier molecules with peptide-based spacers.

TABLE 4
Peptide- and hydrophilic polymer-based
linear amphiphilic carrier molecules

| | | | Size |
|---|---|---|---|
| Composition (C-B-L-H), L = | Net | | (diameter, |
| (Azide-DBCO) | charge | MW | nm) |
| KK-PEG4-(azide-DBCO)- | 2 | 1776.4 | 2427 |
| Ahx-(F′)5 | | | |
| KK-PEG4-(azide-DBCO)- | 2 | 2586.6 | 1112 |
| Ahx-(F′)10 | | | |
| KK-PEG4-(azide-DBCO)- | 2 | 4208.42 | 3618 |
| Ahx-(F′)20 | | | |
| KK-PEG4-(azide-DBCO)- | 2 | 4535.4 | 2650 |
| (Ahx-(F′)10)2 | | | |
| KK-PEG24-(azide-DBCO)- | 2 | 2656.76 | 467 |
| Ahx-(F′)5 | | | |
| KK-PEG24-(azide-DBCO)- | 2 | 3466.96 | 24 |
| Ahx-(F′)10 | | | |
| KK-PEG24-(azide-DBCO)- | 2 | 5088.78 | 2205 |
| Ahx-(F′)20 | | | |
| KK-PEG24-(azide-DBCO)- | 2 | 5415.76 | 1492 |
| (Ahx-(F′)10)2 | | | |
| KKKK-PEG4-(azide- | 4 | 2032.1 | 2069 |
| DBCO)-Ahx-(F′)5 | | | |
| KKKK-PEG4-(azide- | 4 | 2842.3 | 14 |
| DBCO)-Ahx-(F′)10 | | | |
| KKKK-PEG4-(azide- | 4 | 4464.12 | 2890 |
| DBCO)-Ahx-(F′)20 | | | |
| KKKK-PEG4-(azide- | 4 | 4791.1 | 2121 |
| DBCO)-(Ahx-(F′)10)2 | | | |
| KKKK-PEG24-(azide- | 4 | 2913.1 | 1392 |
| DBCO)-Ahx-(F′)5 | | | |
| KKKK-PEG24-(azide- | 4 | 3723.3 | 22 |
| DBCO)-Ahx-(F′)10 | | | |
| KKKK-PEG24-(azide- | 4 | 5345.12 | 103 |
| DBCO)-Ahx-(F′)20 | | | |
| KKKK-PEG24-(azide- | 4 | 5672.1 | 30 |
| DBCO)-(Ahx-(F′)10)2 | | | |
| KKKKKKKK-PEG4-(azide- | 8 | 2544.69 | 1181 |
| DBCO)-Ahx-(F′)5 | | | |
| KKKKKKKK-PEG4-(azide- | 8 | 3354.89 | 11 |
| DBCO)-Ahx-(F′)10 | | | |
| KKKKKKKK-PEG4-(azide- | 8 | 4976.71 | 19 |
| DBCO)-Ahx-(F′)20 | | | |
| KKKKKKKK-PEG4-(azide- | 8 | 5303.69 | 17 |
| DBCO)-(Ahx-(F′)10)2 | | | |
| KKKKKKKK-PEG24-(azide- | 8 | 3425.79 | 49 |
| DBCO)-Ahx-(F′)5 | | | |
| KKKKKKKK-PEG24-(azide- | 8 | 4235.99 | 16 |
| DBCO)-Ahx-(F′)10 | | | |
| KKKKKKKK-PEG24-(azide- | 8 | 5857.81 | 21 |
| DBCO)-Ahx-(F′)20 | | | |
| KKKKKKKK-PEG24-(azide- | 8 | 6184.79 | 26 |
| DBCO)-(Ahx-(F′)10)2 | | | |

Note:
single letter abbreviations for amino acids are used in the above table; and, oligo(lysine) sequences in the above table were linked to the PEG spacer through the N-terminus and are terminated with an amide.

Dendritic Charged Moiety, with Linear PEG (Cone-Shaped)

A series of cone-shaped amphiphilic carriers of formula C-B-L-H, wherein the charged moiety (C) comprises peptides of dendritic structure and the spacer (B) comprises a hydrophilic polymer, i.e. PEG, with varying hydrophobic block composition were evaluated for particle size and stability by dynamic light scattering. The cone-shaped structures exhibited overall similar characteristics to amphiphilic carrier molecules based on linear C-B-L-H, wherein B is a hydrophilic polymer, and, notably required up to +8 net charge to stabilize hydrophobic blocks (H) comprised of 20 hydrophobic amino acids based on F′ (e.g., Ahx-(F′)20) (Table 5).

TABLE 5
Peptide- and hydrophilic polymer-based, cone-shaped amphiphilic
carrier molecules

| | | | Size |
|---|---|---|---|
| | | | (diam- |
| Composition (C-B-L-H), L = | Net | | eter, |
| (Lys(N3-DBCO)) | charge | MW | nm) |
| K2K-PEG4-Lys(N3-DBCO)-Ahx-(F′)5 | 4 | 2033.3 | 1685 |
| K2K-PEG4-Lys(N3-DBCO)-Ahx-(F′)10 | 4 | 2843.5 | 53 |
| K2K-PEG4-Lys(N3-DBCO)-Ahx-(F′)20 | 4 | 4465.32 | 2038 |
| K2K-PEG4-Lys(N3-DBCO)-(Ahx-(F′)10)2 | 4 | 4792.3 | 2000 |
| K2K-PEG24-Lys(N3-DBCO)-Ahx-(F′)5 | 4 | 2913.09 | 3 |
| K2K-PEG24-Lys(N3-DBCO)-Ahx-(F′)10 | 4 | 3723.29 | 24 |
| K2K-PEG24-Lys(N3-DBCO)-Ahx-(F′)20 | 4 | 5345.11 | 5590 |
| K2K-PEG24-Lys(N3-DBCO)-(Ahx-(F′)10)2 | 4 | 5672.09 | 2000 |
| K4K2K-PEG4-Lys(N3-DBCO)-Ahx-(F′)5 | 8 | 2544.7 | 532 |
| K4K2K-PEG4-Lys(N3-DBCO)-Ahx-(F′)10 | 8 | 3354.9 | 10 |
| K4K2K-PEG4-Lys(N3-DBCO)-Ahx-(F′)20 | 8 | 4976.72 | 20 |
| K4K2K-PEG4-Lys(N3-DBCO)-(Ahx-(F′)10)2 | 8 | 5303.7 | 67 |
| K4K2K-PEG24-Lys(N3-DBCO)-Ahx-(F′)5 | 8 | 3425.77 | 892 |
| K4K2K-PEG24-Lys(N3-DBCO)-Ahx-(F′)10 | 8 | 4235.97 | 17 |
| K4K2K-PEG24-Lys(N3-DBCO)-Ahx-(F′)20 | 8 | 5857.79 | 32 |
| K4K2K-PEG24-Lys(N3-DBCO)-(Ahx-(F′)10)2 | 8 | 6184.77 | 2000 |

Note:
single letter abbreviations for amino acids are used in the above table; and, K2K and K4K2K are lysine dendrons comprising 3 and 7 lysines, respectively. For clarity, the structure of K2K (linked to a spacer, B) is shown here for clarity:

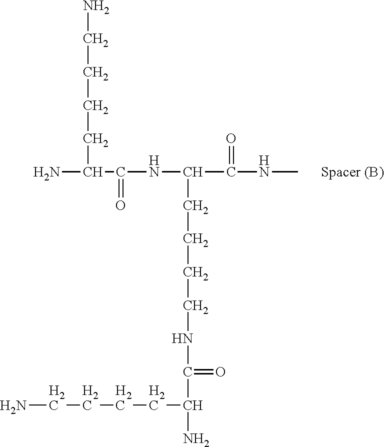 [SMILES: CNC(=O)C(CCCCNC(=O)C(N)CCCCN)NC(=O)C(N)CCCCN]

C-B-L-H with Brush Architecture

Finally, a series of brush amphiphilic carriers of formula (C-B)y19-K-L-H, wherein the charged moiety (C) comprises peptides, the spacer (B) comprises a hydrophilic polymer, i.e. PEG, and K is an amplifying linker having 4 sites of attachment (y19=4) for every one hydrophobic block, with varying hydrophobic block composition were evaluated for particle size and stability by dynamic light scattering.

A striking finding was that the brush amphiphilic carrier molecules required less net charge to form stable nanoparticle micelles as compared with the other compositions and architectures of amphiphilic carrier molecules. For instance, whereas the linear and cone amphiphilic carrier molecule structures with hydrophobic blocks based on Ahx-(F′)20 and (Ahx-(F′)10)2 with a net charge of +4 were found to form aggregates, indicating insufficient charge stabilization, the brush structures of formula (C-B)y19-K-L-H all formed stable nanoparticle micelles without presence of aggregates (Table 6).

TABLE 6
Peptide- and hydrophilic polymer-based, brush-
shaped amphiphilic carrier molecules

| | | | Size |
|---|---|---|---|
| Composition (C-B-L-H), L = | Net | | (diameter, |
| (Lys(N3-DBCO)) | charge | MW | nm) |
| NH2-PEG24-(azide-propargyl)- | 4 | 4234.03 | 8 |
| 4K2K-Lys(N3-DBCO)-Ahx-(F′)10 | | | |
| NH2-PEG24-(azide-propargyl)- | 4 | 5855.85 | 14 |
| 4K2K-Lys(N3-DBCO)-Ahx-(F′)20 | | | |
| NH2-PEG24-(azide-propargyl)- | 4 | 6182.83 | 11 |
| 4K2K-Lys(N3-DBCO)-(Ahx-(F′)10)2 | | | |
| KK-PEG24-(azide-propargyl)- | 8 | 4474.35 | 23 |
| 4K2K-Lys(N3-DBCO)-Ahx-(F′)10 | | | |
| KK-PEG24-(azide-propargyl)- | 8 | 6096.17 | 12 |
| 4K2K-Lys(N3-DBCO)-Ahx-(F′)20 | | | |
| KK-PEG24-(azide-propargyl)- | 8 | 6423.15 | 14 |
| 4K2K-Lys(N3-DBCO)-(Ahx-(F′)10)2 | | | |

Note:
single letter abbreviations for amino acids are used in the above table; and, oligo(lysine) sequences in the above table were linked to the PEG spacer through the N-terminus and are terminated with an amide.

### Example 8—Synthesis of Peptide Antigen Fragments with Alkyl Amines and Aryl Amines

Highly hydrophobic peptide antigens (A) can be challenging to manufacture. However, as disclosed herein, incorporation of amines into peptide sequences can improve manufacturability. Therefore, to improve the manufacturability of peptide antigens (A), amino acids bearing amine and/or guanidine functional groups were placed on the charged moiety (C) and/or extension proximal to the charged moiety and evaluated for manufacturability and ease of handling (e.g., solubility in commonly used solvents).

A native peptide antigen (A), which is challenging to manufacture (see Table 7, second row), was produced as a peptide antigen fragment of formula C-B1-A-B2-X1 with different lengths of charged moieties (C) bearing lysine (Table 7, rows 3 to 5). Notably, the peptide antigen fragments with multiple lysine residues, but not the native antigen, were manufacturable.

TABLE 7
Impact of amines on peptide antigen manufacturability

| Peptide antigen fragment | Confirmed | Successful |
|---|---|---|
| (C-B1-A-B2-X1), K′ = Lys(N3) | MW | synthesis |
| QGTDVVIAIFIILAMSFVPASFVVF | — | No |
| (antigen alone) | | |
| KKKKKKK-VR- | 4431.01 | Yes |
| QGTDVVIAIFIILAMSFVPASFVVF-SPVZ-K′ | | |
| KKKKKKKKK-VR- | 4687.75 | Yes |
| QGTDVVIAIFIILAMSFVPASFVVF-SPBZ-K′ | | |
| KKKKKKKKKK-VR- | 4815.95 | Yes |
| QGTDVVIAIFIILAMSFVPASFVVF-SPVZ-K′ | | |

Note:
single letter abbreviations for amino acids are used in tire above table.

Similarly, peptide antigen fragments that comprise a highly hydrophobic peptide antigen (A) were not manufacturable without the incorporation of either lysine, para-amino-phenylalanine or histidine residues (Table 8), indicating that peptide antigen fragments comprising amines, aryl amines and/or heterocycles with a protonatable nitrogen (e.g., imidazoles, quinolines, etc.) facilitate manufacturing, possibly by improving solubility during synthesis and purification.

TABLE 8
Impact of amines on manufacturability
of peptide antigen fragments

| Peptide antigen fragment | | Successful |
|---|---|---|
| ([C]-[B1]-A-B2-X1), K′ = Lys(N3) | Confirmed MW | synthesis |
| VVIAIFIILV-ZK′ | — | No |
| VVIAIFIIL-SPVZ-K′ | — | No |
| KKKKKK-SLVR- | 2818.29 | Yes |
| VVIAIFIIL-SPVZ-K′ | | |
| VVIAIFIIL- | 2243.17 | Yes |
| SPVZF′F′F′F′F′F′F′F′-K′ | | |
| VVIAIFIIL-SPVZHHHH-K′ | 2142.56 | Yes |

Note:
single letter abbreviations for amino acids are used in the above table.

To expand on these findings, a series of different peptide antigen fragments comprising amine, aryl amine, guandine and/or nitrogen heterocycles (e.g., imidazoles, quinolines, etc.) were synthesized to evaluate manufacturability and suitability for use in immunogenic compositions. A subset of this data is shown in Table 9.

TABLE 9
Examples of peptide antigen fragments bearing aryl amines
and aromatic heterocycles with protonatable nitrogen

| Peptide antigen fragment | Confirmed |
|---|---|
| ([C]-[B1]-A-B2-X1), K′ = Lys(N3) | MW |
| AALLNSAVL-SPVZF′F′F′F′-K′ | 2114.04 |
| AALLNSAVL-SPVZHHHH-K′ | 2013.27 |
| AQLANDVVL-SPVZF′F′F′F′-K′ | 2184.99 |
| AQLANDVVL-SPVZHHHH-K′ | 2084.31 |
| Ac-DDDDD-VZ- | 4920.00 |
| DFTGSNGDPSSPYSLHYLSPTGVNEY-SPVZF′F′F′F′-K′ | |
| Ac-EEEEE-VZ- | 4991.79 |
| DFTGSNGDPSSPYSLHYLSPTGVNEY-SPVZF′F′F′F′-K′ | |
| Ac-EEEEE-VZ- | 5641.19 |
| DFTGSNGDPSSPYSLHYLSPTGVNEY- | |
| SPVZF′F′F′F′F′F′F′F′-K′ | |
| Ac-EEEEEEE-VZ- | 5555.14 |
| GIPVHLELASMTNMELMSSIVHQQVFPT-SPVZF′F′F′F′-K′ | |

Note:
single letter abbreviations for ammo acids are used in the above table.

### Example 9—Hydrophobic Blocks Comprising Amines and/or Aryl Amines

To extend the above findings, the impact that the incorporation of amino acids with amines and/or aryl amines has on the synthesis of hydrophobic blocks (H) was evaluated.

As shown in Table 10, incorporation of amino acids bearing amines but not carboxylic acids (e.g., glutamic acid) led to improved manufacturability of hydrophobic blocks, e.g., a hydrophobic block based on poly(Trp).

TABLE 10
hydrophobic blocks comprising alkyl amines

| | Confirmed | Successful |
|---|---|---|
| Hydrophobic block precursor | MW | synthesis |
| Fmoc-WWWWWEWWWW | — | No |
| Fmoc-WWEWWWWEWW | — | No |
| Fmoc-EWEWEEWEWE | 1758.80 | Yes |
| Fmoc-WWWWWKWWWW | 1821.10 | Yes |
| Fmoc-WWKWWWWKWW | 1985.30 | Yes |
| Fmoc-KWKWKKWKWK | 1753.15 | Yes |
| Fmoc-WWWWWKWWWWW | 2007.30 | Yes |
| Fmoc-KWWKWWKWWKWWKWWK | 2870.42 | Yes |

Note:
single letter abbreviations for amino acids are used in the above table.

Similarly, the incorporation of amino acids bearing aryl amines was found to improve the manufacturability of hydrophobic blocks, e.g., a hydrophobic block based on poly(Trp) (Table 11). Notably, interspersing the amino acids bearing the aryl amines led to improved manufacturability as compared with hydrophobic blocks comprised of blocks of the two different amino acid compositions (see Table 11, rows 5 and 6).

TABLE 11
Hydrophobic blocks comprising and amines

| Hydrophobic block, F′ = aminophenylanine | Confirmed MW | Successful synthesis |
|---|---|---|
| WWWWWWWWWW | — | No |
| F′F′F′F′F′ | 827.90 | Yes |
| F′F′F′F′F′F′F′F′F′F′ | 1638.76 | Yes |
| WWWWWWWWWWF′F′F′F′F′F′F′F′F′F′ | — | No |
| WF′WF′WF′WF′WF′WF′WF′WF′WF′WF′ | 3500.86 | Yes |
| F′F′F′F′F′F′F′F′F′F′F′F′F′F′F′F′F′F′F′F′ | 3260.50 | Yes |
| F′F′F′F′F′F′F′F′F′F′F′F′F′F′F′F′F′F′F′F′F′F′F′F′F′F′F′F′F′F′ | 4882.25 | Yes |

### Example 10—Immunogenic Compositions Based on Peptide Antigen Fragments Comprising Aryl Amines

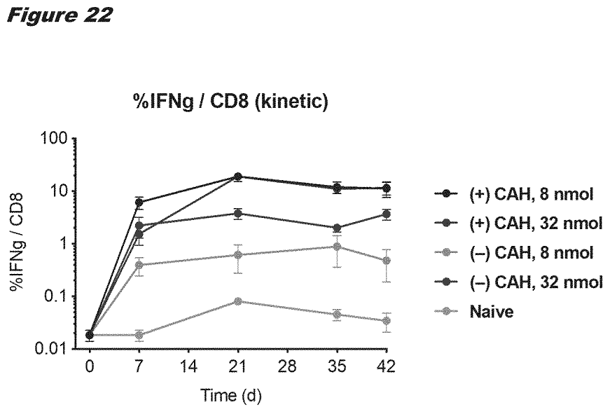

To evaluate the suitability of immunogenic compositions based on peptide antigen fragments comprising aryl amines, mice were either vaccinated with peptide antigen conjugates of formula C-B1-A-B2-L-H, either comprising a charged moiety with positive charge, Lys-Lys-Lys-Lys-Lys-Lys-Lys-Lys-Lys-Val-Arg-Asp-Phe-Thr-Gly-Ser-Asn-Gly-Asp-Pro-Ser-Ser-Pro-Try-Ser-Leu-His-Tyr-Leu-Ser-Pro-Thr-Gly-Val-Asn-Glu-Tyr-Ser-Pro-Val- Cit-Lys(N3-DBCO)-Glu(2B)-Trp-Glu(2B)-Trp-Glu(2B)—NH2 or a charged moiety with negative charge and a B2 comprised of amino acids bearing aryl amines, i.e., Ac-Glu-Glu-Glu-Glu-Glu-Val-Cit-Asp-Phe-Thr-Gly-Ser-Asn-Gly-Asp-Pro-Ser-Ser-Pro-Try-Ser-Leu-His-Tyr-Leu-Ser-Pro-Thr-Gly-Val-Asn-Glu- Tyr-Ser-Pro-Val-Cit-Phe(NH2)-Phe(NH2)-Phe(NH2)-Phe(NH2)-Lys(N3-DBCO)-Glu(2B)-Trp-Glu(2B)-Trp-Glu(2B)-NH2, wherein Phe(NH2) is para-amino-phenylalanine. Importantly, CD8 T cell responses were induced in all animals that received the tris salts of the negatively charged peptide antigen conjugate (FIG. 22). The charge-modified peptide was produced with four para-amino-phenylalanine amino acids as part of the C-terminal extension (B2) to improve the solubility of the peptide during purification. CD8 T cell responses were detected in all vaccinated animals.

### Example 11—Immunogenic Compositions Based on Amphiphilic Carrier Molecules

To evaluate the suitability of immunogenic compositions comprised of peptide antigen conjugates and amphiphilic carrier molecules, two amphiphilic carrier molecule strategies were evaluated and described below.

One approach was to form mosaic particles based on the combination of a peptide antigen conjugate of formula A-B2-L-H with a charged amphiphilic carrier molecule of formula C-L-H. In short, a peptide antigen conjugate of formula, A-L-H, i.e. Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Lys(N3-DBCO)-Glu(2B)-Trp-Glu(2B)-Trp-Glu(2B)-NH2 was admixed in DMSO solution with Lys-Lys-Lys-Lys-Lys-Lys-Lys-Lys-Lys-Lys-Lys(N3-DBCO)-Glu(2B)-Trp-Glu(2B)-Trp-Glu(2B)-NH2 in an equimolar ratio and then suspended in PBS to a concentration of 80 μM.

A second approach was to form mosaic particles based on the combination of a peptide antigen conjugate of formula A-B2-L-H with a charged amphiphilic carrier molecule of formula C-B1-A′-B2-L-H, wherein A′ is a conserved antigen, or, in this particular case, a helper epitope. The peptide antigen conjugate of formula, A-L-H, i.e. Gly-Ile-Pro-Val-His-Leu-Glu-Leu-Ala-Ser-Met-Thr-Asn-Met-Glu-Leu-Met-Ser-Ser-Ile-Val-His-Gln-Gln-Val-Phe-Pro-Thr-Lys(N3-DBCO)-Glu(2B)-Trp-Glu(2B)-Trp-Glu(2B)-NH2 was admixed in DMSO solution with the amphiphilic carrier molecule of formula C-B1-A′-B2-L-H, i.e, Lys-Lys-Lys-Ser-Leu-Val-Ar-Ala-Lys-Phe-Val-Ala-Ala-Trp-Thr-Leu-Lys-Ala-Ala-Ala-Ser-Pro-Val-Cit-(N3-DBCO)-Glu(2B)-Trp-Glu(2B)-Trp-Glu(2B)-NH2 in an equimolar ratio and then suspended in PBS to a concentration of 80 μM.

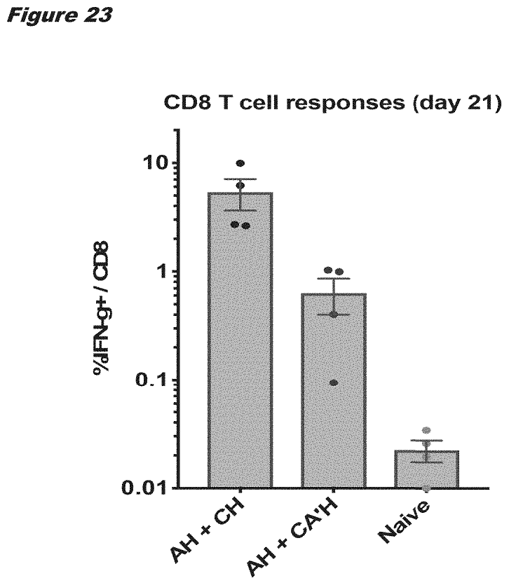

Mice were vaccinated with both immunogenic compositions and CD8 T cell on days 0 and 14 and CD8 T cell responses were assessed on Day 21 (FIG. 23). Importantly, immunogenic compositions comprised of peptide antigen conjugates, without a charged moiety, and charged amphiphilic carrier molecules led to high magnitude CD8 T cell responses (FIG. 23).

### SEQUENCES

The following amino acid sequences are disclosed herein.

(SEQ ID NO: 1)
Ser-Pro-Leu-Arg
(SEQ ID NO: 2)
Gly-Gly-Lys-Leu-Val-Arg
(SEQ ID NO: 3)
Gly-Gly-Lys-Pro-Leu-Arg
(SEQ ID NO: 4)
Gly-Gly-Ser-Leu-Val-Arg
(SEQ ID NO: 5)
Gly-Gly-Ser-Leu-Val-Leu
(SEQ ID NO: 6)
Gly-Gly-Glu-Leu-Val-Arg
(SEQ ID NO: 7)
Gly-Gly-Glu-Leu-Val-Leu
(SEQ ID NO: 8)
Gly-Ser-Leu-Val-Arg
(SEQ ID NO: 9)
Gly-Lys-Pro-Val-Arg
(SEQ ID NO: 10)
Gly-Ser-Leu-Val-Leu
(SEQ ID NO: 11)
Gly-Glu-Leu-Val-Leu
(SEQ ID NO: 26)
Gly-Gly-Ser-Leu-Val-Cit
(SEQ ID NO: 12)
Ser-Leu-Val-Leu
(SEQ ID NO: 27)
Ser-Pro-Val-Cit
(SEQ ID NO: 13)
Glu-Leu-Val-Arg
(SEQ ID NO: 14)
Ser-Pro-Val-Arg
(SEQ ID NO: 15)
Ser-Leu-Val-Arg
(SEQ ID NO: 16)
Lys-Pro-Leu-Arg
(SEQ ID NO: 28)
Glu-Leu-Val-Cit
(SEQ ID NO: 17)
Glu-Leu-Val-Leu
(SEQ ID NO: 18)
Lys-Pro-Leu-Arg-Tyr-Leu-Leu-Leu
(SEQ ID NO: 29)
Gly-Ser-Leu-Val-Cit
(SEQ ID NO: 19)
Ser-Leu-Val-Leu
(SEQ ID NO: 30)
Ser-Pro-Val-Cit
(SEQ ID NO: 20)
Glu-Leu-Val-Arg
(SEQ ID NO: 21)
Ser-Pro-Val-Arg
(SEQ ID NO: 22)
Ser-Leu-Val-Arg
(SEQ ID NO: 23)
Lys-Pro-Leu-Arg
(SEQ ID NO: 24)
Lys-Pro-Val-Arg
(SEQ ID NO: 31)
Glu-Leu-Val-Cit
(SEQ ID NO: 25)
Glu-Leu-Val-Leu
(SEQ ID NO: 32)
Gly-Lys-Pro-Val-Cit
(SEQ ID NO: 33)
Gly-Gly-Ser-Pro-Val-Cit
(SEQ ID NO: 34)
Glu-Pro-Val-Cit,
(SEQ ID NO: 35)
Glu-Gly-Val-Cit.
(SEQ ID NO: 36)
Ser-Leu-Val-Cit
(SEQ ID NO: 37)
Glu-Pro-Val-Cit
(SEQ ID NO: 38)
Lys-Pro-Val-Cit.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A process for producing a peptide antigen conjugate suitable for administration to a mammal, the peptide antigen conjugate comprising a peptide antigen linked to a hydrophobic block, the process comprising:
reacting a hydrophobic block fragment with a peptide antigen fragment comprising the peptide antigen in a pharmaceutically acceptable organic solvent in a hydrophobic block fragment to peptide antigen fragment molar ratio of 1:1 or greater under conditions to directly or indirectly link the peptide antigen to the hydrophobic block; and
obtaining a product solution comprising the peptide antigen conjugate, unreacted hydrophobic block fragment and pharmaceutically acceptable organic solvent.

2. The process according to claim 1, wherein the product solution that is formed comprises unreacted hydrophobic block fragment and the unreacted hydrophobic block fragment is not removed from the product solution.

3. The process according to any one of the preceding claims, further comprising sterile filtering the product solution to obtain a sterile product solution comprising peptide antigen conjugate, any unreacted hydrophobic block fragment and pharmaceutically acceptable organic solvent.

4. The process according to claim 3, further comprising adding an excess volume of aqueous buffer to the sterile product solution followed by mixing to generate a sterile aqueous solution of peptide antigen conjugate particles comprising the peptide antigen conjugate, any unreacted hydrophobic block fragment, pharmaceutically acceptable organic solvent and aqueous buffer.

5. The process according to claim 4, wherein the aqueous solution of peptide antigen conjugate particles comprises unreacted hydrophobic block fragment and the unreacted hydrophobic block fragment is not removed from the aqueous solution of peptide antigen conjugate particles.

6. The process according to either claim 4 or claim 5, wherein the process does not involve removal of the pharmaceutically acceptable organic solvent.

7. The process according to claim 3, further comprising lyophilizing the sterile product solution to obtain a lyophilized sterile product.

8. The process according to claim 7, further comprising adding an excess volume of aqueous buffer to the lyophilized sterile product followed by mixing to generate a sterile aqueous solution of peptide antigen conjugate particles comprising the peptide antigen conjugate, any unreacted hydrophobic block fragment and aqueous buffer.

9. The process according to either claim 1 or claim 2, further comprising purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent.

10. The process according to claim 9, further comprising sterile filtering the purified peptide antigen conjugate solution to obtain a sterile purified peptide antigen conjugate solution comprising the peptide antigen conjugate and pharmaceutically acceptable organic solvent.

11. The process according to claim 10, further comprising adding an excess volume of aqueous buffer to the sterile purified peptide antigen conjugate solution followed by mixing to generate a sterile aqueous solution of peptide antigen conjugate particles comprising the peptide antigen conjugate, pharmaceutically acceptable organic solvent and aqueous buffer.

12. The process according to claim 10, further comprising lyophilizing the sterile purified peptide antigen conjugate solution to obtain a lyophilized sterile purified peptide antigen conjugate.

13. The process according to claim 12, further comprising adding an excess volume of aqueous buffer to the lyophilized sterile purified peptide antigen conjugate followed by mixing to generate a sterile aqueous solution of peptide antigen conjugate particles comprising the peptide antigen conjugate and aqueous buffer.

14. The process according to any of the preceding claims, further comprising analysing the propensity of the product solution, sterile product solution, lyophilized sterile product, lyophilized purified peptide antigen conjugate, purified peptide antigen conjugate solution, sterile purified peptide antigen conjugate solution and/or lyophilized sterile purified peptide antigen conjugate to form aggregated material upon addition of an aqueous buffer, the analysis comprising:
(i) aliquoting a specific volume of the product solution, sterile product solution, purified peptide antigen conjugate solution and/or sterile purified peptide antigen conjugate solution from a first container to a second container, and/or adding a specific mass of the lyophilized sterile product, lyophilized purified peptide antigen conjugate and/or lyophilized sterile purified peptide antigen conjugate from a first container to a second container;
(ii) adding a volume of the aqueous buffer to the second container to obtain an aqueous solution of peptide antigen conjugate particles comprising the peptide antigen conjugate and any unreacted hydrophobic block fragment, wherein the concentration of the peptide antigen conjugate is not lower than 0.01 mg/mL;
(iii) assessing turbidity of the aqueous solution of peptide antigen conjugate particles by measuring absorbance at a wavelength greater than 350 nm; and
(iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

15. The process according to any one of the preceding claims, wherein the pharmaceutically acceptable organic solvent is selected from one or more of the group consisting of dimethyl sulfoxide (DMSO), methanol and ethanol.

16. The process according to claim 15, wherein the pharmaceutically acceptable organic solvent is DMSO.

17. The process according to any one of the preceding claims, wherein the peptide antigen fragment has a formula selected from [C]-[B1]-A-[B2]-X1, [B1]-A-[B2]-X1([C]), X1-[B1]-A-[B2]-[C] or X1([C])-[B1]-A-[B2] where C is a charged moiety, B1 is an N-terminal extension, A is a peptide antigen, B2 is a C-terminal extension, [ ] denotes that the group is optional, and X1 is a linker precursor comprising a first reactive functional group; and the hydrophobic block fragment has a formula selected from X2-H, X2([C])-H or X2-H([C]) where H is a hydrophobic block, C is a charged moiety, [ ] denotes that the group is optional, and X2 is a linker precursor comprising a second reactive functional group that is reactive with the first reactive functional group, and X1 and X2 undergo a reaction to form a covalent bond that results in a Linker L.

18. The process according to claim 17, wherein the peptide antigen conjugate has the formula [C]-[B1]-A-[B2]-L-H.

19. The process according to claim 18, wherein the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

20. The process according to claim 17, wherein the peptide antigen conjugate has the formula H-L-[B1]-A-[B2]-[C].

21. The process according to claim 20, wherein the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

22. The process according to any one of the preceding claims, wherein the hydrophobic block comprises a poly(amino acid)-based polymer.

23. The process according to claim 22, wherein the poly(amino acid)-based polymer comprises aromatic rings or heterocyclic aromatic rings.

24. The process according to claim 23, wherein the poly(amino acid)-based polymer comprises aryl amines.

25. The process according to any one of the preceding claims, wherein the hydrophobic block fragment is reacted with the peptide antigen fragment in a hydrophobic block fragment to peptide antigen fragment molar ratio of from about 1:1 to about 3:1.

26. The process according to claim 25, wherein the hydrophobic block fragment is reacted with the peptide antigen fragment in a hydrophobic block fragment to peptide antigen fragment molar ratio of from 1:1 to about 12:10.

27. The process according to any one of the preceding claims, further comprising forming a peptide antigen conjugate mixture or lyophilized peptide antigen conjugate mixture comprising two or more peptide antigen conjugates, the process comprising:
combining a specific volume of a first product solution comprising a first peptide antigen conjugate, a first purified peptide antigen conjugate solution comprising a first peptide antigen conjugate, a first sterile product solution comprising a first peptide antigen conjugate and/or a first sterile purified peptide antigen conjugate solution comprising a first peptide antigen conjugate with at least a second product solution comprising a second peptide antigen conjugate, a second purified peptide antigen conjugate solution comprising a second peptide antigen conjugate, a second sterile product solution comprising a second peptide antigen conjugate and/or a second sterile purified peptide antigen conjugate solution comprising a second peptide antigen conjugate to obtain a peptide antigen conjugate mixture comprising at least the first peptide antigen conjugate and the second peptide antigen conjugate, any unreacted hydrophobic block fragment and the pharmaceutically acceptable organic solvent; and/or
combining a specific mass of a first lyophilized product comprising a first peptide antigen conjugate, a first lyophilized purified peptide antigen conjugate comprising a first peptide antigen conjugate, a first lyophilized sterile product comprising a first peptide antigen conjugate and/or a first lyophilized sterile purified peptide antigen conjugate comprising a first peptide antigen conjugate with at least a specific mass of a second lyophilized product comprising a second peptide antigen conjugate, a second lyophilized purified peptide antigen conjugate comprising a second peptide antigen conjugate, a second lyophilized sterile product comprising a second peptide antigen conjugate and/or a second lyophilized sterile purified peptide antigen conjugate comprising a second peptide antigen conjugate to obtain a lyophilized peptide antigen conjugate mixture comprising at least the first peptide antigen conjugate and the second peptide antigen conjugate and any unreacted hydrophobic block fragment.

28. The process according to claim 27, wherein the peptide antigen conjugate mixture comprises unreacted hydrophobic block fragment and the unreacted hydrophobic block fragment is not removed from the peptide antigen conjugate mixture.

29. The process according to either claim 27 or claim 28, wherein the step of combining a specific volume of the first product solution, the first purified peptide antigen conjugate solution, the first sterile product solution and/or the first sterile purified peptide antigen conjugate solution with at least the second product solution, the second purified peptide antigen conjugate solution, the second sterile product solution and/or the second sterile purified peptide antigen conjugate solution comprises selecting and transferring a specific volume of solution to transfer from one container to a second container, the process comprising the steps of:
(i) determining the molar concentration of the peptide antigen conjugate in at least the first product solution, the first purified peptide antigen conjugate solution, the first sterile product solution, the first sterile purified peptide antigen conjugate solution, the second product solution, the second purified peptide antigen conjugate solution, the second sterile production solution and/or the second sterile purified peptide antigen conjugate solution;
(ii) aliquoting a specific volume of at least the first product solution, the first purified peptide antigen conjugate solution, the first sterile product solution and/or the first sterile purified peptide antigen conjugate solution and the second product solution, the second purified peptide antigen conjugate solution, the second sterile product solution and/or the second sterile purified peptide antigen conjugate solution from the first container to a second container to obtain a specific molar content of each of the first peptide antigen conjugate and the second peptide antigen conjugate.

30. The process according to claim 29, wherein the process of determining the molar concentration of peptide antigen conjugate in at least the first product solution, the first purified peptide antigen conjugate solution, the first sterile product solution, the first sterile purified peptide antigen conjugate solution, the second product solution, the second purified peptide antigen conjugate solution, the second sterile product solution and/or the second sterile purified peptide antigen conjugate solution comprises measuring UV-Vis absorption of the peptide antigen conjugate at a wavelength between about 300 to about 350 nm.

31. The process according to any one of claims 27 to 30, further comprising adding an excess volume of aqueous buffer to the peptide antigen conjugate mixture followed by mixing to generate an aqueous solution of peptide antigen conjugate particles comprising at least the first peptide antigen conjugate and the second peptide antigen conjugate, any unreacted hydrophobic block fragment, any pharmaceutically acceptable organic solvent and aqueous buffer.

32. The process according to any one of claims 27 to 30, further comprising lyophilization of the peptide antigen conjugate mixture to obtain a lyophilized peptide antigen conjugate mixture.

33. The process according to claim 32, further comprising adding an excess volume of aqueous buffer to the lyophilized peptide antigen conjugate mixture followed by mixing to generate an aqueous solution of peptide antigen conjugate particles comprising at least the first peptide antigen conjugate and the second peptide antigen conjugate, any unreacted hydrophobic block fragment and aqueous buffer.

34. The process according to any one of claims 27 to 30, further comprising sterile filtering the peptide antigen conjugate mixture to obtain a sterile peptide antigen conjugate mixture.

35. The process according to claim 34, further comprising adding an excess volume of aqueous buffer to the sterile peptide antigen conjugate mixture product followed by mixing to generate a sterile aqueous solution of peptide antigen conjugate particles comprising at least the first peptide antigen conjugate and the second peptide antigen conjugate, any unreacted hydrophobic block fragment, pharmaceutically acceptable organic solvent and aqueous buffer.

36. The process according to claim 34, further comprising lyophilization of the sterile peptide antigen conjugate mixture to obtain a lyophilized sterile peptide antigen conjugate mixture.

37. The process according to claim 36, further comprising adding an excess volume of aqueous buffer to the sterile peptide antigen conjugate mixture followed by mixing to generate a sterile aqueous solution of peptide antigen conjugate particles comprising at least the first peptide antigen conjugate and the second peptide antigen conjugate, any unreacted hydrophobic block fragment and aqueous buffer.

38. A solid phase peptide synthesis process for producing a peptide antigen conjugate suitable for administration to a mammal, the peptide antigen conjugate comprising a peptide antigen linked to a hydrophobic block, the process comprising:
providing a solid phase resin bound hydrophobic block fragment;
forming a resin bound peptide antigen conjugate by either sequentially coupling individual amino acids and/or polyamino acid fragments to form a peptide antigen fragment coupled to the resin bound hydrophobic block, or coupling a peptide antigen fragment to the resin bound hydrophobic block;
or, providing a solid phase resin bound peptide antigen fragment; forming a resin bound peptide antigen conjugate by coupling the hydrophobic block fragment to the resin bound peptide antigen fragment to form a resin bound peptide antigen conjugate;
cleaving the peptide antigen conjugate from the resin to obtain a peptide antigen conjugate; and
purifying the peptide antigen conjugate to obtain a purified peptide antigen conjugate as a lyophilized purified peptide antigen conjugate and/or a purified peptide antigen conjugate solution comprising the purified peptide antigen conjugate and a pharmaceutically acceptable organic solvent.

39. The process according to claim 38, further comprising adding an excess volume of aqueous buffer to the lyophilized purified peptide antigen conjugate followed by mixing to generate an aqueous solution of peptide antigen conjugate particles comprising the peptide antigen conjugate and aqueous buffer, or adding an excess volume of aqueous buffer to the purified peptide antigen conjugate solution followed by mixing to generate an aqueous solution of peptide antigen conjugate particles comprising the peptide antigen conjugate, pharmaceutically acceptable organic solvent and aqueous buffer

40. The process according to claim 38, further comprising sterile filtering the purified peptide antigen conjugate solution to obtain a sterile purified peptide antigen conjugate solution comprising peptide antigen conjugate and pharmaceutically acceptable organic solvent.

41. The process according to claim 40, further comprising adding an excess volume of aqueous buffer to the sterile purified peptide antigen conjugate solution followed by mixing to generate a sterile aqueous solution of peptide antigen conjugate particles comprising the peptide antigen conjugate, pharmaceutically acceptable organic solvent and aqueous buffer.

42. The process according to claim 40, further comprising lyophilizing the sterile purified peptide antigen conjugate solution to obtain a lyophilized sterile purified peptide antigen conjugate.

43. The process according to claim 42, further comprising adding an excess volume of aqueous buffer to the lyophilized sterile purified peptide antigen conjugate followed by mixing to generate a sterile aqueous solution of peptide antigen conjugate particles comprising the peptide antigen conjugate and aqueous buffer.

44. The process according to any of claims 38 to 43, further comprising analysing the propensity of the lyophilized purified peptide antigen conjugate, purified peptide antigen conjugate solution, sterile purified peptide antigen conjugate solution and/or lyophilized sterile purified peptide antigen conjugate to form aggregated material upon addition of an aqueous buffer, the analysis comprising the steps of:
(i) aliquoting a specific volume of the purified peptide antigen conjugate solution and/or sterile purified peptide antigen conjugate solution from a first container to a second container, and/or adding a specific mass of the lyophilized purified peptide antigen conjugate and/or lyophilized sterile purified peptide antigen conjugate from a first container to a second container;
(ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL;
(iii) assessing turbidity of the aqueous solution of peptide antigen conjugate particles by measuring absorbance of the aqueous mixture at a wavelength greater than 350 nm; and
(iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

45. The process according to any one of claims 38 to 44, wherein the pharmaceutically acceptable organic solvent is selected from one or more of the group consisting of dimethyl sulfoxide (DMSO), methanol and ethanol.

46. The process according to claim 45, wherein the pharmaceutically acceptable organic solvent is DMSO.

47. The process according to any one of claims 38 to 46, wherein the peptide antigen fragment has a formula selected from [C]-[B1]-A-[B2] or [B1]-A-[B2]-[C], where C is a charged moiety, B1 is an N-terminal extension, A is a peptide antigen, B2 is a C-terminal extension, and [ ] denotes that the group is optional.

48. The process according to any one of claims 38 to 47, wherein the peptide antigen conjugate has the formula [C]-[B1]-A-[B2]-H where H is a hydrophobic block.

49. The process according to claim 48, wherein the peptide antigen conjugate has a formula selected from the group consisting of A-H, C-A-H, B1-A-H, A-B2-H, C-B1-A-H, C-A-B2-H, and C-B1-A-B2-H.

50. The process according to any one of claims 38 to 47, wherein the peptide antigen conjugate has the formula H-[B1]-A-[B2]-[C].

51. The process according to claim 50, wherein the peptide antigen conjugate has a formula selected from the group consisting of H-A, H-A-C, H-B1-A, H-A-B2, H-B1-A-C, H-A-B2-C, and H-B1-A-B2-C.

52. The process according to any one of claims 38 to 51, wherein the hydrophobic block comprises a poly(amino acid)-based polymer.

53. The process according to claim 52, wherein the poly(amino acid)-based polymer comprises aromatic rings or heterocyclic aromatic rings.

54. The process according to claim 53, wherein the poly(amino acid)-based polymer comprises aryl amines.

55. The process according to any one of claims 38 to 54, further comprising forming a peptide antigen conjugate mixture comprising two or more peptide antigen conjugates, the process comprising:
combining a specific volume of a first purified peptide antigen conjugate solution comprising a first peptide antigen conjugate and/or a first sterile purified peptide antigen conjugate solution comprising a first peptide antigen conjugate with at least a second purified peptide antigen conjugate solution comprising a second peptide antigen conjugate and/or a second sterile purified peptide antigen conjugate solution comprising a second peptide antigen conjugate to obtain a peptide antigen conjugate mixture comprising at least the first peptide antigen conjugate and the second peptide antigen conjugate and the pharmaceutically acceptable organic solvent; and/or
combining a specific mass of a first lyophilized purified peptide antigen conjugate comprising a first peptide antigen conjugate and/or a first lyophilized sterile purified peptide antigen conjugate comprising a first peptide antigen conjugate with at least a specific mass of a second lyophilized purified peptide antigen conjugate comprising a second peptide antigen conjugate and/or a second lyophilized sterile purified peptide antigen conjugate comprising a second peptide antigen conjugate to obtain a peptide antigen conjugate mixture comprising at least the first peptide antigen conjugate and the second peptide antigen conjugate.

56. The process according to claim 55, wherein the step of combining a specific volume of the first purified peptide antigen conjugate solution and/or the first sterile purified peptide antigen conjugate solution with at least the second purified peptide antigen conjugate solution and/or the second sterile purified peptide antigen conjugate solution comprises selecting and transferring a specific volume of solution to transfer from one container to a second container, the process comprising the steps of:
(i) determining the molar concentration of the peptide antigen conjugate in at least the first purified peptide antigen conjugate solution, the first sterile purified peptide antigen conjugate solution, the second purified peptide antigen conjugate solution and/or the second sterile purified peptide antigen conjugate solution;
(ii) aliquoting a specific volume of at least the first purified peptide antigen conjugate solution, the first sterile purified peptide antigen conjugate solution and the second purified peptide antigen conjugate solution and/or the second sterile purified peptide antigen conjugate solution from the first container to a second container to obtain a specific molar content of each of the first peptide antigen conjugate and the second peptide antigen conjugate.

57. The process according to claim 56, wherein the process of determining the molar concentration of peptide antigen conjugate in at least the first purified peptide antigen conjugate solution, the first sterile purified peptide antigen conjugate solution, the second purified peptide antigen conjugate solution and/or the second sterile purified peptide antigen conjugate solution comprises measuring UV-Vis absorption of the peptide antigen conjugate at a wavelength between about 300 to about 350 nm.

58. The process according to any one of claims 55 to 57, further comprising adding an excess volume of aqueous buffer to the peptide antigen conjugate mixture followed by mixing to generate an aqueous solution of peptide antigen conjugate particles comprising at least the first peptide antigen conjugate and the second peptide antigen conjugate, any pharmaceutically acceptable organic solvent and aqueous buffer.

59. The process according to any one of claims 55 to 57, further comprising lyophilization of the peptide antigen conjugate mixture to obtain a lyophilized peptide antigen conjugate mixture product.

60. The process according to claim 59, further comprising adding an excess volume of aqueous buffer to the lyophilized peptide antigen conjugate mixture followed by mixing to generate an aqueous solution of peptide antigen conjugate particles comprising at least the first peptide antigen conjugate and the second peptide antigen conjugate and aqueous buffer.

61. The process according to any one of claims 55 to 57, further comprising sterile filtering the peptide antigen conjugate mixture to obtain a sterile peptide antigen conjugate mixture.

62. The process according to claim 61, further comprising adding an excess volume of aqueous buffer to the sterile peptide antigen conjugate mixture followed by mixing to generate a sterile aqueous solution of peptide antigen conjugate particles comprising at least the first peptide antigen conjugate and the second peptide antigen conjugate, pharmaceutically acceptable organic solvent and aqueous buffer.

63. The process according to claim 61, further comprising lyophilization of the sterile peptide antigen conjugate mixture to obtain a lyophilized sterile peptide antigen conjugate mixture.

64. The process according to claim 63, further comprising adding an excess volume of aqueous buffer to the lyophilized sterile peptide antigen conjugate mixture followed by mixing to generate a sterile aqueous solution of peptide antigen conjugate particles comprising at least the first peptide antigen conjugate and the second peptide antigen conjugate and aqueous buffer.

65. A process for producing a sterile aqueous solution of peptide antigen conjugate particles, the process comprising:
a) preparing a peptide antigen conjugate solution comprising a peptide antigen conjugate and a pharmaceutically acceptable organic solvent, said peptide antigen conjugate comprising a peptide antigen linked to a hydrophobic block;
b) sterile-filtering the peptide antigen conjugate solution to produce a sterile peptide antigen conjugate solution; and
c) adding an aqueous buffer to the sterile peptide antigen conjugate solution to produce the sterile aqueous solution of peptide antigen particles.

66. The process according to claim 65, further comprising:
a′) preparing a second peptide antigen conjugate solution comprising a second peptide antigen conjugate and a pharmaceutically acceptable organic solvent, said second peptide antigen conjugate comprising a second peptide antigen linked to a hydrophobic block;
a″) combining a specific volume of each of the peptide antigen conjugate solution and the second peptide antigen conjugate solution to obtain a peptide antigen conjugate mixture comprising two or more different peptide antigen conjugates which is then subjected to steps b) and c).

67. The process according to claim 65, further comprising:
a′) preparing a second peptide antigen conjugate solution comprising a second peptide antigen conjugate and a pharmaceutically acceptable organic solvent, said second peptide antigen conjugate comprising a second peptide antigen linked to a hydrophobic block;
b′) sterile-filtering the second peptide antigen conjugate solution to produce a second sterile peptide antigen conjugate solution; and
b″) combining a specific volume of each of the sterile peptide antigen conjugate solution and the second sterile peptide antigen conjugate solution to obtain a combined sterile peptide antigen conjugate solution which is then subjected to step c).

68. The process according to any of claims 65 to 67, further comprising analysing the propensity of the peptide antigen conjugate solution, the second peptide antigen conjugate solution, the sterile peptide antigen conjugate solution, the second sterile peptide antigen conjugate solution, the peptide antigen conjugate mixture and/or the sterile peptide antigen conjugate mixture to form aggregated material upon addition of an aqueous buffer, the analysis comprising the steps of:
(i) aliquoting a specific volume of the peptide antigen conjugate solution, the second peptide antigen conjugate solution, the sterile peptide antigen conjugate solution, the second sterile peptide antigen conjugate solution, the peptide antigen conjugate mixture and/or the sterile peptide antigen conjugate mixture from a first container to a second container;
(ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL;
(iii) assessing turbidity of the aqueous solution of peptide antigen conjugate particles by measuring absorbance of the aqueous mixture at a wavelength greater than 350 nm; and
(iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

69. The process according to any one of claims 65 to 68, wherein the pharmaceutically acceptable organic solvent is selected from one or more of the group consisting of dimethyl sulfoxide (DMSO), methanol and ethanol.

70. The process according to claim 69, wherein the pharmaceutically acceptable organic solvent is DMSO.

71. The process according to any one of claims 65 to 70, wherein the peptide antigen conjugate has the formula [C]-[B1]-A-[B2]-H where C is a charged moiety, B1 is an N-terminal extension, A is a peptide antigen, B2 is a C-terminal extension, H is a hydrophobic block, and [ ] denotes that the group is optional.

72. The process according to claim 71, wherein the peptide antigen conjugate has a formula selected from the group consisting of A-H, C-A-H, B1-A-H, A-B2-H, C-B1-A-H, C-A-B2-H, and C-B1-A-B2-H.

73. The process according to any one of claims 65 to 70, wherein the peptide antigen conjugate has the formula H-[B1]-A-[B2]-[C] where H is a hydrophobic block, B1 is an N-terminal extension, A is a peptide antigen, B2 is a C-terminal extension, C is a charged moiety, and [ ] denotes that the group is optional.

74. The process according to claim 73, wherein the peptide antigen conjugate has a formula selected from the group consisting of H-A, H-A-C, H-B1-A, H-A-B2, H-B1-A-C, H-A-B2-C, and H-B1-A-B2-C.

75. The process according to any one of claims 65 to 70, wherein the peptide antigen conjugate has the formula [C]-[B1]-A-[B2]-L-H, where C is a charged moiety, B1 is an N-terminal extension, A is a peptide antigen, B2 is a C-terminal extension, H is a hydrophobic block, L is a Linker, and [ ] denotes that the group is optional.

76. The process according to claim 75, wherein the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

77. The process according to any one of claims 65 to 70, wherein the peptide antigen conjugate has the formula H-L-[B1]-A-[B2]-[C], where C is a charged moiety, B1 is an N-terminal extension, A is a peptide antigen, B2 is a C-terminal extension, H is a hydrophobic block, L is a Linker, and [ ] denotes that the group is optional.

78. The process according to claim 77, wherein the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

79. The process according to any one of claims 65 to 78, wherein the hydrophobic block comprises a poly(amino acid)-based polymer.

80. The process according to claim 79, wherein the poly(amino acid)-based polymer comprises aromatic rings or heterocyclic aromatic rings.

81. The process according to claim 80, wherein the poly(amino acid)-based polymer comprises aryl amines.

82. A process for analysing the propensity of a peptide antigen conjugate composition comprising a peptide antigen linked to a hydrophobic block to form aggregated material upon addition of an aqueous buffer, the analysis comprising the steps of:
(i) aliquoting a specific volume of a peptide antigen conjugate solution from a first container to a second container, and/or adding a specific mass of a peptide antigen conjugate from a first container to a second container;
(ii) adding a volume of the aqueous buffer to the second container to obtain an aqueous solution of peptide antigen conjugate particles comprising the peptide antigen conjugate, wherein the concentration of the peptide antigen conjugate is not lower than 0.01 mg/mL;
(iii) assessing turbidity of the aqueous solution of peptide antigen conjugate particles by measuring absorbance of the aqueous mixture at a wavelength greater than 350 nm; and
(iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

83. The process according to claim 82, wherein the peptide antigen conjugate has a formula selected from [C]-[B1]-A-[B2]-H or [B1]-A-[B2]-H([C]) where C is a charged moiety, B1 is an N-terminal extension, A is a peptide antigen, B2 is a C-terminal extension, H is a hydrophobic block, and [ ] denotes that the group is optional.

84. The process according to claim 83, wherein the peptide antigen conjugate has a formula selected from the group consisting of A-H, C-A-H, B1-A-H, A-B2-H, C-B1-A-H, C-A-B2-H, and C-B1-A-B2-H.

85. The process according to claim 82, wherein the peptide antigen conjugate has the formula H-[B1]-A-[B2]-[C] or H([C)]-[B1]-A-[B2] where B1 is an N-terminal extension, A is a peptide antigen, B2 is a C-terminal extension, C is a charged moiety, H is a hydrophobic block, and [ ] denotes that the group is optional.

86. The process according to claim 85, wherein the peptide antigen conjugate has a formula selected from the group consisting of H-A, H-A-C, H-B1-A, H-A-B2, H-B1-A-C, H-A-B2-C, and H-B1-A-B2-C.

87. The process according to claim 82, wherein the peptide antigen conjugate has a formula selected from [C]-[B1]-A-[B2]-L-H, [B1]-A-[B2]-L([C])-H or [B1]-A-[B2]-L-H([C]), where C is a charged moiety, B1 is an N-terminal extension, A is a peptide antigen, B2 is a C-terminal extension, H is a hydrophobic block, L is a Linker, and [ ] denotes that the group is optional.

88. The process according to claim 87, wherein the peptide antigen conjugate has a formula selected from the group consisting of A-L-H, C-A-L-H, B1-A-L-H, A-B2-L-H, C-B1-A-L-H, C-A-B2-L-H, and C-B1-A-B2-L-H.

89. The process according to claim 82, wherein the peptide antigen conjugate has a formula selected from H-L-[B1]-A-[B2]-[C], H([C])-L-[B1]-A-[B2] or H-L([C])-[B1]-A-[B2] where C is a charged moiety, B1 is an N-terminal extension, A is a peptide antigen, B2 is a C-terminal extension, H is a hydrophobic block, L is a Linker, and [ ] denotes that the group is optional.

90. The process according to claim 89, wherein the peptide antigen conjugate has a formula selected from the group consisting of H-L-A, H-L-A-C, H-L-B1-A, H-L-A-B2, H-L-B1-A-C, H-L-A-B2-C, and H-L-B1-A-B2-C.

91. The process according to any one of claims 82 to 90, wherein the hydrophobic block comprises a poly(amino acid)-based polymer.

92. The process according to claim 91, wherein the poly(amino acid)-based polymer comprises aromatic rings or heterocyclic aromatic rings.

93. The process according to claim 92, wherein the poly(amino acid)-based polymer comprises aryl amines.

94. A process for producing a peptide antigen conjugate mixture comprising a first peptide antigen linked to a hydrophobic block and at least a second peptide antigen linked to a hydrophobic block, the process comprising:
preparing a first peptide antigen conjugate solution comprising a first peptide antigen conjugate and a pharmaceutically acceptable organic solvent;
preparing at least a second peptide antigen conjugate solution comprising a second peptide antigen conjugate and a pharmaceutically acceptable organic solvent;
combining a specific volume of the peptide antigen conjugate solutions to obtain a peptide antigen conjugate mixture comprising the first peptide antigen conjugate and the at least second peptide antigen conjugate and a pharmaceutically acceptable organic solvent.

95. The process according to claim 94, wherein the step of combining a specific volume of the peptide antigen conjugate solutions comprises selecting and transferring a specific volume of each peptide antigen conjugate solution to transfer from one container to a second container, the process comprising the steps of:
(i) determining the molar concentration of the peptide antigen conjugate in each of the peptide antigen conjugate solutions;
(ii) aliquoting a specific volume of each peptide antigen conjugate solution from the first container to a second container to obtain a specific molar content of each of the peptide antigen conjugates.

96. The process according to claim 95, wherein the process of determining the molar concentration of peptide antigen conjugate in each of the peptide antigen conjugate solutions comprises measuring UV-Vis absorption of the peptide antigen conjugate at a wavelength between about 300 to about 350 nm.

97. The process according to any one of claims 94 to 96, further comprising adding an excess volume of aqueous buffer to the peptide antigen conjugate mixture followed by mixing to generate an aqueous solution of peptide antigen conjugate particles comprising the first peptide antigen conjugate and at least the second peptide antigen conjugate, pharmaceutically acceptable organic solvent and aqueous buffer.

98. The process according to claim 97, further comprising lyophilization of the peptide antigen conjugate mixture to obtain a lyophilized peptide antigen conjugate mixture.

99. The process according to claim 98, further comprising adding an excess volume of aqueous buffer to the lyophilized peptide antigen conjugate mixture followed by mixing to generate an aqueous solution of peptide antigen conjugate particles comprising the first peptide antigen conjugate and at least the second peptide antigen conjugate, pharmaceutically acceptable organic solvent and aqueous buffer.

100. The process according to any one of claims 94 to 96, further comprising sterile filtering the peptide antigen conjugate mixture to obtain a sterile peptide antigen conjugate mixture.

101. The process according to claim 100, further comprising adding an excess volume of aqueous buffer to the sterile peptide antigen conjugate mixture followed by mixing to generate a sterile aqueous solution of peptide antigen conjugate particles comprising the first peptide antigen conjugate and at least the second peptide antigen conjugate, pharmaceutically acceptable organic solvent and aqueous buffer.

102. The process according to claim 100, further comprising lyophilization of the sterile peptide antigen conjugate mixture to obtain a lyophilized sterile peptide antigen conjugate mixture.

103. The process according to claim 102, further comprising adding an excess volume of aqueous buffer to the lyophilized sterile peptide antigen conjugate mixture followed by mixing to generate a sterile aqueous solution of peptide antigen conjugate particles comprising the first peptide antigen conjugate and at least the second peptide antigen conjugate, pharmaceutically acceptable organic solvent and aqueous buffer.

104. The process according to any one of claims 94 to 103, further comprising analysing the propensity of the peptide antigen conjugate mixture, lyophilized peptide antigen conjugate mixture, sterile peptide antigen conjugate mixture and/or lyophilized sterile peptide antigen conjugate mixture to form aggregated material upon addition of an aqueous buffer, the analysis comprising the steps of:
(i) aliquoting a specific volume of the peptide antigen conjugate mixture and/or sterile peptide antigen conjugate mixture from a first container to a second container, and/or adding a specific mass of the lyophilized peptide antigen conjugate mixture and/or lyophilized sterile peptide antigen conjugate mixture from a first container to a second container;
(ii) adding a volume of the aqueous buffer to the second container to obtain an aqueous solution of peptide antigen conjugate particles comprising the first peptide antigen conjugate and at least the second peptide antigen conjugate, wherein the concentration of the peptide antigen conjugates is not lower than 0.01 mg/mL;
(iii) assessing turbidity of the aqueous solution of peptide antigen conjugate particles by measuring absorbance of the aqueous mixture at a wavelength greater than 350 nm; and
(iv) confirming the presence or absence of aggregated material in the aqueous solution of peptide antigen conjugate particles based on a comparison of the absorbance of the aqueous solution of peptide antigen conjugate particles with the absorbance of aqueous buffer alone.

105. The process according to any one of claims 94 to 104, wherein the process for selecting the composition and volume of the first peptide antigen conjugate and the at least second peptide antigen conjugate to include in the peptide antigen conjugate mixture comprises any one or both of the steps of:
(i) determining the molar concentration of the peptide antigen conjugates in the peptide antigen conjugate solutions;
(ii) determining the propensity of each of the peptide antigen conjugate solutions to form aggregated material upon addition of an excess of aqueous buffer to dilute the peptide antigen conjugates to a concentration no lower than 0.01 mg/mL.

106. The process according to claim 105, wherein the molar concentration of peptide antigen conjugates derived from the peptide antigen conjugate mixture and/or the sterile peptide antigen conjugate mixture that each individually have the propensity to form aggregated material upon addition of the aqueous buffer comprise 60% or less of the total molar content of peptide antigen conjugates in the peptide antigen conjugate mixture.

107. The process according to either claim 105 or claim 106, wherein the process of determining the molar concentration of peptide antigen conjugates in the peptide antigen conjugate mixture and/or the sterile peptide antigen conjugate mixture comprises measuring UV-Vis absorption of the peptide antigen conjugates at a wavelength between about 300 to about 350 nm.

108. A peptide antigen conjugate produced by the process of any one of claims 1 to 64.

109. An immunogenic composition comprising the peptide antigen conjugate of claim 109.

110. A sterile aqueous solution of peptide antigen conjugate particles produced by the process of any one of claims 65 to 81.

111. A peptide antigen conjugate mixture produced by the process of any one of claims 94 to 107.


SEQUENCE LISTING (126 sequences)

SEQ ID NO: 1 - protein, 4 aa, Artificial Sequence
  Synthetic
SPLR

SEQ ID NO: 2 - protein, 6 aa, Artificial Sequence
  Synthetic
GGKLVR

SEQ ID NO: 3 - protein, 6 aa, Artificial Sequence
  Synthetic
GGKPLR

SEQ ID NO: 4 - protein, 6 aa, Artificial Sequence
  Synthetic
GGSLVR

SEQ ID NO: 5 - protein, 6 aa, Artificial Sequence
  Synthetic
GGSLVL

SEQ ID NO: 6 - protein, 6 aa, Artificial Sequence
  Synthetic
GGELVR

SEQ ID NO: 7 - protein, 6 aa, Artificial Sequence
  Synthetic
GGELVL

SEQ ID NO: 8 - protein, 5 aa, Artificial Sequence
  Synthetic
GSLVR

SEQ ID NO: 9 - protein, 5 aa, Artificial Sequence
  Synthetic
GKPVR

SEQ ID NO: 10 - protein, 5 aa, Artificial Sequence
  Synthetic
GSLVL

SEQ ID NO: 11 - protein, 5 aa, Artificial Sequence
  Synthetic
GELVL

SEQ ID NO: 12 - protein, 4 aa, Artificial Sequence
  Synthetic
SLVL

SEQ ID NO: 13 - protein, 4 aa, Artificial Sequence
  Synthetic
ELVR

SEQ ID NO: 14 - protein, 4 aa, Artificial Sequence
  Synthetic
SPVR

SEQ ID NO: 15 - protein, 4 aa, Artificial Sequence
  Synthetic
SLVR

SEQ ID NO: 16 - protein, 4 aa, Artificial Sequence
  Synthetic
KPLR

SEQ ID NO: 17 - protein, 4 aa, Artificial Sequence
  Synthetic
ELVL

SEQ ID NO: 18 - protein, 8 aa, Artificial Sequence
  Synthetic
KPLRYLLL

SEQ ID NO: 19 - protein, 4 aa, Artificial Sequence
  Synthetic
SLVL

SEQ ID NO: 20 - protein, 4 aa, Artificial Sequence
  Synthetic
ELVR

SEQ ID NO: 21 - protein, 4 aa, Artificial Sequence
  Synthetic
SPVR

SEQ ID NO: 22 - protein, 4 aa, Artificial Sequence
  Synthetic
KPLR

SEQ ID NO: 23 - protein, 4 aa, Artificial Sequence
  Synthetic
KPLR

SEQ ID NO: 24 - protein, 4 aa, Artificial Sequence
  Synthetic
KPVR

SEQ ID NO: 25 - protein, 4 aa, Artificial Sequence
  Synthetic
ELVL

SEQ ID NO: 26 - protein, 6 aa, Artificial Sequence
  Synthetic
  misc_feature (6)..(6): Xaa is a citrulline (CIT)
GGSLVX

SEQ ID NO: 27 - protein, 4 aa, Artificial Sequence
  Synthetic
  misc_feature (4)..(4): Xaa is a citrulline (CIT)
SPVX

SEQ ID NO: 28 - protein, 4 aa, Artificial Sequence
  Synthetic
  misc_feature (4)..(4): Xaa is a citrulline (CIT)
ELVX

SEQ ID NO: 29 - protein, 5 aa, Artificial Sequence
  Synthetic
  misc_feature (5)..(5): Xaa is a citrulline (CIT)
GSLVX

SEQ ID NO: 30 - protein, 4 aa, Artificial Sequence
  Synthetic
  misc_feature (4)..(4): Xaa is a citrulline (CIT)
SPVX

SEQ ID NO: 31 - protein, 4 aa, Artificial Sequence
  Synthetic
  misc_feature (4)..(4): Xaa is a citrulline (CIT)
ELVX

SEQ ID NO: 32 - protein, 5 aa, Artificial Sequence
  Synthetic
  misc_feature (5)..(5): Xaa is a citrulline (CIT)
GKPVX

SEQ ID NO: 33 - protein, 6 aa, Artificial Sequence
  Synthetic
  misc_feature (6)..(6): Xaa is a citrulline (CIT)
GGSPVX

SEQ ID NO: 34 - protein, 4 aa, Artificial Sequence
  Synthetic
  misc_feature (4)..(4): Xaa is a citrulline (CIT)
EPVX

SEQ ID NO: 35 - protein, 4 aa, Artificial Sequence
  Synthetic
  misc_feature (4)..(4): Xaa is a citrulline (CIT)
EGVX

SEQ ID NO: 36 - protein, 4 aa, Artificial Sequence
  Synthetic
  misc_feature (4)..(4): Xaa is a citrulline (CIT)
SLVX

SEQ ID NO: 37 - protein, 4 aa, Artificial Sequence
  Synthetic
  misc_feature (4)..(4): Xaa is a citrulline (CIT)
EPVX

SEQ ID NO: 38 - protein, 4 aa, Artificial Sequence
  Synthetic
  misc_feature (4)..(4): Xaa is a citrulline (CIT)
KPVX

SEQ ID NO: 39 - protein, 51 aa, Artificial Sequence
  Synthetic
  misc_feature (1)..(1): Wherein Asp is modified with an acetyl group at the N-terminus
  misc_feature (45)..(45): Xaa is a citrulline (CIT)
  misc_feature (46)..(46): Wherein Xaa is an azido-lysine (Lys(N3)) that is connected to Glu via a N3-DBCO linkage.
  misc_feature (47)..(47): Wherein Glu is modified with a DBCO group at the N-terminus and is further modified to include Compound 1 as described in the specification.
  misc_feature (49)..(49): Wherein Glu is modified to include Compound 1 as described in the specification.
  misc_feature (51)..(51): Wherein Glu is modified to include Compound 1 as described in the specification.
  misc_feature (51)..(51): Wherein Glu is modified with an amide (NH2) at the C-terminus
DDDDDDDDDDDDDGIPVHLELASMTNMELMSSIVHQQVFPTSPVXXEWEWE

SEQ ID NO: 40 - protein, 51 aa, Artificial Sequence
  Synthetic
  misc_feature (1)..(1): Wherein Glu is modified with an acetyl group at the N-terminus
  misc_feature (9)..(9): Xaa is a citrulline (CIT)
  misc_feature (41)..(41): Xaa is a citrulline (CIT)
  misc_feature (42)..(45): Wherein Xaa is a para-amino-phenylalanine.
  misc_feature (46)..(46): Wherein Xaa is a azido-lysine (Lys(N3)) that is connected to Glu via a N3-DBCO linkage.
  misc_feature (47)..(47): Wherein Glu is modified with a DBCO group at the N-terminus and is further modified to include Compound 1 as described in the specification
  misc_feature (49)..(49): Wherein Glu is modified to include Compound 1 as described in the specification
  misc_feature (51)..(51): Wherein Glu is modified with an amide (NH2) group at the C-terminus
  misc_feature (51)..(51): Wherein Glu is modified to include Compound 1 as described in the specification
EEEEEEEVXGIPVHLELASMTNMELMSSIVHQQVFPTSPVXXXXXXEWEWE

SEQ ID NO: 41 - protein, 22 aa, Artificial Sequence
  Synthetic
  misc_feature (1)..(1): Wherein Glu is modified with an acetyl group at the N-terminus
  misc_feature (9)..(9): Xaa is a citrulline (CIT)
  misc_feature (21)..(21): Xaa is a citrulline (CIT)
  misc_feature (22)..(22): Xaa is a azido-lysine(Lys(N3)).
  misc_feature (22)..(22): Wherein Xaa is modified with an amide (NH2) at the C-terminus.
EEEEESLVXAQLNDVVLSPVXX

SEQ ID NO: 42 - protein, 47 aa, Artificial Sequence
  Synthetic
  misc_feature (1)..(1): Wherein Glu is modified with an acetyl group at the N-terminus
  misc_feature (7)..(7): Xaa is a citrulline (CIT)
  misc_feature (37)..(37): Xaa is a citrulline (CIT)
  misc_feature (38)..(41): Wherein Xaa is a para-amino-phenylalanine.
  misc_feature (42)..(42): Wherein Xaa is an azido-lysine (Lys(N3)) that is connected to Glu via a N3-DBCO linkage.
  misc_feature (43)..(43): Wherein Glu is modified with a DBCO group at the N-terminus and is further modified to include Compound 1 as described in the specification.
  misc_feature (45)..(45): Wherein Glu is modified to include Compound 1 as described in the specification.
  misc_feature (47)..(47): Wherein Glu is modified with an amide (NH2) at the C-terminus.
  misc_feature (47)..(47): Wherein Glu is modified to include Compound 1 as described in the specification.
EEEEEVXDFTGSNGDPSSPYSLHYLSPTGVNEYSPVXXXXXXEWEWE

SEQ ID NO: 43 - protein, 26 aa, Artificial Sequence
  Synthetic
  misc_feature (1)..(1): Wherein Glu is modified with an acetyl group at the N-terminus
  misc_feature (7)..(7): Xaa is a citrulline (CIT)
  misc_feature (20)..(20): Xaa is a citrulline (CIT)
  misc_feature (21)..(21): Wherein Xaa is an azido-lysine (Lys(N3)) that is connected to Glu via a N3-DBCO linkage.
  misc_feature (22)..(22): Wherein Glu is modified with a DBCO group at the N-terminus and is further modified to include Compound 1 as described in the specification.
  misc_feature (24)..(24): Wherein Glu is modified to include Compound 1 as described in the specification.
  misc_feature (26)..(26): Wherein Glu is modified with an amide (NH2) group at the C-terminus.
  misc_feature (26)..(26): Wherein Glu is modified to include Compound 1 as described in the specification.
EEEEEVXTAPDNLGYMSPVXXEWEWE

SEQ ID NO: 44 - protein, 13 aa, Artificial Sequence
  Synthetic
AKFVAAWTLKAAA

SEQ ID NO: 45 - protein, 4 aa, Artificial Sequence
  Synthetic
DLVR

SEQ ID NO: 46 - protein, 4 aa, Artificial Sequence
  Synthetic
  misc_feature (4)..(4): Xaa is a citrulline (CIT)
DLVX

SEQ ID NO: 47 - protein, 4 aa, Artificial Sequence
  Synthetic
DLVL

SEQ ID NO: 48 - protein, 4 aa, Artificial Sequence
  Synthetic
DPVR

SEQ ID NO: 49 - protein, 4 aa, Artificial Sequence
  Synthetic
  misc_feature (4)..(4): Xaa is a citrulline (CIT)
DPVX

SEQ ID NO: 50 - protein, 5 aa, Artificial Sequence
  Synthetic
DSDSD

SEQ ID NO: 51 - protein, 7 aa, Artificial Sequence
  Synthetic
DSDSDSD

SEQ ID NO: 52 - protein, 24 aa, Artificial Sequence
  Synthetic
  misc_feature (24)..(24): Wherein the Lys is linked to a dibenzylcyclooctyne (DBCO)
EKSLVRAKFVAAWTLKAAASLVRK

SEQ ID NO: 53 - protein, 5 aa, Artificial Sequence
  Synthetic
GDLVR

SEQ ID NO: 54 - protein, 5 aa, Artificial Sequence
  Synthetic
GDLVL

SEQ ID NO: 55 - protein, 6 aa, Artificial Sequence
  Synthetic
GGDPVR

SEQ ID NO: 56 - protein, 6 aa, Artificial Sequence
  Synthetic
  misc_feature (6)..(6): Xaa is a citrulline (CIT)
GGSPLX

SEQ ID NO: 57 - protein, 6 aa, Artificial Sequence
  Synthetic
GGSPVR

SEQ ID NO: 58 - protein, 34 aa, Artificial Sequence
  Synthetic
  misc_feature (29)..(29): Wherein Xaa is an azido-lysine (Lys(N3)) that is connected to Glu via a N3-DBCO linkage.
  misc_feature (30)..(30): Wherein Glu is modified with a DBCO group at the N-terminus and is further modified to include Compound 1 as described in the specification.
  misc_feature (32)..(32): Wherein Glu is modified to include Compound 1 as described in the specification.
  misc_feature (34)..(34): Wherein Glu is modified with an amide (NH2) group at the C-terminus
  misc_feature (34)..(34): Wherein Glu is modified to include Compound 1 as described in the specification.
GIPVHLELASMTNMELMSSIVHQQVFPTXEWEWE

SEQ ID NO: 59 - protein, 4 aa, Artificial Sequence
  Synthetic
GPGR

SEQ ID NO: 60 - protein, 4 aa, Artificial Sequence
  Synthetic
  misc_feature (4)..(4): Xaa is a citrulline (CIT)
GPGX

SEQ ID NO: 61 - protein, 5 aa, Artificial Sequence
  Synthetic
  misc_feature (5)..(5): Xaa is a citrulline (CIT)
GPVLX

SEQ ID NO: 62 - protein, 6 aa, Artificial Sequence
  Synthetic
GSELVR

SEQ ID NO: 63 - protein, 5 aa, Artificial Sequence
  Synthetic
GSLVR

SEQ ID NO: 64 - protein, 4 aa, Artificial Sequence
  Synthetic
GSVR

SEQ ID NO: 65 - protein, 5 aa, Artificial Sequence
  Synthetic
  misc_feature (5)..(5): Xaa is a citrulline (CIT)
GSVLX

SEQ ID NO: 66 - protein, 4 aa, Artificial Sequence
  Synthetic
KDKD

SEQ ID NO: 67 - protein, 6 aa, Artificial Sequence
  Synthetic
KDKDKD

SEQ ID NO: 68 - protein, 4 aa, Artificial Sequence
  Synthetic
KLVR

SEQ ID NO: 69 - skipped

SEQ ID NO: 70 - protein, 4 aa, Artificial Sequence
  Synthetic
KKKK

SEQ ID NO: 71 - protein, 5 aa, Artificial Sequence
  Synthetic
KKKKK

SEQ ID NO: 72 - protein, 6 aa, Artificial Sequence
  Synthetic
KKKKKK

SEQ ID NO: 73 - protein, 7 aa, Artificial Sequence
  Synthetic
KKKKKKK

SEQ ID NO: 74 - protein, 8 aa, Artificial Sequence
  Synthetic
KKKKKKKK

SEQ ID NO: 75 - protein, 16 aa, Artificial Sequence
  Synthetic
  misc_feature (11)..(11): Wherein Xaa is an azido-lysine (Lys(N3)) that is connected to Glu via a N3-DBCO linkage.
  misc_feature (12)..(12): Wherein Glu is modified with a DBCO group at the N-terminus and is further modified to include Compound 1 as described in the specification
  misc_feature (14)..(14): Wherein Glu is modified to include Compound 1 as described in the specification
  misc_feature (16)..(16): Wherein Glu is modified with an amide (NH2) group at the C-terminus
  misc_feature (16)..(16): Wherein Glu is modified to include Compound 1 as described in the specification
KKKKKKKKKKXEWEWE

SEQ ID NO: 76 - protein, 47 aa, Artificial Sequence
  Synthetic
  misc_feature (41)..(41): Xaa is a citrulline (CIT)
  misc_feature (42)..(42): Wherein Xaa is an azido-lysine (Lys(N3)) that is connected to Glu via a N3-DBCO linkage.
  misc_feature (43)..(43): Wherein Glu is modified with a DBCO group at the N-terminus and is further modified to include Compound 1 as described in the specification.
  misc_feature (45)..(45): Wherein Glu is modified to include Compound 1 as described in the specification.
  misc_feature (47)..(47): Wherein Glu is modified with an amide (NH2) group at the C-terminus
  misc_feature (47)..(47): Wherein Glu is modified to include Compound 1 as described in the specification.
KKKKKKKKKVRDFTGSNGDPSSPYSLHYLSPTGVNEYSPVXXEWEWE

SEQ ID NO: 77 - protein, 49 aa, Artificial Sequence
  Synthetic
  misc_feature (43)..(43): Xaa is a citrulline (CIT)
  misc_feature (44)..(44): Wherein Xaa is an azido-lysine (Lys(N3)) that is connected to Glu via a N3-DBCO linkage.
  misc_feature (45)..(45): Wherein Glu is modified with a DBCO group at the N-terminus and is further modified to include Compound 1 as described in the specification.
  misc_feature (47)..(47): Wherein Glu is modified to include Compound 1 as described in the specification.
  misc_feature (49)..(49): Wherein Glu is modified with an amide (NH2) group at the C-terminus
  misc_feature (49)..(49): Wherein Glu is modified to include Compound 1 as described in the specification.
KKKKKKKKKVRGIPVHLELASMTNMELMSSIVHQQVFPTSPVXXEWEWE

SEQ ID NO: 78 - skipped

SEQ ID NO: 79 - protein, 4 aa, Artificial Sequence
  Synthetic
KPLR

SEQ ID NO: 80 - protein, 5 aa, Artificial Sequence
  Synthetic
KSKSK

SEQ ID NO: 81 - protein, 7 aa, Artificial Sequence
  Synthetic
KSKSKSK

SEQ ID NO: 82 - protein, 4 aa, Artificial Sequence
  Synthetic
  misc_feature (4)..(4): Xaa is a citrulline (CIT)
SGVX

SEQ ID NO: 83 - protein, 24 aa, Artificial Sequence
  Synthetic
  misc_feature (22)..(22): Wherein Xaa is an azido-lysine (Lys(N3)) that is connected to a hydrophobic block (H) via a N3-DBCO linkage.
SLVRAKFVAAWTLKAAASLVRXEK

SEQ ID NO: 84 - protein, 4 aa, Artificial Sequence
  Synthetic
  misc_feature (4)..(4): Xaa is a citrulline (CIT)
SPLX

SEQ ID NO: 85 - protein, 4 aa, Artificial Sequence
  Synthetic
YLLL

SEQ ID NO: 86 - protein, 8 aa, Artificial Sequence
  Synthetic
  misc_feature (4)..(4): Xaa is a citrulline (CIT)
SLVXYLLL

SEQ ID NO: 87 - protein, 16 aa, Artificial Sequence
  Synthetic
DDDDDDDDDDDDDDDD

SEQ ID NO: 88 - protein, 15 aa, Artificial Sequence
  Synthetic
DDDDDDDDDDDDDDD

SEQ ID NO: 89 - protein, 14 aa, Artificial Sequence
  Synthetic
DDDDDDDDDDDDDD

SEQ ID NO: 90 - protein, 13 aa, Artificial Sequence
  Synthetic
DDDDDDDDDDDDD

SEQ ID NO: 91 - protein, 12 aa, Artificial Sequence
  Synthetic
DDDDDDDDDDDD

SEQ ID NO: 92 - protein, 11 aa, Artificial Sequence
  Synthetic
DDDDDDDDDDD

SEQ ID NO: 93 - protein, 10 aa, Artificial Sequence
  Synthetic
DDDDDDDDDD

SEQ ID NO: 94 - protein, 9 aa, Artificial Sequence
  Synthetic
DDDDDDDDD

SEQ ID NO: 95 - protein, 8 aa, Artificial Sequence
  Synthetic
DDDDDDDD

SEQ ID NO: 96 - protein, 7 aa, Artificial Sequence
  Synthetic
DDDDDDD

SEQ ID NO: 97 - protein, 6 aa, Artificial Sequence
  Synthetic
DDDDDD

SEQ ID NO: 98 - protein, 5 aa, Artificial Sequence
  Synthetic
DDDDD

SEQ ID NO: 99 - protein, 4 aa, Artificial Sequence
  Synthetic
DDDD

SEQ ID NO: 100 - protein, 16 aa, Artificial Sequence
  Synthetic
KKKKKKKKKKKKKKKK

SEQ ID NO: 101 - protein, 15 aa, Artificial Sequence
  Synthetic
KKKKKKKKKKKKKKK

SEQ ID NO: 102 - protein, 12 aa, Artificial Sequence
  Synthetic
KKKKKKKKKKKK

SEQ ID NO: 103 - protein, 13 aa, Artificial Sequence
  Synthetic
KKKKKKKKKKKKK

SEQ ID NO: 104 - protein, 14 aa, Artificial Sequence
  Synthetic
KKKKKKKKKKKKKK

SEQ ID NO: 105 - protein, 11 aa, Artificial Sequence
  Synthetic
KKKKKKKKKKK

SEQ ID NO: 106 - protein, 10 aa, Artificial Sequence
  Synthetic
KKKKKKKKKK

SEQ ID NO: 107 - protein, 9 aa, Artificial Sequence
  Synthetic
KKKKKKKKK

SEQ ID NO: 108 - protein, 13 aa, Artificial Sequence
  Synthetic
  misc_feature (1)..(8): Wherein Xaa is a peptide antigen
  misc_feature (13)..(13): Wherein Xaa is an azido-lysine (Lys(N3)) that is connected to a hydrophobic block (H) via a N3-DBCO linkage.
XXXXXXXXSLVRX

SEQ ID NO: 109 - protein, 4 aa, Artificial Sequence
  Synthetic
GFLG

SEQ ID NO: 110 - protein, 30 aa, Artificial Sequence
  Synthetic
  MISC_FEATURE (24)..(24): Xaa is a citrulline (CIT)
  MISC_FEATURE (25)..(25): Wherein Xaa is an azido-lysine (Lys(N3)) that is connected to Glu via a N3-DBCO linkage.
  MISC_FEATURE (26)..(26): Wherein Glu is modified with a DBCO group at the N-terminus and is further modified to include Compound 1 as described in the specification.
  MISC_FEATURE (28)..(28): Wherein Glu is modified to include Compound 1 as described in the specification.
  MISC_FEATURE (30)..(30): Wherein Glu is modified with an amide (NH2) at the C-terminus.
  MISC_FEATURE (30)..(30): Wherein Glu is modified to include Compound 1 as described in the specification.
KKKSLVRAKFVAAWTLKAAASPVXXEWEWE

SEQ ID NO: 111 - protein, 25 aa, Artificial Sequence
  Synthetic Neoantigen in Table 1
ETLGEISFLLSLDLHFTDGDYSAGD

SEQ ID NO: 112 - protein, 25 aa, Artificial Sequence
  Synthetic Neoantigen in Table 1
DDEGDYTCQFTHVENGTNYIVTATR

SEQ ID NO: 113 - protein, 28 aa, Artificial Sequence
  Synthetic Neoantigen in Table 1
GIPVHLELASMTNMELMSSIVHQQVFPT

SEQ ID NO: 114 - protein, 27 aa, Artificial Sequence
  Synthetic Neoantigen in Table 1
VVDRNPQFLDPVLAYLMKGLCEKPLAS

SEQ ID NO: 115 - protein, 27 aa, Artificial Sequence
  Synthetic Neoantigen in Table 1
NIEGIDKLTQLKKPFLVNNKINKIENI

SEQ ID NO: 116 - protein, 25 aa, Artificial Sequence
  Synthetic Neoantigen in Table 1
MAAALTFRRLLTLPRAARGFGVQVS

SEQ ID NO: 117 - protein, 27 aa, Artificial Sequence
  Synthetic Neoantigen in Table 1
GRGHLLGRLAAIVGKQVLLGRKVVVVR

SEQ ID NO: 118 - protein, 24 aa, Artificial Sequence
  Synthetic Neoantigen in Table 1
QGTDVVIAIFILAMSFVPASFVVF

SEQ ID NO: 119 - protein, 25 aa, Artificial Sequence
  Synthetic Neoantigen in Table 1
LKSSPERNDWEPLDKKVDTRKYRAE

SEQ ID NO: 120 - protein, 26 aa, Artificial Sequence
  Synthetic Neoantigen in Table 1
QLRVGNDGIFMLPFFMAFIFINWLGF

SEQ ID NO: 121 - protein, 25 aa, Artificial Sequence
  Synthetic Peptide Antigen in Table 7
QGTDVVIAIFIILAMSFVPASFVVF

SEQ ID NO: 122 - protein, 10 aa, Artificial Sequence
  Synthetic Peptide Antigen in Table 8
VVIAIFIILV

SEQ ID NO: 123 - protein, 9 aa, Artificial Sequence
  Synthetic Peptide Antigen in Table 9
AALLNSAVL

SEQ ID NO: 124 - protein, 9 aa, Artificial Sequence
  Synthetic Peptide Antigen in Table 9
AQLANDVVL

SEQ ID NO: 125 - protein, 26 aa, Artificial Sequence
  Synthetic Peptide Antigen in Table 9
DFTGSNGDPSSPYSLHYLSPTGVNEY

SEQ ID NO: 126 - protein, 28 aa, Artificial Sequence
  Synthetic Peptide Antigen in Table 9
GIPVHLELASMTNMELMSSIVHQQVFPT